(19) United States
(12) Patent Application Publication
(10) Pub. No.: US 2023/0227575 A1
(43) Pub. Date: Jul. 20, 2023

(54) Engineered T Cells

(71) Applicant: Expression Therapeutics, LLC, Tucker, GA (US)

(72) Inventors: Harrison Brown, Atlanta, GA (US); Brian Petrich, Atlanta, GA (US); Gabriela Denning, Atlanta, GA (US); John Kolb, Atlanta, GA (US); Bing Yu, Atlanta, GA (US); David Schofield, Gainsville, GA (US)

(21) Appl. No.: 18/095,484

(22) Filed: Jan. 10, 2023

Related U.S. Application Data
(60) Provisional application No. 63/297,951, filed on Jan. 10, 2022.
(60) Provisional application No. 63/337,591, filed on May 2, 2022.
(60) Provisional application No. 63/339,960, filed on May 9, 2022.
(60) Provisional application No. 63/342,041, filed on May 13, 2022.
(60) Provisional application No. 63/345,354, filed on May 24, 2022.
(60) Provisional application No. 63/345,369, filed on May 24, 2022.
(60) Provisional application No. 63/345,388, filed on May 24, 2022.
(60) Provisional application No. 63/427,128, filed on Nov. 22, 2022.

(51) Int. Cl. C07K 16/30; C07K 16/28; C07K 14/55

(52) U.S. Cl. CPC C07K 16/30; C07K 16/2809; C07K 16/2803; C07K 14/55; C07K 2317/622; C07K 2319/02; C07K 2317/31

(57) ABSTRACT

A therapeutic molecule (single chain-based antibody or ligand-based) optimized for expression and secretion from engineered T cells, which may be gamma delta (gd) T cells. When expressed from engineered gdT cells, the STAR will be secreted and mediate engagement between gdT cells and antigen/receptor on target cells. Binding mediates the formation of a cytolytic synapse between the gdT cell and the target cell leading to activation the gdT cells to release proteolytic enzymes that kill target cells.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “230110_Final_103-3002PCTSeqListing.xml” (˜218 kb), which was created on Jan. 10, 2023 which is incorporated by reference herein.

### BRIEF SUMMARY

Novel T-cell activating bispecific antibody therapeutics. In some variations the bispecific antibodies may be used to engaged cytotoxic T cells against tumor cells. Engineered gamma delta T cells secreting bispecific therapeutics (antibody-based and/or ligand-based) for enhanced cytotoxicity towards various tumor antigens.

### BRIEF DESCRIPTION OF THE DRAWINGS

FIG. 1 provides an exemplary STAR framework.

FIG. 2 provides a schematic representing variable elements of a generic STAR design and exemplary specific element identities.

FIG. 3 demonstrates further schematics representing variable elements of STAR designs.

FIG. 4 demonstrates a schematic of an alternative STAR design capable of binding gamma delta T cells.

FIG. 5 is a LentET STAR schematic.

FIG. 6 provides a schematic of methods of manufacturing and genetically engineered gamma delta T cells.

FIG. 7 is a schematic of STARS Mechanism of action.

FIG. 8 is a flow diagram of a method for gdT cell production.

FIG. 9 is an overview of gdT cell expansion process.

FIG. 10 shows identification of donors with acceptable ex vivo expansion of gdT cells from peripheral blood mononuclear cells (PBMCs).

FIG. 11 shows screening of ex vivo expanded gdT cells to identify donors that generate gdT cells with high cytotoxicity toward K562 human cancer cells.

FIG. 12 is chart of GFP expression in LentET transduced gdT cells.

FIG. 13 is a chart of GFP MFI in LentET transduced gdT cells.

FIG. 14 is a chart of data showing cytotoxicity of secreted media from PTK7 and GD2 STAR expressing 293T cells.

FIG. 15 is data related to mRNA transfected gdT-CMK cytotoxicity.

FIG. 16 is a Western blot analysis of the designated STAR proteins.

FIG. 17 is quantitation of STAR secretion.

FIG. 18 is a characterization of secretion with the albumin linker.

FIG. 19 is a characterization of gdT cells transduced with STAR-encoding lentivirus gain cytotoxic potential against target cells.

FIG. 20. is data characterizing Integrin aV B3 CD3 STAR.

FIG. 21 is data characterizing IL2 CD19 CD3 STAR.

FIG. 22 is data characterizing mSA PTK7 CD3 STAR.

FIG. 23 is data characterizing mSA PTK7 CD3 STAR.

FIG. 24 is data characterizing mSA and native signal peptide hSCF CD3 STARs.

FIG. 25 shows gdT cells (effector [E]) were transfected with mRNA encoding the mSA and native signal peptide versions of the hSCF CD3 STAR and mixed with IMR5 cells (target [T]).

FIG. 26 is data characterizing mSA and IL2 GD2 CD3 STARs.

FIG. 27 is data characterizing mSA and IL2 GD2 CD3 STARs.

FIG. 28 demonstrates IL2 SSTR HL and LH CD3 STAR.

FIG. 29 demonstrates IL2 SSTR HL and LH CD3 STAR.

FIG. 30 demonstrates a humanized/deimmunized version of the CD3 scFv directs gdT mediated killing.

FIG. 31 demonstrates a humanized/deimmunized version of the CD3 scFv directs gdT mediated killing.

FIG. 32 demonstrates Lentiviral delivery of shRNA knocks down HLA Class I and II surface expression.

FIG. 33 demonstrates Lentiviral delivery of shRNA knocks down HLA Class I and II surface expression.

FIG. 34 demonstrates alternative gdT targeting moieties direct gdT mediated cytoxicity.

FIG. 35 demonstrates alternative gdT targeting moieties direct gdT mediated cytoxicity.

FIG. 36 demonstrates Somatostain ligand gdT mediated cytoxicity toward NET cells.

FIG. 37 demonstrates Somatostain ligand gdT mediated cytoxicity toward NET cells.

FIG. 38 demonstrates IL2 TPO BR CD3 STAR expression.

FIG. 39 demonstrates mRNA mediated protein expression correlates with mRNA free energy.

### DETAILED DESCRIPTION

The claimed subject matter is now described with reference to the drawings, wherein like reference numerals are generally used to refer to like elements throughout. In the following description, for the purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The following description of the drawings and the various system, method, and apparatus is not intended to limit the inventive system, methods and apparatus disclosed herein to one variation, but rather to enable any person skilled in the art of project management and/or software development to make and use the inventive system, method and apparatus.

The present disclosure relates to peptides, proteins, nucleic acids and cells for use in immunotherapeutic methods. In a variation, we disclose hematopoietic cells capable of secreting one or more synthetic fusion proteins and/or therapeutics. In particular, the present disclosure relates to the immunotherapy of cancer, including, e.g., B cell malignancies, neuroblastoma, osteosarcoma, neuroendocrine tumors (NETs), and acute myeloid leukemia (AML). The present disclosure furthermore relates to target cell cytotoxicity and secreted T cell actuators (referred to herein as “STARs”).

“STARs” is an umbrella term to describe the proteins genetically engineered to be expressed from gamma delta T cells. The disclosed STARs provide a unique advantage over existing soluble immune-oncology therapies, e.g., cytokines, monoclonal antibodies, and bispecific immune cell engagers. The STARs disclosed herein provide a solution to the side effects encountered by existing soluble immune-oncology therapies (i.e., side effects related to dosing, pharmacokinetics, and pharmacodynamics). The STARs disclosed herein are ECO optimized using a proprietary method of codon optimization. STARs are novel ECO optimized secreted T cell Actuators which are secreted from gamma delta T cells following gene transfer (e.g., viral vector transduction or mRNA electroporation).

We disclose herein novel gamma delta T cells (referred to herein as gdT cells or gdT cells) engineered to secrete proteins that can affect cancer. For example, gdT cells may be engineered to secrete proteins that act to alter the growth, expansion, and viability of a T cell population. In a variation, the gamma delta T cells, secrete bi-specific T-cell actuators. In the past, bi-specific T cell engagers were injected directly into patients via bolus therapy of Fc containing bi-specific antibodies (bsAbs), or continuous infusion of Fc-free bsAbs. We disclose herein a novel method of introducing STARs, T cell actuators and/or other bispecific molecules, and/or other secreted proteins by delivering to a patient gdT cells capable of secreting therapeutic agents of interest. In some variations, gdT cells delivered to the patients can secrete the proteins of interest. In a variation, a gdT T cell expressing STARs, a STAR (which may be but is not limited to a bi-specific T cell engager) is inserted into a patient.

The present disclosure includes STAR designs that target gdT cells to somatostatin receptor 2 (SSTR2)+ tumor cells. A monoclonal antibody targeting SSTR2 was adapted by converting it into several single chain variable fragment (scFv) designs and used them to direct the STARs/gdT cells to the SSTR2+ tumor.

In the present disclosure, the STAR designs can be secreted in vivo from ex vivo modified gdT cells. However, the protein designs may be useful as recombinant proteins injected directly. The gdT cells can be modified to express the STARs by a number of methods, including lentiviral transduction, AAV transduction, mRNA electroporation, mRNA transfection, and non-viral gene transfer technologies, CRISPR knock in, etc.

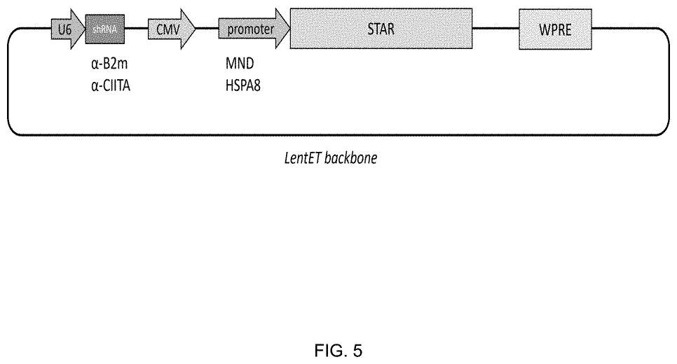

Disclosed herein are amino acid sequences, non-optimized DNA sequences, and Expression Codon Optimized (ECO) for gamma delta T-cell (ECOg) sequences. Additionally disclosed are two unique codon optimization sequence for the IL2 signal peptide which were uniquely ECO optimized for enhanced translation initiation to improve protein expressivity. Optimization of the IL2 signal peptide used a unique method optimization of the scFv-containing domains of the STAR. Further, these sequences in our LentET lentiviral backbone use two different promoters active in gdT cells (see FIG. 5). The first is the synthetic MND promoter; (SEQ ID NO: 152); the second is the human genome derived Heat shock 70 kDa protein 8 promoter HSPA8 (SEQ ID NO: 153). The latter promoter was found to have high activity in gamma delta T cells. Use of this promoter to drive gene expression in gamma delta T-cells, especially from a lentivector, is a novel use of this sequence.

Other systems, methods, features, and advantages of the present disclosure will be, or will become, apparent to one with skill in the art upon examination of the figures and detailed description. It is intended that all such additional systems, methods, features and advantages be included within this description, be within the scope of the present disclosure, and be protected by the following claims.

We disclose T cells secreting a therapeutic, e.g., a STAR, which results in eradication of cancer cells by various routes. A further advantage of this method is that a STAR disclosed can also recruit the patient's T cells to also fight the cancer.

As an additional or alternative technique, molecules can be added which enhance T cell function, for example but not limited to, gamma delta T cell function. Molecules can also be added which improve expansion and survival of T cells in vivo. Some examples of additional molecules are IL-2, IL-15. In some examples, the STAR may be a bi-specific T cell actuator. In other variations, the STAR operates without engaging a T cell to a cancer cell. In some variations, the STAR mediates the expansion of T cells.

We disclose a STAR has a unique property of being a protein secreted from gdT cells. Secretion from gdT cells has not been demonstrated before. In fact, secretion from gdT cells required extensive optimization of the expression construct. To achieve the disclosed construct capable of expression from gdT cells, we optimized the system at several points in the protein expression chain, which will be discussed further below.

A STAR (e.g., in a single chain-based antibody and/or ligand-based format) optimized for expression and secretion from engineered gamma delta (“(gd) T cells” or “gdT”). When expressed from engineered gdT cells, the STAR will be secreted and mediate engagement between gdT cells and antigen/receptor on target cells. Binding mediates the formation of a cytolytic synapse between the gdT cell and the target cell leading to activation the gdT cells to release proteolytic enzymes that kill target cells.

We disclose STARs (e.g., in a scFv-based antibody and/or ligand-based format) optimized for gdT cell expression and secretion (IL-2 signal peptide sequence or another signal peptide).

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration/Administer: To provide or give a subject an agent, such as a therapeutic agent (e.g., a recombinant AAV, recombinant lentivirus, STAR, vector expressing a star, modified gdT cell capable of expressing a STAR), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Antigen Binding Moiety: As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

Antigenic Determinant: As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

Specific Binding: By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art. In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³M).

Affinity: “Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein.

As used herein, the terms “first”, “second” or “third” with respect to Fab molecules etc., are used for convenience of distinguishing when there is more than one of each type of moiety. Use of these terms is not intended to confer a specific order or orientation of the bispecific antibody unless explicitly so stated.

Valent: The term “valent” as used herein denotes the presence of a specified number of antigen binding sites in an antibody. As such, the term “monovalent binding to an antigen” denotes the presence of one (and not more than one) antigen binding site specific for the antigen in the antibody.

Antibody: The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

Antibody Fragment: An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. Single-domain antibodies are antibody fragments comprising all or a portion of the heavy chain variable domain or all or a portion of the light chain variable domain of an antibody. In certain embodiments, a single-domain antibody is a human single-domain antibody. Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kuby Immunology, 6^{th} ed., W.H. Freeman and Co., page 91 (2007). A single VH or VL domain may be sufficient to confer antigen-binding specificity.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present.

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

Gamma delta T cells (γδ T cells) or (gd T) are T cells that have a distinctive T cell receptor (TCR) on their surface. Most T cells are a (alpha beta) T cells with TCR composed of two glycoprotein chains called α (alpha) and β (beta) TCR chains. In contrast, gamma delta (γδ) T cells have a TCR that is made up of one γ (gamma) chain and one δ (delta) chain. This group of T cells is usually less common than αβ T cells.

Hematopoietic cells are cells capable of developing into blood cells through hematopoiesis.

Human peripheral blood mononuclear cells (PBMCs) are immune cells with a single nucleus. PBMCs originate in bone marrow. PBMCs are secreted into peripheral circulation. PBMCs are involved in both humoral and cell-mediated immunity. PBMCs include lymphocytes (T cells, B cells, NK cells) and monocytes.

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3c). The amino acid sequence of human CD3ε is shown in UniProt (www.uniprot.org) accession no. P07766 (version 144), or NCBI (www.ncbi.nlm.nih.gov/) RefSeq NP_000724.1.

The BiTE format, also known as a tandem scFv or (scFv)2, is a small-sized Fc-free molecule composed of two scFvs connected by a flexible linker on a single polypeptide. The in vivo transfer of bsAb-encoding genetic information might be performed using viral and nonviral vectors.

Bispecific: The term “bispecific” (including bi-specific and bsAb) means that the antibody is able to specifically bind to at least two distinct antigenic determinants. Typically, a bispecific antibody comprises two antigen binding sites, each of which is specific for a different antigenic determinant. In certain embodiments the bispecific antibody is capable of simultaneously binding two antigenic determinants, particularly two antigenic determinants expressed on two distinct cells.

Bispecific antibodies include at least one or more antigen binding domains; multimerization core that forms a homo- or hetero-mulitmer; and linkers connecting the elements. The antigen-binding domain may be an antibody fragment, such as a Fab, single-chain garment variable (scFv), or single domain antibody (sdAb), or alternatively, an antibody mimetic. Another approach is the use of extracellular domains of natural receptors or ligands for the design of bsAbs. The multitargeting concept that bsAbs make possible is particularly appealing from a therapeutic point of view because many diseases are multifactorial, involving multiple receptors, ligands, and signaling cascades. T-cell engaging bsAbs (TCE) are designed to simultaneously bind to a selected tumor-associated antigen (TAA) on the tumor cell surface and one of the extracellular CD3 subunits (most commonly CD3e) on the T-cell surface.

cDNA (complementary DNA): A piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

CAI: “CAI” is the codon adaptation index. CAI is used as a quantitative method of predicting the level of expression of a gene based on its codon sequence.

Control: A reference standard. In some embodiments, the control is a negative control sample obtained from a healthy patient. In other embodiments, the control is a positive control sample obtained from a patient diagnosed with cancer. In still other embodiments, the control is a historical control or standard reference value or range of values (such as a previously tested control sample, such as a group of cancer patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Enhancer: A nucleic acid sequence that increases the rate of transcription by increasing the activity of a promoter.

Flanking: Near or next to, also, including adjoining, for instance in a linear or circular polynucleotide, such as a DNA molecule.

Gene: A nucleic acid sequence, typically a DNA sequence, that comprises control and coding sequences necessary for the transcription of an RNA, whether an mRNA or otherwise. For instance, a gene may comprise a promoter, one or more enhancers or silencers, a nucleic acid sequence that encodes an RNA and/or a polypeptide, downstream regulatory sequences and, possibly, other nucleic acid sequences involved in regulation of the expression of an mRNA.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

Gene therapy: The introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects a function of the recipient cell.

Hybridizes: Hybridization assays for the characterization of nucleic acids with a certain level of identity to the nucleic acid sequences as provided herein are well known in the art; see e.g. Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989). The term “hybridization” or “hybridizes” as used herein may relate to hybridizations under stringent or non-stringentconditions. If not further specified, the conditions are preferably non-stringent. Said hybridization conditions may be established according to conventional protocols described, e.g., in Sambrook (2001) loc. cit.; Ausubel (1989) loc. cit., or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single and double stranded forms of DNA. A polynucleotide may include either or both naturally occurring and modified nucleotides linked together by naturally occurring and/or non naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Nucleotide: This term includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

ORF (open reading frame): A series of nucleotide triplets (codons) coding for amino acids. These sequences are usually translatable into a peptide.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed vectors.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as vector compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Preventing, treating or ameliorating a disease: “Preventing” a disease (such as cancer) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g., a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell.

Protein: A biological molecule expressed by a gene or other encoding nucleic acid (e.g., a cDNA) and comprised of amino acids.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

A recombinant virus is one that includes a genome that includes a recombinant nucleic acid molecule. As used herein, “recombinant AAV” refers to an AAV particle in which a recombinant nucleic acid molecule has been packaged.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Response element (RE): A DNA sequence included in a promoter to which one or more transcription factors can bind to and confer an aspect of control of gene expression.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Additionally or alternatively, percent (%) amino acid sequence identity with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. amino acid identity is given in the output alignment header.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals.

Synthetic: Produced by artificial means in a laboratory, for example a synthetic nucleic acid can be chemically synthesized in a laboratory.

TATA box: A DNA sequence found in the promoter region of a gene that can be bound by TATA binding protein and transcription factor II D during DNA unwinding and binding by RNA polymerase II. A TATA box sequence typically includes a TATAAA sequence and often includes additional 3′ adenine nucleotides.

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Transcription factor (TF): A protein that binds to specific DNA sequences and thereby controls the transfer (or transcription) of genetic information from DNA to RNA. TFs perform this function alone or with other proteins in a complex, by promoting (as an activator), or blocking (as a repressor) the recruitment of RNA polymerase (the enzyme that performs the transcription of genetic information from DNA to RNA) to specific genes. The specific DNA sequences to which a TF binds is known as a response element (RE) or regulatory element. Other names include cis-element and cis-acting transcriptional regulatory element.

Transcription factors interact with their binding sites using a combination of electrostatic (of which hydrogen bonds are a special case) and Van der Waals forces. Due to the nature of these chemical interactions, most transcription factors bind DNA in a sequence specific manner. However, not all bases in the transcription factor-binding site may actually interact with the transcription factor. In addition, some of these interactions may be weaker than others. Thus, many transcription factors do not bind just one sequence but are capable of binding a subset of closely related sequences, each with a different strength of interaction.

For example, although the consensus binding site for the TATA-binding protein (TBP) is TATAAAA; however, the TBP transcription factor can also bind similar sequences such as TATATAT or TATATAA.

Transcription factors (TFs) are classified based on many aspects. For example, the secondary, tertiary and quaternary structures of the protein structures DNA-binding sequence and properties, the interaction with the double helix of the DNA, and the metal and other binding characteristics. The JASPAR database and TRANSFAC (TRANSFAC® 7.0 Public 2005) are two web-based transcription factor databases, their experimentally-proven binding sites, and regulated genes.

Transcription Start Site: The location where transcription starts at the 5′ end of a gene sequence.

Therapeutically effective amount: The amount of agent, such as a recombinant AAV vector, that is sufficient to prevent, treat (including prophylaxis), reduce and/or ameliorate the symptoms and/or underlying causes of a disorder or disease, for example to prevent, inhibit, and/or treat cancer. For instance, this can be the amount necessary to inhibit or prevent viral replication or to measurably alter outward symptoms of the disease or condition.

For example, administration of a therapeutically effective amount of a vector as disclosed herein can decrease a symptom by a desired amount, for example by at least 20%, at least 30%, at least 40%, at least 50%, at least 60%, at least 70%, at least 80%, at least 90%, at least 95%, at least 98%, at least 100% or more, as compared to a suitable control.

It is understood that to obtain a therapeutic response to the disease or condition can require multiple administrations of the agent. Thus, a therapeutically effective amount encompasses a fractional dose that contributes in combination with previous or subsequent administrations to attaining a therapeutic outcome in the patient. For example, a therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an adeno-associated virus (AAV) vector. In some embodiments, the vector is a gamma-retroviral vector, a lentiviral vector, or an adenoviral vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Overview

According to the present disclosure, the term STARs refers to secreted T cell Actuators. The term “actuator” is used to encompass T cell engagers and/or activators. The term “actuator” indicates secreted molecules capable of making the gamma delta T cells capable of performing therapeutic functions. STARs may be composed of STAR (scFv-based antibody or ligand-based) on one end and a T cell actuator molecule (scFv-based antibody or ligand-based) on the other end. Each of the components of a STAR construct may be sequence optimized for gamma delta T cell expression and secretion.

In a variation, STARs are a a bispecific and/or a bi-active therapeutic molecule secreted from the engineered gdT cell. STARs mediate binding to CD3 on gdT cells and ligand molecule on target cell to form an immunological synapse which in turn activates gdT cell cytotoxicity.

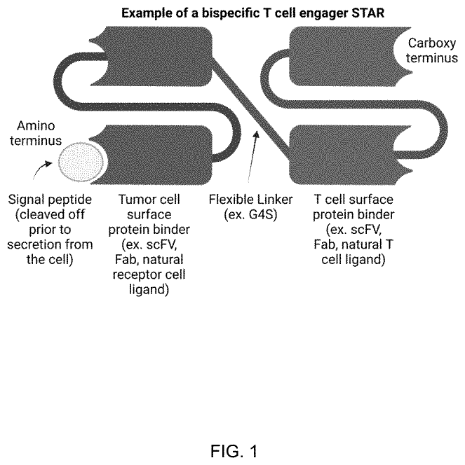

FIG. 1 provides a schematic of an exemplary STAR framework. The STAR may include at least one of the following elements: Starting on the amino terminus and traveling to the carboxy terminus, the STAR may include: (a) a signal peptide, which signal peptide may be cleaved off prior to secretion from the cell (b) a tumor cell surface protein binding (examples follow), (c) a flexible linker (optional), (d) a T cell surface protein binder.

Examples of signal peptides according to the disclosure include but are not limited to IL2, mSA (modified serum albumin) (SEQ ID NO: 3)(SEQ ID NO: 5), and hSCF (human stem cell factor)(SEQ ID NO: 4)(SEQ ID NO: 6).

In a variation, the tumor cell surface binding protein may be one or more of, e.g., an scFv, Fab, or natural cell ligand. The tumor cell surface binding protein may target cancer cell surface protein targets, e.g., CD19, SSTR2, GD2, PTK7, CD5, CD20, CD22, CD110, CD117, CD19 LH scFv, PTK7 HL scFv, GD2 HL scFv, GD2 LH scFv, Integrin aVB3 HL ScFv, SSTR2 HL scFv, SSTR2 LH scFv, 2×SST28 3×G4S, 2×SST28 4×G2s, TPO ligand, hSCF ligand (SEQ ID NO: 4). (Note: HL indicates a heavy chain-light chain orientation and LH indicates a light chain-heavy chain orientation). The tumor cell binding domain may be a single chain antibody variable domain fragment or a tumor cell receptor ligand that binds one selected from the group consisting of SSTR2, PTK7, GD2, SSTR5, CD19, aVB3, CD110, and CD5. The tumor cell binding domain may be SSTR2 scFv (SEQ ID NO: 52-18) (either LH or HL), PTK7 HL scFv (SEQ ID NO: 17-23), SSTR2 HL scFv, CD19 scFv (SEQ ID NO: 7-11) (LH or HL orientation), GD2 scFv (SEQ ID NOS. 38-44) (in LH or HL orientations), Integrin aVB3 ScFv (SEQ ID NOS: 45-51)(in LH or HL orientations), 2×SST28 3×G4S (SEQ ID NOS: 59-65), 2×SST28, 4×G2s (SEQ ID NOS: 66-72), TPO (ligand) (SEQ ID NOS: 80-86), and SCF (ligand) (SEQ ID NOS: 87-93).

In a variation, the flexible linker may be, e.g., G4S (SED ID NO: 155), albumin (SEQ ID NOS: 31-37), Fc (SEQ ID NOS: 87-93). As referenced herein, the linkers may be combined, e.g, G4S refers to AA Sequence (GGGGS)(SEQ ID NO: 155); G4S-albumin-G4S (e.g., (based on AA Seq) SEQ ID NO: 155-SEQ ID NO: 31-SEQ ID NO: 155); G4S-Fc-G4S (e.g., based on AA Seq.) SEQ ID NO: 155-SEQ ID NO: 87-SEQ ID NO: 155).

In a variation, the gamma delta T cell surface protein binding may be one or more of, e.g., scFV, Fab, and/or natural gamma delta T cell ligand. In a variation, an example of gamma delta T cell surface antigen proteins targets disclosed herein include, e.g., CD3 D Q subunits, D Q TCR (T cell receptor) subunits, CD16, NKG2D, FasL, TRAIL. The gamma delta T cell surface antigen protein actuators, engagers and/or activators may be at least one of CD3 HL scFv, CD3 (Hum2) HL scFv, JAML HL scFv, CDXAR ligand, gd-c (V1) HL scFv, gd-c (V6) HL scFv.

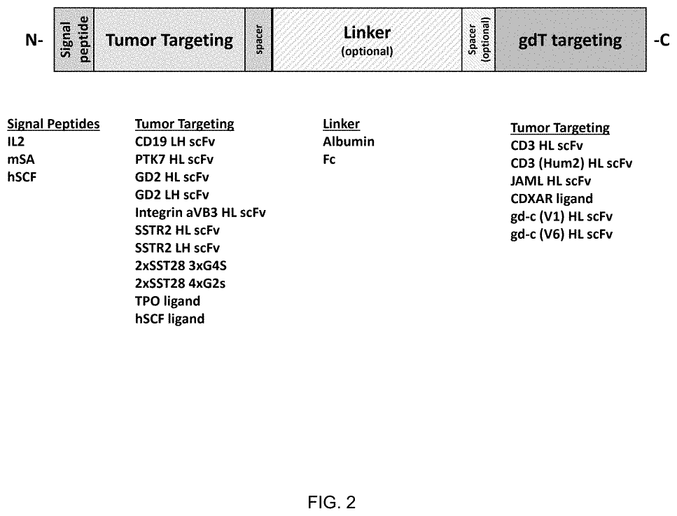

FIG. 2 provides a schematic representing variable elements of a generic STAR design and exemplary specific element identities.

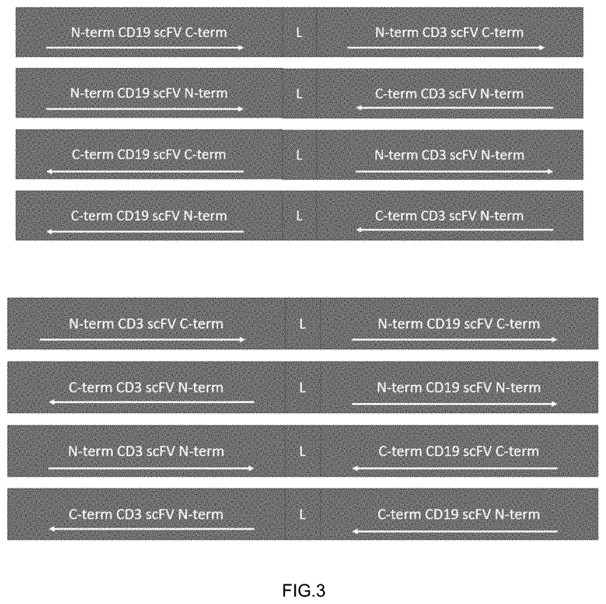

FIG. 3 demonstrates further schematics representing variable elements of STAR designs. In the present disclosure, the N terminus vs C terminus arrangement of the elements can be arbitrary. For instance, an exemplary CD19/CD3 molecule can be arranged with CD19 scFV at/toward the N terminus and CD3 at/toward the C terminus. However, the CD3 could likely be located at the N terminus and the CD19 at the C terminus. The IL2 signal sequence (or alternative) might always be at the N terminus. This general scheme may apply to all the described molecules according to the present disclosure. In addition, it is possible that the protein sequences may be reversed relative to their location within the protein.

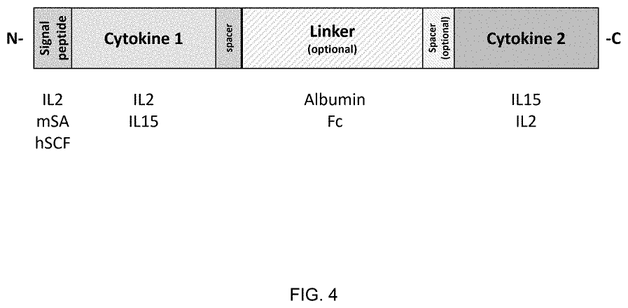

FIG. 4 demonstrates a schematic of an alternative STAR design capable of binding gamma delta T cells. The ligand based STAR is shown with cytokind actuators on each end of the construct. In this exemplary STAR, the signal peptide can be, e.g., IL2, mSA (SEQ ID NO: 3), or hSCF (SEQ ID NO: 4). Cytokine 1 may be IL2 or IL15. The optional linker may be albumin (e.g., SEQ ID NOS: 31-37) or Fc (SEQ ID NOS: 87-93), Cytokine 2 may be IL 15 or IL2.

FIG. 5 is a LentET STAR schematic. It shows the elements that may be variably present in the transgene design on a lentiviral cassette for the expression of a STAR from a target cell. The transgene may include one or more of an (optional) shRNA cassette, internal promoter (e.g., MND or HSPA8), and WPRE sequence (optional). LentET is a lentiviral packaging system. In a variation, the transgene packaging plasmid is driven by an external CMV promoter which drives the expression of a transgene cassette RNA including one or more of: 1) the cis-viral elements necessary for viral assembly and packaging, 2) an internal promoter sufficient for gene expression in the target tissue (e.g, the MND promoter (SEQ ID NO: 152) or HSPA8 promoter (SEQ ID NO: 153)), 3) an ECO optimized STAR-encoding cDNA sequence, 4) optionally a mutated version of the Woodchuck Hepatitis Virus Posttranscriptional Regulatory element (WPREmut) and 5) optionally an shRNA expression cassette driving the expression of on shRNA directed against a component of the major histocompatibility class (MHC) I or II complexes.

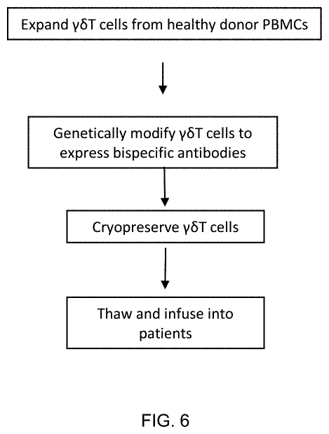

FIG. 6 provides a schematic of methods of manufacturing and genetically engineered gamma delta T cells. Donor peripheral blood mononuclear cells (PBMCs) of autologous or allogenic sources that can be pre-screened for disease-specific profiles may be used as starting material for the expansion, genetic engineering, and purification of genetically modified gamma delta T cells. Final products can include various compositions of gamma delta T cells which may include, e.g., gamma delta 1+ and gamma delta 2+ T cells, at various ratios for the adoptive transfer and treatment of various blood cancers and solid tumors.

Also disclosed are methods and compositions of gamma delta T cells which may include, e.g., gamma delta 1+ and gamma delta 2+ T cells genetically engineered to express and/or secret therapeutics against tumor antigens for the treatment of various blood cancers and solid tumors. Gamma delta T cells can be expanded from autologous or allogeneic donors under serum-free conditions. Donors can be selected from a set of screening criteria that include, but are not limited to, disease-specific/target specific profiles such as cytotoxicity assay in the presence or absence of other drugs and/or immunotherapies. Expansion of gamma delta T cells can be performed under serum-free conditions in a two-phase expansion procedure using PBMCs of autologous or allogenic source as starting material. Briefly, in one variation, PBMCs may be divided into two cultures for 1) gamma delta 2+ T cell expansion in the presence of zoledronic acid and IL-2 and 2) gamma delta 1+ T cell expansion by delta 1 monoclonal antibody-based activation in the presence of IL-2. Both expansion procedures may include two-phases: Phase 1 in the presence of the indicated supplements followed by an alpha beta T cell depletion; and Phase 2 in the presence of only IL-2. Alternatively, gamma delta 1+ T cell expansion can also be performed by exposure to concanavalin A (Con A) or phytohaemagglutinin (PHA) stimulation in the presence of IL-2.

Genetic modification may be performed during Phase 1 or Phase 2 of expansion. Phase 1 modifications can be achieved using modalities that heritably modify chromosomal DNA using, for example, viral or non-viral approaches. These approaches include but are not limited to lentivirus, gamma retrovirus, CRISPR, TALENs, etc. Modification of the genome at this phase can pass the modification to all daughter cells produced during further expansion. Phase 2 modifications can be achieved using non-integrating approaches such as AAV or mRNA which are not passed to daughter cells.

In the present disclosure, STARs may be secreted from genetically engineered gamma delta T cell. Actuation may occur through several mechanisms occurring alone or in combination. STARs may mediate binding to CD3 (or other T cell ligands) on gamma delta T cells and ligand molecules on target cells to form an immunological synapse which in turn activates T cell cytotoxicity. The secreted STARs can also mediate engagement between non-genetically modified gamma delta T cells and ligand molecules on target cells to form an immunological synapse which in turn activates T cell cytotoxicity.

In the present disclosure, genetic modification can take place with STARs and/or chimeric antigen receptors (CARs) in gamma delta 1+ and gamma delta 2+ T cells in combination or separately.

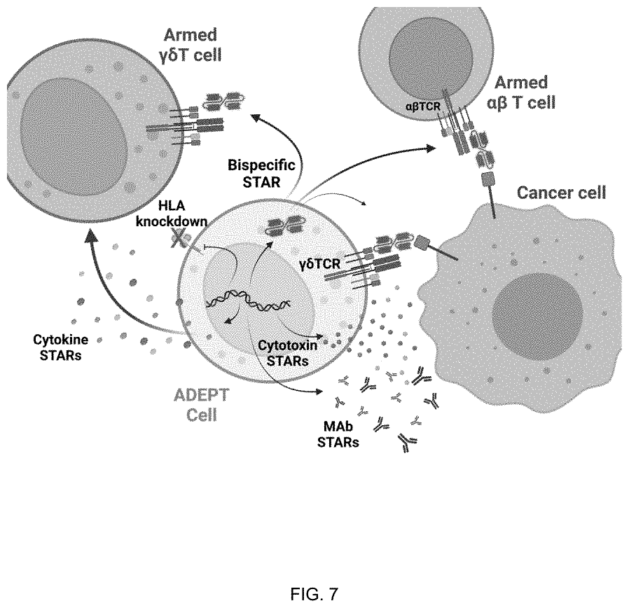

FIG. 7 is a schematic of STARS Mechanism of action. The mechanism of action can include STARs expression and secretion from genetically engineered T cells which mediates engagement between T cells and antigen/receptors on target cells. The formation of a cytolytic synapse between the T cell and the target cell by the STARs leads to T cell activation and the release of proteolytic enzymes that mediates cytotoxicity of target cells. Secretion of STARs from genetically modified T cells also leads to the engagement of non-modified T cells with target cells, resulting in enhanced cytotoxicity. Genetic modification of T cells with STARs can be combined with other immunotherapeutic approaches (such as, but not limited to, chimeric antigen receptors or CARs, monoclonal antibodies, and/or cytotoxic enhancing molecules). The present molecules could also be produced and purified from in vitro expression systems and delivered as a recombinant protein product.

By generating a final drug product of various gamma delta 1+/gamma delta 2+ T cell ratios, enhanced tumor specific targeting and cytotoxicity is mediated by the unique features of each gamma delta T cell type as summarized in Table 1:

TABLE 1
Features of human gd T cells for Immunotherapy

| Feature | Vd1+ T cells | Vd2+ T cells |
|---|---|---|
| Tumor Migration | Enhanced (CCR5 and | Only CCR5 expression |
| | CCR2 expression) | |
| Activation-Induced | Lower susceptibility | — |
| Cell Death (AICD) | | |
| Persistence | Greater | Less |
| | persistence in vivo | persistence in vivo |
| MHC Unrestricted TCR | Yes | Yes |
| Anti-Tumor Toxicity | Broad | Broad |
| Peripheral Circulation | Stable with age | Decreases with age |

In aspects of the present disclosure, gd T cell codon optimized scFv and ligand nucleic acid sequences are provided as SEQ ID NOS: 1-155. In the present disclosure, T cells may be genetically engineered via plasmid, mRNA, AAV, lentivirus, retrovirus. According to the present disclosure, the engineered T cells may provide endogenous expression and secretion of STARs for enhanced cytotoxicity and be used for adoptive cell transfer.

According to the present disclosure, the production of recombinant STARs can be used as a therapeutic agent for cancers and solid tumors, including neuroendocrine tumors (NETs) and neuroblastoma. The therapeutic mechanism may include sequence optimized expression and secretion of STARs from the engineered T cells for autocrine/paracrine engagement between T cells and target tumor cells.

According to the present disclosure, the STARs may be a recombinant protein product or a recombinant purified molecule for direct use as a therapeutic agent. The present disclosure may include the endogenous expression of STARs and/or adoptive cell transfer of T cells to enhance cytotoxic function of not only adoptively transferred cells but also endogenous T cells.

According to the present disclosure, there can be provided IL-2 and other leader sequences for secretion from genetically modified T cells.

According to the present disclosure, the treatment of cancers and tumors expressing a target antigen can use T cell therapeutics expressing and secreting STARs which can be combined with other immunotherapeutic agents. The present disclosure can be a recombinant protein that can be delivered directly, and which can be combined with other immunotherapeutic agents.

According to the present disclosure, a product can include one or more autologous or allogeneic derived T cells, STARs, and/or other immunotherapeutic agents expressed from genetically modified gamma delta T cells or co-administered with genetically modified gamma delta T cells.

We disclose herein an engineered T cell, specifically, gamma delta T cells, secreting a STARagent.

We disclose herein a gamma delta T cells (gdT cells) cell secreting one or more synthetic fusion proteins.

We disclose herein an engineered gamma delta T cells (gdT cells) secreting one or more synthetic fusion proteins targeted for secretion by inclusion of a modified serum albumin (mSA) signal peptide or stem cell factor signal peptide to enhance fusion protein production.

We disclose herein an engineered gamma delta T cells (gdT cells) cell secreting one or more synthetic fusion proteins that have been expression cassette optimized.

We disclose herein an engineered gamma delta T cells (gdT cells) secreting one or more synthetic fusion proteins that have been expression cassette optimized for expression in gamma delta T cells (gdT cells).

We disclose herein an engineered gamma delta T cells (gdT cells) secreting one or more synthetic fusion proteins that have been expression cassette optimized for expression in gamma delta T cells possessing gamma 9 and delta 2 T cell receptor subunits.

We disclose herein an engineered gamma delta T cells (gdT cells) secreting a STARagent, where the therapeutic agent is a bispecific T cell actuator.

We disclose herein gamma delta T cells (gdT cells) secreting one or more synthetic fusion proteins, where the synthetic fusion proteins are bispecific T cell actuators.

We disclose herein an engineered gamma delta T cells (gdT cells) secreting one or more synthetic fusion proteins where the synthetic fusion proteins are bispecific T cell actuators including an anti-CD3 scFv fused to an scFv capable of binding at least one of CD19, PTK7, GD2, SSTR2, and/or alpha-V beta-3 integrin.

We disclose an engineered gamma delta T cells (gdT cells) secreting one or more synthetic fusion proteins where the synthetic fusion proteins are bispecific T cell actuators comprised of an anti-CD3 scFv fused to a cognate receptor ligand domain.

We disclose an engineered gamma delta T cells (gdT cells) secreting synthetic fusion proteins where the synthetic fusion proteins are bispecific T cell actuators including an anti-CD3 scFv fused to the receptor ligand domain from stem cell factor (SCF), thrombopoietin (TPO), SSTR2, or SSTR5.

We disclose an engineered gamma delta T cells (gdT cells) secreting synthetic fusion proteins are bispecific T cell actuators including an anti-CD3 scFv fused to two or more copies of the receptor ligand SSTR2 and/or SSTR5.

We disclose an engineered gamma delta T cells (gdT cells) secreting synthetic fusion proteins are bispecific T cell actuators including an anti-gamma delta TCR scFv fused to an scFv that binds at least one of CD19, PTK7, GD2, SSTR2, and/or alpha-V beta-3 integrin.

We disclose an engineered gamma delta T cells secreting synthetic fusion proteins are bispecific T cell actuators including an anti-gamma delta TCR scFv fused to a cognate receptor ligand domain.

We disclose an engineered gamma delta T cells secreting synthetic fusion proteins are bispecific T cell actuators an anti-gamma delta TCR scFv fused to the receptor ligand domain from stem cell factor (SCF), thrombopoietin (TPO), SSTR2, or SSTR5.

We disclose an engineered gamma delta T cells secreting synthetic fusion proteins are bispecific T cell actuators including an anti-JAML scFv fused to an scFv that binds CD19, PTK7, GD2, SSTR2, or alpha-V beta-3 integrin.

We disclose an engineered T cell secreting one or more synthetic fusion proteins where the synthetic fusion proteins are bispecific T cell actuators including an anti-JAML scFv fused to a cognate receptor ligand domain.

We disclose an engineered T cell secreting one or more synthetic fusion proteins where the synthetic fusion proteins are bispecific T cell actuators including an anti-JAML scFv fused to the receptor ligand domain from stem cell factor (SCF), thrombopoietin (TPO), SSTR2, or SSTR5.

We disclose an engineered T cell secreting one or more synthetic fusion proteins where the synthetic fusion proteins are bispecific T cell actuators including an anti-JAML scFv fused to two or more copies of the receptor ligand SSTR2 or SSTR5.

We disclose an engineered T cell secreting one or more synthetic fusion proteins where the synthetic fusion proteins are bispecific T cell actuators including an a cognate JAML ligand fused to an scFv that binds CD19, PTK7, GD2, SSTR2, or alpha-V beta-3 integrin.

We disclose an engineered T cell secreting one or more synthetic fusion proteins where the synthetic fusion proteins are bispecific T cell actuators including a cognate JAML ligand fused to a cognate receptor ligand domain.

We disclose an engineered T cell secreting one or more synthetic fusion proteins where the synthetic fusion proteins are bispecific T cell actuators including a cognate JAML ligand fused to the receptor ligand domain from stem cell factor (SCF), thrombopoietin (TPO), SSTR2, or SSTR5.

We disclose an engineered T cell secreting one or more synthetic fusion proteins where the synthetic fusion proteins are bispecific T cell actuators including a cognate JAML ligand fused to two or more copies of the receptor ligand of SSTR2 or SSTR5.

We disclose herein an engineered T cell secreting a STARagent, where the therapeutic agent is a cytokine.

We disclose an engineered T cell secreting one or more synthetic fusion proteins where the synthetic fusion proteins include a dual cytokine.

We disclose an engineered T cell secreting one or more synthetic fusion proteins where the synthetic fusion proteins include a dual cytokine, the dual cytokine being one or more of IL2 and/or IL15 (e.g., IL2-IL2, IL2-IL15, IL15-IL15).

We disclose an engineered T cell secreting one or more synthetic fusion proteins where the engineered T cell is a gamma delta T cell.

We disclose an engineered T cell secreting one or more synthetic fusion proteins where the engineered T cell is a gamma delta T cell and the gamma delta T cell has gamma 9 and delta 2 T cell receptor subunits.

We disclose an engineered T cell secreting one or more synthetic fusion proteins where the engineered T cell is a gamma delta T cell with gamma 9 and delta 2 T cell receptor subunits produced by ex vivo expansion of health donor peripheral blood mononuclear cells using a using a two-stage culture method.

We disclose an engineered T cell secreting one or more synthetic fusion proteins where the engineered T cell, which may be a gamma delta T cell, and which may further be with a gamma delta T cell gamma 9 and delta 2 T cell receptor subunits, is modified for synthetic protein production by lentiviral or Retroviral vector transduction or mRNA transfection.

We disclose herein an engineered hematopoietic cell secreting one or more synthetic fusion proteins that have been expression cassette optimized

We disclose optimized expression and secretion of a bispecific scFv-based antibody or soluble ligand from engineered gdT cells for autocrine/paracrine engagement between gdT cells and target expression tumor cells.

We disclose localized expression and secretion of a bispecific scFv-based antibody or soluble ligand from engineered gdT cells.

We disclose Endogenous expression bispecific scFv-based antibody or soluble ligand from gdT cells and adoptive cell transfer of gdT cells to increase T cell cytotoxic function.

We disclose unique features include optimized sequence for gdT cell expression, and IL-2 leader sequence for secretion from genetically modified gdT cells as seen in the attached figures.

We disclose treatment of target positive tumors using off-the-shelf gdT cell therapeutics expressing a bispecific scFv-based antibody or soluble ligand.

We disclose a recombinant vector encoding one or more synthetic fusion proteins disclosed herein.

We disclose a recombinant vector, which may be any vector known by one of skill in the art including but not limited to a recombinant lentiviral or recombinant retroviral vector, encoding one or more synthetic fusion proteins as disclosed herein. A recombinant vector (e.g., lentiviral or retroviral) may include an internal promoter. The internal promoter may be a e.g., MND or HSPA8 promoter.

We disclose a recombinant lentiviral or retroviral vector encoding one or more fusion proteins as disclosed herein and additionally encoding short hairpin RNAs targeting beta 2 microglobulin (B2M) (SEQ ID NO: 150) and/or class II transcriptional transactivator (CIITA) (SEQ ID NO: 151). The vector may optionally include the internal promoter MND or HSPA8.

We disclose a method of preparing an engineered T cell secreting one or more synthetic fusion proteins including the steps of (a) expansion of healthy donor immune cells (e.g., g9d2 T cells)(See FIG. 6).

The disclosed engineered cells may be applied as a cancer therapeutic agent capable of treating cancers including but not limited to B cell malignancies, neuroblastoma, osteosarcoma, neuroendocrine tumors (NETs), and acute myeloid leukemia (AML).

We disclose an expression vector for an engineered cell expressing a therapeutic agent or synthetic fusion protein disclosed herein.

We disclose a method of treating cancers including the steps of (a) third-party, healthy donor PBMCs are culture and expanded ex vivo in the presence of IL-2 and zoledronate to enrich for gdT cells, (b) enriched gdT cells are genetically modified ex vivo for STAR expression and secretion. Genetically modified gdT cells are qualified and can be frozen to generate a bank for subsequent use or immediately transfused into recipient to control and/or reduce tumor growth.

### Experimental Data

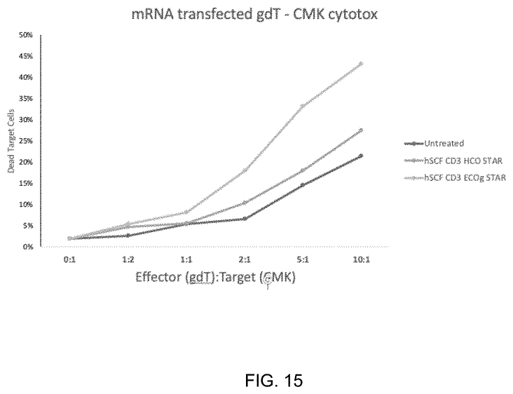

We provide optimized STAR contructs capable of improved expression from gamma delta T cells. We have shown that our proprietary ECOg optimized molecules offer improved expression in gamma delta T cells over standard human codon optimization. FIG. 15. Improved expression applied to a gamma delta T cell is reflected in improved or increased cytotoxicity. ECOg offers improvement over standard human codon optimization in gamma delta mediated gene expression. Here, gdT cells were transfected with mRNA carrying hSCF-coding STARs that had been ECOg or human (HCO) optimized. 4 hours post transfection gdT cells (effector) were co-incubated with CMK cells (target) and the cytotoxic potential of the transfected gdTs against CMK cells was measured.

This ECOg optimization was applied to optimize elements of the STAR relating to multiple steps along the expression pathway to provide highly efficient therapeutic effect through improved cytotoxicity over non-optimized cells. FIG. 1 provides a schematic of an exemplary STAR framework. The STARs disclosed by construct and through the Sequence Listings provided below, are optimized for improved expression over corresponding, human codon optimized and/or non-optimized constructs. The STAR constructs provided were optimized specifically for improved expression in gamma delta T cells. This optimization improved the ability of gamma delta T cells to more efficiently produce and express therapeutic proteins, as demonstrated by improved cytotoxicity.

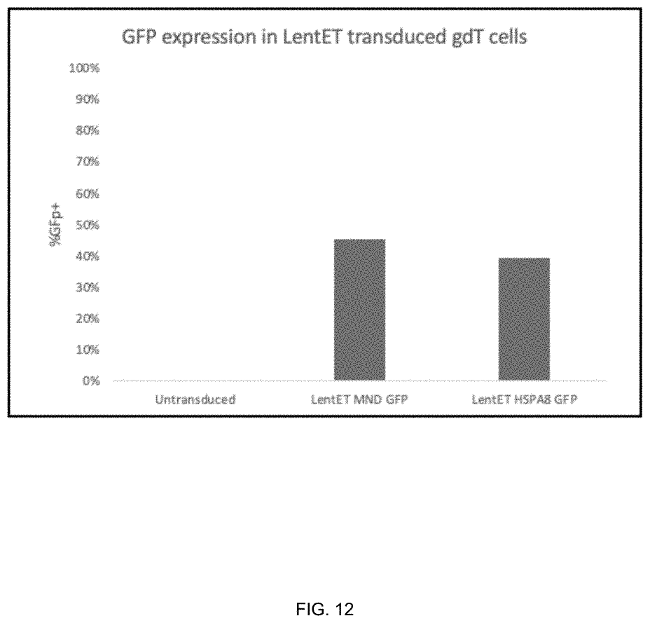

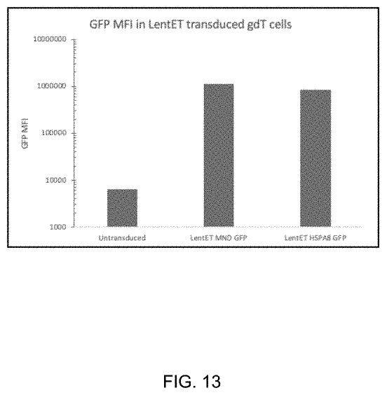

The optimizations targeted each step of the protein production pathway to increase translation and expression in the gamma delta T cell environment. For example, the promoter element of the STAR has been optimized for improved gamma delta T cell expression of therapeutic molecules. For example, we provide an ECO optimized MND promoter (SEQ ID NO: 152). In a variation, we provide a non-viral promoter, the HSP8 promoter (SEQ ID NO: 153), which has been ECO optimized. See FIG. 12 and FIG. 13 for a data demonstrating the retained expression of GFP in cells driven by the novel disclosed HSP8 promoters when compared to the MND promoters. MND and HSPA8 GFP expression in lentivirally transduced gdT cells. Fresh gdT cells were transduced with lentiviral particles carrying a GFP expression cassette driven by either the MND (myeloproliferative sarcoma virus MPSV enhancer, negative control region NCR deletion) or HSPA8 (Heat shock 70 kDa protein 8) (FIG. 12) The percent of GFP+ cells was measured at day 6 of gdT expansion. (FIG. 13) The mean fluorescent intensity (MFI) of all cells was measured at day 6 of gdT expansion.

We disclose herein the novel HSPA8 promoter (SEQ ID NO: 153) which was optimized to provide a non-viral promoter capable of reaching expression levels comparable to that of the viral promoter MND. With the HSPA8 promoter, we disclose a STAR with a preferable composition which, by using a non-viral promoter, reduces known problems created by the MND viral promoter. As shown in FIG. 12 and FIG. 13, the novel HSPA8 promoter was optimized to reach expression levels comparable to the MND promoter while providing a desirable non-viral promoter.

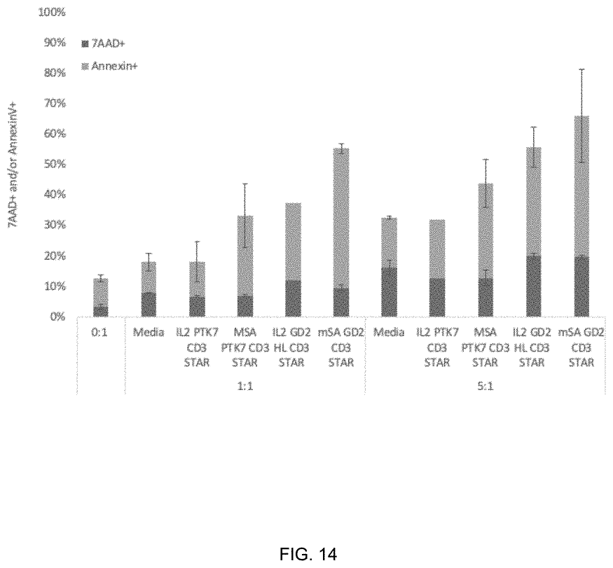

STARs were specialized for expression in gamma delta T cells through optimization of the signal peptide components. The increased expression generated by the optimized signal peptide components were demonstrated by improved cytotoxicity of the STAR expressing cells. In a variation, the mSA signal peptide (SEQ ID NO: 3) was optimized for gamma delta T cell expression. The disclosed signal peptides improved cytotoxicity of the gamma delta T cells through increased expression. FIG. 14 shows that the mSA signal peptide improves cytotoxicity of secreted media from PTK7 and GD2 STAR expressing 293T cells. Here, 293T cells were transfected with the respective STARs and conditioned media was collected 48 hours post-transfection. Gamma delta cells (effector) and IMR5 cells (target) were co-incubated in the presence of the conditioned media and the percent killing of IMR5 cells was measured after 4 hours. Ratios of effector:target cells are shown on the X axis label.

In a variation of the STAR provides expression optimized linkers. The linkers were optimized specifically for improved ability to be secreted from gamma delta T cells. We provide two optimized linkers, albumin (SEQ ID NO:SEQ ID NOS; 31-37) and Fc (SEQ NOS: 87-93).

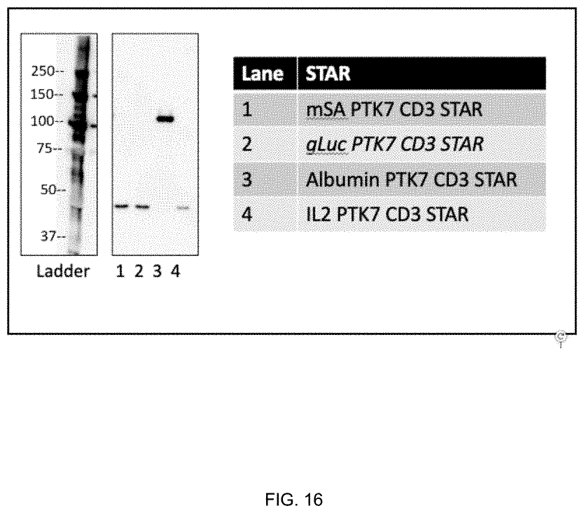

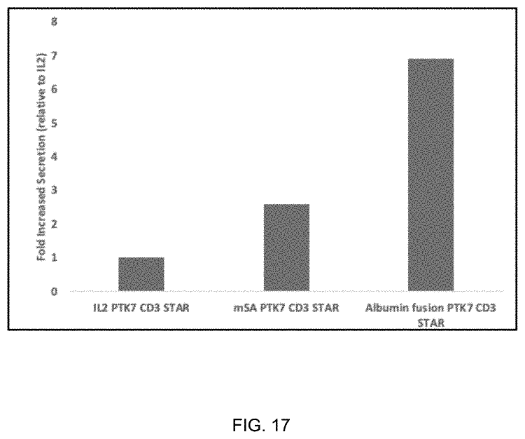

FIG. 16 and FIG. 17 demonstrate the improvements provided by the disclosed linkers. Here, albumin fusion and mSA signal peptide increase STAR secretion over IL2 design. Various STAR designs were transfected into 293T cells and conditioned media was collected 48 hours post transfection. FIG. 16 provides Western blot analysis of the designated STAR proteins. FIG. 17 shows quantitation of STAR secretion in the media normalized to the IL2 PTK7 CD3 STAR design.

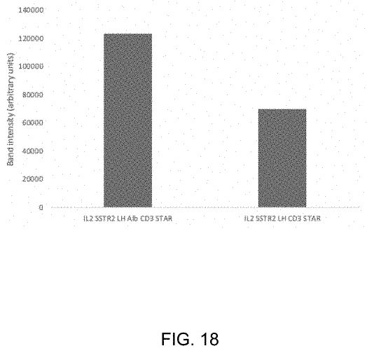

FIG. 18 further characterizes improvements provided by the disclosed optimized albumin linker, referred to as albumin fusion. FIG. 18 demonstrates that the central albumin fusion improves STAR secretion. To remedy the potential for steric hinderance of an N-terminus albumin fusion on STAR proteins, the albumin fusion was tested positioned between the tumor targeting end gdT targeting end of the STAR. 293T cells were transfected with the IL2 SSTR2 LH STAR with and without a centrally located albumin molecule. Conditioned media was collected 48 hours post-transfection and subjected to quantitative western blot.

The following data demonstrate the cytotoxic activity of the disclosed STAR variations.

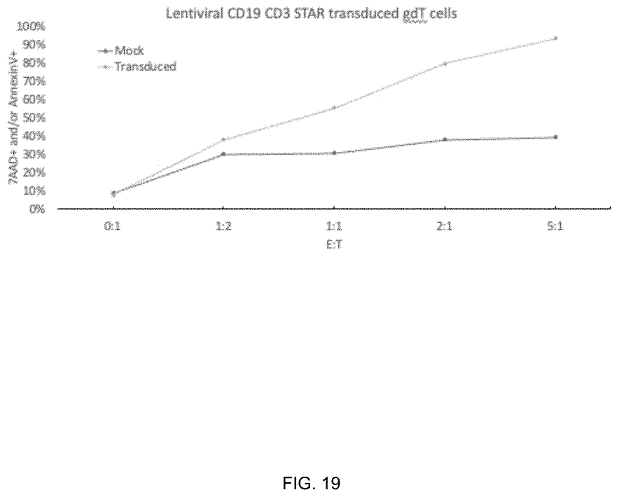

FIG. 19. gdT cells transduced with STAR-encoding lentivirus gain cytotoxic potential against target cells. Here, Fresh gdT cells were transduced with lentiviral particles carrying the IL2 CD19 LH CD3 STAR (e.g., SEQ ID NOS: 1-2). After several days of expansion the transduced and mock-transduced gdT cells (Effector, [E]) were coincubated with 697 cells (Target [T]) and toxicity toward the 697 cells was measured. Ratios of E:T cells are shown on the X axis. This demonstrates that gdT cells transduced with STAR-encoding lentivirus gain cytotoxic potential against target cells.

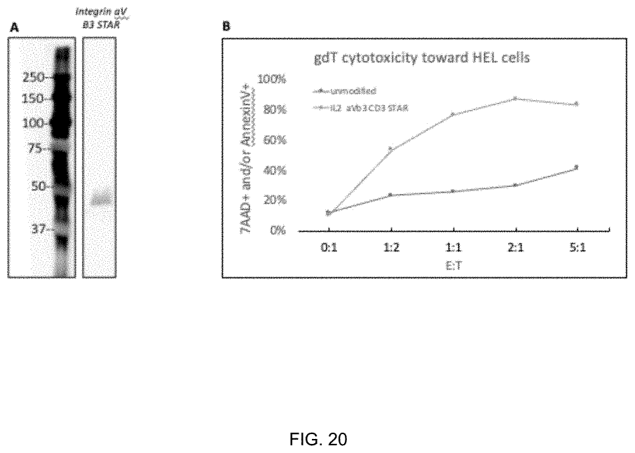

FIG. 20. Integrin aV B3 CD3 STAR (e.g., SEQ ID NOS: 45-51) promotes killing of target cells. (A) Plasmid expressing the integrin aV B3 CD3 STAR was transfected into 293T cells and conditioned supernatant was collected 48 hours post transfection. Western blot of the conditioned media shows the correct size of the integrin aV B3 STAR. (B) Fresh gdT cells were transfected with RNA encoding the integrin aV B3 CD3 STAR. gdT (effector, [E]) cells were mixed with Human ErythroLeukemia (HEL) cells (target, [T]) and cytotoxicity against the HEL cells was measured. Ratios of effector to target cells are shown along the X axis.

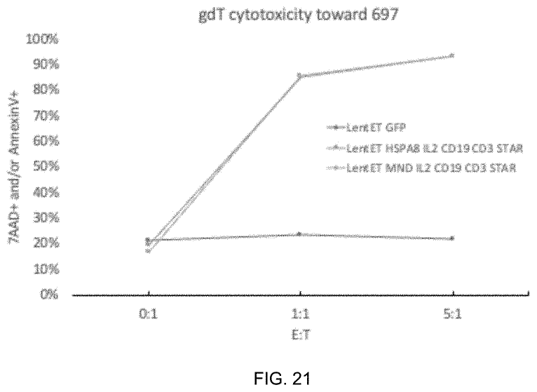

FIG. 21. IL2 CD19 CD3 STAR (e.g., SEQ ID NOS: 1-2) promotes killing of target cells. Here, 293T cells were transduced with lentivirus encoding GFP or the IL2 CD19 CD3 STAR (e.g., SEQ ID NOS: 1-2) driven by either the HSPA8 or MND promoters. Conditioned media was collected 48 hours after transduction. gdT cells (effector [E]) and 697 cells (target [T]) were mixed and conditioned media was added and cytotoxicity against the target cells was measured. Effector to target ratios are shown along the X axis. This figure shows both the efficacy of the IL2 CD19 CD3 STAR (e.g., SEQ ID NOS: 1-2) variations and demonstrates that the novel HSPA8 promoter provides a comparable non-viral option to the MND promoter.

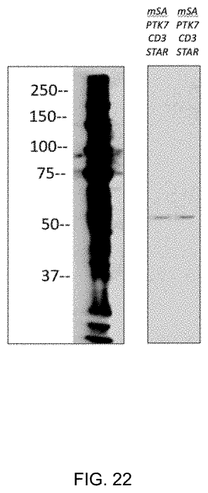

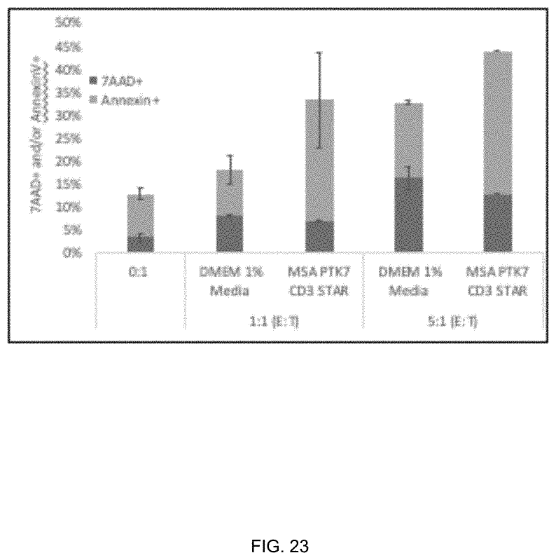

FIG. 22 and FIG. 23 demonstrate mSA PTK7 CD3 STAR (e.g., SEQ ID NO: 3, SEQ ID NOS: 17-23) promotes killing of target cells. Here, 293T cells were transfected with plasmid expressing the mSA PTK7 CD3 STAR (FIG. 22). Conditioned media was collected 48 hours after transfection. Western blot of the conditioned media shows a product of the expected size. In FIG. 23 gdT cells (effector [E]) and IMR5 cells (target [T]) were mixed and conditioned media was added and cytotoxicity against the target cells was measured. Effector to target ratios are shown along the X axis.

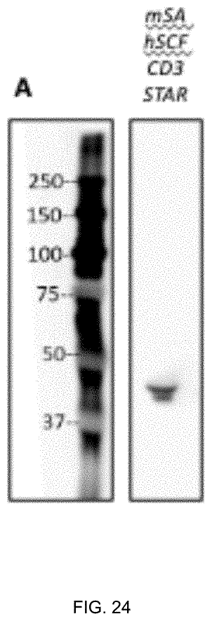

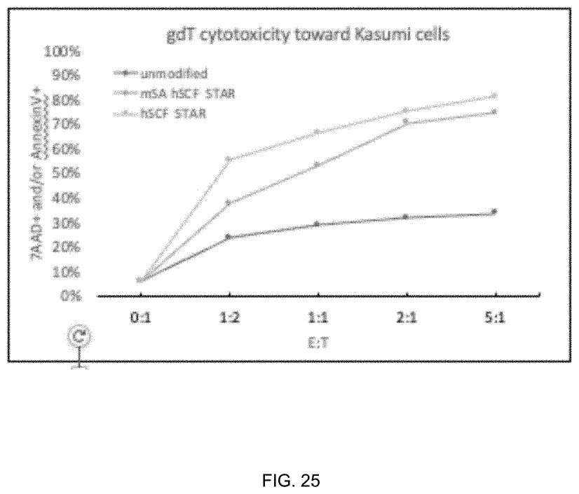

FIG. 24 and FIG. 25 show mSA (SEQ ID NO: 3) and native signal peptide hSCF CD3 STARs promote killing of target cells. FIG. 24 shows 293T cells were transfected with plasmid expressing the SCF CD3 STAR using the native SCF signal peptide (hSCF STAR). Conditioned media was collected 48 hours after transfection. Western blot of the conditioned media shows a product of the expected size. FIG. 25 shows gdT cells (effector [E]) were transfected with mRNA encoding the mSA (SEQ ID NO: 3) and native signal peptide versions of the hSCF CD3 STAR and mixed with IMR5 cells (target [T]). Cytotoxicity against the target cells was measured. Effector to target ratios are shown along the X axis.

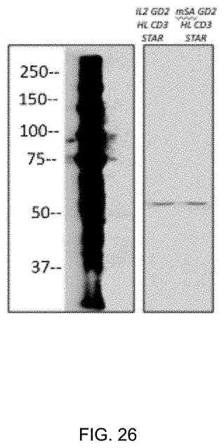

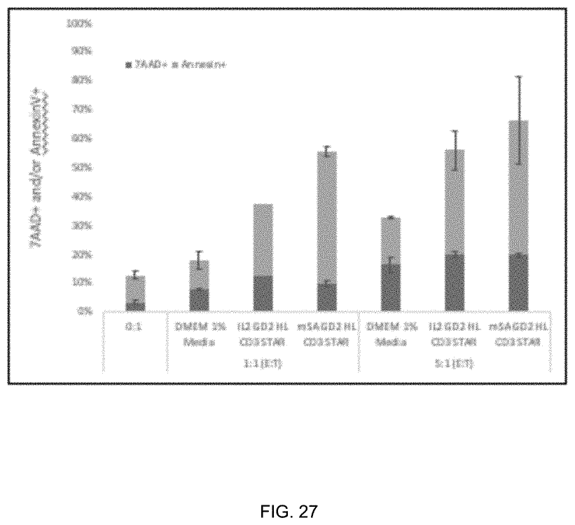

FIG. 26 and FIG. 27 demonstrate mSA and IL2 GD2 (e.g., SEQ ID NOS: 38-44) CD3 STARs promote killing of target cells. In FIG. 26, 293T cells were transfected with plasmid expressing the mSA or IL2 GD2 HL CD3 STAR. Conditioned media was collected 48 hours after transfection. Western blot of the conditioned media shows a product of the expected size. In FIG. 27, gdT cells (effector [E]) and IMR5 cells (target [T]) were mixed and conditioned media was added and cytotoxicity against the target cells was measured. Effector to target ratios are shown along the X axis.

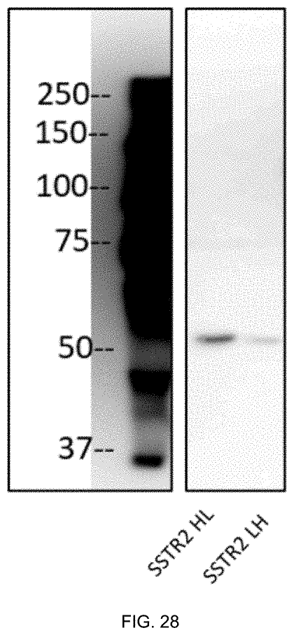

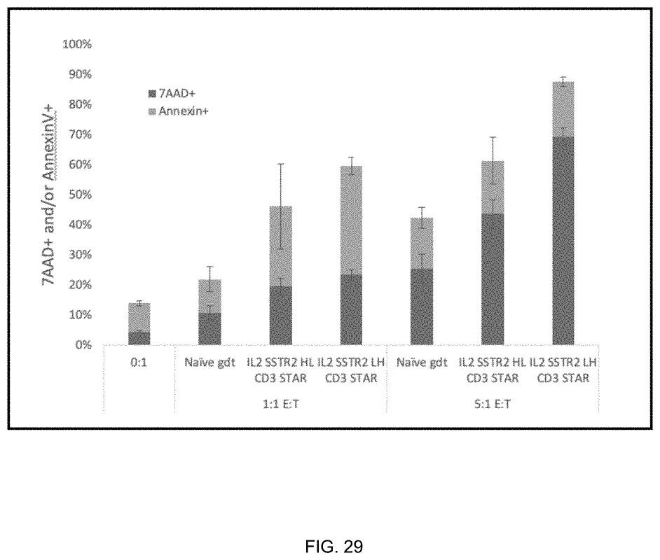

FIG. 28 and FIG. 29 demonstrate IL2 SSTR HL and LH CD3 STAR promote killing of target cells. In FIG. 28, Plasmids expressing the heavy/light (HL) and light/heavy (LH) arrangement of the SSTR2 scFv in the IL2 SSTR2 CD3 STAR chassis were transfected into 293T cells. Conditioned media was collected 48 hours post transfection where western blot detected bands of the appropriate size. In FIG. 29, mRNA encoding the IL2 SSTR2 HL or LH CD3 STAR were transfected into gdT cells. The transfected cells gdT cells (effectors [E]) were mixed with IMR5 cells (target [T] and the resulting cytotoxicity against the target cells were measured.

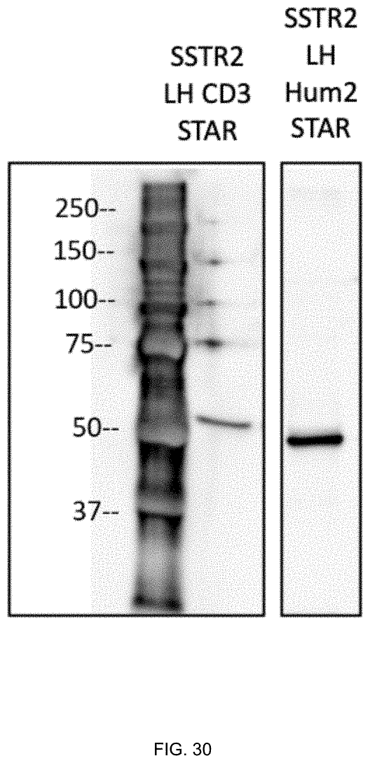

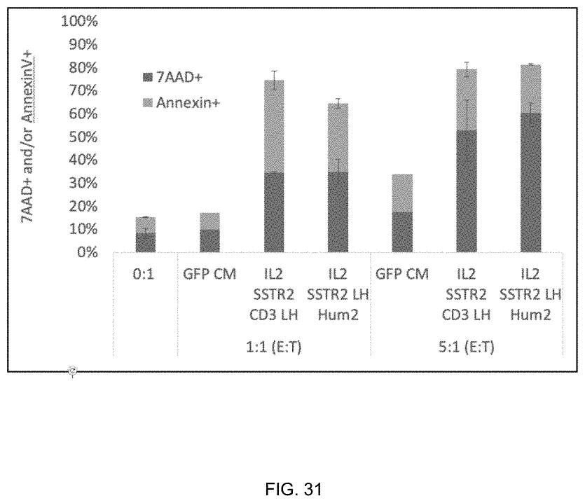

FIG. 30 and FIG. 31 demonstrate a humanized/deimmunized version of the CD3 scFv directs gdT mediated killing. In FIG. 30, 293T cells were transfected with plasmid expressing the CD3 (control) and Hum2 scFV (e.g., SEQ ID NOS: 94-100)(humanized/deimmunized) versions of the IL2 SSTR2 LH STAR. Conditioned media was collected 48 hours after transfection. Western blot of the conditioned media shows products of the expected sizes. In FIG. 31, gdT cells (effector [E]) were co-incubated with IMR5 cells (target [T]) in the presence of the conditioned media. Cytotoxicity against the target cells was measured. Effector to target ratios are shown along the X axis.

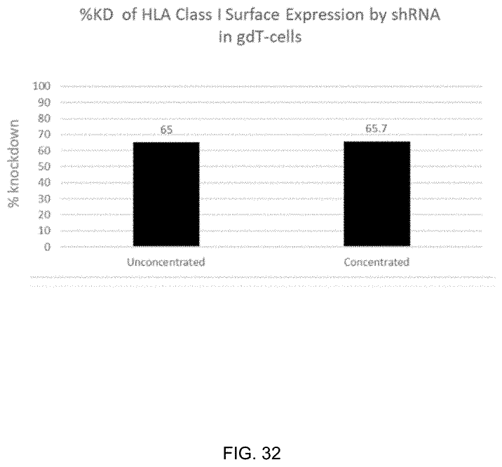

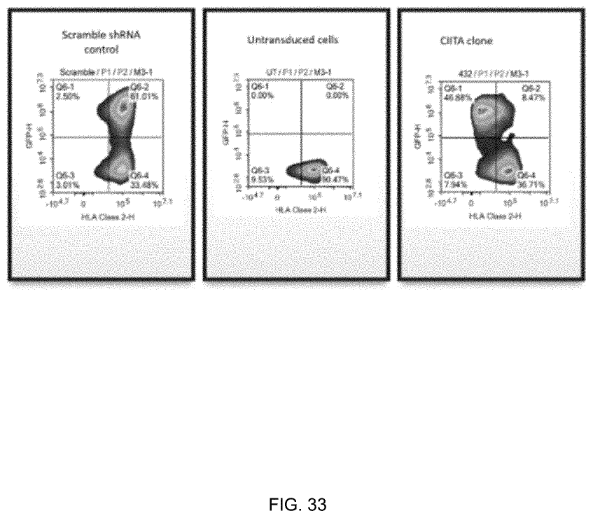

FIG. 32 and FIG. 33 demonstrate Lentiviral delivery of shRNA knocks down HLA Class I and II surface expression. In FIG. 32, gdT cells were transduced with concentrated and unconcentrated LentET vector carrying a cassette driving the expression of an anti B2m shRNA (B2m shRNA1) and percent knockdown of B2m (HLA Class I) was measured by flow cytometry. In FIG. 33, gdT cells were transduced with LentET vector carrying a cassette driving the expression of an anti CIITA shRNA (CIITA shRNA7) and knockdown of CIITA (HLA Class II) was measured by flow cytometry.

Our disclosed STARs are uniquely modifed to preemptively address and avoid HLA mismatch to improve chances of graft survival. In a variation, our disclosed gdT-cell product (STAR) is allogeneic, meaning they are derived from non-donor PBMCs. It is generally observed that graft vs host disease (GVHD) is not a major concern for gdT cells as they kill in an MHC-independent manner. However, in an abundance of caution we preemptively try to circumvent immunogenicity ever being an issue my making our cell product more “universal” given that these gdT-cell therapies will probably be administered to patients who are already severely immunosuppressed.

In current literature, reduction in immunogenicity has been accomplished by reducing (siRNA/shRNA) or eliminating (CRISPR knockout) the presence of HLA I & II complexes on the donor cell surface. This poses a unique hurdle as there are multiple HLA genes, most of which are highly polymorphic. There are, however, non-polymorphic protein targets whose expression, if abrogated, would result in loss/reduction of expression of HLA at the cell's surface. For HLA I, we are using a lentiviral vector expressing an shRNA targeting the non-polymorphic beta chain of all HLA class I surface complexes called beta-2-microglobulin. By reducing levels of B2M, an essential component of the HLA I complexes, we are also reducing HLA I expression on the cell surface. Reduction of HLA class II expression is not as straightforward. There is no common structural element to all HLA II complexes to provide a target for knockdown, but the literature has shown that by targeting the “Class II Major Histocompatibility Complex Transcriptional Transactivator” (CIITA), it is possible to universally decrease or eliminate expression of HLA II on the cell surface. CIITA is necessary for transcription of the HLA II genes, and without it, transcription cannot take place. As with HLA I, we are using a lentiviral vector encoding an shRNA against CIITA to reduce HLA II surface expression. Ultimately, we would want to have a lentiviral vector that produces a STAR as well as both shRNAs against B2M and CIITA so that we can reduce HLA I & II levels on our gdT cell surface resulting in a safer and more effective therapeutic, siLentET.

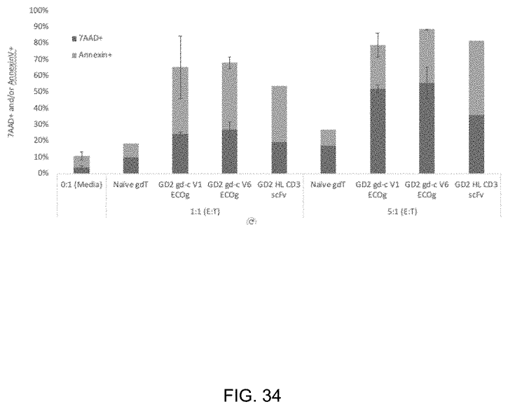

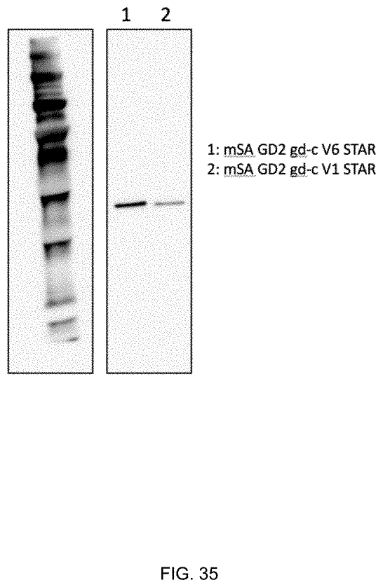

FIG. 34 and FIG. 35 demonstrate alternative gdT targeting moieties direct gdT mediated cytoxicity. In FIG. 34, shows 293T cells were transfected with plasmid expressing STARs directed toward target cells with an anti GD2 scFv and directed toward gdT cells with anti-gdT TCR scFv (gd-c V1 and gc-c V6) or anti CD3 scFv. Conditioned media was collected 48 hours after transfection. In FIG. 35, gdT cells (effector [E]) were co-incubated with IMR5 cells (target [T]) in the presence of the conditioned media. Cytotoxicity against the target cells was measured. Effector to target ratios are shown along the X axis.

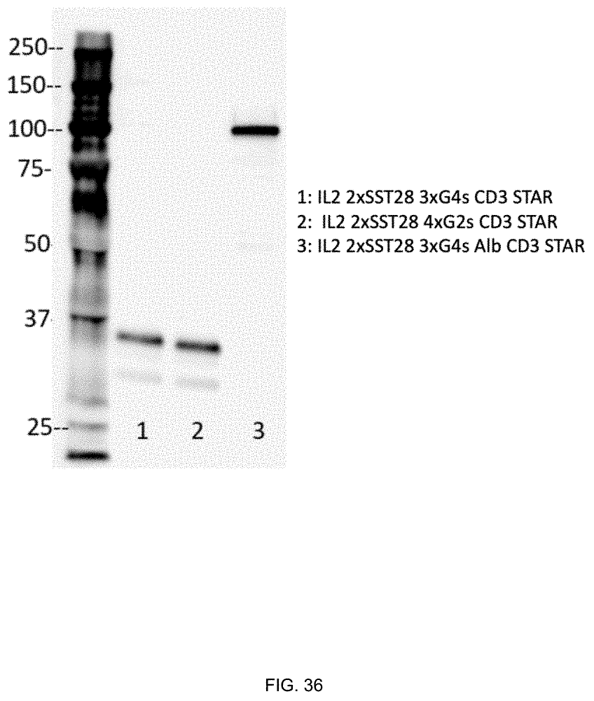

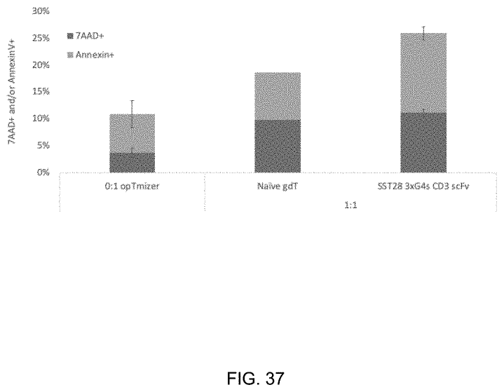

FIG. 36 and FIG. 37 demonstrates Somatostain ligand gdT mediated cytoxicity toward NET cells. In FIG. 36, 293T cells were transfected with plasmid expressing STARs directed toward target cells with somatostain ligand (SST28) and directed toward gdT cells with anti CD3 scFv. Conditioned media was collected 48 hours after transfection. In FIG. 37, gdT cells (effector [E]) were co-incubated with IMR5 cells (target [T]) in the presence of the conditioned media. Cytotoxicity against the target cells was measured. Effector to target ratios are shown along the X axis.

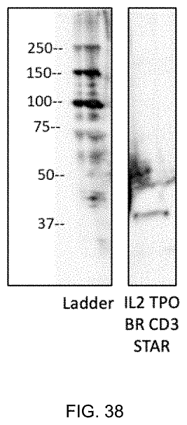

FIG. 38 demonstrates IL2 TPO BR CD3 STAR expression. Here, plasmid carrying the IL2 TPO BR CD3 STAR transfected into 293T cells express the expected protein product as detected by western blot.

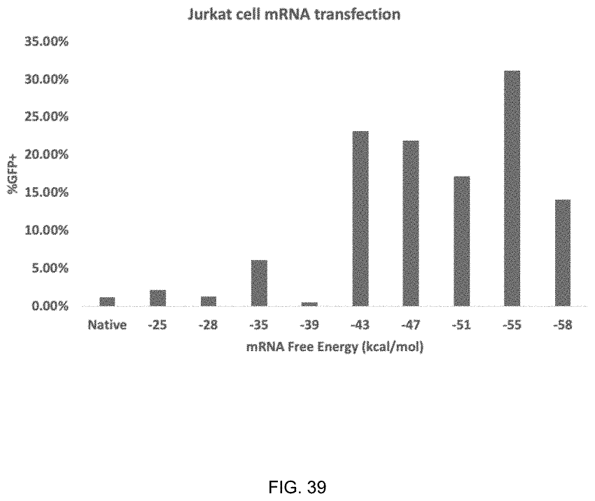

FIG. 39 demonstrates mRNA mediated protein expression correlates with mRNA free energy. Here, Jurkat cells were transfected with a panel of ECOg optimized mRNA constructs encoding varying from low to high mRNA free energy. GFP expression was measured 8 hours post transfection

Methods

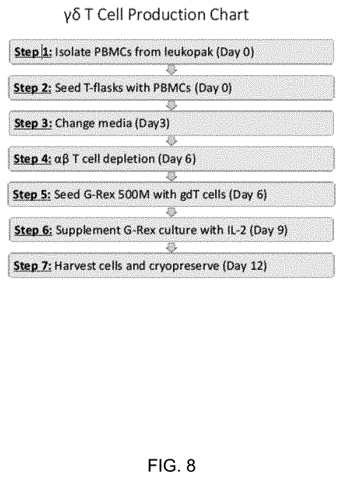

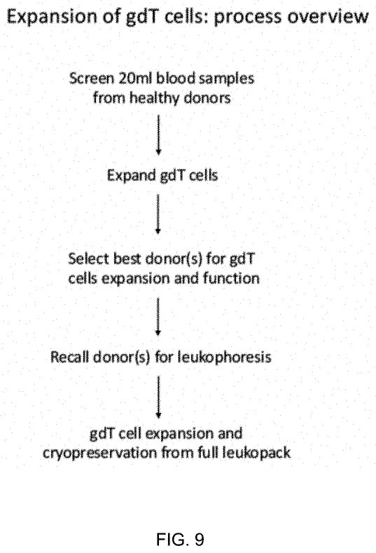

In the present disclosure, methods and compositions of gamma delta T cells genetically engineered to express and secret STARs for the treatment of various cancers and solid tumors, are provided. Turning to FIG. 9. Gamma delta T cells can be expanded from autologous or allogeneic donors under serum-free conditions. Donors may be selected from a set of screening criteria that include, but are not limited to, disease-specific/target specific profiles such as cytotoxicity assay in the presence or absence of other drugs and/or immunotherapies. (FIG. 8) Expansion of gamma delta T cells can be performed under serum-free conditions in a two-phase expansion procedure consisting of Days 0-6 in IL-2 and zoledronic acid in T-flask based culture (Phase 1) and Days 6-12 in IL-2 in Bioreactor based culture (Phase 2). An alpha beta T cell depletion step can be performed at Day 6 of culture, prior to Bioreactor based culture. Viral based genetic modification (lentiviral and/or gamma retroviral) can be performed on Phase 1 or Phase 2 of gamma delta T cell expansion. Alternatively, AAV-based or mRNA based genetic modification can be performed on Phase 2 of gamma delta T cell expansion for expression and secretion of STARs and/or other immunomodulators such as IL2-IL5 bispecific molecules.

In the present disclosure, the manufacturing of genetically engineered gamma delta T cells from autologous or allogeneic donor PBMCs under serum-free conditions can be in a two-phased expansion procedure. Genetic modification can take place during either phase 1 or phase 2 of gamma delta T cell expansion.

Donor pre-screening may be performed under a set of criteria to allow for optimal expansion, genetic modification, and cytotoxicity. Donor pre-screening may be based on disease-specific selection criteria such as disease/target specific cytotoxicity assay in the presence or absence of other drugs and/or immunotherapies.

According to the present disclosure, the manufacturing method can solve the problems of: serum-free expansion conditions; screening of donors for optimal expansion, genetic modification, and cytotoxicity towards disease specific target. According to the present disclosure, a two-phase expansion procedure can include an alpha beta depletion step for optimal product safety profile. Genetic modification may occur either in phase 1 or phase 2 or a combination thereof for optimal STARs secretion and/or other immunomodulators such as IL2-IL5 bispecific molecules secretion.

In aspects of the present disclosure, the expansion and genetic modification of gamma delta T cells can be capable of expression and secretion of STARs and/or other immunomodulators for disease-specific enhanced efficacy.

According to the present disclosure, a method can include: serum-free expansion conditions in a two-phased expansion method, genetic modification in either phase of expansion for combinatorial engineering with STARs and/or immunomodulators, expression and secretion of immunomodulators that promote gamma delta T cell expansion and viability both in vitro and in vivo.

An autologous or allogeneic gamma delta T cell genetically engineered with STARs harboring target tumor antigen for the treatment of cancers and solid tumors can be provided according to the present disclosure.

According to the present disclosure, the following can be provided: two phased expansions; genetic modification in either or both phases of the expansion; serum-free expansion conditions; and IL2-IL5 bispecific molecule expressed and secreted endogenously to enhance expansion, viability, and function.

In FIG. 8, a method according to the present disclosure is disclosed and summarized below.

Step 1: Isolate PBMCs from leukopak (Day 0)

Step 2: Seed T-flasks with PBMCs (Day 0)

Step 3: Change media (Day3)

Step 4: αβ T cell depletion (Day 6)

Step 5: Seed Bioreactor with gdT cells (Day 6)

Step 6: IL-2 supplementation (Day 9)

Step 7: Harvest cells and cryopreserve (Day 12)

Full leukopak from American Red Cross, typically 1-1.5e10 total nucleated cells per leukopak and typically 150-400 ml total volume.

Procedure:

Day 0

Step 1: Isolate PBMCs from leukopak (Day 0). This step includes the following protocol: Obtain 22 ml whole blood, Add 18 ml of Ficoll-Paque to 50 ml conical tubes (2 per donor), Remove blood from collection tubes and pipette into a 50 mL conical tube for each donor, Record the starting volume of blood. This volume does not include the PBS washing volume in the next step. Wash collection tubes with 4 ml PBS to remove residual blood and cells off the sides of the tube, then put into 50 mL conical tube with the rest of the blood. Add additional PBS to make final PBS:Blood 1:1 ratio by volume. Total diluted blood volume/donor=44 ml. Mix the blood gently by inverting the 50 mL conical tube. Carefully add 22 ml of diluted blood to the top of the 18 ml of Ficoll-Paque media. (do not mix). Spin the conicals, 400×g, 35 min 20 C. Remove the top layer (plasma) with a pipette. Collect PBMC layer with a pipette and move into a new 50 mL conical tube pooling samples from the same donor. Wash the cells by resuspending the pellet in 3 volumes, ˜45 ml PBS, then spin 500×g 10 min 20 C. Resuspend with 10 ml PBS to wash again. spin, 200×g 5 min 20 C. Resuspend with 10m PBS to wash again. Spin, 200×g 5 min 20 C.

Step 2: Seed T-flasks with PBMCs (Day 0). This step involves the following protocol: Resuspend cell pellet in 10 mL OpTmizer media. Count cells. Remove 1 ml (500K) cells for immunotyping CD3/gdTCR. Include gdTCR FMO control from pooled samples. Use ˜100K cells/flow tube. Bring final cell density to 1.5×10⁶ cells/mL in OpTmizer. Add IL2 and zoledronate to make concentrations of these 500 IU/mL IL-2 and 5 μmol/L zoledronic acid. Culture cells at 37 C/5% CO2 tilting flask (propped) to concentrate cell culture to bottom half of T-75 flask.

Day 3. Step 3: Change media (Day3). This step includes the following protocol: Transfer cells to 50 mL conical and gently pipette up/down with a 10 ml pipette to break up cell aggregates. Centrifuge cells at 250×g 10 min RT. Resuspend cells in OpTmizer media. Count cells. Bring final cell density to 1.5×10⁶ cells/mL in complete OpTmizer supplemented with 500 IU/mL of IL-2 and 5 μmol/L zoledronic acid and replating in the same T-75 flask. Culture cells at 37 C/5% CO2 tilting flask (propped) to concentrate cell culture to bottom half of T-75 flask. Lentiviral/Retroviral transduction. After Step 4, bring cells to a final density of 1.5×10⁶ cells/mL in complete OpTmizer supplemented with 500 IU/mL of IL-2 and 5 μmol/L zoledronic acid and lentivirus/retrovirus at desired TU/mL in the presence of transduction enhancers and replate in the same T-75 flask. Culture cells at 37 C/5% CO2 tilting flask (propped) to concentrate cell culture to bottom half of T-75 flask.

Day 4. This step includes the following protocol. Perform a second round of transduction at same TU/mL by removing half of the cells and transferring to a 50 mL conical tube. Centrifuge cells at 250×g 10 min RT. Bring cells to a final density of 1.5×10⁶ cells/mL in complete OpTmizer supplemented with 500 IU/mL of IL-2 and 5 μmol/L zoledronic acid and lentivirus/retrovirus at desired TU/mL in the presence of transduction enhancers and replate in the same T-75 flask. Culture cells at 37 C/5% CO2 tilting flask (propped) to concentrate cell culture to bottom half of T-75 flask.

Day 5. This step includes the following protocol. Transfer cells to 50 mL conical tube. Centrifuge cells at 250×g 10 min RT. Resuspend cells in complete OpTmizer supplemented with 500 IU/mL of IL-2 and 5 μmol/L zoledronic acid and replating in the same T-75 flask. Culture cells at 37 C/5% CO2 tilting flask (propped) to concentrate cell culture to bottom half of T-75 flask.

Day 6. Step 4: αβ T cell depletion (Day 6). This step includes the following protocol. Transfer cells to sterile container. Count cells. Remove 5e6 cells and save for flow immunotyping. Proceed with CliniMACS Plus depletion of αβ T cells. Step 5: Seed Bioreactor 500M-CS with gdT cells (Day 6). This step includes the following protocol. Resuspend cells in OpTmizer media. Count cells. Add 2e9 total live cells to each Bioreactor 500M-CS device containing 2.5 L of complete OpTmizer media supplemented 1000 IU/mL of IL-2. Culture cells at 37 C/5% CO2.

Day 9 Step 6: IL-2 supplementation (Day 9). This step includes the following protocol. Add 1000 U/ml IL-2 to each Bioreactor device through the needle port/tubing on the top of the device. Return GRex devices to 37 C/5% CO2 incubator.

Day 11. AAV Transduction. This step includes the following protocol. Reduce media in GRex devices by ½ to ¾ of culture volume. Add desired AAV VG/cell supplemented with desired transduction enhancers directly into the GRex. Return GRex devices to 37 C/5% CO2 incubator.

Day 12. Step 7: Harvest cells and cryopreserve (Day 12). This step includes the following protocol. Remove top 2 L (80%) of media from each Bioreactor device without disturbing the cell layer on the Bioreactor membrane using the Gather-Rex pump. Recover cells in remaining 500 ml media and transfer cells to a 500 ml centrifuge bottle. Remove a sample of the cells for immunotyping analysis and count live cell numbers and determine viability by trypan blue. Centrifuge cells at 300×g 20 min RT. Resuspend cells in cryopreservation solution (PBS+5% HSA+10% DMSO) at 10e6 cells/ml. Distribute cells into cryobags. Load bags into control rate freezer and run specified freezing program. When freezing program is complete move cryovials to liquid nitrogen for storage.

mRNA electroporation. This step includes the following protocol. After Step 7 #4 (centrifugation), resuspend cells in electroporation buffer and add mRNA. Electroporate cells using appropriate instrument settings. Cells are either returned to complete media for 2 hrs for incubation at 37 C/5% CO2 prior to, or immediately resuspended in cryomedia for freezing as described in Step 7 #5.

### Examples

Example 1. Gamma Delta T cells were thawed. Cells were incubated for 2 hours at 37 C in complete media. Cells (1e7) were electroporated with 15 ug mRNA. 24 hours later, set up 697 cell cytotoxicity assay with gdT cells+50% conditioned media in assay tube.

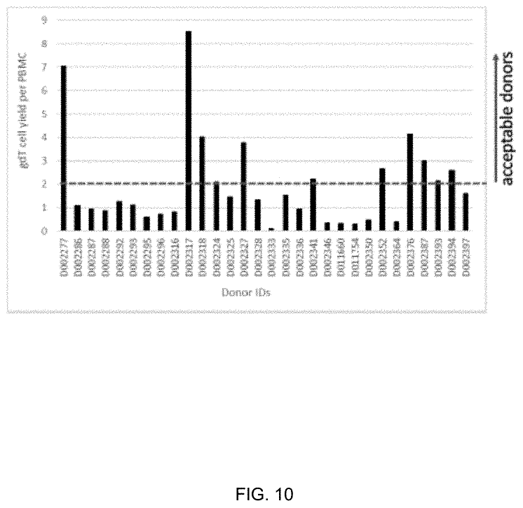

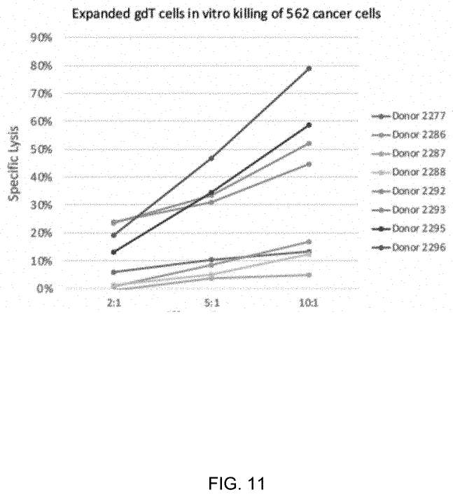

FIGS. 9-11 show experimental results. FIG. 9 is an overview of gdT cell expansion process. FIG. 10 shows identification of donors with acceptable ex vivo expansion of gdT cells from peripheral blood mononuclear cells (PBMCs). FIG. 11 shows screening of ex vivo expanded gdT cells to identify donors that generate gdT cells with high cytotoxicity toward K562 human cancer cells.

Protocol includes:

Purpose: Test the cytotoxicity of gdT cells transfected with CD19-CD3 STAR mRNA toward 697 cells, Nalm6 cells and 697-CD19KO cells.

Experimental Outline: Thawed gdT cells transfected with CD19-CD3 STAR mRNA=˜24 hrs later cytotox with 697 and Nalm6 cells

Materials:

Thawed day 12 gdT cells from 2 ARC2387 20e6 vials

HSA (25%) from Grifols

Sterile PBS

Nalm6 cells

697L cells

CD19_CD3 STAR mRNA

CTS OpTmizer T cell expansion kit, Life Technologies, Cat #A1048501
Need ˜25 ml complete gdT media (OpTmizer+supplement+glutamine+1000 IU/ml IL-2)

Combine 40 mL of OpTmizer T-cell basal expansion medium with 1.106 mL of OpTmizer supplement and add 400 μL of 200 mM L-Glutamine (Gibco, Cat. #16777-162).

Prepare freshly and store for only 1 week at 4 degrees.

IL-2 (1000 U/ul), stored at −80

Reconstitute in 250 μg of IL-2 (Peprotech, Cat. #AF-200-02) in 100 mM acetic acid, add 16.25 mL of sterile 1% BSA. Aliquot and store at −80 degrees for long-term storage (up to 1 year) and store at −20 degrees for short-term storage during expansion.

For 100 mM acetic acid, dissolve 14.4 μL of glacial acetic acid (Sigma, Cat. #A6283) in 2.486 mL of PBS (Gibco, cat. #14190250).

For 1% BSA, dissolve 200 mg of BSA (Sigma, Cat. #A9647) to a final volume of 20 mL in PBS.

CD19/CD3 bispecific antibody BPS Biosciences, item 100441-1, 0.82 mg/ml, for this experiment the ab was taken from a 10 ul aliquot frozen at −80. Dilute this stock 1:100 in OpTmizer media, Add 2.4 ul of ab to each 200 ul reaction tube to give a final concentration of 100 ng/ml.

OpTiMem media (Fisher cat #31-985-062)

4 mm cuvette

Violet Proliferation Dye (VPD450, BD Cat. 562158)

Procedure:

1 vial of 25e6 cells thawed into 5 ml of 5% HSA/PBS:

Prepare thawing media: 2 ml of 25% HSA into 8 ml PBS. Warm to 37 C.

take vial directly from −80 C to 37 C water bath to thaw

thaw until just a small chunk of ice remains.

Transfer cells to 15m conical tube.

Add 1 ml of prewarmed thawing media to the cells slowly (dropwise over 30 sec) while mixing.

Add an additional 3 ml of thawing media

spin 250×g 5 min RT

Resuspended cells in 8 ml OpTmizer+15.1 ug/ml IL2 in a T25 flask.

Incubated in TC incubator 2 hrs.

2 hrs after thaw collect cells and disperse into single cells by pipetting up/down.

Count cells and determine viability

Electroporation:

Prewarm and equilibrate 3 ml Optimizer complete media containing 1000 IU/ml IL-2 in each well of a 6 well plate.

Centrifuge gdT cells counted in step 8 above 250×g 5 min RT.

Resuspend cells to 10e6/ml in Optimem media

Add 1 ml of cells (=10e6 cells) to each of 2 1.5 ml epi tubes.

Centrifuged epi tubes noted above 250×g 5 min, RT

Remove supernate completely

Resuspend cells in 100 ul Optimem. Pipette cells up/down 3-5 times to break up cell pellet.

For the CD19-CD3 STAR group—Add 10 ul mRNA (=15 ug). Mix and put the cell/mRNA mixture into a 4 mm cuvette. (loaded the entire mixture, ˜130 ul)

Electroporate using the following settings:

Square wave

500V

5 ms pulse length

1 pulse

4 mm Cuvette

Add ˜500 ul warmed media (from 6 well plate) to cuvette and transfer media/cells back to 6 well plate.

For untransfected group, used 3 ml media from 6 well to resuspend cells in epi tube and transferred to 6 well plate.

TC Incubator o/n.

Cytotoxicity Assay

Staining Target Cells (697 Cells and Nalm6 Cells)

Place 5e6 target cells into a 15 ml conical tube and spin down.

Wash cells 2× with PBS to remove any serum proteins. Remove ⅕^{th} of cells for unstained control cells.

Prepare VPD450: Add 1 μL of 1 mM VPD450 stock to 1 ml PBS and mix.

Resuspend cells with 500 ul VPD450/PBS. Resuspend unstained control cells in PBS.

Incubate for 10 min in 37° C. water bath

Add 9 ml PBS and spin cells down 300×g 5 min.

Resuspend in 10 ml PBS and spin cells down 300×g 5 min.

Resuspend cells in 2 ml complete ptimizer+1000 U/ml IL2.

Count live cells using trypan blue.

Add enough additional OpTmizer+IL2 to bring cell concentration to 1e6 cells/ml.

Incubate target and effector cells in a total volume of 200 μL in flow tubes per table below:

Preparation of gdT Cells

Collect gdT cells and conditioned media from the multiwell plate and put into a 15 ml conical tube.

Wash flask with PBS and collect this in a separate 15 ml conical tube.

Spin 300×g 5 min RT.

Remove the conditioned media from the cell pellet and transfer to fresh conical tube.

Spin the conditioned media 500×g for 10 min to remove and cell debri.

Filter conditioned media with a 0.45 um filter attached to a 5 ml syringe.

Store the filtered conditioned media at −20 C.

Resuspend and pool the cell pellets (from the cell pellet under the conditioned media and the PBS wash of the well). Resuspend in 300 ul of OpTmizer (a volume small enough to ensure >5e6 cells/ml)

break up cell aggregates by pipetting up/down with a P1000 pipette.

Take 10 ul of the cells and dilute 1:10 by adding to 90 ul of PBS.

Count this 1:10 dilution of cells with trypan blue

Add enough OpTmizer media to bring the cell concentration of each group to 5e6 cells/ml.

Flow Cytometry Analysis of Cell Killing

Incubate target and effector cells for 4 hours at 37 degrees in flow tubes covered in aluminum foil.

Setup comp tubes! See below.

Wash cells with 1 mL of FACS buffer.

Wash cells 1× with 500 ul AnnexinV binding buffer.

resuspend in 100 μL of 1× ANNEXIN V BINDING BUFFER containing 2.5 ul Annexin V-APC for 20 min at RT.

Add 3 ul 7-ADD per tube and incubate 10 min longer.

Add 1 ml Annexin V binding buffer per r×n to wash.

spin down 300×g 5 min.

Decant and resuspend in 200 ul AnnexinV binding buffer (NOT FACS buffer!)

Run flow to determine % apoptotic cells and dead target cells by gating on VPD450 positive cells.

Compensation Tubes:

Comp1: Unstained—target cells that have not been stained with VPD450.

Comp2: VPD450 stained target cells (mix of 1:1 stained and unstained target cells).

Comp3: 7-ADD stained target cells (mix 1:1 of live and dead target cells).

Comp4: Annexin V-APC stained target cells (1:1 of live and dead target cells).

Sequence Product Listings

SEQ ID NO: 1 is the sequence name for IL2 CD19 CD3 STAR. It is a Complete STAR construct. It is an Amino Acid sequence. The sequence is:

MYRMQLLSCIALSLALVTNSDIQLTQSPASLAVSL
GQRATISCKASQSVDYDGDSYLNWYQQIPGQPPKL
LIYDASNLVSGIPPRFSGSGSGTDFTLNIHPVEKV
DAATYHCQQSTEDPWTFGGGTKLEIKGGGGSGGGG
SGGGGSQVQLQQSGAELVRPGSSVKISCKASGYAF
SSYWMNWVKQRPGQGLEWIGQIWPGDGDTNYNGKF
KGKATLTADESSSTAYMQLSSLASEDSAVYFCARR
ETTTVGRYYYAMDYWGQGTTVTVSSGGGGSDIKLQ
QSGAELARPGASVKMSCKTSGYTFTRYTMHWVKQR
PGQGLEWIGYINPSRGYTNYNQKFKDKATLTTDKS
SSTAYMQLSSLTSEDSAVYYCARYYDDHYCLDYWG
QGTTLTVSSVEGGSGGSGGSGGSGGVDDIQLTQSP
AIMSASPGEKVTMTCRASSSVSYMNWYQQKSGTSP
KRWIYDTSKVASGVPYRFSGSGSGTSYSLTISSME
AEDAATYYCQQWSSNPLTFGAGTKLELK.

SEQ ID NO: 1 includes Signal Peptide: 1-20; Target scFv: 21-270; Target VL: 21-131; Target linker: 132-146; Target VH: 147-270; Central Linker: 271-275; gdT scFv: 276-518; gdT VH: 397-410; gdT Linker: 397-410; gdT VL: 411-518.

SEQ ID NO: 2 is the sequence name for IL2 CD19 CD3 STAR. It is a Complete STAR construct. It is a DNA sequence. The sequence is:

ATGTACAGGATGCAGCTGCTGTCCTGCATTGCCCT
GTCCCTGGCCCTGGTGACCAACTCCGACATTCAAT
TGACACAAAGCCCGGCAAGCCTGGCTGTGTCCCTG
GGCCAGCGGGCGACTATTTCATGTAAAGCAAGTCA
GAGCGTAGACTACGATGGTGATAGCTATCTGAACT
GGTATCAGCAGATTCCAGGACAACCTCCCAAATTG
CTGATTTACGACGCCTCGAACCTGGTCAGCGGGAT
TCCACCACGATTCTCTGGAAGCGGCAGTGGAACCG
ATTTTACGCTGAATATACACCCAGTGGAAAAGGTG
GACGCTGCGACTTATCATTGTCAGCAGTCTACCGA
GGACCCATGGACCTTCGGCGGGGGAACAAAGCTGG
AGATCAAGGGTGGTGGTGGAAGCGGTGGTGGTGGA
TCTGGCGGTGGTGGAAGTCAGGTGCAGCTTCAGCA
GTCCGGAGCCGAACTGGTGCGCCCCGGCAGTAGCG
TGAAGATAAGCTGCAAGGCTAGCGGATACGCTTTC
TCTTCCTACTGGATGAACTGGGTGAAGCAGCGCCC
TGGCCAAGGGCTTGAGTGGATCGGCCAGATATGGC
CTGGTGATGGTGACACCAACTATAATGGCAAGTTT
AAGGGAAAGGCTACACTCACTGCCGATGAAAGTTC
ATCCACTGCCTACATGCAGCTCTCCAGTCTGGCAA
GTGAAGACTCTGCTGTCTATTTCTGCGCTAGACGA
GAGACTACAACTGTCGGGAGGTACTACTATGCAAT
GGATTACTGGGGTCAGGGAACCACAGTGACAGTGA
GCTCAGGAGGTGGTGGGTCTGACATTAAGCTACAA
CAGTCTGGCGCCGAGTTGGCCAGGCCTGGGGCCAG
CGTGAAGATGTCTTGCAAGACCTCAGGTTATACCT
TCACGAGATACACCATGCATTGGGTAAAACAGAGA
CCTGGGCAAGGCTTGGAATGGATCGGCTACATCAA
CCCCAGTCGCGGATACACAAACTACAATCAGAAAT
TTAAAGACAAGGCGACTCTCACCACAGATAAGTCC
TCCTCTACCGCCTACATGCAGCTGTCATCTCTCAC
AAGCGAAGACTCTGCCGTGTATTATTGCGCAAGGT
ATTACGATGACCACTATTGTTTAGATTATTGGGGG
CAAGGAACTACACTCACTGTCAGCTCAGTTGAGGG
TGGAAGTGGGGGATCTGGTGGTTCAGGCGGATCTG
GTGGGGTCGACGACATCCAGCTGACCCAGAGTCCC
GCCATCATGTCAGCTAGTCCCGGGGAGAAAGTGAC
TATGACATGCAGGGCATCTAGCAGCGTTTCGTACA
TGAATTGGTATCAGCAGAAATCAGGTACCTCCCCG
AAACGTTGGATTTATGACACGAGCAAGGTTGCAAG
CGGTGTTCCATACCGGTTTTCGGGCTCCGGTTCCG
GCACCTCCTACTCTCTCACGATCTCCAGTATGGAA
GCCGAGGATGCCGCAACCTATTACTGTCAGCAATG
GTCATCCAATCCCTTAACCTTCGGAGCCGGAACAA
AACTGGAGCTCAAA.

SEQ ID NO: 2 includes Signal Peptide: 1-60; Target scFv: 61-810; Target VL: 61-393; Target linker: 394-438; Target VH: 439-810; Central Linker: 811-825; gdT scFv: 826-1544; gdT VH: 1189-1230; gdT Linker: 1189-1230; gdT VL: 1231-1544.

SEQ ID NO: 3 is the sequence name for modified serum albumin (mSA). It is a Signal Peptide construct. It is an AA sequence. The sequence is: MKWVTFISLLFLFSSSSRA.

SEQ ID NO: 3 includes Signal Peptide: 1-19.

SEQ ID NO: 4 is the sequence name for human stem cell factor (hSCF). It is a Signal Peptide construct. It is an AA sequence. The sequence is: MKKTQTWILTCIYLQLLLFNPLVKT.

SEQ ID NO: 4 includes Signal Peptide: 1-25.

SEQ ID NO: 5 is the sequence name for modified serum albumin (mSA). It is a Signal Peptide construct. It is a DNA sequence. The sequence is:

ATGAAATGGGTTACTTTTATTAGTTTATTATTCCT
GTTCAGCTCCAGCTCCAGGGCC.

SEQ ID NO: 5 includes Signal Peptide: 1-57.

SEQ ID NO: 6 is the sequence name for human stem cell factor (hSCF). It is a Signal Peptide construct. It is a DNA sequence. The sequence is:

ATGAAGAAAACTCAAACTTGGATACTAACTTGCAT
CTACCTGCAGCTGCTGCTCTTCAACCCCTTGGTGA
AGACG.

SEQ ID NO: 6 includes Signal Peptide: 1-75.

SEQ ID NO: 7 is the sequence name for CD19 scFv ECOg (154). It is a Tumor Targeting scFv construct. It is a DNA sequence. The sequence is:

GACATCCAGCTCACCCAGAGCCCCGCATCCCTGGC
CGTGTCCCTGGGGCAGCGCGCAACCATCTCCTGCA
AGGCTTCCCAGTCCGTGGACTACGACGGGGACTCC
TACCTGAACTGGTACCAGCAGATCCCCGGGCAGCC
ACCGAAGCTGCTGATCTACGACGCCTCCAACCTGG
TGTCCGGGATTCCTCCGCGGTTCTCCGGGAGCGGG
TCCGGGACCGACTTCACCCTGAACATCCATCCCGT
GGAGAAGGTGGACGCCGCCACCTACCACTGCCAGC
AGTCCACCGAGGACCCCTGGACCTTCGGCGGCGGC
ACCAAGCTGGAGATCAAAGGTGGCGGAGGCTCCGG
TGGCGGAGGTTCCGGTGGCGGCGGCTCCCAGGTGC
AGCTGCAGCAGTCAGGGGCCGAGCTGGTGAGGCCC
GGGAGCTCCGTGAAGATCTCCTGCAAGGCCTCCGG
GTACGCCTTCTCCTCCTACTGGATGAACTGGGTGA
AGCAGAGGCCCGGGCAGGGGCTGGAGTGGATCGGG
CAGATCTGGCCCGGGGACGGGGACACCAACTACAA
CGGGAAGTTCAAGGGGAAGGCTACCCTCACCGCCG
ACGAGAGCTCCTCCACCGCCTACATGCAGCTGAGC
TCCCTCGCCTCCGAGGACTCCGCCGTGTACTTCTG
CGCCCGGCGCGAGACCACCACCGTGGGGCGCTACT
ACTACGCCATGGACTACTGGGGCCAGGGGACCACC
GTGACCGTGAGCTCC.

SEQ ID NO: 7 includes Target scFv: 1-750; Target VL: 1-333; Target linker: 334-378; Target VH: 379-750; Free energy: −345.2; gdT CAI: 0.89467859593316; ORF count: 1.

SEQ ID NO: 8 is the sequence name for CD19 scFv ECOg (94). It is a Tumor Targeting scFv construct. It is a DNA sequence. The sequence is:

GACATCCAGCTGACCCAGTCGCCCGCTAGCCTGGC
GGTCTCGCTGGGCCAGCGAGCCACCATCAGCTGCA
AGGCCAGCCAGTCGGTCGACTACGACGGCGACAGC
TACCTCAACTGGTACCAGCAGATCCCCGGCCAGCC
ACCGAAGCTGCTGATCTACGACGCCAGCAACCTGG
TGAGCGGGATACCACCGCGGTTCTCGGGGTCGGGG
TCGGGGACCGACTTCACCCTCAACATCCATCCCGT
CGAGAAGGTCGACGCCGCCACCTACCACTGCCAGC
AGTCGACCGAGGACCCCTGGACCTTCGGCGGCGGC
ACCAAGCTGGAGATCAAAGGTGGCGGTGGCTCCGG
TGGCGGTGGTAGCGGTGGCGGCGGCTCCCAGGTCC
AGCTCCAGCAGTCTGGGGCCGAGCTGGTGCGACCC
GGGAGCTCGGTCAAGATCAGCTGCAAGGCCAGCGG
CTACGCCTTCTCCAGCTACTGGATGAACTGGGTCA
AGCAGCGACCCGGCCAGGGGCTGGAGTGGATCGGC
CAGATCTGGCCCGGCGACGGCGACACCAACTACAA
CGGCAAGTTCAAGGGCAAGGCCACGCTGACCGCCG
ACGAGTCGAGCTCGACCGCCTACATGCAGCTGAGC
TCGCTCGCCAGCGAGGACTCCGCGGTCTACTTCTG
CGCCCGGCGCGAGACCACCACCGTGGGGCGCTACT
ACTACGCGATGGACTACTGGGGCCAGGGGACCACG
GTGACCGTGAGCTCG

SEQ ID NO: 8 includes Target scFv: 1-750; Target VL: 1-333; Target linker: 334-378; Target VH: 379-750; Free energy: −360.7; gdT CAI: 0.767359962199379; ORF count: 2.

SEQ ID NO: 9 is the sequence name for CD19 scFv ECOg (139). It is a Tumor Targeting scFv construct. It is a DNA sequence. The sequence is:

GACATCCAGCTGACGCAGTCGCCCGCGTCGCTCGC
CGTGTCGCTCGGGCAACGCGCGACGATCTCGTGCA
AGGCGTCGCAGTCCGTCGACTACGACGGCGACTCG
TACCTGAACTGGTACCAGCAGATCCCCGGGCAGCC
GCCGAAGCTGCTGATCTACGACGCGAGCAACCTCG
TGTCCGGCATTCCGCCGCGGTTCAGCGGCAGCGGC
AGCGGCACCGACTTCACGCTGAACATTCACCCCGT
CGAGAAGGTCGACGCCGCGACGTACCACTGCCAGC
AGTCCACCGAGGACCCGTGGACGTTCGGCGGCGGC
ACGAAGCTGGAGATCAAAGGTGGCGGCGGTTCCGG
TGGCGGTGGTTCCGGTGGCGGCGGCTCGCAGGTGC
AGCTGCAGCAGTCTGGCGCCGAGCTCGTGCGACCC
GGGTCGTCCGTGAAGATCTCGTGCAAGGCGTCCGG
GTACGCATTCTCGTCGTACTGGATGAACTGGGTGA
AGCAGCGACCCGGGCAGGGGCTGGAGTGGATCGGG
CAGATCTGGCCCGGCGACGGCGACACGAACTACAA
CGGCAAGTTCAAGGGCAAGGCGACGCTGACCGCCG
ACGAGTCGTCGTCCACCGCGTACATGCAGCTGTCG
TCGCTCGCGAGCGAGGACAGCGCCGTGTACTTCTG
CGCGCGGCGCGAGACGACGACCGTCGGGCGCTACT
ACTACGCGATGGACTACTGGGGCCAGGGCACGACC
GTGACCGTGTCCAGC.

SEQ ID NO: 9 includes Target scFv: 1-750; Target VL: 1-333; Target linker: 334-378; Target VH: 379-750; Free energy: −357.7; gdT CAI: 0.724573751544555; ORF count: 1.

SEQ ID NO: 10 is the sequence name for CD19 scFv ECOg (195). It is a Tumor Targeting scFv construct. It is a DNA sequence. The sequence is:

GACATCCAGCTGACCCAGAGCCCCGCGAGCCTGGC
CGTGAGCCTGGGGCAGAGGGCCACCATCAGCTGCA
AGGCGTCCCAGAGCGTGGACTACGACGGGGACAGC
TACCTGAACTGGTACCAGCAGATCCCCGGGCAGCC
GCCGAAGCTGCTGATCTACGACGCCTCCAACCTGG
TGTCCGGGATACCGCCGCGGTTCTCCGGGTCCGGG
TCCGGGACCGACTTCACCCTGAACATTCACCCCGT
GGAGAAGGTGGACGCCGCCACCTACCACTGCCAGC
AGAGCACCGAGGACCCCTGGACCTTCGGCGGCGGC
ACCAAGCTGGAGATCAAAGGTGGCGGAGGCTCCGG
TGGCGGTGGTAGCGGTGGCGGCGGCTCCCAGGTGC
AGCTGCAGCAGTCTGGGGCCGAGCTGGTGCGACCC
GGGTCCAGCGTGAAGATCAGCTGCAAGGCCAGCGG
GTACGCCTTCAGCTCCTACTGGATGAACTGGGTGA
AGCAGAGGCCCGGGCAGGGGCTGGAGTGGATCGGG
CAGATCTGGCCCGGGGACGGGGACACCAACTACAA
CGGGAAGTTCAAGGGGAAGGCCACGCTGACCGCCG
ACGAGAGCAGCTCCACCGCCTACATGCAGCTGTCC
AGCCTGGCCTCCGAGGACAGCGCCGTGTACTTCTG
CGCCCGGCGCGAGACCACCACCGTGGGGCGCTACT
ACTACGCCATGGACTACTGGGGCCAGGGGACCACC
GTGACCGTGAGCTCC.

SEQ ID NO: 10 includes Target scFv: 1-750; Target VL: 1-333; Target linker: 334-378; Target VH: 379-750; Free energy: −356; gdT CAI: 0.858340915819879; ORF count: 2.

SEQ ID NO: 11 is the sequence name for CD19 scFv ECOg (160). It is a Tumor Targeting scFv construct. It is a DNA sequence. The sequence is:

GACATCCAGCTGACCCAGTCACCCGCTAGCCTGGC
CGTGTCCCTGGGCCAGCGAGCCACGATCTCCTGCA
AGGCCAGCCAGTCCGTGGACTACGACGGGGACTCC
TACCTCAACTGGTACCAGCAGATCCCCGGCCAGCC
ACCGAAGCTGCTGATCTACGACGCCTCCAACCTGG
TGAGCGGGATTCCGCCGCGGTTCAGCGGGTCCGGG
TCCGGGACCGACTTCACCCTCAACATCCATCCCGT
GGAGAAGGTGGACGCCGCCACCTACCACTGCCAGC
AGTCCACCGAGGACCCCTGGACCTTCGGCGGCGGC
ACCAAGCTGGAGATCAAAGGCGGTGGTGGCTCCGG
AGGCGGTGGCTCTGGTGGCGGCGGCTCCCAGGTGC
AGCTCCAGCAGAGCGGGGCTGAGCTGGTGAGGCCC
GGGTCCTCCGTGAAGATCTCCTGCAAGGCCTCCGG
GTACGCCTTCTCCAGCTACTGGATGAACTGGGTGA
AGCAGAGGCCCGGGCAGGGGCTGGAGTGGATCGGG
CAGATCTGGCCCGGGGACGGGGACACCAACTACAA
CGGGAAGTTCAAGGGGAAGGCCACGCTGACCGCCG
ACGAGAGCAGCTCCACCGCCTACATGCAGCTGTCC
AGCCTGGCCTCCGAGGACTCCGCCGTGTACTTCTG
CGCCCGGCGCGAGACCACCACCGTGGGGCGCTACT
ACTACGCCATGGACTACTGGGGCCAGGGGACCACC
GTGACCGTGTCCAGC.

SEQ ID NO: 11 includes Target scFv: 1-750; Target VL: 1-333; Target linker: 334-378; Target VH: 379-750; Free energy: −354.8; gdT CAI: 0.876843948859594; ORF count: 2.

SEQ ID NO: 12 is the sequence name for CD3 scFv ECOg (70). It is a gdT Targeting scFv construct. It is a DNA sequence. The sequence is:

GATATCAAGCTCCAGCAGTCCGGGGCTGAGCTGGC
TAGGCCCGGGGCCTCCGTGAAGATGTCCTGCAAGA
CCTCCGGGTACACCTTCACCAGGTACACCATGCAC
TGGGTGAAGCAGAGGCCCGGGCAGGGGCTGGAGTG
GATCGGGTACATCAACCCCAGCCGCGGGTACACCA
ACTACAACCAGAAGTTCAAGGACAAGGCCACCTTG
ACCACCGACAAGTCCTCCTCCACCGCCTACATGCA
GCTGAGCTCCCTGACCTCCGAGGACTCCGCCGTCT
ACTACTGCGCCAGGTACTACGACGACCACTACTGC
CTGGACTACTGGGGCCAGGGGACTACCCTGACCGT
GAGCTCCGTGGAAGGTGGCTCCGGCGGCTCCGGAG
GTTCCGGAGGCTCCGGCGGCGTGGACGACATCCAG
CTGACCCAGTCCCCAGCTATCATGTCCGCCAGCCC
CGGGGAGAAGGTGACCATGACCTGCCGGGCCTCCT
CCTCCGTGAGCTACATGAACTGGTACCAGCAGAAG
TCCGGGACCTCTCCCAAGAGGTGGATCTACGACAC
CTCCAAGGTGGCCTCCGGGGTCCCCTACAGGTTCT
CCGGGTCCGGGTCCGGGACCTCCTACTCCCTGACC
ATCTCCTCCATGGAGGCCGAGGACGCCGCCACCTA
CTACTGCCAGCAGTGGAGCTCCAATCCCCTGACCT
TCGGGGCCGGGACCAAGCTGGAGCTGAAG.

SEQ ID NO: 12 includes gdT scFv: 1-729; gdT VH: 364-405; gdT Linker: 364-405; gdT VL: 406-729; Free energy: −320.1; gdT CAI: 0.924743; ORF count: 1.

SEQ ID NO: 13 is the sequence name for CD3 scFv ECOg (197). It is a gdT Targeting scFv construct. It is a DNA sequence. The sequence is:

GACATCAAGCTCCAGCAGTCCGGGGCTGAGCTTGC
TCGCCCCGGGGCCAGCGTGAAGATGTCCTGCAAGA
CCTCGGGGTACACCTTCACCAGGTACACCATGCAC
TGGGTGAAGCAGCGCCCAGGGCAGGGCCTGGAGTG
GATAGGGTACATCAACCCCAGCCGCGGGTACACAA
ACTACAACCAGAAGTTCAAGGACAAGGCCACGCTG
ACCACGGACAAGTCCTCGTCCACGGCGTACATGCA
GCTGTCCTCGCTGACCTCCGAGGACAGCGCGGTGT
ACTACTGCGCGCGGTACTACGACGACCACTACTGC
CTGGACTACTGGGGCCAGGGGACAACGCTGACCGT
GAGCAGCGTGGAAGGCGGCAGCGGCGGCAGCGGAG
GTAGCGGAGGCTCCGGCGGCGTGGACGACATCCAG
CTCACCCAGAGCCCGGCGATCATGTCCGCCAGCCC
CGGGGAGAAGGTGACCATGACCTGCCGGGCGTCGT
CCTCGGTGAGCTACATGAACTGGTACCAGCAGAAG
TCCGGGACCTCGCCCAAGCGCTGGATCTACGACAC
CAGCAAGGTGGCCTCCGGGGTGCCCTACCGCTTCT
CCGGGTCGGGGTCGGGGACATCGTACTCCCTGACC
ATCTCCAGCATGGAGGCCGAGGACGCCGCGACGTA
CTACTGCCAGCAGTGGTCGTCCAACCCGCTGACCT
TCGGCGCGGGGACAAAGCTGGAGCTTAAG.

SEQ ID NO: 13 includes gdT scFv: 1-729; gdT VH: 364-405; gdT Linker: 364-405; gdT VL: 406-729; Free energy: −341.1; gdT CAI: 0.803206301402276; ORF count: 2.

SEQ ID NO: 14 is the sequence name for CD3 scFv ECOg (109). It is a gdT Targeting scFv construct. It is a DNA sequence. The sequence is:

GACATCAAGCTGCAGCAGAGCGGCGCTGAGCTCGC
GCGACCAGGCGCGAGCGTGAAGATGAGCTGCAAGA
CGAGCGGCTACACGTTCACGCGCTACACGATGCAC
TGGGTGAAGCAGCGTCCCGGGCAGGGGCTGGAGTG
GATCGGCTACATCAACCCGTCGCGCGGCTACACGA
ACTACAACCAGAAGTTCAAGGACAAGGCGACGCTG
ACGACCGACAAGAGCAGCAGCACCGCGTACATGCA
GCTGAGCTCGCTGACGAGCGAGGACAGCGCCGTGT
ACTACTGCGCGCGCTACTACGACGACCACTACTGC
CTCGACTACTGGGGCCAGGGCACGACGCTGACCGT
GAGCAGCGTCGAAGGCGGCAGCGGCGGCAGCGGTG
GAAGCGGTGGCAGCGGCGGCGTCGACGACATCCAG
CTGACGCAGTCGCCCGCGATCATGAGCGCGTCGCC
CGGCGAGAAGGTGACGATGACGTGCCGCGCGAGCA
GCAGCGTGTCGTACATGAACTGGTACCAGCAGAAG
AGCGGCACGTCGCCGAAGCGCTGGATCTACGACAC
GTCGAAGGTCGCGAGCGGCGTGCCGTACCGCTTCA
GCGGCAGCGGCAGCGGCACGTCGTACTCGCTGACG
ATCAGCAGCATGGAGGCCGAGGACGCCGCGACGTA
CTACTGCCAGCAGTGGAGCTCGAACCCGCTGACGT
TCGGCGCCGGCACGAAGCTGGAGCTGAAG.

SEQ ID NO: 14 includes gdT scFv: 1-729; gdT VH: 364-405; gdT Linker: 364-405; gdT VL: 406-729; Free energy: −334.7; gdT CAI: 0.70167404462703; ORF count: 1.

SEQ ID NO: 15 is the sequence name for CD3 scFv ECOg (119). It is a gdT Targeting scFv construct. It is a DNA sequence. The sequence is:

GACATCAAGCTGCAGCAGAGCGGCGCGGAATTAGC
GCGCCCCGGGGCGTCCGTCAAGATGTCCTGCAAGA
CCTCCGGGTACACCTTCACGCGGTACACCATGCAC
TGGGTGAAGCAACGCCCCGGGCAGGGCCTGGAGTG
GATCGGGTACATCAACCCGTCGCGGGGCTACACGA
ACTACAACCAGAAGTTCAAGGACAAGGCGACCCTC
ACGACCGACAAGTCGAGCAGCACGGCGTACATGCA
GCTCTCCTCGCTGACCAGCGAGGACTCCGCGGTGT
ACTACTGCGCGCGGTACTACGACGACCACTACTGC
CTGGACTACTGGGGCCAGGGCACCACGCTGACCGT
CAGCTCGGTGGAAGGCGGCTCCGGCGGCTCCGGAG
GTTCCGGAGGTTCCGGCGGAGTGGACGACATCCAG
CTCACCCAGAGCCCCGCGATTATGAGCGCCTCGCC
CGGCGAGAAGGTCACCATGACCTGCCGGGCGAGCA
GCTCGGTGAGCTACATGAACTGGTACCAGCAGAAG
AGCGGCACGTCGCCCAAGCGGTGGATCTACGATAC
CAGCAAGGTGGCGAGCGGCGTGCCGTACCGCTTCT
CGGGGAGCGGGTCCGGCACGTCGTACAGCCTGACG
ATCAGCAGCATGGAGGCGGAGGACGCCGCGACGTA
CTACTGCCAGCAGTGGAGCAGCAACCCGCTGACCT
TCGGGGCCGGCACGAAGCTCGAACTGAAG.

SEQ ID NO: 15 includes gdT scFv: 1-729; gdT VH: 364-405; gdT Linker: 364-405; gdT VL: 406-729; Free energy: −328.1; gdT CAI: 0.739432850740138; ORF count: 0.

SEQ ID NO: 16 is the sequence name for CD3 scFv ECOg (45). It is a gdT Targeting scFv construct. It is a DNA sequence. The sequence is:

GACATCAAGCTGCAGCAGTCCGGAGCCGAGTTGGC
ACGGCCCGGGGCCTCCGTGAAGATGTCCTGCAAGA
CGTCCGGGTACACGTTCACCCGGTACACCATGCAC
TGGGTGAAGCAGCGGCCCGGGCAGGGGCTGGAATG
GATCGGGTACATCAACCCCAGCCGCGGGTACACCA
ACTACAACCAGAAGTTCAAGGACAAGGCCACGCTG
ACCACCGACAAGTCGTCGTCGACCGCCTACATGCA
GCTGAGCTCGCTGACCAGCGAGGACAGCGCCGTCT
ACTACTGCGCCCGCTACTACGACGACCACTACTGC
CTGGACTACTGGGGCCAGGGGACCACGCTGACAGT
GAGCAGCGTGGAAGGCGGCTCCGGCGGCTCCGGAG
GTTCCGGAGGCAGCGGCGGCGTCGACGACATCCAG
CTCACCCAGTCCCCGGCCATCATGTCGGCGAGCCC
CGGCGAGAAGGTGACCATGACGTGCCGGGCCTCCA
GCTCGGTGTCCTACATGAACTGGTACCAGCAGAAG
TCGGGGACCAGCCCCAAGCGGTGGATCTACGACAC
CAGCAAGGTGGCCAGCGGGGTCCCCTACCGCTTCT
CGGGGTCCGGGTCCGGGACCTCGTACTCGCTGACC
ATCTCCAGCATGGAGGCCGAGGACGCCGCCACCTA
CTACTGCCAGCAGTGGTCCTCGAACCCGCTCACCT
TCGGGGCCGGCACCAAGCTGGAGCTGAAG.

SEQ ID NO: 16 includes gdT scFv: 1-729; gdT VH: 364-405; gdT Linker: 364-405; gdT VL: 406-729; Free energy: −323.7; gdT CAI: 0.817020513172323; ORF count: 0.

SEQ ID NO: 17 is the sequence name for PTK7 scFv. It is a Tumor Targeting scFv construct. It is an AA sequence. The sequence is:

EVQLVQSGGGLVHPGGSLRLSCAGSGFTFSTYLMY
WVRQAPGKTLEWVSAIGSGGDTYYADSVKGRFTIS
RDNAKNSLYLQMNSLRAEDMAVYYCARGLGYWGQG
TLVTVSSGGGGSGGGGSGGGGSEIVLTQSPGTLSL
SPGERATLSCRASQSVSSSYLAWYQQKPGQAPRLL
IYGASSRATGIPDRFSGSGSGTDFTLTISRLEPED
FAVYYCQQYGSSPMYTFGQGTKLEIK.

SEQ ID NO: 17 includes Target scFv: 1-236; Target VL: 128-236; Target linker: 113-127; Target VH: 1-236.

SEQ ID NO: 18 is the sequence name for PTK7 scFv. It is a Tumor Targeting scFv construct. It is a DNA sequence. The sequence is:

GAGGTGCAGCTGGTGCAGTCCGGAGGAGGTTTAGT
TCACCCAGGAGGCTCGCTGCGACTGTCCTGTGCAG
GGAGCGGCTTTACCTTCAGCACATATCTGATGTAC
TGGGTGAGACAGGCCCCCGGCAAGACGCTGGAGTG
GGTCTCAGCTATCGGATCAGGTGGCGACACCTATT
ACGCTGATAGCGTGAAGGGCCGGTTTACCATAAGC
CGCGACAACGCCAAAAATAGTCTTTACCTGCAGAT
GAACAGCCTCCGAGCAGAGGATATGGCCGTCTATT
ACTGCGCTCGGGGACTCGGGTATTGGGGGCAGGGC
ACCTTGGTGACTGTCTCTTCAGGGGGAGGTGGTTC
AGGTGGTGGAGGTTCTGGCGGCGGCGGTTCCGAAA
TTGTACTAACCCAATCTCCTGGCACACTTAGTCTG
TCTCCTGGAGAACGTGCAACGTTGAGCTGCCGCGC
TAGCCAGTCCGTGTCTTCCTCGTACCTCGCCTGGT
ACCAGCAAAAGCCAGGGCAAGCTCCCAGGTTGCTG
ATTTACGGAGCCTCAAGTAGGGCGACTGGAATCCC
TGACAGATTCAGTGGGAGCGGGTCCGGGACTGATT
TTACTCTCACCATTTCTAGACTTGAGCCCGAAGAC
TTCGCCGTTTATTACTGTCAGCAGTATGGCTCTTC
CCCGATGTACACATTCGGCCAGGGCACAAAACTGG
AAATCAAG.

SEQ ID NO: 18 includes Target scFv: 1-708; Target VL: 382-708; Target linker: 337-381; Target VH: 1-336; Free energy: −268.3; gdT CAI: 0.722372746155158; ORF count: 6.

SEQ ID NO: 19 is the sequence name for PTK7 ECOg (74). It is a Tumor Targeting scFv construct. It is a DNA sequence. The sequence is:

GAGGTGCAGCTCGTGCAGTCCGGCGGAGGCCTCGT
GCACCCAGGCGGCTCCCTGCGCCTGTCCTGCGCCG
GGTCAGGCTTCACCTTCTCCACGTACCTCATGTAC
TGGGTGCGGCAGGCCCCAGGGAAGACCCTGGAGTG
GGTGTCCGCCATCGGGTCCGGCGGCGACACGTACT
ACGCCGACTCCGTGAAGGGGCGCTTCACCATCTCC
CGGGACAACGCCAAGAACTCCCTGTACCTGCAGAT
GAACTCCCTGCGGGCCGAGGACATGGCCGTGTACT
ACTGCGCCAGGGGCCTGGGGTACTGGGGCCAGGGG
ACCCTCGTGACCGTGTCCTCCGGCGGCGGAGGCTC
AGGAGGAGGAGGCTCAGGAGGCGGCGGCAGCGAGA
TCGTCCTGACGCAGAGCCCCGGGACCCTGTCCCTG
AGCCCCGGGGAGAGGGCGACCCTGTCCTGCCGGGC
CAGCCAGTCCGTGTCCTCCTCCTACCTGGCCTGGT
ACCAGCAGAAGCCCGGGCAAGCCCCACGCCTCCTC
ATCTACGGGGCTTCCTCCAGGGCCACGGGCATCCC
CGACCGCTTCTCCGGGTCCGGGTCCGGGACGGACT
TCACCCTGACCATCTCCCGGCTGGAGCCCGAGGAC
TTCGCCGTGTACTACTGCCAGCAGTACGGGTCCTC
GCCCATGTACACCTTCGGGCAGGGGACGAAGCTGG
AGATCAAG.

SEQ ID NO: 19 includes Target scFv: 1-708; Target VL: 382-708; Target linker: 337-381; Target VH: 1-336; Free energy: −336.8; gdT CAI: 0.861158; ORF count: 0.

SEQ ID NO: 20 is the sequence name for PTK7 ECOg (18). It is a Tumor Targeting scFv construct. It is a DNA sequence. The sequence is:

GAGGTCCAGCTGGTGCAGTCCGGCGGTGGCCTGGT
GCACCCAGGCGGCAGCCTGCGGCTGTCGTGCGCCG
GCAGTGGGTTCACCTTCTCCACCTACCTGATGTAC
TGGGTGCGCCAGGCCCCAGGGAAGACCCTGGAGTG
GGTGTCCGCCATCGGCTCCGGCGGCGACACCTACT
ACGCCGATAGCGTCAAGGGGCGCTTCACCATCTCC
CGCGACAACGCCAAGAACTCCCTCTACCTCCAGAT
GAACTCCCTGCGGGCCGAGGACATGGCCGTCTACT
ACTGCGCCAGGGGCCTGGGCTACTGGGGCCAGGGG
ACCCTGGTGACCGTGTCCTCCGGCGGCGGTGGCTC
TGGTGGTGGTGGCTCTGGTGGCGGCGGCAGCGAGA
TCGTGCTGACCCAGTCGCCCGGGACCCTGTCGCTG
TCGCCCGGCGAGCGAGCGACCCTCAGCTGCCGCGC
GAGCCAGAGCGTGAGCTCCAGCTACCTCGCGTGGT
ACCAGCAGAAGCCCGGGCAAGCCCCACGGCTGCTG
ATCTACGGCGCTAGCTCCAGGGCCACCGGCATCCC
CGATCGCTTCAGCGGCAGCGGCAGCGGGACCGACT
TCACCCTGACGATCTCCCGGCTGGAGCCCGAGGAC
TTCGCCGTCTACTACTGCCAGCAGTACGGCTCCAG
CCCCATGTACACCTTCGGCCAGGGGACCAAGCTGG
AGATCAAG.

SEQ ID NO: 20 includes Target scFv: 1-708; Target VL: 382-708; Target linker: 337-381; Target VH: 1-336; Free energy: −356; gdT CAI: 0.803927502492027; ORF count: 4.

SEQ ID NO: 21 is the sequence name for PTK7 ECOg (70). It is a Tumor Targeting scFv construct. It is a DNA sequence. The sequence is:

GAGGTGCAGCTGGTGCAGAGCGGCGGAGGCCTGGT
GCACCCAGGCGGCAGCCTGCGGCTGAGCTGCGCTG
GCTCCGGCTTCACCTTCAGCACCTACCTGATGTAC
TGGGTGCGGCAGGCCCCAGGCAAGACCCTGGAGTG
GGTGTCCGCCATCGGCAGCGGCGGCGACACCTACT
ACGCCGACAGCGTGAAGGGGCGCTTCACCATCAGC
CGGGACAACGCCAAGAACAGCCTGTACCTGCAGAT
GAACAGCCTGCGGGCCGAGGACATGGCCGTGTACT
ACTGCGCCAGGGGCCTGGGCTACTGGGGCCAGGGC
ACCCTGGTGACCGTGAGCAGCGGCGGCGGTGGCTC
TGGTGGAGGAGGCTCTGGTGGCGGCGGCTCCGAGA
TCGTGCTGACCCAGAGCCCCGGCACCCTGAGCCTG
AGCCCCGGGGAGCGGGCTACCCTGAGCTGCCGGGC
TTCCCAGAGCGTGAGCAGCTCCTACCTGGCCTGGT
ACCAGCAGAAGCCCGGGCAGGCACCACGGCTGCTG
ATCTACGGGGCTAGCAGCCGGGCTACCGGCATCCC
CGACCGCTTCTCCGGCAGCGGCAGCGGCACCGACT
TCACCCTGACCATCTCCAGGCTGGAGCCCGAGGAC
TTCGCCGTGTACTACTGCCAGCAGTACGGCAGCTC
TCCCATGTACACCTTCGGGCAGGGCACCAAGCTGG
AGATCAAG.

SEQ ID NO: 21 includes Target scFv: 1-708; Target VL: 382-708; Target linker: 337-381; Target VH: 1-336; Free energy: −355.6; gdT CAI: 0.830356865758289; ORF count: 3.

SEQ ID NO: 22 is the sequence name for PTK7 ECOg (68). It is a Tumor Targeting scFv construct. It is a DNA sequence. The sequence is:

GAGGTCCAGCTGGTCCAGTCCGGCGGCGGCCTGGT
TCATCCCGGCGGCAGCCTGCGGCTGAGCTGCGCCG
GGTCTGGCTTCACCTTCAGCACCTACCTGATGTAC
TGGGTCCGGCAGGCGCCCGGCAAGACCCTGGAGTG
GGTGTCGGCCATCGGGTCCGGCGGCGACACCTACT
ACGCCGACTCGGTCAAGGGCCGGTTCACCATCAGC
CGGGACAACGCCAAGAACAGCCTGTACCTGCAGAT
GAACAGCCTGCGGGCCGAGGACATGGCCGTCTACT
ACTGCGCCCGCGGGCTGGGTTACTGGGGCCAGGGG
ACCCTGGTGACCGTCAGCTCCGGCGGCGGCGGATC
TGGTGGTGGCGGATCTGGTGGCGGCGGCTCCGAGA
TCGTGCTGACCCAGTCGCCCGGGACCCTGAGCCTG
AGCCCCGGCGAGCGGGCTACCCTGAGCTGCCGGGC
CAGCCAGTCGGTCAGCTCCAGCTACCTGGCCTGGT
ACCAGCAGAAACCCGGCCAGGCTCCCCGGCTGCTG
ATCTACGGGGCTAGTAGCCGGGCCACCGGCATCCC
CGATCGGTTCAGCGGGTCCGGGTCCGGGACCGACT
TCACCCTGACCATCAGCCGGCTGGAGCCCGAGGAC
TTCGCCGTCTACTACTGCCAGCAGTACGGGTCCAG
CCCGATGTACACCTTCGGCCAGGGGACCAAGCTGG
AGATCAAG.

SEQ ID NO: 22 includes Target scFv: 1-708; Target VL: 382-708; Target linker: 337-381; Target VH: 1-336; Free energy: −355.5; gdT CAI: 0.811107619741586; ORF count: 2.

SEQ ID NO: 23 is the sequence name for PTK7 ECOg (2). It is a Tumor Targeting scFv construct. It is a DNA sequence. The sequence is:

GAGGTGCAGCTGGTGCAGAGCGGCGGAGGCCTGGT
GCACCCAGGCGGCTCCCTGCGGCTCAGCTGCGCCG
GTTCCGGGTTCACCTTTTCCACGTATCTGATGTAC
TGGGTGCGCCAGGCCCCGGGGAAGACCCTGGAATG
GGTCTCCGCCATCGGCTCCGGCGGCGACACCTACT
ACGCCGACTCCGTGAAGGGGCGCTTCACCATTTCC
CGGGACAACGCGAAGAATTCCCTGTACCTGCAGAT
GAACAGTCTCCGCGCCGAGGACATGGCCGTGTACT
ACTGTGCCCGGGGCCTCGGATACTGGGGCCAGGGG
ACCCTGGTCACCGTGTCCAGCGGCGGCGGAGGCTC
AGGAGGAGGAGGCTCAGGAGGCGGCGGCTCCGAGA
TCGTGTTGACCCAGAGCCCCGGGACCCTGTCCCTG
AGCCCCGGGGAACGGGCCACGCTGAGCTGCCGCGC
CTCGCAGAGCGTCTCCTCCTCGTACCTGGCCTGGT
ACCAGCAGAAGCCGGGGCAGGCTCCTCGGCTCCTC
ATCTACGGGGCCTCTTCCCGGGCCACGGGTATCCC
CGACAGGTTCAGCGGCTCCGGGTCCGGGACCGATT
TCACCTTGACCATTAGCCGCCTGGAACCCGAGGAC
TTCGCCGTGTACTATTGTCAGCAGTACGGGTCCTC
GCCCATGTACACCTTTGGCCAGGGGACCAAGCTGG
AGATCAAG.

SEQ ID NO: 23 includes Target scFv: 1-708; Target VL: 382-708; Target linker: 337-381; Target VH: 1-336; Free energy: −350.2; gdT CAI: 0.803914913406133; ORF count: 2.

SEQ ID NO: 24 is the sequence name for hSCF ligand. It is a Tumor Targeting Ligand construct. It is an AA sequence. The sequence is:

EGICRNRVTNNVKDVTKLVANLPKDYMITLKYVPG
MDVLPSHCWISEMVVQLSDSLTDLLDKFSNISEGL
SNYSIIDKLVNIVDDLVECVKENSSKDLKKSFKSP
EPRLFTPEEFFRIFNRSIDAFKDFVVASETSDCVV
SS.

SEQ ID NO: 24 includes Target Ligand: 1-142.

SEQ ID NO: 25 is the sequence name for hSCF ligand. It is a Tumor Targeting Ligand construct. It is a DNA sequence. The sequence is:

GAGGGGATCTGCAGGAACAGGGTGACCAACAATGT
GAAGGATGTGACCAAGCTGGTGGCCAACCTGCCCA
AGGACTACATGATCACCCTGAAGTATGTGCCAGGG
ATGGATGTGCTGCCCAGCCACTGCTGGATCTCTGA
GATGGTGGTGCAGCTGTCTGACTCCCTGACAGACC
TGCTGGACAAGTTCTCCAACATCTCAGAGGGGCTG
TCCAACTACTCCATCATTGACAAGCTGGTGAACAT
AGTGGATGACCTGGTGGAGTGTGTGAAGGAGAACT
CCTCCAAGGACCTGAAGAAGTCCTTCAAGTCCCCT
GAGCCCAGGCTGTTCACCCCTGAGGAGTTCTTCAG
GATCTTCAACAGGTCCATTGATGCCTTCAAGGACT
TTGTGGTGGCCTCTGAGACCTCTGACTGTGTGGTG
TCCTCA.

SEQ ID NO: 25 includes Target Ligand: 1-426; Free energy: −157.8; gdT CAI: 0.937866445503855; ORF count: 3.

SEQ ID NO: 26 is the sequence name for hSCF ligand ECOg (87). It is a Tumor Targeting Ligand construct. It is a DNA sequence. The sequence is:

GAGGGGATCTGCCGGAACCGGGTGACCAACAACGT
GAAGGACGTGACCAAGCTGGTGGCCAACCTGCCCA
AGGACTACATGATCACCCTGAAGTACGTGCCCGGG
ATGGACGTGCTGCCCAGCCACTGCTGGATCAGCGA
GATGGTGGTGCAGCTGTCCGACTCCCTGACCGACC
TGCTGGACAAGTTCTCCAACATCAGCGAGGGGCTG
AGCAACTACTCCATCATCGACAAGCTGGTGAACAT
CGTGGACGACCTGGTGGAGTGCGTGAAGGAGAACA
GCAGCAAGGACCTGAAGAAGTCCTTCAAGAGCCCC
GAGCCCCGGCTGTTCACGCCCGAGGAGTTCTTCCG
GATCTTCAACCGGAGCATCGACGCCTTCAAGGACT
TCGTGGTGGCCAGCGAGACCAGCGACTGCGTGGTG
TCCTCC.

SEQ ID NO: 26 includes Target Ligand: 1-426; Free energy: −160.7; gdT CAI: 0.928424; ORF count: 0.

SEQ ID NO: 27 is the sequence name for hSCF ligand ECOg (62). It is a Tumor Targeting Ligand construct. It is a DNA sequence. The sequence is:

GAGGGGATCTGCCGGAACCGGGTGACCAACAATGT
GAAGGACGTCACAAAGTTGGTCGCCAATCTGCCGA
AGGACTACATGATTACGCTGAAGTACGTCCCCGGA
ATGGATGTGCTGCCCAGCCACTGCTGGATTTCGGA
GATGGTGGTGCAGCTGTCCGACAGTCTGACCGATC
TGCTGGACAAGTTCAGCAACATCTCCGAAGGGCTG
TCCAACTACAGCATCATCGATAAGCTGGTCAACAT
CGTCGACGATCTGGTGGAGTGCGTCAAAGAGAACA
GCAGCAAAGATCTGAAGAAGTCGTTCAAATCGCCG
GAGCCGCGGCTGTTCACACCGGAGGAGTTCTTCCG
GATCTTCAATCGGTCGATCGACGCCTTCAAAGATT
TTGTGGTGGCCAGCGAAACCAGCGACTGCGTCGTC
AGCAGC.

SEQ ID NO: 27 includes Target Ligand: 1-426; Free energy: −164.6; gdT CAI: 0.805174371566857; ORF count: 2.

SEQ ID NO: 28 is the sequence name for hSCF ligand ECOg (61). It is a Tumor Targeting Ligand construct. It is a DNA sequence. The sequence is:

GAGGGGATCTGCCGGAACCGCGTGACGAACAACGT
GAAGGACGTCACGAAGCTCGTCGCGAACCTGCCGA
AGGACTACATGATCACCCTGAAGTACGTCCCCGGG
ATGGACGTGCTCCCCTCGCACTGCTGGATCTCCGA
GATGGTCGTCCAGCTGTCCGACTCCCTGACGGACC
TCCTCGACAAGTTCTCCAACATCTCCGAGGGGCTC
TCCAACTACTCGATCATCGACAAGCTGGTGAACAT
CGTGGACGACCTCGTGGAGTGCGTCAAGGAGAACT
CCTCGAAGGACCTCAAGAAGAGCTTCAAGTCGCCC
GAGCCGCGGCTCTTCACGCCCGAGGAGTTCTTCCG
GATCTTCAACCGGAGCATCGACGCCTTCAAGGACT
TCGTGGTGGCCTCCGAGACGTCCGACTGCGTCGTG
TCGAGC.

SEQ ID NO: 28 includes Target Ligand: 1-426; Free energy: −163.9; gdT CAI: 0.83416441300645; ORF count: 0.

SEQ ID NO: 29 is the sequence name for hSCF ligand ECOg (2). It is a Tumor Targeting Ligand construct. It is a DNA sequence. The sequence is:

GAGGGGATCTGCCGGAACCGGGTGACCAACAACGT
GAAGGACGTCACCAAGCTGGTCGCCAACCTGCCCA
AGGACTACATGATCACCTTGAAGTACGTGCCCGGC
ATGGACGTCCTGCCCAGCCACTGCTGGATCTCCGA
GATGGTCGTCCAGCTCAGCGACTCCCTGACCGACC
TCCTCGACAAGTTCTCCAACATCTCCGAGGGGCTC
AGCAACTACTCCATCATCGACAAGCTCGTGAACAT
AGTGGATGACCTCGTGGAGTGCGTGAAGGAGAACA
GCTCCAAGGACTTGAAGAAGTCCTTCAAGTCCCCG
GAGCCCAGGCTGTTCACGCCCGAGGAGTTCTTCAG
GATCTTCAACCGATCCATTGACGCCTTCAAGGACT
TCGTGGTGGCCTCCGAGACCAGCGACTGCGTGGTG
TCCTCC.

SEQ ID NO: 29 includes Target Ligand: 1-426; Free energy: −163.7; gdT CAI: 0.886419970682739; ORF count: 2.

SEQ ID NO: 30 is the sequence name for hSCF ligand ECOg (48). It is a Tumor Targeting Ligand construct. It is a DNA sequence. The sequence is:

GAGGGGATCTGCCGCAACCGCGTGACGAACAACGT
GAAGGACGTGACGAAGCTCGTGGCGAACCTGCCCA
AGGACTACATGATCACCCTGAAGTACGTCCCCGGG
ATGGACGTGCTGCCCAGCCACTGCTGGATCTCCGA
GATGGTGGTGCAGCTGAGCGACAGCCTGACGGACC
TGCTGGACAAGTTCAGCAACATCTCCGAGGGGCTG
AGCAACTACAGCATCATCGACAAGCTGGTGAACAT
CGTGGACGACCTGGTGGAGTGCGTGAAGGAGAACA
GCTCCAAGGACCTGAAGAAGAGCTTCAAGTCGCCC
GAGCCCCGGCTGTTCACGCCCGAGGAGTTCTTCCG
GATCTTCAACCGGAGCATCGACGCCTTCAAGGACT
TCGTCGTGGCGAGCGAGACGTCCGACTGCGTCGTG
TCCTCC.

SEQ ID NO: 30 includes Target Ligand: 1-426; Free energy: −163.5; gdT CAI: 0.876702550243938; ORF count: 0.

SEQ ID NO: 31 is the sequence name for Albumin. It is a Fusion Moety construct. It is an AA sequence. The sequence is:

DAHKSEVAHRFKDLGEENFKALVLIAFAQYLQQCP
FEDHVKLVNEVTEFAKTCVADESAENCDKSLHTLF
GDKLCTVATLRETYGEMADCCAKQEPERNECFLQH
KDDNPNLPRLVRPEVDVMCTAFHDNEETFLKKYLY
EIARRHPYFYAPELLFFAKRYKAAFTECCQAADKA
ACLLPKLDELRDEGKASSAKQRLKCASLQKFGERA
FKAWAVARLSQRFPKAEFAEVSKLVTDLTKVHTEC
CHGDLLECADDRADLAKYICENQDSISSKLKECCE
KPLLEKSHCIAEVENDEMPADLPSLAADFVESKDV
CKNYAEAKDVFLGMFLYEYARRHPDYSVVLLLRLA
KTYETTLEKCCAAADPHECYAKVFDEFKPLVEEPQ
NLIKQNCELFEQLGEYKFQNALLVRYTKKVPQVST
PTLVEVSRNLGKVGSKCCKHPEAKRMPCAEDYLSV
VLNQLCVLHEKTPVSDRVTKCCTESLVNRRPCFSA
LEVDETYVPKEFNAETFTFHADICTLSEKERQIKK
QTALVELVKHKPKATKEQLKAVMDDFAAFVEKCCK
ADDKETCFAEEGKKLVAASQAALGLGGGGS.

SEQ ID NO: 31 includes Fusion Moeity: 1-590.

SEQ ID NO: 32 is the sequence name for Albumin. It is a Fusion Moety construct. It is a DNA sequence. The sequence is:

GACGCTCATAAGTCCGAGGTGGCCCATCGCTTTAA
GGATCTGGGTGAGGAAAATTTTAAGGCGCTGGTGT
TGATTGCATTTGCGCAGTATTTGCAGCAGTGTCCA
TTCGAGGATCACGTGAAGTTAGTGAACGAGGTGAC
TGAGTTCGCAAAGACCTGTGTCGCGGATGAGAGCG
CTGAGAACTGTGATAAATCCCTTCACACCCTGTTC
GGCGATAAACTTTGCACTGTGGCCACATTGCGCGA
AACTTACGGAGAGATGGCCGACTGCTGCGCTAAAC
AGGAGCCCGAGAGGAACGAGTGCTTCTTACAGCAT
AAGGATGACAACCCCAATCTGCCAAGACTGGTCAG
GCCTGAAGTGGACGTCATGTGTACAGCCTTCCATG
ACAACGAGGAGACGTTCCTCAAGAAATACTTGTAC
GAGATCGCTCGACGTCACCCTTATTTCTACGCACC
TGAATTGCTGTTTTTTGCCAAGCGGTATAAAGCAG
CTTTCACAGAGTGCTGCCAGGCTGCAGACAAGGCT
GCCTGCTTGCTTCCAAAATTGGACGAGCTGAGAGA
TGAGGGCAAGGCATCCTCAGCTAAACAGCGGCTGA
AATGTGCCTCTCTGCAAAAGTTCGGGGAGCGGGCT
TTCAAGGCCTGGGCCGTTGCGCGGCTGAGCCAGAG
ATTTCCCAAGGCCGAGTTCGCTGAAGTTTCAAAGC
TCGTCACCGACCTTACCAAGGTTCATACTGAATGT
TGCCATGGCGACCTGCTAGAATGCGCAGACGACAG
GGCGGATCTGGCCAAGTACATCTGTGAGAATCAAG
ACTCTATTAGTAGCAAGCTCAAAGAGTGTTGCGAG
AAGCCATTACTCGAAAAGTCCCATTGCATCGCAGA
GGTAGAAAACGATGAAATGCCTGCCGACCTTCCCA
GTCTCGCCGCCGACTTTGTGGAGAGCAAAGATGTG
TGCAAAAATTACGCAGAGGCCAAGGATGTATTCCT
GGGGATGTTTCTCTACGAGTATGCCAGAAGACACC
CCGATTATAGTGTGGTGCTCCTTCTGCGCTTAGCC
AAAACATACGAGACCACCCTGGAGAAGTGTTGTGC
AGCCGCCGATCCACACGAGTGCTATGCTAAGGTCT
TTGATGAATTTAAACCACTAGTCGAAGAGCCCCAG
AATCTCATAAAGCAGAATTGCGAACTTTTTGAACA
GCTGGGCGAATATAAATTCCAAAATGCTCTGCTAG
TACGATATACCAAGAAAGTGCCCCAAGTAAGTACT
CCTACGTTGGTTGAGGTGAGCCGTAACCTGGGGAA
GGTGGGATCAAAGTGCTGTAAACACCCTGAAGCGA
AAAGGATGCCGTGCGCAGAAGATTACCTTTCTGTG
GTACTCAACCAGCTCTGCGTGTTACACGAGAAGAC
TCCGGTGTCTGACCGAGTTACAAAATGTTGCACCG
AGTCACTCGTCAATCGCCGGCCCTGCTTCAGCGCT
CTAGAAGTCGACGAAACCTACGTGCCTAAGGAATT
CAACGCCGAGACGTTCACCTTTCACGCCGACATCT
GTACTCTCTCCGAGAAGGAGAGGCAGATTAAGAAA
CAAACAGCACTGGTGGAACTGGTTAAACACAAACC
AAAGGCTACAAAGGAACAGCTGAAAGCGGTTATGG
ATGACTTCGCCGCCTTTGTCGAAAAGTGTTGTAAA
GCAGACGATAAAGAAACATGTTTTGCTGAAGAAGG
TAAGAAACTGGTCGCCGCCTCGCAGGCAGCTCTGG
GACTGGGAGGTGGTGGGTCT.

SEQ ID NO: 32 includes Fusion Moeity: 1-1770; Free energy: −557.6; gdT CAI: 0.787786351918606; ORF count: 21.

SEQ ID NO: 33 is the sequence name for Albumin ECOg (8). It is a Fusion Moety construct. It is a DNA sequence. The sequence is:

GACGCCCACAAGTCCGAGGTGGCCCACAGGTTCAA
GGACCTGGGCGAGGAGAACTTCAAGGCCCTGGTGC
TGATCGCCTTCGCCCAGTACCTGCAGCAGTGCCCC
TTCGAGGACCACGTGAAGTTGGTGAACGAGGTGAC
CGAGTTCGCCAAGACCTGCGTGGCCGACGAGAGCG
CCGAGAACTGCGACAAGTCCCTGCACACCCTGTTC
GGGGACAAGCTGTGCACCGTGGCCACGCTGCGGGA
GACCTACGGGGAGATGGCCGACTGCTGCGCCAAGC
AGGAGCCCGAGCGGAACGAGTGCTTCCTGCAGCAC
AAGGACGACAACCCCAACCTGCCCCGACTGGTCCG
GCCCGAGGTGGACGTGATGTGCACCGCCTTCCACG
ACAACGAGGAGACCTTCCTGAAGAAGTACCTGTAC
GAGATCGCCCGGCGGCACCCCTACTTCTACGCGCC
CGAGCTGCTGTTCTTCGCCAAGCGGTACAAGGCCG
CCTTCACCGAGTGCTGCCAGGCCGCCGACAAGGCC
GCCTGCCTGCTGCCCAAGCTGGACGAGCTGCGGGA
CGAGGGGAAGGCCTCCAGCGCCAAGCAGCGGCTGA
AGTGCGCGTCCCTGCAGAAGTTCGGGGAGCGGGCC
TTCAAGGCTTGGGCCGTGGCCCGGCTGTCCCAACG
GTTCCCCAAGGCCGAGTTCGCCGAGGTGAGCAAGT
TGGTGACCGACCTGACCAAGGTGCACACCGAGTGC
TGCCACGGGGACCTGCTGGAGTGCGCCGACGACCG
GGCCGATCTGGCCAAGTACATCTGCGAGAACCAGG
ACAGCATCAGCTCCAAGCTGAAGGAGTGCTGCGAG
AAGCCCCTGCTGGAGAAGTCCCACTGCATCGCCGA
GGTGGAGAACGACGAGATGCCGGCCGATCTGCCCT
CGCTGGCCGCCGACTTCGTGGAGAGCAAGGACGTG
TGCAAGAACTACGCCGAGGCCAAGGACGTGTTCCT
GGGGATGTTCCTGTACGAGTACGCCCGGCGGCACC
CCGACTACTCCGTGGTGCTGCTGCTGCGGCTGGCC
AAGACCTACGAGACCACCTTGGAGAAGTGCTGCGC
CGCGGCCGATCCGCACGAGTGCTACGCCAAGGTGT
TCGACGAGTTCAAGCCCCTGGTGGAGGAGCCCCAG
AACCTGATCAAGCAGAACTGCGAGCTGTTCGAGCA
GCTCGGGGAGTACAAGTTCCAGAACGCCCTGCTGG
TGCGGTACACCAAGAAGGTGCCCCAGGTGAGCACC
CCGACCCTGGTGGAGGTGAGCCGGAACCTGGGGAA
GGTCGGGTCCAAGTGCTGCAAGCACCCCGAGGCCA
AGCGGATGCCCTGCGCCGAGGACTACCTGTCCGTG
GTGCTGAACCAGCTGTGCGTGCTGCACGAGAAGAC
GCCCGTGTCCGACCGGGTGACCAAGTGCTGCACCG
AGAGCTTGGTGAACCGGCGGCCCTGCTTCAGCGCC
CTGGAGGTGGACGAGACCTACGTGCCCAAGGAGTT
CAACGCCGAGACCTTCACCTTCCACGCCGACATCT
GCACCCTGAGCGAGAAGGAGCGGCAGATCAAGAAG
CAGACGGCCCTGGTGGAGCTGGTGAAGCACAAGCC
CAAGGCCACCAAGGAGCAGCTGAAGGCCGTGATGG
ACGACTTCGCCGCCTTCGTGGAGAAGTGCTGCAAG
GCCGACGACAAGGAGACCTGCTTCGCCGAAGAGGG
CAAGAAGTTGGTGGCCGCGTCCCAGGCCGCACTGG
GTCTGGGAGGTGGTGGGTCT.

SEQ ID NO: 33 includes Fusion Moeity: 1-1770; Free energy: −755.4; gdT CAI: 0.932865573516391; ORF count: 0.

SEQ ID NO: 34 is the sequence name for Albumin ECOg (60). It is a Fusion Moety construct. It is a DNA sequence. The sequence is:

GACGCCCACAAGAGCGAGGTGGCGCACCGCTTCAA
GGACCTGGGCGAGGAGAACTTCAAGGCGCTGGTGC
TGATCGCGTTCGCCCAGTACCTCCAGCAGTGCCCC
TTCGAGGACCACGTGAAGCTGGTGAACGAGGTGAC
CGAGTTCGCCAAGACCTGCGTGGCCGACGAGAGCG
CCGAGAACTGCGACAAGAGCCTCCACACCCTGTTC
GGGGACAAGCTGTGCACCGTGGCCACGCTGCGCGA
GACCTACGGGGAGATGGCCGACTGCTGCGCCAAGC
AGGAGCCCGAGCGCAACGAGTGCTTCCTCCAGCAC
AAGGACGACAACCCCAACCTACCCCGTCTGGTGCG
CCCCGAGGTGGACGTGATGTGCACCGCGTTCCACG
ACAACGAGGAGACCTTCCTCAAGAAGTACCTCTAC
GAGATCGCGCGGCGGCACCCCTACTTCTACGCGCC
CGAGCTGCTGTTCTTCGCCAAGCGCTACAAGGCCG
CGTTCACCGAGTGCTGCCAGGCCGCCGACAAGGCC
GCGTGCCTGCTCCCCAAGCTCGACGAGCTGCGCGA
CGAGGGGAAGGCGTCGAGCGCCAAGCAGAGGCTCA
AGTGCGCCTCGCTCCAGAAGTTCGGGGAGCGCGCG
TTCAAGGCGTGGGCCGTGGCGAGGCTCTCCCAGCG
GTTCCCCAAGGCCGAGTTCGCCGAGGTGAGCAAGC
TGGTGACCGACCTCACCAAGGTGCACACCGAGTGC
TGCCACGGGGACCTGCTGGAGTGCGCCGACGACCG
CGCCGATCTCGCCAAGTACATCTGCGAGAACCAGG
ACAGCATCTCCTCCAAGCTCAAGGAGTGCTGCGAG
AAGCCCCTGCTGGAGAAGAGCCACTGCATCGCCGA
GGTGGAGAACGACGAGATGCCAGCCGATCTCCCCT
CGCTCGCCGCCGACTTCGTGGAGAGCAAGGACGTG
TGCAAGAACTACGCCGAGGCCAAGGACGTGTTCCT
GGGGATGTTCCTCTACGAGTACGCGCGGCGGCACC
CCGACTACAGCGTGGTGCTGCTGCTGCGGCTCGCC
AAGACCTACGAGACCACGCTGGAGAAGTGCTGCGC
CGCCGCCGATCCCCACGAGTGCTACGCCAAGGTGT
TCGACGAGTTCAAGCCCCTGGTGGAGGAGCCCCAG
AACCTGATCAAGCAGAACTGCGAGCTGTTCGAGCA
GCTCGGGGAGTACAAGTTCCAGAACGCGCTGCTGG
TGCGCTACACCAAGAAGGTGCCCCAGGTGAGCACC
CCGACCCTGGTGGAGGTGAGCCGCAACCTGGGGAA
GGTCGGGAGCAAGTGCTGCAAGCACCCCGAGGCCA
AGCGGATGCCCTGCGCCGAGGACTACCTCTCGGTG
GTGCTCAACCAGCTGTGCGTGCTCCACGAGAAGAC
CCCGGTGAGCGACAGGGTGACCAAGTGCTGCACCG
AGTCGCTGGTGAACCGGAGGCCCTGCTTCTCCGCG
CTGGAGGTGGACGAGACCTACGTGCCCAAGGAGTT
CAACGCCGAGACCTTCACCTTCCACGCCGACATCT
GCACCCTCTCCGAGAAGGAGCGCCAGATCAAGAAG
CAGACCGCGCTGGTGGAGCTGGTGAAGCACAAGCC
CAAGGCCACCAAGGAGCAGCTCAAGGCGGTGATGG
ACGACTTCGCCGCGTTCGTGGAGAAGTGCTGCAAG
GCCGACGACAAGGAGACCTGCTTCGCCGAAGAGGG
GAAGAAGCTGGTGGCCGCGAGCCAAGCCGCTCTGG
GGCTGGGAGGTGGTGGGTCT.

SEQ ID NO: 34 includes Fusion Moeity: 1-1770; Free energy: −753.1; gdT CAI: 0.893692649517376; ORF count: 0.

SEQ ID NO: 35 is the sequence name for Albumin ECOg (91). It is a Fusion Moety construct. It is a DNA sequence. The sequence is:

GACGCCCACAAGTCTGAGGTCGCCCATCGGTTCAA
GGATCTCGGCGAGGAGAACTTCAAGGCCCTCGTGC
TGATCGCCTTCGCGCAGTACCTCCAGCAGTGCCCC
TTCGAGGACCACGTGAAGCTGGTCAACGAGGTGAC
CGAGTTCGCCAAGACCTGCGTGGCCGACGAGAGCG
CCGAGAACTGCGATAAGAGCCTGCACACCCTGTTC
GGCGACAAGCTGTGCACCGTGGCGACCCTCAGGGA
GACCTACGGGGAGATGGCCGACTGCTGCGCCAAGC
AGGAGCCCGAGCGGAACGAGTGCTTCCTGCAGCAC
AAGGATGACAACCCCAATCTGCCCCGCTTGGTGCG
CCCCGAGGTGGACGTGATGTGCACCGCGTTCCACG
ACAACGAGGAGACCTTCCTGAAGAAGTACCTCTAC
GAGATCGCCAGGCGGCACCCCTACTTCTACGCTCC
CGAGCTGCTGTTCTTCGCCAAGCGGTACAAGGCGG
CCTTCACGGAGTGCTGCCAGGCGGCCGACAAGGCG
GCCTGCCTGCTGCCCAAGCTGGACGAGCTGCGCGA
CGAGGGGAAGGCCAGCAGCGCCAAGCAGAGACTGA
AGTGCGCCTCTCTGCAGAAGTTCGGCGAGCGGGCC
TTCAAGGCGTGGGCCGTCGCTCGGCTCTCCCAGCG
GTTCCCCAAGGCGGAGTTCGCCGAGGTGTCCAAGC
TGGTGACCGACCTGACCAAGGTGCACACCGAGTGC
TGCCACGGGGACCTCCTGGAGTGCGCCGACGATCG
GGCCGACCTGGCGAAGTACATCTGCGAGAACCAGG
ATAGCATCAGCTCCAAGCTGAAGGAGTGCTGCGAG
AAGCCCCTGCTGGAGAAGAGCCACTGCATCGCCGA
GGTGGAGAACGACGAGATGCCCGCAGATCTGCCCA
GCCTGGCGGCCGACTTCGTGGAGAGCAAGGATGTC
TGCAAGAATTACGCCGAGGCGAAGGACGTGTTCCT
CGGGATGTTCCTCTACGAGTACGCCCGGCGGCACC
CCGATTACAGCGTGGTGCTCCTCCTGCGGCTCGCC
AAGACCTACGAGACAACCCTGGAGAAGTGCTGCGC
TGCCGCCGATCCCCACGAGTGCTACGCCAAGGTGT
TCGACGAGTTCAAGCCCCTCGTGGAGGAGCCCCAG
AATCTCATCAAGCAGAACTGCGAGCTGTTCGAGCA
GCTCGGGGAGTACAAGTTCCAGAACGCCCTGCTGG
TGAGGTACACCAAGAAGGTGCCCCAGGTGAGCACC
CCGACCCTCGTGGAGGTGAGCCGCAACCTGGGGAA
GGTCGGCTCTAAGTGCTGCAAGCACCCCGAGGCCA
AGCGGATGCCCTGCGCGGAGGACTACCTGTCCGTG
GTGCTCAACCAGCTGTGCGTGCTGCACGAGAAGAC
ACCCGTGTCCGACCGGGTGACCAAGTGCTGCACGG
AGTCCCTGGTGAACCGGCGGCCCTGCTTCTCCGCT
CTGGAGGTCGACGAGACCTACGTGCCCAAGGAGTT
CAACGCCGAGACGTTCACCTTCCACGCGGATATCT
GCACGCTGAGCGAGAAGGAGAGGCAGATCAAGAAG
CAGACGGCCCTCGTGGAGCTCGTGAAGCACAAGCC
CAAGGCCACCAAGGAGCAGCTGAAGGCCGTGATGG
ACGACTTCGCGGCCTTCGTGGAGAAGTGCTGCAAG
GCGGACGACAAGGAGACCTGCTTCGCCGAGGAAGG
CAAGAAGCTGGTGGCCGCCAGTCAGGCTGCCCTGG
GGCTCGGAGGTGGTGGGTCT.

SEQ ID NO: 35 includes Fusion Moeity: 1-1770; Free energy: −750.9; gdT CAI: 0.909026600521571; ORF count: 7.

SEQ ID NO: 36 is the sequence name for Albumin ECOg (51). It is a Fusion Moety construct. It is a DNA sequence. The sequence is:

GACGCCCACAAGTCCGAGGTGGCGCACCGCTTCAA
GGACCTCGGGGAAGAGAACTTCAAGGCCCTGGTGC
TGATCGCCTTCGCGCAGTACCTGCAGCAGTGCCCC
TTCGAGGACCACGTGAAGCTCGTGAACGAGGTCAC
GGAGTTCGCCAAGACCTGCGTCGCCGACGAGAGCG
CCGAGAACTGCGACAAGAGCCTCCACACCCTGTTC
GGGGACAAGCTCTGCACCGTGGCGACGCTGCGGGA
GACCTACGGCGAGATGGCGGACTGCTGCGCCAAGC
AGGAGCCCGAGCGCAACGAGTGCTTCCTGCAGCAC
AAGGACGACAACCCCAACCTGCCGCGGCTCGTGAG
GCCCGAGGTCGACGTGATGTGCACCGCGTTCCACG
ACAACGAGGAGACGTTCCTGAAGAAGTACCTCTAC
GAGATCGCGAGGCGGCACCCCTACTTCTACGCCCC
GGAGCTGCTGTTCTTCGCGAAGCGCTACAAGGCCG
CCTTCACGGAGTGCTGCCAGGCCGCCGACAAGGCC
GCGTGCCTGCTGCCGAAGCTCGACGAGCTCCGGGA
CGAGGGCAAGGCCTCCAGCGCCAAGCAGCGCCTCA
AGTGCGCCTCGCTGCAGAAGTTCGGGGAGCGGGCC
TTCAAGGCCTGGGCCGTGGCGCGGCTGTCACAGCG
GTTCCCCAAGGCCGAGTTCGCCGAGGTCTCGAAGC
TCGTGACGGACCTCACCAAGGTCCACACCGAGTGC
TGCCACGGGGACCTCCTGGAGTGCGCCGACGACAG
GGCCGACCTCGCGAAGTACATCTGCGAGAACCAGG
ACTCCATCAGCTCCAAGCTCAAGGAGTGCTGCGAG
AAGCCCCTGCTGGAGAAGTCCCACTGCATCGCCGA
GGTCGAGAACGACGAGATGCCCGCGGATCTCCCGA
GCCTCGCCGCGGACTTCGTGGAGTCCAAGGACGTC
TGCAAGAACTACGCCGAGGCCAAGGACGTGTTCCT
GGGGATGTTCCTGTACGAGTACGCGAGGCGGCACC
CCGACTACTCCGTGGTGCTGCTGCTGCGGCTCGCC
AAGACCTACGAGACGACCCTGGAGAAGTGCTGCGC
CGCCGCAGATCCCCACGAGTGCTACGCCAAGGTCT
TCGACGAGTTCAAGCCCCTGGTGGAGGAGCCCCAG
AACCTCATCAAGCAGAACTGCGAGCTCTTCGAGCA
GCTCGGGGAGTACAAGTTCCAGAACGCGCTGCTGG
TGCGGTACACCAAGAAGGTGCCCCAGGTCTCGACC
CCGACCCTGGTGGAGGTGTCCCGGAACCTCGGGAA
GGTCGGGTCCAAGTGCTGCAAGCACCCCGAGGCGA
AGCGGATGCCCTGCGCCGAGGACTACCTCTCCGTG
GTGCTGAACCAGCTCTGCGTGCTGCACGAGAAGAC
CCCGGTGTCGGACCGCGTCACGAAGTGCTGCACCG
AGTCCCTGGTGAACCGGAGGCCCTGCTTCAGCGCC
CTGGAGGTCGACGAGACCTACGTCCCCAAGGAGTT
CAACGCCGAGACCTTCACGTTCCACGCCGACATCT
GCACCCTGTCCGAGAAGGAGCGCCAGATCAAGAAG
CAGACGGCCCTGGTGGAGCTCGTGAAGCACAAGCC
CAAGGCCACCAAGGAGCAGCTCAAGGCCGTGATGG
ACGACTTCGCGGCCTTCGTGGAGAAGTGCTGCAAG
GCCGACGACAAGGAGACCTGCTTCGCCGAGGAAGG
CAAGAAGCTCGTGGCGGCGTCCCAGGCGGCTCTGG
GACTCGGAGGTGGTGGGTCT.

SEQ ID NO: 36 includes Fusion Moeity: 1-1770; Free energy: −749.1; gdT CAI: 0.879685715389261; ORF count: 0.

SEQ ID NO: 37 is the sequence name for Albumin ECOg (62). It is a Fusion Moety construct. It is a DNA sequence. The sequence is:

GACGCCCACAAGTCCGAGGTGGCGCACCGCTTCAA
GGACCTGGGCGAGGAGAACTTCAAGGCCCTGGTGC
TGATCGCCTTCGCCCAGTACCTGCAGCAGTGCCCC
TTCGAGGACCACGTGAAGCTGGTGAACGAGGTGAC
CGAGTTCGCCAAGACCTGCGTGGCCGACGAGTCCG
CCGAGAACTGCGACAAGTCCCTGCACACCCTGTTC
GGGGACAAGCTGTGCACCGTGGCCACGCTGCGGGA
GACCTACGGGGAGATGGCCGACTGCTGCGCCAAGC
AGGAGCCCGAGCGGAACGAGTGCTTCCTGCAGCAC
AAGGACGACAACCCCAACCTGCCCAGGCTGGTGCG
GCCCGAGGTGGACGTGATGTGCACCGCCTTCCACG
ACAACGAGGAGACCTTCCTGAAGAAGTACCTGTAC
GAGATCGCCCGGCGGCACCCCTACTTCTACGCGCC
CGAGCTGCTGTTCTTCGCCAAGCGGTACAAGGCCG
CCTTCACCGAGTGCTGCCAGGCCGCCGACAAGGCC
GCCTGCCTGCTGCCCAAGCTGGACGAGCTGCGGGA
CGAGGGGAAGGCCTCCTCCGCCAAGCAGCGGCTGA
AGTGCGCCAGCCTGCAGAAGTTCGGGGAGCGGGCC
TTCAAGGCTTGGGCCGTGGCCCGGCTGTCCCAGAG
GTTCCCCAAGGCCGAGTTCGCCGAGGTGTCCAAGC
TGGTGACCGACCTGACCAAGGTGCACACCGAGTGC
TGCCACGGGGACCTGCTGGAGTGCGCCGACGACCG
GGCTGACCTGGCCAAGTACATCTGCGAGAACCAGG
ACTCCATCTCCTCCAAGCTGAAGGAGTGCTGCGAG
AAGCCCCTGCTGGAGAAGTCCCACTGCATCGCCGA
GGTGGAGAACGACGAGATGCCGGCTGACCTGCCCA
GCCTGGCCGCCGACTTCGTGGAGTCCAAGGACGTG
TGCAAGAACTACGCCGAGGCCAAGGACGTGTTCCT
GGGGATGTTCCTGTACGAGTACGCCCGGCGGCACC
CCGACTACTCCGTGGTGCTGCTGCTGCGGCTGGCC
AAGACCTACGAGACGACCCTGGAGAAGTGCTGCGC
CGCCGCCGATCCCCACGAGTGCTACGCCAAGGTGT
TCGACGAGTTCAAGCCCCTGGTGGAGGAGCCCCAG
AACCTGATCAAGCAGAACTGCGAGCTGTTCGAGCA
GCTGGGCGAGTACAAGTTCCAGAACGCCCTGCTGG
TGCGGTACACCAAGAAGGTGCCCCAGGTGAGCACC
CCGACCCTGGTGGAGGTGTCCCGGAACCTGGGGAA
GGTCGGGTCCAAGTGCTGCAAGCACCCCGAGGCCA
AGCGGATGCCCTGCGCCGAGGACTACCTGTCCGTG
GTGCTGAACCAGCTGTGCGTGCTGCACGAGAAGAC
GCCCGTGTCCGACCGGGTGACCAAGTGCTGCACCG
AGTCCCTGGTGAACCGGCGGCCCTGCTTCAGCGCC
CTGGAGGTGGACGAGACCTACGTGCCCAAGGAGTT
CAACGCCGAGACCTTCACCTTCCACGCCGACATCT
GCACCCTGTCCGAGAAGGAGCGGCAGATCAAGAAG
CAGACGGCCCTGGTGGAGCTGGTGAAGCACAAGCC
CAAGGCCACCAAGGAGCAGCTGAAGGCCGTGATGG
ACGACTTCGCCGCCTTCGTGGAGAAGTGCTGCAAG
GCCGACGACAAGGAGACCTGCTTCGCCGAAGAGGG
GAAGAAGCTGGTGGCCGCCAGCCAGGCTGCCCTGG
GACTGGGAGGTGGTGGGTCT.

SEQ ID NO: 37 includes Fusion Moeity: 1-1770; Free energy: −745.2; gdT CAI: 0.955655916201255; ORF count: 4.

SEQ ID NO: 38 is the sequence name for GD2 scFv. It is a Tumor targeting scFv construct. It is an AA sequence. The sequence is:

EVQLLQSGPELEKPGASVMISCKASGSSFTGYNMN
WVRQNIGKSLEWIGAIDPYYGGTSYNQKFKGRATL
TVDKSSSTAYMHLKSLTSEDSAVYYCVSGMKYWGQ
GTSVTVSSGGGGSGGGGSGGGGSDVVMTQTPLSLP
VSLGDQASISCRSSQSLVHRNGNTYLHWYLQKPGQ
SPKLLIHKVSNRFSGVPDRFSGSGSGTDFTLKISR
VEAEDLGVYFCSQSTHVPPLTFGAGTKLELKRAD.

SEQ ID NO: 38 includes Target scFv: 1-244; Target VL: 129-244; Target linker: 114-128; Target VH: 1-113.

SEQ ID NO: 39 is the sequence name for GD2 scFv. It is a Tumor targeting scFv construct. It is a DNA sequence. The sequence is:

GAAGTGCAGTTGCTGCAGTCTGGACCCGAGCTGGA
GAAGCCTGGGGCTTCAGTGATGATAAGTTGTAAAG
CGAGCGGCTCTTCGTTCACGGGATACAACATGAAT
TGGGTAAGACAGAACATCGGAAAGTCCCTCGAATG
GATTGGTGCAATCGATCCATACTATGGGGGCACAA
GCTATAATCAGAAGTTTAAAGGCAGGGCCACTCTG
ACCGTCGACAAATCCTCATCCACCGCTTATATGCA
CTTAAAGAGTCTTACTTCTGAAGACAGCGCCGTTT
ATTACTGCGTGTCAGGCATGAAGTACTGGGGTCAA
GGGACAAGCGTGACCGTCAGTTCCGGTGGTGGTGG
AAGCGGTGGTGGTGGATCTGGCGGTGGTGGAAGTG
ATGTAGTGATGACCCAGACTCCTCTGAGTCTGCCC
GTTAGTTTAGGTGACCAGGCCAGCATCAGCTGCAG
GTCCAGCCAGTCATTGGTTCATAGAAACGGCAATA
CCTACCTGCACTGGTACCTGCAAAAACCCGGGCAA
TCTCCTAAACTTCTCATACACAAGGTCTCTAACCG
TTTCTCGGGGGTCCCGGATCGGTTTAGCGGATCAG
GCTCCGGAACAGATTTTACGTTGAAGATTTCTCGC
GTGGAGGCCGAGGACCTTGGTGTGTATTTCTGTTC
CCAGTCCACTCATGTGCCACCACTGACATTCGGCG
CTGGGACCAAACTCGAACTAAAGCGAGCAGAC.

SEQ ID NO: 39 includes Target scFv: 1-732; Target VL: 385-732; Target linker: 340-384; Target VH: 1-339; Free energy: −243.8; gdT CAI: 0.70651634854966; ORF count: 9.

SEQ ID NO: 40 is the sequence name for GD2 scFv ECOg (18). It is a Tumor targeting scFv construct. It is a DNA sequence. The sequence is:

GAGGTCCAGCTCCTCCAGTCCGGGCCCGAGCTGGA
GAAGCCCGGGGCCTCCGTGATGATCTCCTGCAAGG
CCTCCGGGTCCTCCTTCACCGGGTACAACATGAAC
TGGGTCCGCCAGAACATCGGGAAGAGCCTGGAGTG
GATCGGGGCCATCGACCCCTACTACGGAGGCACCT
CCTACAACCAGAAGTTCAAGGGCCGGGCCACTCTG
ACCGTGGACAAGTCCTCCTCCACCGCCTACATGCA
CCTGAAGAGCCTGACCTCCGAGGACTCCGCCGTCT
ACTACTGCGTCTCCGGGATGAAGTACTGGGGCCAG
GGGACCTCCGTGACCGTCTCCTCCGGAGGAGGAGG
CTCCGGAGGAGGAGGCTCCGGAGGAGGAGGCTCCG
ACGTGGTGATGACCCAGACCCCTCTGAGCCTGCCC
GTGAGCCTCGGGGACCAGGCCTCCATCAGCTGCAG
GAGCTCCCAGAGCCTGGTCCACAGGAACGGGAACA
CCTACCTCCACTGGTACCTCCAGAAGCCCGGGCAG
AGCCCCAAGCTCCTGATCCACAAGGTCTCCAACAG
GTTCTCCGGGGTCCCCGACCGCTTCAGCGGGTCCG
GGTCCGGGACCGACTTCACCCTGAAGATCTCCAGA
GTGGAGGCCGAGGACCTCGGGGTCTACTTCTGCTC
CCAGAGCACCCACGTGCCTCCTCTGACCTTCGGGG
CCGGGACCAAGCTGGAGCTCAAGAGGGCCGAC.

SEQ ID NO: 40 includes Target scFv: 1-732; Target VL: 385-732; Target linker: 340-384; Target VH: 1-339; Free energy: −328.1; gdT CAI: 0.877232; ORF count: 0.

SEQ ID NO: 41 is the sequence name for GD2 scFv ECOg (84). It is a Tumor targeting scFv construct. It is a DNA sequence. The sequence is:

GAGGTCCAGCTGCTGCAGTCGGGGCCCGAGCTGGA
GAAGCCCGGGGCCTCCGTCATGATCTCGTGCAAGG
CCTCCGGGTCCTCGTTCACCGGGTACAACATGAAC
TGGGTGCGCCAGAACATCGGGAAGTCGCTGGAGTG
GATCGGGGCCATCGACCCCTACTACGGCGGCACCA
GCTACAACCAGAAGTTCAAGGGGCGCGCCACGCTG
ACGGTGGACAAGTCGTCGTCGACCGCCTACATGCA
CCTGAAGTCGCTGACGTCGGAGGACTCCGCCGTCT
ACTACTGCGTCAGCGGGATGAAGTACTGGGGCCAG
GGGACCTCGGTCACCGTGTCCTCCGGCGGCGGAGG
AAGCGGAGGAGGAGGCTCCGGCGGAGGAGGCTCCG
ACGTCGTGATGACGCAGACCCCGCTGTCGCTCCCG
GTGTCCCTCGGGGACCAGGCCTCCATCAGCTGCCG
GAGCTCGCAGTCCCTGGTGCACCGGAACGGGAACA
CCTACCTCCACTGGTACCTGCAGAAGCCGGGGCAG
TCGCCCAAGCTGCTGATCCACAAGGTGTCCAACAG
GTTCTCCGGGGTCCCCGACCGCTTCAGCGGGAGCG
GGAGCGGGACCGACTTCACCCTGAAGATCTCCCGC
GTGGAGGCCGAGGACCTCGGGGTCTACTTCTGCAG
CCAGAGCACCCACGTGCCACCGCTGACCTTCGGCG
CCGGCACCAAGCTGGAGCTCAAGCGGGCCGAC.

SEQ ID NO: 41 includes Target scFv: 1-732; Target VL: 385-732; Target linker: 340-384; Target VH: 1-339; Free energy: −351.8; gdT CAI: 0.797734389193656; ORF count: 0.

SEQ ID NO: 42 is the sequence name for GD2 scFv ECOg (88). It is a Tumor targeting scFv construct. It is a DNA sequence. The sequence is:

GAGGTCCAGCTGCTCCAGTCCGGGCCCGAGCTGGA
GAAGCCCGGGGCCTCGGTGATGATCAGCTGCAAGG
CCTCCGGGTCGAGCTTCACCGGGTACAACATGAAC
TGGGTCCGGCAGAACATCGGGAAGTCGCTGGAGTG
GATCGGGGCGATCGACCCCTACTACGGCGGCACCA
GCTACAACCAGAAGTTCAAGGGGCGCGCGACCCTG
ACCGTCGACAAGTCGAGCTCGACCGCCTACATGCA
CCTGAAGTCGCTGACCTCCGAGGACTCCGCGGTCT
ACTACTGCGTGAGCGGGATGAAGTACTGGGGCCAG
GGGACCTCGGTGACCGTGAGCTCCGGCGGCGGCGG
ATCTGGTGGTGGCGGTTCCGGCGGTGGCGGTTCCG
ACGTGGTGATGACCCAGACCCCGCTCTCGCTCCCG
GTCTCGCTCGGGGACCAGGCCTCGATCAGCTGCCG
GAGCTCCCAGTCGCTGGTCCACCGGAACGGGAACA
CCTACCTCCACTGGTACCTCCAGAAGCCCGGGCAG
TCGCCCAAGCTGCTGATCCACAAGGTGAGCAACCG
CTTCTCCGGGGTCCCCGACCGCTTCTCCGGGTCCG
GGTCCGGGACCGACTTCACCCTGAAGATCTCCCGG
GTCGAGGCCGAGGACCTCGGGGTCTACTTCTGCTC
CCAGTCGACCCACGTGCCGCCACTGACCTTCGGGG
CCGGGACCAAGCTGGAGCTGAAGCGGGCCGAC.

SEQ ID NO: 42 includes Target scFv: 1-732; Target VL: 385-732; Target linker: 340-384; Target VH: 1-339; Free energy: −340; gdT CAI: 0.799370908165938; ORF count: 1.

SEQ ID NO: 43 is the sequence name for GD2 scFv ECOg (2). It is a Tumor targeting scFv construct. It is a DNA sequence. The sequence is:

GAGGTGCAGCTGCTGCAGTCCGGCCCCGAGCTGGA
GAAGCCCGGGGCCAGCGTGATGATCAGCTGCAAGG
CCAGCGGGTCCAGCTTCACGGGGTACAACATGAAC
TGGGTGCGGCAGAACATCGGGAAGAGCCTGGAGTG
GATCGGGGCCATCGACCCGTACTACGGCGGCACGT
CGTACAACCAGAAGTTCAAGGGGCGCGCCACGCTG
ACCGTGGACAAGTCCAGCAGCACGGCCTACATGCA
CCTCAAGTCGCTGACCAGCGAGGACAGCGCGGTGT
ACTACTGCGTGTCCGGCATGAAGTACTGGGGCCAG
GGGACCAGCGTGACGGTGAGCAGCGGCGGTGGTGG
CAGCGGTGGTGGCGGTAGCGGCGGTGGTGGCAGCG
ACGTGGTGATGACCCAGACGCCCCTCAGCCTGCCG
GTCAGCCTGGGCGACCAGGCCAGCATTTCGTGCCG
CAGCTCGCAGAGCCTGGTGCACCGCAACGGGAACA
CCTACCTGCACTGGTACCTGCAGAAGCCCGGGCAG
AGCCCCAAGCTGCTGATCCACAAGGTGTCCAACCG
CTTCAGCGGGGTGCCCGACCGCTTCAGCGGGTCGG
GCAGCGGGACGGACTTCACCCTGAAGATCAGCCGC
GTGGAGGCCGAGGACCTCGGGGTGTACTTCTGCTC
GCAGAGCACCCACGTGCCGCCGCTTACCTTCGGGG
CCGGCACCAAGCTGGAGCTCAAGCGGGCCGAC.

SEQ ID NO: 43 includes Target scFv: 1-732; Target VL: 385-732; Target linker: 340-384; Target VH: 1-339; Free energy: −339.9; gdT CAI: 0.78785010580307; ORF count: 2.

SEQ ID NO: 44 is the sequence name for GD2 scFv ECOg (86). It is a Tumor targeting scFv construct. It is a DNA sequence. The sequence is:

GAGGTCCAGCTCCTCCAGTCCGGGCCCGAGCTGGA
GAAGCCCGGGGCCTCCGTGATGATCTCCTGCAAGG
CCTCCGGGTCCTCGTTCACCGGGTACAACATGAAC
TGGGTGAGGCAGAACATCGGGAAGAGCCTGGAGTG
GATCGGGGCCATCGACCCCTACTACGGCGGAACCT
CGTACAACCAGAAGTTCAAGGGGAGAGCCACGCTG
ACCGTGGACAAGTCCTCGTCCACCGCGTACATGCA
CCTGAAGAGCCTGACCTCCGAGGACTCCGCCGTCT
ACTACTGCGTCTCCGGGATGAAGTACTGGGGACAG
GGGACCTCCGTGACCGTCTCCTCCGGCGGAGGAGG
CTCAGGCGGAGGAGGCTCCGGCGGAGGAGGCTCCG
ACGTGGTGATGACCCAGACTCCCCTCTCCCTGCCC
GTCTCCCTCGGCGACCAGGCGAGCATCTCCTGCCG
GTCCTCTCAGTCCCTCGTCCACCGGAACGGGAACA
CCTACCTCCACTGGTACCTGCAGAAGCCCGGGCAG
TCGCCCAAGCTGCTCATCCACAAGGTGAGCAACCG
CTTCTCCGGGGTCCCCGACCGCTTCTCCGGGTCCG
GGTCCGGGACCGACTTCACCCTGAAGATCTCCCGG
GTCGAGGCCGAGGACCTCGGCGTCTACTTCTGCTC
CCAGTCTACCCACGTTCCTCCGCTGACCTTCGGCG
CGGGGACGAAGCTGGAGCTGAAGAGGGCCGAT.

SEQ ID NO: 44 includes Target scFv: 1-732; Target VL: 385-732; Target linker: 340-384; Target VH: 1-339; Free energy: −336.5; gdT CAI: 0.849533769168508; ORF count: 0.

SEQ ID NO: 45 is the sequence name for integrin aVb3 scFv. It is a Tumor targeting scFv construct. It is an AA sequence. The sequence is:

EVQLEESGGGLVKPGGSLKLSCAASGFAFSSYDMS
WVRQIPEKRLEWVAKVSSGGGSTYYLDTVQGRFTI
SRDNAKNTLYLQMSSLNSEDTAMYYCARHNYGSFA
YWGQGTLVTVSAAKGGGGSGGGGSGGGGSELVMTQ
TPATLSVTPGDSVSLSCRASQSISNHLHWYQQKSH
ESPRLLIKYASQSISGIPSRFSGSGSGTDFTLSIN
SVETEDFGMYFCQQSNSWPHTFGGGTKLEIK.

SEQ ID NO: 45 includes Target scFv: 1-241; Target VL: 135-241; Target linker: 120-134; Target VH: 1-119.

SEQ ID NO: 46 is the sequence name for integrin aVb3 scFv. It is a Tumor targeting scFv construct. It is a DNA sequence. The sequence is:

GAGGTGCAGCTGGAGGAGTCTGGAGGTGGCCTGGT
GAAGCCAGGAGGCAGCCTGAAGCTGAGCTGTGCTG
CCTCTGGGTTTGCCTTCAGCTCCTATGACATGAGC
TGGGTGAGGCAGATCCCTGAGAAGAGGCTGGAGTG
GGTAGCTAAGGTGAGCTCTGGAGGTGGCAGCACCT
ACTACCTGGACACAGTGCAGGGCAGGTTCACCATC
AGCAGGGACAATGCTAAGAACACCCTGTACCTGCA
GATGAGCAGCCTGAACTCTGAGGACACAGCTATGT
ACTACTGTGCCAGGCACAACTATGGGTCCTTTGCC
TACTGGGGCCAGGGGACCCTGGTGACAGTGTCTGC
AGCTAAAGGTGGAGGTGGCTCTGGAGGTGGAGGCT
CTGGAGGTGGAGGCTCTGAGCTGGTGATGACCCAG
ACCCCAGCTACCCTGAGTGTGACCCCAGGGGACTC
TGTGTCCCTGAGCTGCAGGGCCAGCCAGTCCATCT
CCAACCACCTGCACTGGTACCAGCAGAAGTCCCAT
GAGAGCCCCAGGCTGCTGATCAAGTATGCCTCTCA
GTCCATCTCTGGCATCCCCAGCAGGTTCTCAGGGT
CAGGGTCAGGGACAGACTTCACCCTGAGCATCAAC
TCTGTGGAGACAGAGGACTTTGGGATGTACTTCTG
CCAGCAGTCCAACTCCTGGCCCCACACCTTTGGAG
GTGGCACCAAGCTGGAGATCAAG.

SEQ ID NO: 46 includes Target scFv: 1-723; Target VL: 403-723; Target linker: 358-402; Target VH: 1-357; Free energy: −326.8; gdT CAI: 0.836939021276497; ORF count: 8.

SEQ ID NO: 47 is the sequence name for integrin aVb3 scFv ECOg (62). It is a Tumor targeting scFv construct. It is a DNA sequence. The sequence is:

GAGGTGCAGCTGGAGGAGTCCGGCGGCGGCTTGGTGAAGCCCGGCGGCTC
CCTGAAGCTGTCCTGCGCCGCCTCCGGGTTCGCCTTCTCCAGCTACGACA
TGTCCTGGGTGCGGCAGATCCCCGAGAAGCGGCTGGAGTGGGTCGCCAAG
GTGTCCTCCGGCGGCGGCTCCACCTACTACCTGGACACCGTGCAGGGGCG
CTTCACCATCTCCCGGGACAACGCCAAGAACACCCTGTACCTGCAGATGA
GCTCCCTGAACAGCGAGGACACCGCCATGTACTACTGCGCCCGGCACAAC
TACGGGTCCTTCGCCTACTGGGGCCAGGGGACCCTGGTGACCGTGTCCGC
CGCCAAAGGAGGCGGCGGCTCCGGAGGAGGTGGCTCAGGCGGCGGAGGCA
GCGAGCTGGTGATGACCCAGACCCCGGCCACGCTGTCCGTGACGCCCGGG
GACTCCGTGTCCCTGAGCTGCCGGGCCAGCCAGTCCATCAGCAACCACCT
GCACTGGTACCAGCAGAAGTCCCACGAGAGCCCGCGGCTGCTGATCAAGT
ACGCCAGCCAGTCCATCTCCGGCATCCCCAGCCGGTTCTCCGGGTCCGGG
TCCGGGACCGACTTCACCCTGTCCATCAACTCCGTGGAGACCGAGGACTT
CGGGATGTACTTCTGCCAGCAGAGCAACAGCTGGCCCCACACCTTCGGCG
GCGGCACCAAGCTGGAGATCAAG.

SEQ ID NO: 47 includes Target scFv: 1-723; Target VL: 403-723; Target linker: 358-402; Target VH: 1-357; Free energy: −324.4; gdT CAI: 0.868908; ORF count: 1.

SEQ ID NO: 48 is the sequence name for integrin aVb3 scFv ECOg (26). It is a Tumor targeting scFv construct. It is a DNA sequence. The sequence is:

GAAGTCCAGCTGGAGGAGAGCGGCGGTGGCCTCGTGAAGCCCGGCGGTTC
GCTCAAGCTGAGCTGCGCGGCCAGCGGGTTCGCCTTCAGCTCGTACGACA
TGAGCTGGGTGCGGCAGATCCCCGAGAAGCGCCTGGAGTGGGTCGCCAAG
GTGTCCTCCGGCGGCGGCTCGACGTACTACCTGGACACCGTGCAGGGGCG
CTTCACGATCAGCCGGGACAACGCGAAGAACACCCTGTACCTCCAGATGT
CCTCGCTGAACTCCGAGGACACCGCGATGTACTACTGCGCGCGGCACAAC
TACGGGAGCTTCGCCTACTGGGGCCAGGGCACCCTGGTCACCGTGAGCGC
GGCGAAAGGCGGTGGCGGCAGTGGTGGCGGAGGCTCTGGCGGCGGTGGCT
CCGAGCTGGTGATGACCCAGACCCCGGCGACGCTCTCCGTGACGCCCGGG
GACTCCGTGAGCCTGTCCTGCCGGGCCTCGCAGAGCATCTCCAACCACCT
GCACTGGTACCAGCAGAAGAGCCACGAGTCGCCCCGGCTGCTCATCAAGT
ACGCCTCGCAGAGCATCTCCGGGATTCCCTCGCGGTTCTCCGGGAGCGGC
TCGGGGACGGACTTCACGCTGTCCATCAACTCCGTGGAGACCGAGGACTT
CGGGATGTACTTCTGCCAGCAGAGCAACTCCTGGCCGCACACCTTCGGCG
GCGGCACCAAGCTGGAGATCAAG.

SEQ ID NO: 48 includes Target scFv: 1-723; Target VL: 403-723; Target linker: 358-402; Target VH: 1-357; Free energy: −342.6; gdT CAI: 0.773341369143768; ORF count: 1.

SEQ ID NO: 49 is the sequence name for integrin aVb3 scFv ECOg (12). It is a Tumor targeting scFv construct. It is a DNA sequence. The sequence is:

GAGGTCCAGCTGGAGGAATCCGGCGGCGGCCTCGTGAAGCCAGGCGGCAG
CCTGAAGCTGTCGTGCGCGGCCTCCGGGTTCGCCTTCAGCTCGTACGACA
TGAGCTGGGTGCGCCAGATCCCCGAGAAGCGCCTGGAGTGGGTCGCCAAG
GTGTCCTCCGGCGGCGGCAGCACGTACTACTTGGACACCGTGCAGGGGCG
CTTCACGATCTCGCGGGACAACGCGAAGAACACGCTGTACCTCCAGATGT
CCTCGCTGAACTCGGAGGACACCGCGATGTACTACTGCGCGCGGCACAAC
TATGGGTCCTTCGCCTACTGGGGCCAGGGGACCCTGGTGACCGTGAGCGC
GGCCAAAGGTGGCGGCGGCTCAGGTGGCGGAGGCAGCGGTGGCGGAGGCA
GCGAGCTCGTGATGACGCAGACGCCGGCGACCCTCAGTGTGACCCCAGGG
GACTCCGTCAGCCTGAGCTGCCGGGCGTCCCAGTCGATCTCGAACCACCT
GCACTGGTACCAGCAGAAGTCCCACGAGAGCCCCAGGTTGCTCATCAAGT
ACGCCAGCCAGTCGATCTCCGGGATTCCCTCTCGGTTCTCGGGGTCCGGC
AGCGGGACCGACTTCACGCTGTCGATCAACAGCGTGGAGACCGAGGACTT
CGGGATGTACTTCTGCCAGCAGTCCAACAGTTGGCCCCACACGTTCGGCG
GCGGCACCAAGCTGGAGATCAAG.

SEQ ID NO: 49 includes Target scFv: 1-723; Target VL: 403-723; Target linker: 358-402; Target VH: 1-357; Free energy: −339.8; gdT CAI: 0.762733907249084; ORF count: 2.

SEQ ID NO: 50 is the sequence name for integrin aVb3 scFv ECOg (48). It is a Tumor targeting scFv construct. It is a DNA sequence. The sequence is:

GAGGTCCAGCTGGAGGAGTCCGGCGGAGGCCTGGTCAAGCCCGGCGGCAG
CCTGAAGCTGTCCTGCGCCGCCTCCGGCTTCGCCTTCAGCAGCTACGACA
TGTCCTGGGTCCGGCAGATCCCGGAGAAGCGGCTGGAGTGGGTCGCCAAG
GTCAGCTCCGGCGGCGGCAGCACCTACTACCTGGACACCGTCCAGGGCCG
GTTCACCATCAGCCGGGACAACGCCAAGAACACCCTGTACCTGCAGATGT
CCAGCCTGAACTCCGAGGACACGGCCATGTACTACTGCGCCCGGCACAAC
TACGGCAGCTTCGCCTACTGGGGCCAGGGGACCCTGGTGACCGTGTCGGC
CGCCAAAGGCGGAGGCGGCTCAGGAGGCGGAGGTTCCGGCGGCGGAGGCT
CCGAGCTGGTCATGACCCAGACGCCGGCCACGCTGTCCGTGACGCCCGGC
GACTCCGTCAGCCTGTCCTGCCGGGCCAGCCAGTCCATCAGCAACCACCT
GCACTGGTACCAGCAGAAGTCCCACGAGTCGCCCCGGCTGCTGATCAAGT
ACGCCAGCCAGTCCATCTCCGGCATCCCCAGCCGGTTCTCCGGGTCCGGG
TCCGGGACCGACTTCACCCTGTCCATCAACTCCGTCGAGACCGAGGACTT
CGGCATGTACTTCTGCCAGCAGTCCAACAGCTGGCCGCACACCTTCGGCG
GCGGCACCAAGCTGGAGATCAAG.

SEQ ID NO: 50 includes Target scFv: 1-723; Target VL: 403-723; Target linker: 358-402; Target VH: 1-357; Free energy: −329.4; gdT CAI: 0.82265589760209; ORF count: 0.

SEQ ID NO: 51 is the sequence name for integrin aVb3 scFv ECOg (97). It is a Tumor targeting scFv construct. It is a DNA sequence. The sequence is:

GAGGTCCAGCTGGAGGAGTCCGGAGGAGGACTCGTGAAGCCCGGAGGCTC
CCTGAAGCTCTCCTGCGCCGCCTCCGGGTTCGCCTTCTCCTCCTACGACA
TGTCCTGGGTCCGGCAGATCCCCGAGAAGAGGCTGGAGTGGGTCGCGAAG
GTCTCCTCCGGAGGAGGCTCGACCTACTATCTCGACACGGTCCAGGGCCG
GTTCACGATCTCCCGGGACAACGCGAAGAACACGCTCTACCTCCAGATGT
CGAGCCTGAACTCCGAGGACACCGCGATGTACTACTGCGCCCGGCACAAC
TACGGGAGCTTCGCCTACTGGGGCCAGGGGACTCTCGTGACCGTCTCCGC
CGCGAAAGGCGGAGGAGGCTCCGGAGGAGGAGGCTCCGGAGGAGGAGGCT
CCGAGCTCGTGATGACCCAGACCCCGGCGACCCTCTCCGTGACGCCCGGG
GACTCCGTCTCCCTCTCCTGTCGCGCGAGCCAGTCGATCTCGAACCATCT
CCACTGGTACCAGCAGAAGAGTCACGAGTCTCCCCGGCTCCTGATCAAGT
ACGCGAGCCAGTCGATCTCCGGGATTCCCTCGCGGTTCTCCGGGTCCGGG
TCCGGGACCGACTTCACGCTCTCGATCAACTCCGTCGAGACCGAGGACTT
CGGGATGTACTTCTGCCAGCAGTCGAACTCCTGGCCCCACACGTTCGGCG
GCGGCACGAAGCTGGAGATCAAG.

SEQ ID NO: 51 includes Target scFv: 1-723; Target VL: 403-723; Target linker: 358-402; Target VH: 1-357; Free energy: −329.3; gdT CAI: 0.777965026449684; ORF count: 0.

SEQ ID NO: 52 is the sequence name for SSTR2 scFv. It is a Tumor targeting scFv construct. It is an AA sequence. The sequence is:

DIVMTQSPDSLAVSLGERATINCKSSQSLLNSRNRKNYLAWYQQKPDQSP
KLLIYWASTRESGVPDRFSGSGSGTDFTLTISSLQAEDVAVYYCKQSYYL
WTFGGGTKVEIKGGGGSGGGGSGGGGSEVQLVESGGGLVQPGGSLRLSCA
ASGFTFSDYGMAWFRQAPGKGLEWVSFISNLGYSIYYADSVKGRFTISRD
NAKNSLYLQMNSLRAEDTAVYYCARAPYDYDSFDPMDYWGQGTLVTVS.

SEQ ID NO: 52 includes Target scFv: 1-248; Target VL: 1-112; Target linker: 113-127; Target VH: 128-248.

SEQ ID NO: 53 is the sequence name for SSTR2 scFv. It is a Tumor targeting scFv construct. It is a DNA sequence. The sequence is:

GACATTGTGATGACCCAGAGCCCAGACTCCCTGGCTGTGAGCCTAGGGGA
GAGGGCCACCATCAACTGCAAGTCCTCTCAGAGCCTCCTCAACTCCAGGA
ACAGGAAGAACTACCTGGCCTGGTACCAGCAGAAGCCAGACCAGAGCCCC
AAGCTGCTCATCTACTGGGCCTCCACCAGGGAGTCTGGGGTGCCTGACAG
GTTCTCTGGGTCTGGGTCTGGGACTGACTTCACCCTGACCATCAGCTCCC
TGCAGGCTGAGGATGTGGCTGTGTACTACTGCAAGCAGAGCTACTACCTG
TGGACCTTTGGTGGAGGCACCAAGGTGGAGATCAAAGGAGGTGGAGGCTC
TGGTGGTGGAGGCTCTGGTGGTGGAGGCTCTGAGGTGCAGCTGGTGGAGT
CTGGTGGTGGCCTGGTCCAGCCAGGAGGCTCCCTGAGGCTGAGCTGTGCT
GCCTCTGGGTTCACCTTCTCAGACTATGGGATGGCCTGGTTCAGGCAGGC
CCCAGGGAAGGGCCTGGAGTGGGTGAGCTTCATCTCCAACCTGGGCTACT
CCATCTACTATGCTGACTCTGTGAAGGGCAGGTTCACCATCAGCAGGGAC
AATGCCAAGAACAGCCTGTACCTGCAGATGAACTCCCTGAGGGCTGAGGA
CACAGCTGTGTACTACTGTGCCAGGGCCCCATATGACTATGACAGCTTTG
ACCCCATGGACTACTGGGGCCAGGGGACCCTGGTGACTGTGAGC.

SEQ ID NO: 53 includes Target scFv: 1-744; Target VL: 1-336; Target linker: 337-381; Target VH: 382-744; Free energy: −344; gdT CAI: 0.841055073258909; ORF count: 7.

SEQ ID NO: 54 is the sequence name for SSTR2 svFv ECOg (72). It is a Tumor targeting scFv construct. It is a DNA sequence. The sequence is:

GACATCGTGATGACCCAGAGCCCCGACTCCCTGGCCGTGAGCCTCGGGGA
GAGGGCCACGATCAACTGCAAGAGCTCCCAGAGCCTGCTCAACTCCCGGA
ACCGGAAGAACTACCTGGCCTGGTACCAGCAGAAGCCCGACCAGTCGCCC
AAGCTGCTCATCTACTGGGCCTCCACCAGGGAGAGCGGGGTGCCCGACCG
CTTCTCCGGGAGCGGGTCCGGGACCGACTTCACCCTGACCATCTCCTCTC
TGCAGGCCGAGGACGTGGCCGTGTACTACTGCAAGCAGAGCTACTACCTG
TGGACATTCGGCGGCGGCACCAAGGTGGAGATCAAAGGCGGCGGCGGCAG
TGGTGGAGGAGGCAGCGGCGGCGGTGGCTCAGAGGTGCAGCTGGTGGAGA
GCGGAGGCGGCCTGGTGCAGCCAGGCGGCTCTCTGCGCCTGTCCTGCGCC
GCCTCTGGGTTCACCTTCTCCGACTACGGGATGGCCTGGTTCCGCCAGGC
CCCAGGGAAGGGGCTGGAGTGGGTGTCCTTCATCTCCAACCTGGGGTACA
GCATCTACTACGCCGACTCCGTGAAGGGCCGGTTCACCATCAGCCGGGAC
AACGCCAAGAACAGCCTGTACCTGCAGATGAACTCCCTGCGCGCCGAGGA
CACCGCCGTGTACTACTGCGCCCGGGCTCCCTACGACTACGACAGCTTCG
ACCCCATGGACTACTGGGGCCAGGGGACACTGGTCACCGTGTCC.

SEQ ID NO: 54 includes Target scFv: 1-744; Target VL: 1-336; Target linker: 337-381; Target VH: 382-744; Free energy: −325.1; gdT CAI: 0.865568; ORF count: 1.

SEQ ID NO: 55 is the sequence name for SSTR2 svFv ECOg (64). It is a Tumor targeting scFv construct. It is a DNA sequence. The sequence is:

GACATCGTGATGACGCAGAGCCCCGACTCTTTGGCGGTGAGCCTCGGGGA
GAGGGCCACCATCAACTGCAAGTCGTCGCAGAGCCTCCTCAACAGCCGCA
ACCGGAAGAACTACCTCGCCTGGTATCAGCAGAAGCCCGACCAAAGCCCC
AAGCTGCTGATCTACTGGGCCTCCACACGGGAGTCTGGGGTGCCCGACCG
CTTCAGCGGCAGCGGCAGCGGCACCGACTTCACCCTCACCATCAGCAGCC
TGCAGGCGGAGGATGTGGCGGTGTACTACTGCAAACAGTCCTACTATCTG
TGGACCTTCGGCGGCGGCACAAAGGTGGAGATCAAAGGCGGCGGCGGCAG
TGGAGGAGGAGGCAGCGGCGGCGGAGGCTCTGAGGTGCAGCTGGTGGAGA
GCGGCGGCGGTTTGGTGCAGCCCGGCGGCAGCCTCCGACTCAGCTGCGCC
GCCTCCGGCTTCACCTTCTCCGACTACGGGATGGCGTGGTTTCGGCAGGC
CCCGGGGAAGGGGTTGGAGTGGGTGAGCTTCATCTCCAACTTGGGGTACA
GCATCTACTACGCCGACTCTGTGAAGGGGCGCTTCACCATCAGCCGCGAC
AACGCCAAAAACAGCCTCTATCTGCAGATGAACAGCCTCCGCGCTGAGGA
CACAGCGGTGTACTACTGCGCCCGCGCCCCATACGACTACGACTCCTTCG
ACCCCATGGACTACTGGGGTCAGGGGACGTTGGTGACGGTGTCG.

SEQ ID NO: 55 includes Target scFv: 1-744; Target VL: 1-336; Target linker: 337-381; Target VH: 382-744; Free energy: −333.3; gdT CAI: 0.782038165701074; ORF count: 4.

SEQ ID NO: 56 is the sequence name for SSTR2 svFv ECOg (15). It is a Tumor targeting scFv construct. It is a DNA sequence. The sequence is:

GACATCGTCATGACCCAGTCACCTGACAGCCTGGCCGTCAGCCTGGGCGA
ACGGGCCACCATCAACTGTAAGTCATCTCAGAGCCTGCTGAACAGCCGGA
ACCGGAAGAACTACCTGGCCTGGTATCAGCAGAAGCCTGATCAGTCACCT
AAGCTGCTGATCTACTGGGCCTCAACCAGAGAGTCCGGCGTGCCTGACAG
GTTCAGCGGGTCCGGGTCCGGGACCGACTTCACCCTGACCATCAGCAGCC
TGCAGGCCGAGGACGTGGCCGTCTATTACTGTAAGCAGTCTTATTACCTG
TGGACCTTCGGCGGCGGCACCAAGGTCGAGATCAAAGGCGGCGGCGGCTC
AGGTGGTGGTGGCTCCGGCGGCGGTGGCTCTGAGGTCCAGCTGGTCGAGT
CCGGCGGTGGCCTGGTCCAGCCAGGCGGCAGCCTGAGGCTGTCCTGCGCC
GCCTCCGGCTTCACCTTCTCTGACTACGGCATGGCCTGGTTCAGACAGGC
CCCAGGCAAGGGCCTGGAGTGGGTCAGCTTCATCAGCAACCTGGGCTACA
GCATCTATTACGCCGACAGCGTCAAGGGCCGGTTCACCATCAGCCGGGAC
AACGCCAAGAACAGCCTGTACCTGCAGATGAACAGCCTGAGGGCCGAGGA
CACGGCCGTCTATTACTGCGCCCGGGCCCCTTATGACTATGACAGCTTTG
ACCCCATGGACTACTGGGGCCAGGGGACCCTGGTGACCGTGTCA.

SEQ ID NO: 56 includes Target scFv: 1-744; Target VL: 1-336; Target linker: 337-381; Target VH: 382-744; Free energy: −331.6; gdT CAI: 0.812422023876491; ORF count: 8.

SEQ ID NO: 57 is the sequence name for SSTR2 svFv ECOg (50). It is a Tumor targeting scFv construct. It is a DNA sequence. The sequence is:

GACATCGTGATGACCCAGAGCCCCGACTCCCTGGCCGTGTCCCTCGGGGA
GAGGGCCACCATCAACTGCAAGAGCTCCCAGTCCCTGCTGAACTCCCGGA
ACCGGAAGAACTACCTGGCCTGGTACCAGCAGAAGCCCGACCAGAGCCCC
AAGCTGCTGATCTACTGGGCCAGCACCAGGGAGAGCGGGGTGCCCGACCG
CTTCTCCGGGTCCGGGTCCGGGACCGACTTCACCCTGACCATCAGCTCCC
TGCAGGCCGAGGACGTGGCCGTGTACTACTGCAAGCAGAGCTACTACCTG
TGGACCTTCGGCGGCGGCACCAAGGTGGAGATCAAAGGCGGCGGCGGCTC
AGGAGGAGGTGGCTCCGGCGGCGGAGGCTCTGAGGTGCAGCTGGTGGAGA
GCGGCGGCGGACTGGTGCAGCCAGGCGGCTCCCTGCGGCTGAGCTGCGCC
GCCTCTGGGTTCACCTTCTCCGACTACGGGATGGCCTGGTTCCGGCAGGC
CCCAGGGAAGGGGCTGGAGTGGGTGAGCTTCATCTCCAACCTGGGGTACT
CCATCTACTACGCCGACTCCGTGAAGGGGCGCTTCACCATCTCCCGGGAC
AACGCCAAGAACTCCCTGTACCTGCAGATGAACTCCCTGCGGGCCGAGGA
CACCGCGGTGTACTACTGCGCCCGGGCCCCGTACGACTACGACAGCTTCG
ACCCCATGGACTACTGGGGCCAGGGGACCCTGGTGACCGTGAGC.

SEQ ID NO: 57 includes Target scFv: 1-744; Target VL: 1-336; Target linker: 337-381; Target VH: 382-744; Free energy: −328.9; gdT CAI: 0.878055710303915; ORF count: 3.

SEQ ID NO: 58 is the sequence name for SSTR2 svFv ECOg (42). It is a Tumor targeting scFv construct. It is a DNA sequence. The sequence is:

GATATCGTGATGACCCAGTCCCCGGACTCCCTGGCAGTGTCCCTCGGGGA
GCGGGCCACCATCAACTGCAAGAGCTCCCAGTCCCTGCTGAACTCCCGGA
ACCGGAAGAACTACCTGGCCTGGTACCAGCAGAAGCCCGACCAGTCCCCG
AAGCTGCTGATCTACTGGGCCAGCACCCGGGAATCCGGGGTGCCCGACCG
CTTCTCCGGGTCCGGGTCCGGGACCGACTTCACCCTGACCATCAGCTCCC
TGCAGGCCGAGGACGTGGCAGTGTACTACTGCAAGCAGTCCTACTACCTG
TGGACCTTCGGCGGCGGCACCAAGGTGGAGATCAAAGGCGGCGGCGGCTC
TGGAGGAGGAGGCTCCGGCGGCGGAGGTTCCGAGGTGCAGCTGGTGGAGT
CCGGCGGAGGACTGGTGCAGCCCGGCGGCTCCCTGCGACTGTCCTGCGCC
GCCTCCGGGTTCACCTTCTCCGACTACGGGATGGCCTGGTTCCGGCAGGC
TCCCGGGAAGGGGCTGGAGTGGGTGTCCTTCATCTCCAACCTGGGGTACT
CCATCTACTACGCCGACTCCGTGAAGGGCCGGTTCACCATCTCCCGGGAC
AACGCCAAGAACTCCCTGTACCTGCAGATGAACTCCCTGCGGGCCGAGGA
CACCGCAGTGTACTACTGCGCCCGGGCCCCGTACGACTACGACTCCTTCG
ACCCCATGGACTACTGGGGCCAGGGGACCCTGGTGACCGTGTCC.

SEQ ID NO: 58 includes Target scFv: 1-744; Target VL: 1-336; Target linker: 337-381; Target VH: 382-744; Free energy: −328.6; gdT CAI: 0.886975497635559; ORF count: 1.

SEQ ID NO: 59 is the sequence name for 2×SST28 3×G4S ligand. It is a Tumor targeting ligand construct. It is an AA sequence. The sequence is:

SANSNPAMAPRERKAGCKNFFWKTFTSCGGGGSGGGGSGGGGSSANSNPA
MAPRERKAGCKNFFWKTFTSC.

SEQ ID NO: 59 includes Target Ligand: 1-71.

SEQ ID NO: 60 is the sequence name for 2×SST28 3×G4S ligand. It is a Tumor Targeting Ligand construct. It is a DNA sequence. The sequence is:

AGCGCGAACAGCAACCCGGCGATGGCGCCGCGCGAACGCAAAGCGGGCTG
CAAAAACTTTTTTTGGAAAACCTTTACCAGCTGCGGCGGCGGCGGCAGCG
GCGGCGGCGGCAGCGGCGGCGGCGGCAGCAGCGCGAACAGCAACCCGGCG
ATGGCGCCGCGCGAACGCAAAGCGGGCTGCAAAAACTTTTTTTGGAAAAC
CTTTACCAGCTGCGGCGGCGGCGGCAGCGGCGGCGGCGGCAGCGGCGGCG
GCGGCAGC.

SEQ ID NO: 60 includes Target Ligand: 1-213; Free energy: −117; gdT CAI: 0.603084331934136; ORF count: 0.

SEQ ID NO: 61 is the sequence name for 2×SST28 3×G4S ligand ECOg (192). It is a Tumor Targeting Ligand construct. It is a DNA sequence. The sequence is:

TCTGCCAACTCCAACCCCGCTATGGCTCCCAGGGAGCGGAAGGCCGGGTG
CAAGAACTTCTTCTGGAAGACCTTCACCTCCTGCGGAGGCGGAGGCTCAG
GAGGCGGAGGCTCCGGAGGAGGCGGCTCCTCCGCCAACTCCAACCCCGCT
ATGGCTCCCAGGGAGCGGAAGGCCGGGTGCAAGAACTTCTTCTGGAAGAC
CTTCACCTCCTGCGGAGGCGGAGGCTCCGGAGGAGGCGGCTCAGGAGGCG
GCGGCAGC.

SEQ ID NO: 61 includes Target Ligand: 1-213; Free energy: −138.2; gdT CAI: 0.800017929945784; ORF count: 0.

SEQ ID NO: 62 is the sequence name for 2×SST28 3×G4S ligand ECOg (141). It is a Tumor Targeting Ligand construct. It is a DNA sequence. The sequence is:

TCCGCCAACTCCAACCCCGCTATGGCTCCCCGGGA
GCGGAAGGCTGGGTGCAAGAACTTCTTCTGGAAGA
CCTTCACCTCCTGCGGAGGCGGAGGTTCCGGAGGC
GGAGGTTCCGGCGGAGGCGGCTCCTCCGCCAACTC
CAACCCCGCGATGGCTCCCAGGGAGCGGAAGGCCG
GGTGCAAGAACTTCTTCTGGAAGACCTTCACCTCC
TGCGGAGGCGGAGGTTCCGGCGGAGGAGGTTCCGG
AGGCGGCGGCTCC.

SEQ ID NO: 62 includes Target Ligand: 1-213; Free energy: −146.7; gdT CAI: 0.786043171489395; ORF count: 0.

SEQ ID NO: 63 is the sequence name for 2×SST28 3×G4S ligand ECOg (241). It is a Tumor Targeting Ligand construct. It is a DNA sequence. The sequence is:

AGCGCCAACTCCAACCCCGCTATGGCTCCCCGGGA
GCGCAAGGCTGGGTGCAAGAACTTCTTCTGGAAGA
CCTTCACCTCCTGCGGAGGCGGAGGCTCTGGAGGC
GGAGGCTCTGGCGGAGGCGGCTCCTCCGCCAACTC
CAACCCCGCGATGGCTCCCAGGGAGCGCAAGGCCG
GGTGCAAGAACTTCTTCTGGAAGACCTTCACCTCC
TGCGGAGGCGGAGGCTCTGGCGGAGGAGGCTCTGG
AGGCGGCGGCTCC.

SEQ ID NO: 63 includes Target Ligand: 1-213; Free energy: −142.6; gdT CAI: 0.788525759670669; ORF count: 0.

SEQ ID NO: 64 is the sequence name for 2×SST28 3×G4S ligand ECOg (172). It is a Tumor Targeting Ligand construct. It is a DNA sequence. The sequence is:

TCCGCCAACTCCAACCCGGCCATGGCTCCCCGGGA
GAGGAAGGCCGGGTGCAAGAACTTCTTCTGGAAGA
CCTTCACCTCCTGCGGAGGAGGAGGAAGCGGAGGA
GGAGGAAGCGGAGGAGGAGGCTCCTCGGCCAACTC
CAACCCGGCCATGGCTCCCCGGGAGAGGAAGGCCG
GGTGCAAGAACTTCTTCTGGAAGACCTTCACCTCC
TGCGGAGGAGGAGGAAGCGGAGGAGGAGGAAGCGG
AGGAGGAGGATCT.

SEQ ID NO: 64 includes Target Ligand: 1-213; Free energy: −140; gdT CAI: 0.719392416533176; ORF count: 0.

SEQ ID NO: 65 is the sequence name for 2×SST28 3×G4S ligand ECOg (266). It is a Tumor Targeting Ligand construct. It is a DNA sequence. The sequence is:

TCCGCCAACTCCAACCCCGCTATGGCTCCCCGGGA
GCGGAAGGCTGGGTGCAAGAACTTCTTCTGGAAGA
CCTTCACCTCCTGTGGCGGCGGCGGATCTGGCGGA
GGAGGCTCTGGCGGAGGCGGCTCCTCCGCCAACTC
CAACCCCGCTATGGCTCCCAGGGAGCGGAAGGCCG
GGTGCAAGAACTTCTTCTGGAAGACCTTCACCTCC
TGTGGCGGCGGCGGATCTGGCGGAGGAGGCTCTGG
CGGAGGCGGAAGC.

SEQ ID NO: 65 includes Target Ligand: 1-213; Free energy: −135.3; gdT CAI: 0.799023029714678; ORF count: 0.

SEQ ID NO: 66 is the sequence name for 2×SST28 2×G4S ligand. It is a Tumor Targeting Ligand construct. It is an AA sequence. The sequence is:

SANSNPAMAPRERKAGCKNFFWKTFTSCGGSGGSG
GSGGSGGSANSNPAMAPRERKAGCKNFFWKTFTSC
GGSGGSGGSGGSGG.

SEQ ID NO: 66 includes Target Ligand: 1-70; Central Linker: 71-84.

SEQ ID NO: 67 is the sequence name for 2×SST28 2×G4S ligand. It is a Tumor Targeting Ligand construct. It is a DNA sequence. The sequence is:

AGCGCGAACAGCAACCCGGCGATGGCGCCGCGCGA
ACGCAAAGCGGGCTGCAAAAACTTTTTTTGGAAAA
CCTTTACCAGCTGCGGCGGCAGCGGCGGCAGCGGC
GGCAGCGGCGGCAGCGGCGGCAGCGCGAACAGCAA
CCCGGCGATGGCGCCGCGCGAACGCAAAGCGGGCT
GCAAAAACTTTTTTTGGAAAACCTTTACCAGCTGC
GGCGGCAGCGGCGGCAGCGGCGGCAGCGGCGGCAG
CGGCGGC.

SEQ ID NO: 67 includes Target Ligand: 1-210; Central Linker: 221-252; Free energy: −110.4; gdT CAI: 0.602525074392084; ORF count: 0.

SEQ ID NO: 68 is the sequence name for 2×SST28 2×G4S ligand ECOg (114). It is a Tumor Targeting Ligand construct. It is a DNA sequence. The sequence is:

TCCGCCAACTCCAACCCCGCAATGGCTCCCCGGGA
GAGGAAGGCCGGGTGCAAGAACTTCTTCTGGAAGA
CCTTCACCAGCTGCGGAGGCTCCGGAGGCTCCGGA
GGCTCCGGAGGCTCCGGAGGCTCCGCCAACTCCAA
CCCCGCAATGGCTCCCCGGGAGAGGAAGGCCGGGT
GCAAGAACTTCTTCTGGAAGACCTTCACCAGCTGC
GGAGGCTCCGGAGGCTCCGGAGGCTCCGGAGGCTC
CGGAGGC.

SEQ ID NO: 68 includes Target Ligand: 1-210; Central Linker: 221-252; Free energy: −140.6; gdT CAI: 0.815587600211903; ORF count: 0.

SEQ ID NO: 69 is the sequence name for 2×SST28 2×G4S ligand ECOg (86). It is a Tumor Targeting Ligand construct. It is a DNA sequence. The sequence is:

TCTGCCAATTCCAACCCCGCGATGGCCCCTCGGGA
GAGGAAGGCCGGGTGCAAGAACTTCTTCTGGAAGA
CCTTCACCTCCTGCGGAGGCTCCGGAGGCTCCGGA
GGCTCCGGAGGCTCCGGAGGCTCTGCCAATTCCAA
CCCCGCGATGGCCCCTCGGGAGAGGAAGGCCGGGT
GCAAGAACTTCTTCTGGAAGACCTTCACCTCCTGC
GGAGGCTCCGGAGGCTCCGGAGGCTCCGGAGGCTC
CGGAGGC.

SEQ ID NO: 69 includes Target Ligand: 1-210; Central Linker: 221-252; Free energy: −146.5; gdT CAI: 0.793448781755408; ORF count: 0.

SEQ ID NO: 70 is the sequence name for 2×SST28 2×G4S ligand ECOg (132). It is a Tumor Targeting Ligand construct. It is a DNA sequence. The sequence is:

TCCGCGAACTCCAACCCCGCGATGGCTCCCCGGGA
GAGGAAGGCCGGGTGCAAGAACTTCTTCTGGAAGA
CGTTCACGTCCTGCGGAGGCTCCGGAGGCTCCGGA
GGCTCCGGAGGCTCCGGAGGCTCCGCGAACTCCAA
CCCCGCGATGGCTCCCCGGGAGAGGAAGGCCGGGT
GCAAGAACTTCTTCTGGAAGACGTTCACGTCCTGC
GGAGGCTCCGGAGGCTCCGGAGGCTCCGGAGGCTC
CGGAGGA.

SEQ ID NO: 70 includes Target Ligand: 1-210; Central Linker: 221-252; Free energy: −141.6; gdT CAI: 0.768520163043281; ORF count: 0.

SEQ ID NO: 71 is the sequence name for 2×SST28 2×G4S ligand ECOg (131). It is a Tumor Targeting Ligand construct. It is a DNA sequence. The sequence is:

AGCGCCAACTCCAACCCCGCGATGGCTCCCAGGGA
GCGGAAGGCCGGGTGCAAGAACTTCTTCTGGAAGA
CCTTCACCTCCTGCGGAGGCTCCGGAGGCTCCGGA
GGCTCCGGAGGCTCCGGAGGCTCCGCCAACTCCAA
CCCCGCGATGGCTCCCAGGGAGCGGAAGGCCGGGT
GCAAGAACTTCTTCTGGAAGACCTTCACCTCCTGC
GGAGGCTCCGGAGGCTCCGGAGGCTCCGGAGGCTC
CGGTGGC.

SEQ ID NO: 71 includes Target Ligand: 1-210; Central Linker: 221-252; Free energy: −140.8; gdT CAI: 0.805786107124917; ORF count: 0.

SEQ ID NO: 72 is the sequence name for 2×SST28 2×G4S ligand ECOg (137). It is a Tumor Targeting Ligand construct. It is a DNA sequence. The sequence is:

TCCGCCAACTCCAACCCCGCTATGGCCCCTAGGGA
GAGGAAGGCCGGGTGCAAGAACTTCTTCTGGAAGA
CCTTCACCTCCTGCGGAGGCTCCGGAGGCTCCGGA
GGCTCCGGAGGCTCCGGAGGCTCCGCCAACTCCAA
CCCCGCTATGGCCCCTAGGGAGAGGAAGGCCGGGT
GCAAGAACTTCTTCTGGAAGACCTTCACCTCCTGC
GGAGGCTCCGGAGGCTCCGGAGGCTCCGGAGGCTC
CGGAGGA.

SEQ ID NO: 72 includes Target Ligand: 1-210; Central Linker: 221-252; Free energy: −140; gdT CAI: 0.822267579371957; ORF count: 1.

SEQ ID NO: 73 is the sequence name for SST28 ligand. It is a Tumor Targeting Ligand construct. It is an AA sequence. The sequence is: SANSNPAMAPRERKAGCKNFFWKTFTSC.

SEQ ID NO: 73 includes Target Ligand: 1-28.

SEQ ID NO: 74 is the sequence name for SST28 ligand. It is a Tumor Targeting Ligand construct. It is a DNA sequence. The sequence is:

TCCGCCAACTCCAACCCGGCCATGGCTCCCCGGGA
GAGGAAGGCCGGGTGCAAGAACTTCTTCTGGAAGA
CCTTCACCAGCTGC.

SEQ ID NO: 74 includes Target Ligand: 1-84; Free energy: −26.1; gdT CAI: 0.909139392619506; ORF count: 0.

SEQ ID NO: 75 is the sequence name for SST28 ligand ECOg (10). It is a Tumor Targeting Ligand construct. It is a DNA sequence. The sequence is:

AGCGCCAACAGCAACCCCGCTATGGCTCCCAGGGA
GCGCAAGGCCGGGTGCAAGAACTTCTTCTGGAAGA
CCTTCACCTCTTGC.

SEQ ID NO: 75 includes Target Ligand: 1-84; Free energy: −27.4; gdT CAI: 0.925222356313033; ORF count: 0.

SEQ ID NO: 76 is the sequence name for SST28 ligand ECOg (172). It is a Tumor Targeting Ligand construct. It is a DNA sequence. The sequence is:

AGCGCGAACAGCAACCCGGCCATGGCCCCTCGCGA
GCGAAAGGCCGGGTGCAAGAACTTCTTCTGGAAGA
CCTTCACCTCGTGC.

SEQ ID NO: 76 includes Target Ligand: 1-84; Free energy: −31.1; gdT CAI: 0.762381535851502; ORF count: 0.

SEQ ID NO: 77 is the sequence name for SST28 ligand ECOg (38). It is a Tumor Targeting Ligand construct. It is a DNA sequence. The sequence is:

AGCGCCAACTCCAACCCGGCTATGGCGCCCAGGGA
GAGGAAGGCCGGCTGCAAGAACTTCTTCTGGAAGA
CCTTCACCTCCTGC.

SEQ ID NO: 77 includes Target Ligand: 1-84; Free energy: −31; gdT CAI: 0.866223933524215; ORF count: 0.

SEQ ID NO: 78 is the sequence name for SST28 ligand ECOg (5). It is a Tumor Targeting Ligand construct. It is a DNA sequence. The sequence is:

AGTGCGAACTCGAACCCGGCCATGGCGCCCAGAGAGCGCAAGGCCGGGT
GCAAGAACTTTTTCTGGAAAACGTTCACATCGTGC.

SEQ ID NO: 78 includes Target Ligand: 1-84; Free energy: −30.7; gdT CAI: 0.714442287269154; ORF count: 0.

SEQ ID NO: 79 is the sequence name for SST28 ligand ECOg (44). It is a Tumor Targeting Ligand construct. It is a DNA sequence. The sequence is:

AGCGCGAACTCGAACCCGGCCATGGCTCCGCGCGAGCGTAAGGCCGGGT
GCAAGAACTTTTTCTGGAAAACCTTCACGAGTTGT.

SEQ ID NO: 79 includes Target Ligand: 1-84; Free energy: −30.1; gdT CAI: 0.688444421590185; ORF count: 0.

SEQ ID NO: 80 is the sequence name for TPO. It is a Tumor Targeting Ligand construct. It is an AA sequence. The sequence is:

SPAPPACDLRVLSKLLRDSHVLHSRLSQCPEVHPLPTPVLLPAVDFSLG
EWKTQMEETKAQDILGAVTLLLEGVMAARGQLGPTCLSSLLGQLSGQVR
LLLGALQSLLGTQGRTTAHKDPNAIFLSFQHLLRGKVRFLMLVGGSTLC
VRRAPPTTAVPSRTSLVLTLNELG.

SEQ ID NO: 80 includes Target Ligand: 1-171.

SEQ ID NO: 81 is the sequence name for TPO. It is a Tumor Targeting Ligand construct. It is a DNA sequence. The sequence is:

TCCCCAGCTCCACCAGCCTGTGACCTGAGGGTGCTGAGCAAGCTCCTGA
GGGACTCCCATGTGCTGCACAGCAGGCTGAGCCAGTGCCCTGAGGTGCA
TCCCCTGCCAACCCCTGTGCTGCTGCCAGCTGTGGACTTCTCCCTTGGG
GAGTGGAAGACCCAGATGGAGGAGACCAAGGCCCAGGACATCCTTGGGG
CTGTGACCCTGCTGCTGGAAGGGGTGATGGCTGCCAGGGGCCAGCTGGG
GCCAACCTGCCTCAGCTCCCTGCTGGGGCAGCTGTCAGGGCAGGTGAGG
CTGCTGCTGGGAGCCCTGCAGTCCCTGCTGGGGACCCAGGGCAGGACCA
CAGCCCACAAGGACCCCAATGCCATCTTCCTGAGCTTCCAGCACCTGCT
GAGGGGCAAGGTGAGGTTCCTGATGCTGGTTGGAGGCAGCACCCTGTGT
GTCAGGAGAGCTCCACCAACCACAGCTGTGCCCAGCAGGACCAGCCTGG
TGCTGACCCTGAATGAGCTTGGA.

SEQ ID NO: 81 includes Target Ligand: 1-513; Free energy: −251.6; gdT CAI: 0.86805332586369; ORF count: 4.

SEQ ID NO: 82 is the sequence name for TPO ECOg (6). It is a Tumor Targeting Ligand construct. It is a DNA sequence. The sequence is:

AGCCCCGCACCACCTGCCTGCGACCTGCGGGTGCTGTCCAAGCTGCTGC
GGGACAGCCACGTGCTGCACAGCAGGCTGTCCCAGTGCCCCGAGGTGCA
CCCACTGCCCACGCCCGTGCTGCTGCCCGCTGTGGACTTCTCCCTGGGC
GAGTGGAAGACACAGATGGAGGAGACCAAGGCCCAGGACATCCTGGGCG
CCGTGACCCTGCTGCTGGAAGGGGTGATGGCCGCCAGAGGGCAGCTGGG
GCCAACGTGCCTGTCCTCACTGCTGGGGCAGCTGTCCGGGCAGGTGCGG
CTGCTGCTGGGCGCCCTGCAGTCCCTGCTGGGCACCCAGGGGCGCACCA
CAGCTCACAAGGACCCCAACGCCATCTTCCTGTCCTTCCAGCACCTGCT
GCGGGGCAAGGTGCGGTTCCTGATGCTGGTCGGCGGCAGCACCCTGTGC
GTGCGCAGGGCACCACCGACCACAGCTGTGCCCAGCAGGACCTCACTGG
TGCTGACCCTGAACGAGCTGGGC.

SEQ ID NO: 82 includes Target Ligand: 1-513; Free energy: −258.8; gdT CAI: 0.891232089689473; ORF count: 1.

SEQ ID NO: 83 is the sequence name for TPO ECOg (42). It is a Tumor Targeting Ligand construct. It is a DNA sequence. The sequence is:

AGCCCGGCTCCTCCGGCCTGCGACCTGCGCGTGCTGAGCAAGCTCCTGC
GGGACTCGCACGTGCTGCACTCGCGCCTGAGCCAGTGCCCCGAGGTGCA
TCCCCTGCCTACCCCGGTGCTCCTGCCCGCGGTGGACTTCTCGCTCGGG
GAGTGGAAGACCCAGATGGAGGAGACCAAGGCCCAGGACATACTCGGGG
CCGTGACCCTGCTCCTGGAAGGGGTCATGGCAGCTCGGGGCCAGCTCGG
GCCTACGTGCCTGAGCTCCCTGCTCGGGCAGCTGTCCGGGCAGGTCCGG
CTCCTGCTCGGGGCCCTGCAGAGCCTGCTCGGGACCCAGGGCCGGACCA
CGGCTCACAAGGACCCGAACGCGATCTTCCTGAGCTTCCAGCACCTGCT
CCGGGGCAAGGTCAGGTTCCTGATGCTGGTCGGAGGCTCGACCCTGTGC
GTGCGCAGGGCTCCTCCGACCACGGCCGTGCCCTCGCGCACGAGCCTGG
TCCTGACCCTGAACGAGCTCGGG.

SEQ ID NO: 83 includes Target Ligand: 1-513; Free energy: −273.6; gdT CAI: 0.779630301042555; ORF count: 0.

SEQ ID NO: 84 is the sequence name for TPO ECOg (19). It is a Tumor Targeting Ligand construct. It is a DNA sequence. The sequence is:

TCTCCGGCTCCGCCTGCCTGCGACCTGCGGGTGCTGTCGAAGCTGCTGC
GGGACAGCCACGTCCTCCACAGCCGCCTGAGCCAGTGCCCGGAGGTGCA
CCCGCTGCCTACGCCGGTGCTGCTGCCGGCCGTGGACTTCAGCCTCGGG
GAGTGGAAGACGCAGATGGAGGAGACCAAGGCCCAGGACATCCTCGGGG
CCGTGACCCTGCTCCTGGAAGGGGTGATGGCAGCGCGAGGGCAGCTGGG
GCCTACCTGCCTCAGCTCCCTGCTGGGGCAGCTGTCGGGGCAGGTGCGG
CTGCTGCTCGGGGCCCTGCAGTCCCTGCTCGGGACCCAGGGCCGGACCA
CAGCCCACAAGGACCCCAACGCCATCTTCCTCTCCTTCCAGCACCTGCT
CCGGGGCAAGGTCCGGTTCCTGATGCTGGTCGGCGGCAGCACCCTGTGC
GTGAGACGGGCTCCGCCTACCACGGCCGTGCCCTCGCGCACGAGCCTGG
TCCTGACCCTGAACGAGCTCGGG.

SEQ ID NO: 84 includes Target Ligand: 1-513; Free energy: −269.4; gdT CAI: 0.79850475511585; ORF count: 1.

SEQ ID NO: 85 is the sequence name for TPO ECOg (53). It is a Tumor Targeting Ligand construct. It is a DNA sequence. The sequence is:

AGCCCCGCGCCGCCAGCGTGCGATCTGCGCGTGCTGAGCAAGCTGCTGC
GCGACTCGCACGTGCTGCACTCGCGGCTCTCGCAGTGCCCCGAGGTGCA
CCCGCTGCCCACACCCGTGCTGCTGCCCGCGGTGGACTTCTCGCTCGGC
GAGTGGAAGACGCAGATGGAGGAGACGAAAGCGCAGGACATCCTCGGCG
CGGTGACGCTGCTGCTCGAAGGCGTGATGGCTGCTCGCGGGCAGCTCGG
GCCTACGTGCCTGAGCTCGCTGCTCGGGCAGCTGAGCGGGCAGGTGCGG
CTGCTGCTCGGCGCGCTGCAGTCGCTGCTCGGCACGCAGGGGCGCACCA
CAGCGCACAAGGACCCGAACGCGATCTTCCTGAGCTTCCAGCACCTGCT
GCGCGGGAAGGTGCGCTTCCTGATGCTCGTCGGCGGCAGCACGCTGTGC
GTGCGCAGAGCGCCTCCGACCACCGCGGTGCCCTCGCGCACCTCGCTCG
TGCTCACGCTGAACGAGCTCGGC.

SEQ ID NO: 85 includes Target Ligand: 1-513; Free energy: −268.8; gdT CAI: 0.737843607302898; ORF count: 1.

SEQ ID NO: 86 is the sequence name for TPO ECOg (57). It is a Tumor Targeting Ligand construct. It is a DNA sequence. The sequence is:

AGCCCCGCTCCGCCAGCCTGCGACCTGCGGGTGCTGAGCAAGCTGCTGC
GGGACAGCCACGTGCTGCACAGCCGGCTGAGCCAGTGCCCCGAGGTGCA
CCCGCTGCCCACGCCCGTGCTGCTGCCCGCTGTGGACTTCAGCCTGGGC
GAGTGGAAGACCCAGATGGAGGAGACCAAGGCCCAGGACATCCTGGGCG
CCGTGACCCTGCTGCTGGAAGGGGTGATGGCCGCTAGGGGCCAGCTGGG
GCCTACCTGCCTGAGCAGCCTGCTGGGGCAGCTGAGCGGGCAGGTGCGG
CTGCTGCTGGGCGCCCTGCAGAGCCTGCTGGGCACCCAGGGGCGCACCA
CAGCCCACAAGGACCCCAACGCCATCTTCCTGAGCTTCCAGCACCTGCT
GCGGGGCAAGGTGCGCTTCCTGATGCTGGTCGGCGGCAGCACCCTGTGC
GTGCGCAGGGCTCCGCCTACCACCGCCGTGCCCAGCCGCACCAGCCTGG
TGCTGACCCTGAACGAGCTCGGC.

SEQ ID NO: 86 includes Target Ligand: 1-513; Free energy: −267.9; gdT CAI: 0.853835989559969; ORF count: 2.

SEQ ID NO: 87 is the sequence name for Fc. It is a Fusion Moety construct. It is an AA sequence. The sequence is:

DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

SEQ ID NO: 87 includes Fusion Moeity: 1-227.

SEQ ID NO: 88 is the sequence name for Fc. It is a Fusion Moety construct. It is a DNA sequence. The sequence is:

GACAAGACACACACCTGCCCCCCATGTCCCGCGCCTGAACTCCTGGGGG
GGCCCTCTGTCTTCCTGTTCCCACCGAAGCCGAAAGACACCCTTATGAT
ATCTCGGACCCCTGAGGTAACTTGTGTAGTAGTTGATGTGTCTCACGAA
GATCCAGAAGTGAAGTTTAATTGGTATGTGGATGGAGTGGAAGTTCACA
ACGCTAAGACCAAGCCCAGGGAGGAACAATACAATTCCACATACCGAGT
TGTTTCAGTGCTCACTGTGTTGCATCAGGACTGGCTCAACGGCAAGGAG
TATAAATGCAAAGTCAGTAACAAAGCCCTGCCCGCCCCCATCGAGAAAA
CCATCTCGAAGGCAAAAGGCCAGCCTCGCGAGCCACAGGTCTACACCCT
GCCTCCCTCACGTGACGAGCTGACAAAGAATCAGGTCAGCTTGACGTGT
TTAGTGAAAGGATTTTATCCAAGCGACATTGCCGTGGAATGGGAGAGCA
ATGGCCAGCCTGAGAACAATTACAAAACAACTCCACCTGTGCTCGACAG
TGATGGGTCTTTCTTCTTATACTCCAAACTAACGGTCGATAAGAGCAGA
TGGCAGCAAGGTAACGTGTTTTCCTGCTCAGTGATGCATGAGGCTCTGC
ATAACCACTATACTCAGAAGTCCCTTAGTCTGAGCCCGGGTAAG.

SEQ ID NO: 88 includes Fusion Moeity: 1-681; Free energy: −199.9; gdT CAI: 0.747707161534413; ORF count: 6.

SEQ ID NO: 89 is the sequence name for FC ECOg (85). It is a Fusion Moety construct. It is a DNA sequence. The sequence is:

GATAAGACCCACACCTGTCCGCCGTGCCCCGCACCCGAGCTTCTCGGCG
GCCCCAGCGTGTTCCTGTTTCCGCCGAAGCCCAAGGACACCCTGATGAT
CTCCCGGACGCCCGAGGTGACCTGCGTGGTGGTGGACGTGTCCCACGAG
GACCCCGAGGTGAAGTTCAACTGGTACGTGGACGGGGTCGAGGTGCACA
ACGCCAAGACCAAGCCCCGCGAGGAGCAGTACAACTCCACCTACCGGGT
CGTGTCCGTGCTGACCGTGCTGCACCAGGACTGGCTGAACGGGAAGGAG
TACAAGTGCAAGGTGTCCAACAAGGCCCTGCCCGCGCCCATCGAGAAGA
CCATCTCCAAGGCCAAGGGGCAGCCCAGGGAGCCCCAGGTGTACACCCT
GCCTCCGTCCCGGGACGAGCTGACCAAGAACCAGGTGTCCCTGACCTGC
CTGGTGAAGGGGTTCTACCCCAGCGACATCGCCGTGGAGTGGGAGTCCA
ACGGGCAGCCCGAGAACAACTACAAGACCACGCCGCCGGTGCTGGACTC
CGACGGGAGCTTCTTCCTGTACTCCAAGCTGACCGTGGACAAGTCCCGG
TGGCAGCAGGGGAACGTGTTCAGCTGCAGCGTGATGCACGAGGCCCTGC
ACAACCACTACACCCAGAAGTCCCTGAGCCTGAGCCCCGGGAAG.

SEQ ID NO: 89 includes Fusion Moeity: 1-681; Free energy: −269.2; gdT CAI: 0.894515513769793; ORF count: 0.

SEQ ID NO: 90 is the sequence name for FC ECOg (59). It is a Fusion Moety construct. It is a DNA sequence. The sequence is:

GACAAGACGCACACGTGTCCGCCGTGCCCCGCACCCGAACTGCTCGGCG
GCCCCAGCGTGTTCCTGTTTCCGCCGAAGCCCAAGGACACGCTGATGAT
CTCGCGCACGCCCGAGGTGACGTGCGTCGTCGTCGACGTGTCGCACGAG
GACCCCGAGGTGAAGTTCAACTGGTACGTCGACGGCGTCGAGGTGCACA
ACGCCAAGACGAAGCCGCGCGAGGAGCAGTACAACAGCACGTACCGCGT
CGTGAGCGTGCTGACCGTGCTGCACCAGGACTGGCTGAACGGCAAGGAG
TACAAGTGCAAGGTGAGCAACAAGGCGCTGCCCGCGCCGATCGAGAAGA
CGATCAGCAAGGCCAAGGGGCAGCCACGCGAGCCGCAGGTGTACACGCT
GCCGCCGTCGCGCGACGAGCTGACGAAGAACCAGGTGTCGCTGACGTGC
CTCGTGAAGGGCTTCTACCCCAGCGACATCGCCGTCGAGTGGGAGAGCA
ACGGGCAGCCCGAGAACAACTACAAGACGACGCCGCCGGTGCTCGACAG
CGACGGCAGCTTCTTCCTGTACAGCAAGCTGACCGTCGACAAGTCGCGC
TGGCAGCAGGGCAACGTGTTCAGCTGCAGCGTGATGCACGAGGCGCTGC
ACAACCACTACACGCAGAAGTCGCTGTCGCTGTCGCCCGGCAAG.

SEQ ID NO: 90 includes Fusion Moeity: 1-681; Free energy: −299.3; gdT CAI: 0.760430405503452; ORF count: 0.

SEQ ID NO: 91 is the sequence name for FC ECOg (5). It is a Fusion Moety construct. It is a DNA sequence. The sequence is:

GACAAGACGCACACCTGTCCGCCGTGCCCGGCTCCCGAACTGCTCGGCG
GCCCGTCCGTCTTCCTGTTTCCGCCGAAGCCGAAGGACACCCTGATGAT
CAGCCGGACGCCGGAGGTGACCTGCGTCGTCGTCGACGTCAGCCACGAG
GACCCCGAGGTCAAGTTCAACTGGTACGTCGACGGCGTCGAGGTCCACA
ACGCCAAGACGAAGCCCCGCGAGGAGCAGTACAACTCGACGTACCGGGT
CGTCTCCGTCCTGACCGTCCTCCACCAGGACTGGCTGAACGGCAAGGAG
TACAAGTGCAAGGTCAGCAACAAGGCCCTGCCGGCCCCGATCGAGAAGA
CGATCTCGAAGGCCAAGGGCCAGCCTCGGGAGCCCCAGGTCTACACGCT
GCCGCCGTCCCGGGACGAGCTGACCAAGAACCAGGTCAGCCTGACCTGC
CTGGTCAAGGGCTTCTACCCGTCCGACATCGCCGTCGAGTGGGAGTCGA
ACGGCCAGCCCGAGAACAACTACAAGACGACGCCGCCGGTCCTGGACTC
CGACGGCAGCTTCTTCCTGTACAGCAAGCTGACCGTCGACAAGTCCCGG
TGGCAGCAGGGCAACGTCTTCAGCTGCTCCGTCATGCACGAGGCCCTGC
ACAACCACTACACCCAGAAGTCCCTGTCCCTGTCGCCCGGCAAG.

SEQ ID NO: 91 includes Fusion Moeity: 1-681; Free energy: −278.1; gdT CAI: 0.766513451450202; ORF count: 0.

SEQ ID NO: 92 is the sequence name for FC ECOg (23). It is a Fusion Moety construct. It is a DNA sequence. The sequence is:

GACAAGACCCATACCTGTCCGCCGTGCCCGGCTCCGGAACTGCTCGGCG
GCCCGTCCGTCTTCCTGTTTCCGCCGAAGCCGAAGGACACCCTGATGAT
CAGCCGGACGCCGGAGGTGACCTGCGTCGTCGTCGACGTCAGCCACGAG
GACCCGGAGGTCAAGTTCAACTGGTACGTCGACGGCGTCGAGGTCCACA
ACGCCAAGACCAAGCCCCGGGAAGAGCAGTACAACAGCACCTACCGGGT
CGTGTCCGTCCTGACCGTCCTGCACCAGGACTGGCTGAACGGCAAGGAG
TACAAGTGCAAGGTCAGCAACAAGGCCCTGCCGGCCCCGATCGAGAAGA
CCATCAGCAAGGCCAAGGGCCAGCCCAGGGAGCCGCAGGTCTACACCCT
GCCGCCGTCCCGGGACGAGCTGACCAAGAACCAGGTCAGCCTGACCTGC
CTGGTCAAGGGCTTCTACCCGTCCGACATCGCCGTCGAATGGGAGTCCA
ACGGCCAGCCGGAGAACAACTACAAGACGACGCCGCCGGTCCTGGACTC
CGACGGCAGCTTCTTCCTGTACAGCAAGCTGACCGTCGACAAGTCCCGG
TGGCAGCAGGGCAACGTCTTCAGCTGCTCCGTCATGCACGAGGCCCTGC
ACAACCACTACACCCAGAAGTCCCTGTCCCTGTCGCCCGGCAAG.

SEQ ID NO: 92 includes Fusion Moeity: 1-681; Free energy: −276.3; gdT CAI: 0.769448727316174; ORF count: 0.

SEQ ID NO: 93 is the sequence name for FC ECOg (56). It is a Fusion Moety construct. It is a DNA sequence. The sequence is:

GACAAGACGCACACGTGTCCGCCGTGCCCAGCCCCGGAGCTTCTCGGCGG
CCCCTCGGTGTTCCTGTTTCCGCCGAAGCCCAAGGACACGCTGATGATCT
CCCGGACCCCGGAGGTGACCTGCGTCGTCGTCGACGTGTCCCACGAGGAC
CCCGAGGTGAAGTTCAACTGGTACGTCGACGGCGTCGAGGTGCACAACGC
GAAGACGAAGCCCCGCGAGGAGCAGTACAACTCCACGTACCGCGTCGTCT
CCGTGCTCACCGTGCTGCACCAGGACTGGCTCAACGGCAAGGAGTACAAG
TGCAAGGTGTCCAACAAGGCGCTGCCCGCGCCCATCGAGAAGACCATCTC
CAAGGCCAAGGGGCAGCCCCGGGAACCCCAGGTGTACACGCTGCCGCCGA
GCCGCGACGAGCTGACCAAGAACCAGGTGTCCCTGACCTGCCTCGTCAAG
GGGTTCTACCCCTCGGACATCGCCGTGGAGTGGGAGTCCAACGGGCAGCC
CGAGAACAACTACAAGACGACGCCGCCGGTGCTCGACTCCGACGGGTCCT
TCTTCCTCTACTCGAAGCTCACCGTGGACAAGTCCCGGTGGCAGCAGGGC
AACGTGTTCAGCTGCTCCGTGATGCACGAGGCGCTGCACAACCACTACAC
GCAGAAGTCCCTGTCCCTGAGCCCCGGGAAG.

SEQ ID NO: 93 includes Fusion Moeity: 1-681; Free energy: −275.4; gdT CAI: 0.816569307205555; ORF count: 0.

SEQ ID NO: 94 is the sequence name for Hum2 scFv. It is a gdT Targeting scFv construct. It is an AA sequence. The sequence is:

IQLVQSGAEVKKPGASVKVSCKASGYTFTRYTMHWVRQAPGQGLEWIGYI
NPSRGYTNYNQKFKDRATLTTDKSTSTAYMELSSLRSEDTAVYYCARYYD
DHYCLDYWGQGTLVTVSSGGSGGSGGSGGSGGDIQLTQSPSSLSASVGDR
VTITCRASSSVSYMNWYQQKPGKAPKRWIYDTSKVASGAPSRFTGSGSGT
DYTLTISSLQPEDFATYYCQQWSSNPLTFGGGTKLEIK.

SEQ ID NO: 94 includes gdT scFv: 1-238; gdT VH: 119-132; gdT Linker: 119-132; gdT VL: 133-238.

SEQ ID NO: 95 is the sequence name for Hum2 scFv ECOg (0). It is a gdT Targeting scFv construct. It is a DNA sequence. The sequence is:

ATCCAGCTGGTGCAGTCCGGGGCCGAGGTGAAGAAGCCCGGGGCCTCCGT
GAAGGTGTCCTGCAAGGCCTCCGGGTACACCTTCACCCGGTACACCATGC
ACTGGGTGAGGCAGGCTCCCGGGCAGGGGCTGGAGTGGATCGGGTACATC
AACCCCAGCCGCGGGTACACCAACTACAACCAGAAGTTCAAGGACCGGGC
CACGCTGACCACCGACAAGTCCACCTCCACCGCCTACATGGAGCTGAGCT
CCCTGCGGTCCGAGGACACCGCCGTGTACTACTGCGCCCGGTACTACGAC
GACCACTACTGCCTGGACTACTGGGGCCAGGGGACCCTGGTGACCGTGTC
CTCCGGCGGCTCAGGCGGCTCAGGAGGCTCAGGCGGCTCAGGCGGCGACA
TCCAGCTGACCCAGAGCCCCAGCTCCCTGTCCGCCTCCGTCGGGGACCGG
GTGACCATCACCTGCCGGGCCTCCTCCTCCGTGTCCTACATGAACTGGTA
CCAGCAGAAGCCCGGGAAGGCGCCCAAGCGGTGGATCTACGACACCTCCA
AGGTGGCCTCCGGGGCTCCCTCACGGTTCACCGGGTCCGGGTCCGGGACC
GACTACACCCTGACCATCAGCTCCCTGCAGCCCGAGGACTTCGCCACCTA
CTACTGCCAGCAGTGGTCCTCCAACCCGCTGACCTTCGGCGGCGGCACCA
AGCTGGAGATCAAG.

SEQ ID NO: 95 includes gdT scFv: 1-714; gdT VH: 355-396; gdT Linker: 355-396; gdT VL: 397-714; Free energy: −325.7; gdT CAI: 0.910798811448247; ORF count: 0.

SEQ ID NO: 96 is the sequence name for Hum2 scFv ECOg (183). It is a gdT Targeting scFv construct. It is a DNA sequence. The sequence is:

ATCCAGCTCGTGCAGAGCGGGGCCGAGGTGAAGAAGCCCGGGGCGAGCGT
GAAGGTGTCGTGCAAGGCGAGCGGGTACACCTTCACGCGCTACACCATGC
ACTGGGTGCGCCAAGCTCCCGGGCAGGGGCTGGAGTGGATCGGGTACATC
AACCCCTCGCGCGGGTACACCAACTACAACCAGAAGTTCAAGGACCGCGC
CACGCTCACCACCGACAAGTCCACCTCCACCGCCTACATGGAGCTCTCCT
CGCTGCGCTCCGAGGACACCGCGGTGTACTACTGCGCGCGCTACTACGAC
GACCACTACTGCCTCGACTACTGGGGCCAGGGGACCCTCGTGACCGTGTC
GAGTGGTGGTAGTGGTGGTAGTGGTGGTAGTGGTGGTAGTGGTGGCGACA
TCCAGCTCACCCAGAGCCCCAGCTCGTTGAGCGCGAGCGTCGGGGACCGC
GTGACCATCACGTGCCGCGCCTCGTCGAGCGTGAGCTACATGAACTGGTA
CCAGCAGAAGCCCGGGAAGGCCCCGAAGAGGTGGATCTACGACACCTCCA
AGGTGGCGAGCGGGGCTCCATCGCGCTTCACGGGGAGCGGGAGCGGGACC
GACTACACCCTCACCATCTCCTCGCTCCAGCCCGAGGACTTCGCCACCTA
CTACTGCCAGCAGTGGAGCTCCAACCCGCTCACCTTCGGCGGCGGCACCA
AGTTGGAGATCAAG.

SEQ ID NO: 96 includes gdT scFv: 1-714; gdT VH: 355-396; gdT Linker: 355-396; gdT VL: 397-714; Free energy: −331.6; gdT CAI: 0.766742326650226; ORF count: 2.

SEQ ID NO: 97 is the sequence name for Hum2 scFv ECOg (6). It is a gdT Targeting scFv construct. It is a DNA sequence. The sequence is:

ATCCAGCTGGTGCAGAGCGGCGCCGAGGTGAAGAAGCCCGGGGCCTCGGT
GAAGGTCTCCTGCAAGGCCTCGGGGTACACCTTCACCCGGTACACCATGC
ACTGGGTGCGCCAAGCCCCAGGGCAGGGCCTGGAGTGGATCGGGTACATC
AACCCCAGCCGCGGGTACACCAACTACAACCAGAAGTTCAAGGACCGGGC
CACGCTGACCACGGACAAGTCCACGTCCACGGCGTACATGGAGCTGAGCT
CCCTGCGCTCCGAGGACACCGCGGTGTACTACTGCGCCCGGTACTACGAC
GACCACTACTGCCTGGACTACTGGGGCCAGGGGACCCTGGTCACCGTGTC
CTCCGGAGGCTCCGGAGGCTCCGGAGGCTCCGGAGGCTCCGGAGGCGACA
TCCAGCTGACCCAGAGCCCCAGCTCCCTCAGCGCCTCCGTCGGGGACCGG
GTGACCATCACCTGCCGCGCCTCGTCCTCGGTGTCCTACATGAACTGGTA
CCAGCAGAAGCCCGGGAAGGCCCCGAAGCGGTGGATCTACGACACGTCCA
AGGTCGCCTCCGGGGCTCCGTCGAGGTTCACCGGCTCCGGCTCCGGGACG
GACTACACCCTGACCATCTCCTCGCTGCAGCCCGAGGACTTCGCCACGTA
CTACTGCCAGCAGTGGTCCTCGAATCCCCTGACCTTCGGCGGCGGCACGA
AGCTGGAGATTAAG.

SEQ ID NO: 97 includes gdT scFv: 1-714; gdT VH: 355-396; gdT Linker: 355-396; gdT VL: 397-714; Free energy: −331; gdT CAI: 0.825626192459745; ORF count: 1.

SEQ ID NO: 98 is the sequence name for Hum2 scFv ECOg (42). It is a gdT Targeting scFv construct. It is a DNA sequence. The sequence is:

ATCCAGCTGGTCCAGTCCGGGGCCGAGGTGAAGAAGCCCGGGGCCTCCGT
GAAGGTCTCCTGCAAGGCCTCCGGGTACACCTTCACCCGGTACACCATGC
ACTGGGTCCGGCAGGCTCCAGGGCAGGGGCTGGAGTGGATCGGGTACATC
AACCCCAGCCGCGGGTACACCAACTACAACCAGAAGTTCAAGGACCGGGC
CACGCTGACCACCGACAAGTCCACCTCCACCGCCTACATGGAGCTGAGCT
CCCTGCGGTCCGAGGACACCGCCGTCTACTACTGCGCCCGGTACTACGAC
GACCACTACTGCCTGGACTACTGGGGCCAGGGGACCCTGGTGACCGTCTC
CTCCGGAGGCTCCGGAGGCTCCGGAGGCTCCGGAGGCTCCGGAGGCGACA
TCCAGCTGACCCAGAGCCCCAGCTCCCTCTCCGCCTCCGTCGGGGACCGG
GTGACCATCACCTGCCGGGCCTCCTCCTCCGTCTCCTACATGAACTGGTA
CCAGCAGAAGCCCGGGAAGGCCCCGAAGCGGTGGATCTACGACACCTCCA
AGGTGGCCTCCGGGGCCCCTAGCAGGTTCACCGGGTCCGGGTCCGGGACC
GACTACACCCTGACCATCTCCAGCCTCCAGCCCGAGGACTTCGCCACCTA
CTACTGCCAGCAGTGGTCCTCCAACCCGCTGACCTTCGGCGGCGGCACCA
AGCTGGAGATCAAG.

SEQ ID NO: 98 includes gdT scFv: 1-714; gdT VH: 355-396; gdT Linker: 355-396; gdT VL: 397-714; Free energy: −330; gdT CAI: 0.887375945709965; ORF count: 1.

SEQ ID NO: 99 is the sequence name for Hum2 scFv ECOg (196). It is a gdT Targeting scFv construct. It is a DNA sequence. The sequence is:

ATCCAGCTGGTGCAGTCCGGGGCCGAGGTGAAGAAGCCCGGGGCCTCCGT
GAAGGTGTCCTGCAAGGCCTCCGGGTACACCTTCACCCGGTACACCATGC
ACTGGGTGAGGCAGGCTCCCGGGCAGGGGCTGGAGTGGATCGGGTACATC
AACCCCTCGCGCGGGTACACCAACTACAACCAGAAGTTCAAGGACCGGGC
CACGCTGACCACCGACAAGAGCACCTCCACCGCCTACATGGAGCTGTCCA
GCCTGCGGTCCGAGGACACCGCCGTGTACTACTGCGCCCGGTACTACGAC
GACCACTACTGCCTGGACTACTGGGGCCAGGGGACCCTGGTGACCGTGTC
CAGCGGCGGCTCTGGCGGCTCTGGAGGCTCCGGCGGCTCTGGAGGCGACA
TCCAGCTGACCCAGTCCCCGTCCAGCCTGTCCGCCAGCGTCGGGGACCGG
GTGACCATCACCTGCCGGGCCTCCTCCAGCGTGTCCTACATGAACTGGTA
CCAGCAGAAGCCCGGGAAGGCGCCCAAGCGGTGGATCTACGACACCTCCA
AGGTGGCCTCCGGGGCTCCCAGCCGTTTCACCGGGTCCGGGTCCGGGACC
GACTACACCCTGACCATCTCCAGCCTGCAGCCCGAGGACTTCGCCACCTA
CTACTGCCAGCAGTGGTCCAGCAACCCGCTGACCTTCGGCGGCGGCACCA
AGCTGGAGATCAAG.

SEQ ID NO: 99 includes gdT scFv: 1-714; gdT VH: 355-396; gdT Linker: 355-396; gdT VL: 397-714; Free energy: −324.7; gdT CAI: 0.892144940351689; ORF count: 1.

SEQ ID NO: 100 is the sequence name for Hum2 scFv ECOg (172). It is a gdT Targeting scFv construct. It is a DNA sequence. The sequence is:

ATCCAGCTCGTCCAGTCCGGGGCCGAGGTGAAGAAGCCCGGGGCGTCCGT
GAAGGTGTCCTGCAAGGCGTCCGGGTACACGTTCACCCGGTACACGATGC
ACTGGGTCCGTCAGGCTCCCGGGCAGGGGCTGGAGTGGATCGGGTACATC
AACCCGTCCCGCGGGTACACGAACTACAACCAGAAGTTCAAGGACCGGGC
GACCCTGACGACCGACAAGTCCACGTCCACCGCGTACATGGAGCTGTCGT
CCCTGCGGTCCGAGGACACCGCCGTGTACTACTGCGCCCGGTACTACGAC
GACCACTACTGCCTCGACTACTGGGGCCAGGGGACCCTCGTGACCGTGTC
CTCCGGAGGTTCCGGCGGCTCCGGAGGATCTGGCGGCTCCGGCGGCGACA
TCCAGCTGACCCAGTCGCCCTCGTCCCTGTCCGCGTCCGTCGGGGACCGG
GTGACGATCACGTGCCGGGCGTCCTCCTCCGTGTCCTACATGAACTGGTA
CCAGCAGAAGCCCGGGAAGGCCCCGAAGAGGTGGATCTACGACACGTCCA
AGGTCGCGTCCGGGGCTCCATCCCGGTTCACCGGGTCCGGGTCCGGGACC
GACTACACCCTGACGATCTCCTCGCTCCAGCCCGAGGACTTCGCGACGTA
CTACTGCCAGCAGTGGTCCTCGAATCCCCTGACGTTCGGCGGCGGCACGA
AGCTGGAGATCAAG.

SEQ ID NO: 100 includes gdT scFv: 1-714; gdT VH: 355-396; gdT Linker: 355-396; gdT VL: 397-714; Free energy: −324.5; gdT CAI: 0.827859696643083; ORF count: 0.

SEQ ID NO: 101 is the sequence name for gd-c V6 scFv. It is a gdT Targeting scFv construct. It is an AA sequence. The sequence is:

QVQLQQSGPGLVKPSQTLSLTCAISGDSVSSNRAAWNWIRQSPSKGLEWL
GRTYYRSKWYNEYAASVKSRMSINPDTSKNQFSLQLNSVTPEDTALYYCA
RDLWELREACDIWGQGTMVTVSSGGSGGSGGSGGSGGDIVMTQSPSFLST
FVGDRVTITCRASQGISSYLAWYQQKPGKVPKLLIYVASTLQSGVPSRFS
GSGSGTEFTLTISSLQPEDFATYYCQQLNSYPFTFGPGTKVDIK.

SEQ ID NO: 101 includes gdT scFv: 1-244; gdT VH: 124-137; gdT Linker: 124-137; gdT VL: 138-244.

SEQ ID NO: 102 is the sequence name for gd-C V6 scFv. It is a gdT Targeting scFv construct. It is a DNA sequence. The sequence is:

CAGGTGCAGCTGCAGCAGTCCGGGCCCGGGCTGGTGAAGCCCAGCCAGAC
CCTGAGCCTGACCTGCGCCATCAGCGGGGACTCCGTCAGCTCCAACCGGG
CCGCCTGGAACTGGATCAGGCAGTCCCCGTCCAAGGGGCTGGAGTGGCTC
GGCCGGACCTACTACCGCTCCAAGTGGTACAACGAGTACGCCGCCTCCGT
GAAGTCCCGGATGAGCATCAACCCCGATACCTCCAAGAACCAGTTCAGCC
TGCAGCTGAACTCCGTGACGCCCGAGGATACGGCCCTGTACTACTGCGCC
AGGGACCTGTGGGAGCTGCGCGAGGCCTGCGACATCTGGGGCCAGGGGAC
CATGGTGACCGTGTCCTCCGGCGGCTCAGGCGGCTCAGGTGGCTCCGGCG
GATCAGGCGGCGACATCGTGATGACCCAGTCCCCATCCTTCCTGTCCACC
TTCGTCGGGGACCGGGTGACCATCACCTGCCGGGCCAGCCAGGGGATCAG
CTCCTACCTGGCCTGGTACCAGCAGAAGCCCGGGAAGGTGCCCAAGCTGC
TGATCTACGTGGCCTCAACCCTGCAGTCCGGGGTGCCCAGCCGGTTCAGC
GGGTCCGGGTCCGGGACCGAGTTCACCCTGACCATCAGCTCCCTGCAGCC
CGAGGACTTCGCCACCTACTACTGCCAGCAGCTGAACTCCTACCCCTTCA
CCTTCGGGCCCGGGACCAAGGTGGACATCAAG.

SEQ ID NO: 102 includes gdT scFv: 1-732; gdT VH: 369-411; gdT Linker: 369-411; gdT VL: 412-732; Free energy: −325.2; gdT CAI: 0.901042; ORF count: 1.

SEQ ID NO: 103 is the sequence name for gd-c V6 scFv ECOg (69). It is a gdT Targeting scFv construct. It is a DNA sequence. The sequence is:

CAGGTGCAGCTGCAGCAGTCCGGGCCCGGGCTGGTGAAGCCTTCCCAGAC
CCTCTCCCTCACCTGCGCCATCTCCGGGGATTCCGTGTCCTCCAACCGCG
CCGCCTGGAACTGGATCAGGCAGTCCCCTTCCAAGGGGCTGGAGTGGCTG
GGGCGCACGTACTACCGCTCCAAGTGGTACAACGAGTACGCCGCTTCCGT
GAAGTCCCGCATGAGCATCAACCCCGATACCTCCAAGAACCAGTTCTCCC
TGCAGCTGAACTCCGTGACCCCGGAGGATACCGCGCTGTACTACTGCGCC
CGGGACCTGTGGGAGCTGCGGGAAGCCTGCGACATCTGGGGCCAGGGGAC
CATGGTGACCGTGTCCTCCGGAGGCTCCGGAGGCTCCGGAGGTTCCGGAG
GCTCCGGCGGCGACATCGTGATGACGCAGTCCCCTTCCTTCCTGAGCACC
TTCGTAGGGGACCGCGTTACCATCACCTGCAGGGCTTCCCAGGGGATCTC
CTCCTACCTCGCATGGTACCAGCAGAAGCCCGGGAAGGTTCCCAAGCTGC
TGATCTACGTGGCTTCCACGCTGCAGTCCGGGGTGCCTTCCCGGTTCTCC
GGGAGCGGGAGCGGGACGGAGTTCACCCTCACCATCAGCTCCCTGCAGCC
CGAGGACTTCGCAACGTACTACTGCCAGCAGCTGAACTCCTACCCCTTCA
CCTTCGGGCCCGGGACGAAGGTGGACATCAAG.

SEQ ID NO: 103 includes gdT scFv: 1-732; gdT VH: 369-411; gdT Linker: 369-411; gdT VL: 412-732; Free energy: −328.2; gdT CAI: 0.885003557188278; ORF count: 0.

SEQ ID NO: 104 is the sequence name for gd-c V6 scFv ECOg (34). It is a gdT Targeting scFv construct. It is a DNA sequence. The sequence is:

CAAGTGCAGCTGCAGCAGAGCGGGCCCGGGCTGGTGAAGCCGTCGCAGAC
GCTGTCGCTGACGTGCGCCATCAGCGGCGACAGCGTGAGCAGCAACCGCG
CCGCGTGGAACTGGATCAGGCAGTCGCCCAGCAAGGGGCTGGAGTGGCTG
GGGCGCACGTACTACCGCAGCAAGTGGTACAACGAGTACGCCGCCAGCGT
GAAGAGCCGCATGAGCATCAACCCCGACACCAGCAAGAACCAGTTCTCGC
TGCAGCTGAACAGCGTGACGCCCGAGGACACCGCGCTGTACTACTGCGCG
CGCGACTTGTGGGAGCTGCGCGAGGCGTGCGACATCTGGGGCCAGGGCAC
CATGGTGACCGTGAGCAGCGGCGGCAGTGGTGGCAGCGGTGGTAGCGGCG
GTAGCGGCGGCGACATCGTGATGACGCAGTCGCCGTCGTTCTTGAGCACG
TTCGTGGGCGACCGCGTGACCATCACGTGCCGCGCGTCGCAGGGCATCAG
CAGCTACTTGGCGTGGTACCAGCAGAAGCCCGGCAAGGTGCCCAAGCTGC
TGATCTACGTGGCCAGCACGCTGCAGAGCGGCGTGCCGTCGCGCTTCAGC
GGCAGCGGCAGCGGCACCGAGTTCACGCTGACCATCAGCAGCCTGCAGCC
CGAGGACTTCGCCACGTACTACTGCCAGCAGCTGAACAGCTACCCGTTCA
CGTTCGGGCCCGGCACCAAGGTGGACATCAAG.

SEQ ID NO: 104 includes gdT scFv: 1-732; gdT VH: 369-411; gdT Linker: 369-411; gdT VL: 412-732; Free energy: −353.6; gdT CAI: 0.744822337548797; ORF count: 2.

SEQ ID NO: 105 is the sequence name for gd-c V6 scFv ECOg (55). It is a gdT Targeting scFv construct. It is a DNA sequence. The sequence is:

CAGGTGCAGCTGCAGCAGAGCGGCCCCGGGCTCGTGAAGCCGTCGCAGAC
CCTGAGCCTCACCTGCGCCATCTCCGGGGACTCCGTGTCCTCGAACCGCG
CCGCGTGGAACTGGATTCGGCAGAGCCCCAGCAAGGGCCTGGAGTGGCTG
GGGCGCACCTACTACCGCTCCAAGTGGTACAACGAGTACGCCGCCTCGGT
GAAGTCCCGGATGAGCATCAACCCCGACACCTCGAAGAACCAGTTCTCGC
TGCAGCTGAACTCCGTGACCCCGGAGGACACGGCGCTGTACTACTGCGCC
CGGGACCTCTGGGAGCTCCGCGAGGCCTGCGACATCTGGGGCCAGGGGAC
CATGGTCACCGTGTCCTCCGGAGGCTCCGGAGGCTCCGGAGGCTCCGGAG
GCTCCGGAGGCGACATCGTGATGACCCAGAGCCCCAGCTTCCTGAGCACC
TTCGTCGGGGACCGGGTCACCATCACGTGCCGCGCGTCCCAGGGGATCTC
CTCGTACCTGGCCTGGTACCAGCAGAAGCCCGGGAAGGTGCCGAAGCTGC
TGATCTACGTGGCCTCGACGCTGCAGTCCGGGGTCCCGAGCCGCTTCAGC
GGCTCCGGGTCCGGGACCGAGTTCACCCTGACCATCTCGTCGCTGCAGCC
CGAGGACTTCGCCACGTACTACTGCCAGCAGCTGAACAGCTACCCCTTCA
CCTTCGGCCCCGGGACCAAGGTGGACATCAAG.

SEQ ID NO: 105 includes gdT scFv: 1-732; gdT VH: 369-411; gdT Linker: 369-411; gdT VL: 412-732; Free energy: −351.8; gdT CAI: 0.812038056353435; ORF count: 0. SEQ ID NO: 106 is the sequence name for gd-c V6 scFv ECOg (21). It is a gdT Targeting scFv construct. It is a DNA sequence. The sequence is:

CAGGTGCAGCTGCAGCAGAGCGGGCCCGGGCTGGTGAAGCCCAGCCAGAC
CCTGAGCCTGACCTGCGCCATCTCCGGGGACAGCGTGAGCAGCAACCGGG
CCGCCTGGAACTGGATCAGGCAGAGCCCCAGCAAGGGGCTGGAGTGGCTG
GGGCGCACATACTACCGCTCCAAGTGGTACAATGAGTATGCCGCCAGCGT
GAAGAGCCGCATGAGCATCAACCCCGACACCTCCAAGAACCAGTTCAGCC
TGCAGCTGAACAGCGTGACGCCCGAGGACACTGCCCTGTACTACTGCGCC
AGGGACCTGTGGGAGCTGCGCGAGGCCTGCGACATCTGGGGCCAGGGCAC
CATGGTGACAGTGTCCTCCGGCGGCTCAGGAGGCTCCGGAGGCTCTGGCG
GCTCAGGCGGCGACATCGTGATGACCCAGAGCCCCAGCTTCCTGAGCACC
TTCGTGGGCGACAGGGTGACCATCACCTGCAGGGCCAGCCAGGGCATCAG
CAGCTACCTGGCCTGGTACCAGCAGAAGCCCGGCAAGGTGCCCAAGCTGC
TGATCTATGTGGCCAGCACCCTGCAGAGCGGGGTGCCCAGCCGCTTCAGC
GGCAGCGGCAGCGGCACAGAGTTCACCCTGACCATCAGCAGCCTGCAGCC
CGAGGACTTCGCCACATACTACTGCCAGCAGCTGAACAGCTACCCCTTCA
CCTTCGGGCCCGGCACCAAGGTGGACATCAAG.

SEQ ID NO: 106 includes gdT scFv: 1-732; gdT VH: 369-411; gdT Linker: 369-411; gdT VL: 412-732; Free energy: −338.7; gdT CAI: 0.860184134678756; ORF count: 3.

SEQ ID NO: 107 is the sequence name for gd-c V6 scFv ECOg (99). It is a gdT Targeting scFv construct. It is a DNA sequence. The sequence is:

CAGGTGCAGCTGCAGCAGAGCGGGCCCGGGCTCGTGAAGCCCTCGCAGAC
CCTCTCCCTCACGTGCGCGATCTCCGGGGACTCCGTGTCCTCCAACCGCG
CCGCGTGGAACTGGATACGGCAGAGCCCCTCGAAGGGGCTGGAGTGGCTG
GGGCGCACGTACTACCGCTCCAAGTGGTACAACGAGTACGCCGCCTCCGT
GAAGTCCCGCATGAGCATCAACCCCGACACCTCCAAGAACCAGTTCTCCC
TGCAGCTGAACTCCGTGACTCCCGAGGACACCGCGCTGTACTACTGCGCG
CGGGACCTGTGGGAGCTGCGCGAGGCGTGCGACATCTGGGGACAGGGGAC
CATGGTGACCGTGTCCTCCGGAGGAAGCGGAGGAAGCGGAGGAAGCGGAG
GAAGCGGAGGAGACATCGTGATGACGCAGTCCCCTTCCTTCCTCTCCACC
TTCGTGGGAGACCGCGTGACCATCACGTGCCGCGCTTCCCAGGGGATCTC
CTCCTACCTCGCGTGGTACCAGCAGAAGCCCGGGAAGGTGCCCAAGCTCC
TCATCTACGTGGCCTCCACGCTGCAGAGCGGGGTGCCCTCGCGCTTCTCC
GGGAGCGGGAGCGGGACGGAGTTCACCCTCACCATCTCTTCCCTGCAGCC
CGAGGACTTCGCCACGTACTACTGCCAGCAGCTGAACTCCTACCCCTTCA
CCTTCGGGCCCGGGACGAAGGTGGACATCAAG.

SEQ ID NO: 107 includes gdT scFv: 1-732; gdT VH: 369-411; gdT Linker: 369-411; gdT VL: 412-732; Free energy: −335; gdT CAI: 0.835639819762746; ORF count: 0.

SEQ ID NO: 108 is the sequence name for gd-c V1 HL scFv. It is a gdT Targeting scFv construct. It is an AA sequence. The sequence is:

EVQLLESGGGLVKPGGSLRLSCAASRFTLSSYDMNWVRQAPGKGLEWVS
SISSSSSYIYYADSVKGRFTISRDNAKNSLYLQMNSLRAEDTAVYYCAR
DRGVGGTDYYYYGLDVWGQGTTVTVSSGGSGGSGGSGGSGGEIVMTQSP
GTLSLSPGERATLSCRASQSVSSSYLAWYQQKPGQAPRLLIYGASSRAT
GIPDRFSGSGSGTDFTLTISRLEPEDFAVYYCQQYGSSPPYTFGQGTKV
EIK.

SEQ ID NO: 108 includes gdT scFv: 1-248; gdT VH: 126-139; gdT Linker: 126-139; gdT VL: 140-248.

SEQ ID NO: 109 is the sequence name for gd-c V1 HL scFv. It is a gdT Targeting scFv construct. It is a DNA sequence. The sequence is:

GAGGTGCAGCTGCTGGAGAGCGGCGGCGGCCTGGTGAAACCCGGCGGCT
CCCTGCGGCTGTCCTGCGCCGCCTCCAGGTTCACCCTGTCCAGCTACGA
CATGAACTGGGTGAGGCAGGCTCCCGGGAAGGGGCTGGAGTGGGTGTCC
TCCATCTCCTCCAGCTCCAGCTACATCTACTACGCCGATTCCGTGAAGG
GGAGATTCACCATCTCCAGGGACAACGCCAAGAACTCCCTGTACCTGCA
GATGAACTCCCTGCGGGCCGAGGACACCGCCGTGTACTACTGCGCCAGG
GACAGGGGCGTTGGCGGCACCGACTACTACTACTACGGGCTGGACGTGT
GGGGCCAGGGGACCACCGTGACCGTGTCCAGCGGCGGCTCTGGCGGCTC
TGGAGGCTCTGGCGGCTCTGGCGGCGAGATCGTGATGACCCAGTCTCCC
GGGACCCTGTCCCTGTCTCCCGGGGAGAGGGCTACCCTGTCCTGCAGGG
CCAGCCAGTCCGTGTCCTCCAGCTACCTGGCCTGGTACCAGCAGAAACC
CGGGCAGGCTCCCCGGCTGCTGATCTACGGGGCCTCTTCCAGGGCCACC
GGCATCCCCGACAGGTTCTCCGGGTCCGGGTCCGGGACCGACTTCACCC
TGACCATCTCCAGGCTGGAGCCCGAGGACTTCGCCGTGTACTACTGCCA
GCAGTACGGGTCCTCTCCACCTTACACCTTCGGGCAGGGGACCAAGGTG
GAGATCAAG.

SEQ ID NO: 109 includes gdT scFv: 1-744; gdT VH: 376-417; gdT Linker: 376-417; gdT VL: 418-744; Free energy: −341.2; gdT CAI: 0.91246469035254; ORF count: 2.

SEQ ID NO: 110 is the sequence name for gd-c V1 HL scFv (63). It is a gdT Targeting scFv construct. It is a DNA sequence. The sequence is:

GAGGTCCAGCTCCTGGAGAGCGGAGGAGGCCTGGTGAAGCCCGGAGGCT
CCCTGAGGCTCTCCTGCGCCGCCTCCAGGTTCACCCTCTCCTCCTACGA
CATGAACTGGGTGAGGCAGGCCCCGGGGAAGGGGCTGGAGTGGGTCTCC
TCCATCTCCTCCTCCTCCTCCTACATCTACTACGCCGACTCCGTGAAGG
GGCGCTTCACCATCTCCCGGGACAACGCCAAGAACTCCCTCTACCTCCA
GATGAACTCCCTGAGGGCCGAGGACACGGCCGTCTACTACTGCGCCCGG
GACCGAGGGGTAGGAGGCACCGACTACTACTACTACGGGCTGGACGTCT
GGGGCCAGGGGACCACCGTGACCGTCTCCTCCGGAGGCTCCGGAGGCTC
CGGAGGCTCCGGAGGCTCCGGAGGAGAGATCGTGATGACCCAGAGCCCC
GGGACCCTCTCCCTGAGCCCCGGGGAGAGGGCTACCCTCTCCTGCCGGG
CCAGCCAGAGCGTCTCCTCCTCCTACCTGGCCTGGTACCAGCAGAAGCC
GGGGCAGGCCCCTAGGCTCCTGATCTACGGGGCCTCCTCTAGGGCCACC
GGCATCCCGGACCGCTTCTCCGGGTCCGGGTCCGGGACCGACTTCACCC
TCACCATCTCCCGGCTGGAGCCGGAGGACTTCGCCGTCTACTACTGCCA
GCAGTACGGGTCCTCTCCTCCTTACACCTTCGGGCAGGGGACCAAGGTG
GAGATCAAG.

SEQ ID NO: 110 includes gdT scFv: 1-744; gdT VH: 376-417; gdT Linker: 376-417; gdT VL: 418-744; Free energy: −362.6; gdT CAI: 0.854971378798751; ORF count: 1.

SEQ ID NO: 111 is the sequence name for gd-c V1 HL scFv (72). It is a gdT Targeting scFv construct. It is a DNA sequence. The sequence is:

GAGGTGCAGCTGCTGGAGTCCGGCGGCGGCCTGGTGAAACCCGGCGGCA
GCCTGCGGCTGTCCTGCGCGGCAAGCCGCTTCACGCTGTCCAGCTACGA
CATGAACTGGGTGCGCCAGGCACCCGGCAAGGGGCTGGAGTGGGTGTCC
AGCATATCCAGCTCGTCAAGCTACATATACTACGCGGACAGCGTGAAGG
GCCGGTTTACCATCTCGCGGGATAACGCCAAGAACAGCCTGTACCTGCA
GATGAACAGCCTGCGGGCCGAGGACACCGCGGTGTACTACTGCGCAAGG
GACCGCGGGGTAGGCGGCACGGATTACTACTACTACGGGCTGGACGTGT
GGGGCCAGGGGACCACCGTGACGGTGTCCTCCGGCGGCTCAGGCGGTTC
CGGTGGCTCTGGCGGCTCAGGCGGCGAGATTGTCATGACGCAGTCACCC
GGCACGCTTAGCCTGTCGCCCGGGGAACGCGCCACGCTGTCCTGCCGGG
CCAGCCAGTCGGTGTCCAGCAGCTACCTGGCGTGGTACCAGCAGAAACC
CGGCCAGGCGCCCCGGCTGCTTATCTACGGGGCGTCTAGCCGGGCAACC
GGCATCCCGGACCGCTTCAGCGGGTCGGGCAGCGGGACGGACTTCACGC
TGACAATCAGCCGGCTGGAGCCCGAGGACTTCGCGGTGTACTACTGCCA
GCAGTACGGCAGCTCGCCGCCTTACACGTTTGGCCAGGGCACCAAGGTG
GAAATCAAG.

SEQ ID NO: 111 includes gdT scFv: 1-744; gdT VH: 376-417; gdT Linker: 376-417; gdT VL: 418-744; Free energy: −366.6; gdT CAI: 0.761156065773582; ORF count: 4.

SEQ ID NO: 112 is the sequence name for gd-c V1 HL scFv (14). It is a gdT Targeting scFv construct. It is a DNA sequence. The sequence is:

GAGGTCCAGCTGCTGGAGAGCGGCGGCGGCCTGGTCAAACCCGGCGGCT
CGCTGCGGCTGAGCTGCGCCGCCAGCAGGTTCACCCTGAGCTCCTACGA
CATGAACTGGGTGCGCCAGGCCCCAGGCAAGGGGCTGGAGTGGGTGAGC
TCGATCAGCTCGTCGTCGAGCTACATCTACTACGCCGACAGCGTCAAGG
GGCGCTTCACCATCTCGCGCGACAACGCCAAGAACTCGCTCTACCTCCA
GATGAACTCGCTGCGGGCCGAGGACACCGCCGTCTACTACTGCGCCCGA
GATCGCGGGGTTGGCGGCACCGACTACTACTACTACGGGCTCGACGTGT
GGGGCCAGGGGACCACCGTGACCGTGAGCTCCGGCGGCTCTGGCGGCTC
AGGTGGTAGCGGCGGCTCTGGCGGCGAGATCGTGATGACCCAGAGCCCC
GGGACCTTGTCGCTGAGCCCCGGCGAGAGGGCCACGCTGAGCTGCCGGG
CCAGCCAGAGCGTGAGCTCCAGCTACCTGGCCTGGTACCAGCAGAAACC
CGGCCAGGCGCCCCGGCTGTTGATCTACGGGGCCTCATCTCGGGCCACC
GGCATCCCCGACAGGTTCTCGGGGTCGGGGTCGGGGACCGACTTCACCC
TGACCATCTCGCGGCTGGAGCCCGAGGACTTCGCCGTCTACTACTGCCA
GCAGTACGGGAGCTCGCCGCCGTACACCTTCGGCCAGGGGACCAAGGTC
GAGATCAAG.

SEQ ID NO: 112 includes gdT scFv: 1-744; gdT VH: 376-417; gdT Linker: 376-417; gdT VL: 418-744; Free energy: −366.1; gdT CAI: 0.774531811261147; ORF count: 3.

SEQ ID NO: 113 is the sequence name for gd-c V1 HL scFv (22). It is a gdT Targeting scFv construct. It is a DNA sequence. The sequence is:

GAGGTGCAGCTGCTGGAGTCCGGCGGCGGCCTGGTGAAACCCGGCGGCT
CCCTGCGGCTGTCCTGCGCCGCCAGCAGGTTCACCCTCTCCTCCTACGA
CATGAACTGGGTGCGGCAGGCCCCAGGGAAGGGCCTGGAGTGGGTGAGC
AGCATCAGCAGCAGCAGCAGCTACATCTACTACGCCGACAGCGTCAAGG
GGCGCTTCACCATCAGCAGGGACAACGCCAAGAACAGCCTGTACCTGCA
GATGAACAGCCTCCGGGCCGAGGACACGGCCGTGTACTACTGCGCCCGT
GATCGTGGCGTCGGCGGCACCGACTACTACTACTACGGGCTGGACGTGT
GGGGCCAGGGGACGACCGTGACCGTCTCCTCCGGCGGCTCCGGAGGCTC
TGGTGGTTCTGGCGGCTCCGGCGGCGAGATCGTCATGACCCAGAGCCCC
GGGACCCTGTCCCTGAGCCCCGGGGAGAGGGCCACGCTCTCCTGCCGGG
CCAGCCAGTCCGTGTCCTCCTCCTACCTGGCCTGGTACCAGCAGAAACC
CGGCCAGGCGCCCAGGCTGCTGATCTACGGGGCCTCTTCTCGCGCCACG
GGCATCCCCGACCGCTTCTCCGGGAGCGGCTCCGGGACGGACTTCACCC
TGACCATCAGCCGCCTGGAGCCCGAGGACTTCGCCGTGTACTACTGCCA
GCAGTACGGGTCCTCGCCGCCGTACACCTTCGGGCAGGGGACGAAGGTG
GAGATCAAG.

SEQ ID NO: 113 includes gdT scFv: 1-744; gdT VH: 376-417; gdT Linker: 376-417; gdT VL: 418-744; Free energy: −359.3; gdT CAI: 0.831881016856322; ORF count: 3.

SEQ ID NO: 114 is the sequence name for gd-c V1 HL scFv (11). It is a gdT Targeting scFv construct. It is a DNA sequence. The sequence is:

GAGGTGCAGCTGCTGGAGAGCGGCGGCGGCCTCGTGAAACCCGGCGGCT
CGCTGCGGCTCTCGTGCGCCGCTTCGCGCTTCACGCTCTCGTCGTACGA
CATGAACTGGGTGCGGCAGGCTCCCGGGAAGGGGCTGGAGTGGGTCTCG
TCGATCTCGTCGTCGTCGAGCTACATCTACTACGCCGACTCCGTGAAGG
GGCGCTTCACGATCTCGCGCGACAACGCGAAGAACTCGCTCTACCTGCA
GATGAACTCGCTGCGCGCCGAGGACACCGCCGTCTACTACTGCGCTCGC
GATCGCGGAGTCGGCGGCACCGACTACTACTACTACGGGCTCGACGTCT
GGGGCCAGGGGACGACCGTGACCGTGTCGAGCGGCGGAAGCGGCGGAAG
CGGAGGAAGCGGCGGAAGCGGCGGCGAGATCGTGATGACGCAGTCGCCC
GGGACGCTCTCGCTCTCGCCCGGCGAGCGCGCTACGCTCTCGTGCCGCG
CGTCGCAGAGCGTCTCGTCGAGCTACCTCGCGTGGTACCAGCAGAAACC
CGGGCAGGCGCCGCGGCTGCTTATCTACGGCGCTAGCTCTCGCGCGACC
GGCATCCCCGACCGCTTCTCCGGCTCCGGCTCCGGGACCGACTTCACGC
TGACGATCTCGCGGCTGGAGCCCGAGGACTTCGCCGTCTACTACTGCCA
GCAGTACGGCTCGTCGCCGCCGTACACGTTCGGGCAGGGGACGAAGGTC
GAGATCAAG.

SEQ ID NO: 114 includes gdT scFv: 1-744; gdT VH: 376-417; gdT Linker: 376-417; gdT VL: 418-744; Free energy: −358.3; gdT CAI: 0.705025041042151; ORF count: 1.

SEQ ID NO: 115 is the sequence name for JAML scFv. It is a gdT Targeting scFv construct. It is an AA sequence. The sequence is:

DVQLVESGAELVRPGASKLSCKALAYTFTDYEMHWVKQTPVHGLEWIGI
IHPGSGGTVYNQKFKGKATLTADKSSSTAYMELSSLTSEDSTVYYCTRR
RYYGSSYNWYFDVWGAGNGGSGGSGGSGGSGGVLTQSPASLAASVGETV
TITCRASENIYYSLAWYQQKQGKSPQLLIYNANSLEDGVPSRFSGSGSG
TQYSLKINSMQPEDTATYFCEQTYDVPLTFGAGTKLEL.

SEQ ID NO: 115 includes gdT scFv: 1-234; gdT VH: 117-130; gdT Linker: 117-130; gdT VL: 131-234; Free energy: −328.2; gdT CAI: 0.850906150801958; ORF count: 2.

SEQ ID NO: 116 is the sequence name for JAML scFv. It is a gdT Targeting scFv construct. It is a DNA sequence. The sequence is:

GATGTCCAGCTGGTGGAGTCTGGGGCTGAGCTGGTGAGGCCTGGGGCCA
GCAAGCTGTCCTGCAAGGCCCTGGCCTACACCTTCACAGACTATGAGAT
GCACTGGGTGAAGCAGACCCCAGTGCATGGGCTGGAGTGGATTGGGATC
ATCCATCCAGGCTCTGGTGGCACAGTCTACAACCAGAAGTTCAAGGGGA
AGGCCACACTCACAGCTGACAAGTCCAGCTCCACAGCCTACATGGAGCT
GTCCAGCCTGACCTCTGAGGACTCCACAGTCTACTACTGCACCAGGAGG
AGGTACTATGGCTCCAGCTACAACTGGTACTTTGATGTGTGGGGAGCTG
GGAATGGTGGCTCTGGTGGCTCTGGTGGCTCTGGTGGCTCTGGTGGAGT
GCTGACCCAGTCCCCAGCCAGCCTGGCTGCCTCTGTTGGGGAGACAGTC
ACCATCACCTGCAGGGCCTCTGAGAACATCTACTACAGCCTGGCCTGGT
ACCAGCAGAAGCAGGGGAAGTCTCCCCAGCTGCTGATCTACAATGCCAA
CAGCCTGGAGGATGGGGTGCCCAGCAGGTTCTCTGGGTCTGGGTCTGGC
ACCCAGTACTCCCTCAAGATCAACAGCATGCAGCCAGAGGACACAGCCA
CCTACTTCTGTGAGCAGACCTATGATGTGCCCCTCACCTTTGGGGCTGG
CACCAAGCTGGAGCTC.

SEQ ID NO: 116 includes gdT scFv: 1-702; gdT VH: 349-390; gdT Linker: 349-390; gdT VL: 391-702; Free energy: −302; gdT CAI: 0.868315362715317; ORF count: 10.

SEQ ID NO: 117 is the sequence name for JAML scFv (88). It is a gdT Targeting scFv construct. It is a DNA sequence. The sequence is:

GACGTGCAGCTGGTGGAGTCCGGGGCTGAGCTGGTGAGGCCCGGGGCCT
CCAAGCTGAGCTGCAAGGCCCTGGCCTACACCTTCACCGACTACGAGAT
GCACTGGGTGAAGCAGACCCCGGTGCACGGGCTGGAGTGGATCGGGATC
ATCCATCCCGGGTCCGGCGGCACCGTGTACAACCAGAAGTTCAAGGGGA
AGGCCACGCTGACCGCCGACAAGTCCAGCTCCACCGCCTACATGGAGCT
GTCCAGCCTGACCTCCGAGGACTCCACCGTGTACTACTGCACCCGGCGG
CGGTACTACGGGTCCTCCTACAACTGGTACTTCGACGTGTGGGGCGCCG
GGAACGGCGGATCAGGCGGCTCCGGAGGCTCAGGAGGCTCCGGCGGCGT
GCTGACCCAGAGCCCCGCTAGCCTGGCCGCCTCCGTCGGGGAGACCGTG
ACCATCACCTGCCGGGCCTCCGAGAACATCTACTACTCCCTGGCCTGGT
ACCAGCAGAAGCAGGGGAAGTCTCCCCAGCTGCTGATCTACAACGCCAA
CTCCCTGGAGGACGGGGTGCCCAGCCGGTTCTCCGGGTCCGGGTCCGGG
ACCCAGTACAGCCTGAAGATCAACAGCATGCAGCCCGAGGACACCGCCA
CCTACTTCTGCGAGCAGACCTACGACGTGCCCCTGACCTTCGGGGCCGG
GACCAAGCTGGAGCTG.

SEQ ID NO: 117 includes gdT scFv: 1-702; gdT VH: 349-390; gdT Linker: 349-390; gdT VL: 391-702; Free energy: −323.6; gdT CAI: 0.915740281407777; ORF count: 1.

SEQ ID NO: 118 is the sequence name for JAML scFv (84). It is a gdT Targeting scFv construct. It is a DNA sequence. The sequence is:

GATGTCCAGCTCGTGGAGTCCGGGGCTGAGCTCGTGAGGCCCGGGGCCT
CGAAGCTGAGCTGCAAGGCCCTGGCCTACACCTTCACGGACTACGAGAT
GCACTGGGTGAAGCAGACCCCGGTGCACGGCCTGGAGTGGATCGGGATC
ATTCACCCCGGGTCCGGCGGCACCGTGTACAACCAGAAGTTCAAGGGCA
AGGCCACGCTGACCGCGGACAAGTCCTCGTCCACGGCGTACATGGAGCT
GAGCTCCCTGACCTCCGAGGACTCCACGGTGTACTACTGCACACGGCGG
CGGTACTACGGGAGCTCCTACAACTGGTACTTCGACGTCTGGGGCGCCG
GGAACGGAGGCTCTGGAGGCTCCGGAGGCTCTGGAGGCTCCGGCGGCGT
CCTGACCCAGAGCCCCGCATCCCTGGCGGCCTCCGTCGGGGAGACCGTG
ACCATCACCTGCAGGGCCTCGGAGAACATCTACTACTCCCTGGCGTGGT
ACCAGCAGAAGCAGGGGAAGTCCCCGCAGCTCCTGATCTACAACGCCAA
CTCCCTGGAGGACGGGGTGCCCTCGCGGTTCTCGGGGTCCGGGTCCGGG
ACCCAGTACTCCCTGAAGATCAACTCCATGCAGCCCGAGGACACCGCGA
CGTACTTCTGCGAGCAGACCTACGACGTGCCCCTGACCTTCGGCGCCGG
GACAAAGCTGGAGCTC.

SEQ ID NO: 118 includes gdT scFv: 1-702; gdT VH: 349-390; gdT Linker: 349-390; gdT VL: 391-702; Free energy: −328.2; gdT CAI: 0.850906150801958; ORF count: 2.

SEQ ID NO: 119 is the sequence name for JAML scFv (44). It is a gdT Targeting scFv construct. It is a DNA sequence. The sequence is:

GATGTGCAGCTGGTGGAGTCCGGGGCTGAGCTGGTGAGGCCCGGGGCCT
CCAAGCTGTCCTGCAAGGCCCTGGCCTACACCTTCACCGACTACGAGAT
GCACTGGGTGAAGCAGACGCCCGTGCACGGGCTGGAGTGGATCGGGATC
ATTCACCCCGGGTCCGGCGGCACCGTGTACAACCAGAAGTTCAAGGGGA
AGGCCACGCTGACCGCCGACAAGTCCTCCTCCACCGCCTACATGGAGCT
GAGCTCCCTGACCTCCGAGGACTCCACCGTGTACTACTGCACCCGGCGG
CGGTACTACGGGTCCTCCTACAACTGGTACTTCGACGTGTGGGGCGCCG
GGAACGGCGGATCTGGCGGCTCCGGCGGATCTGGCGGCTCTGGCGGCGT
GCTGACCCAGAGCCCCGCTTCCCTGGCCGCCTCCGTCGGGGAGACCGTG
ACCATCACCTGCCGGGCCTCCGAGAACATCTACTACTCCCTGGCCTGGT
ACCAGCAGAAGCAGGGGAAGTCTCCCCAGCTGCTGATCTACAACGCCAA
CTCCCTGGAGGACGGGGTGCCCAGCCGGTTCTCCGGGTCCGGGTCCGGG
ACCCAGTACTCCCTGAAGATCAACTCCATGCAGCCCGAGGACACCGCCA
CCTACTTCTGCGAGCAGACCTACGACGTGCCCCTGACCTTCGGGGCCGG
GACCAAGCTGGAGCTG.

SEQ ID NO: 119 includes gdT scFv: 1-702; gdT VH: 349-390; gdT Linker: 349-390; gdT VL: 391-702; Free energy: −318.2; gdT CAI: 0.934766339108284; ORF count: 2.

SEQ ID NO: 120 is the sequence name for JAML scFv (23). It is a gdT Targeting scFv construct. It is a DNA sequence. The sequence is:

GACGTGCAGCTGGTGGAGTCCGGGGCCGAGCTGGTACGGCCAGGCGCCA
GCAAGCTGTCCTGCAAGGCGCTCGCCTACACCTTCACCGACTACGAGAT
GCACTGGGTGAAGCAGACGCCCGTGCACGGGCTGGAGTGGATCGGGATC
ATACACCCCGGGTCCGGCGGCACCGTCTACAACCAGAAGTTCAAGGGGA
AGGCTACCCTTACGGCCGACAAGTCCTCCTCTACCGCCTACATGGAGCT
GTCCAGCCTGACGAGCGAGGACTCTACCGTCTACTACTGCACCCGCAGG
CGGTACTACGGGTCCAGCTACAACTGGTACTTCGACGTGTGGGGCGCCG
GGAACGGCGGTAGTGGCGGTAGCGGCGGTAGTGGCGGTAGCGGCGGCGT
GCTGACCCAGTCGCCCGCTAGCCTGGCCGCTAGCGTCGGGGAGACCGTC
ACCATCACCTGCCGGGCGAGCGAGAACATCTACTACTCGCTCGCCTGGT
ACCAGCAGAAGCAGGGGAAGAGCCCGCAGCTGCTGATCTATAACGCCAA
CAGCCTGGAGGACGGGGTGCCCAGCCGGTTCAGCGGGAGCGGGAGCGGG
ACCCAGTACTCCCTCAAGATCAACAGCATGCAGCCGGAGGATACCGCCA
CCTACTTCTGCGAGCAGACGTACGACGTGCCCCTCACGTTCGGGGCCGG
CACCAAGCTGGAGCTG.

SEQ ID NO: 120 includes gdT scFv: 1-702; gdT VH: 349-390; gdT Linker: 349-390; gdT VL: 391-702; Free energy: −314.5; gdT CAI: 0.828248130876822; ORF count: 3.

SEQ ID NO: 121 is the sequence name for JAML scFv (78). It is a gdT Targeting scFv construct. It is a DNA sequence. The sequence is:

GACGTGCAGCTCGTGGAGAGCGGCGCAGAGCTTGTGCGCCCCGGGGCCT
CCAAGCTGTCCTGCAAGGCGCTCGCCTACACCTTCACGGACTACGAGAT
GCACTGGGTGAAGCAGACCCCTGTGCACGGCCTGGAGTGGATCGGGATC
ATTCACCCCGGGTCAGGAGGCACCGTGTACAACCAGAAGTTCAAGGGGA
AGGCCACGCTGACCGCCGACAAGTCCTCCTCCACGGCCTACATGGAGCT
CTCCTCGCTGACCTCCGAGGACTCCACGGTGTACTACTGCACGCGGAGG
AGGTACTACGGGTCCTCCTACAACTGGTACTTCGACGTGTGGGGCGCGG
GGAATGGCGGCTCAGGAGGCTCAGGAGGCTCAGGAGGCTCAGGCGGCGT
GCTCACGCAGAGCCCCGCAAGCCTCGCGGCGTCCGTCGGGGAGACCGTG
ACGATCACCTGCAGGGCCTCCGAGAACATCTACTACTCCCTCGCGTGGT
ACCAGCAGAAGCAGGGGAAGTCCCCTCAGCTCCTCATCTACAACGCGAA
CTCCCTGGAGGACGGGGTGCCCTCAAGGTTCTCCGGGTCGGGGAGCGGC
ACGCAGTACTCCCTGAAGATCAACTCCATGCAGCCCGAGGACACCGCGA
CGTACTTCTGCGAGCAGACGTACGACGTGCCCCTCACCTTCGGGGCCGG
CACGAAGCTCGAACTG.

SEQ ID NO: 121 includes gdT scFv: 1-702; gdT VH: 349-390; gdT Linker: 349-390; gdT VL: 391-702; Free energy: −314.4; gdT CAI: 0.837369130789258; ORF count: 3.

SEQ ID NO: 122 is the sequence name for CDXAR ligand. It is a gdT Targeting ligand construct. It is an AA sequence. The sequence is:

LSITTPEEMIEKAKGETAYLPCKFTLSPEDQGPLDIEWLISPADNQKVD
QVIILYSGDKIYDDYYPDLKGRVHFTSNDLKSGDASINVTNLQLSDIGT
YQCKVKKAPGVANKKIHLVVLVKPSGARCYVDGSEEIGSDFKIKCEPKE
GSLPLQYEWQKLSDSQKMPTSWLAGKMCHLQRAVRPLPEATSAVIIHPW
GPCLLPTWKDIPRLSITKYQVKTLNALLRVRLSHLLR.

SEQ ID NO: 122 includes gdT ligand: 1-233.

SEQ ID NO: 123 is the sequence name for CDXAR ligand. It is a gdT Targeting ligand construct. It is a DNA sequence. The sequence is:

CTGAGCATCACTACCCCTGAGGAGATGATTGAGAAGGCTAAGGGTGAGA
CAGCCTACCTGCCCTGCAAGTTCACCCTGAGCCCTGAGGACCAGGGGCC
CCTGGACATTGAGTGGCTGATCAGCCCAGCTGACAACCAGAAGGTGGAC
CAGGTCATCATCCTGTACTCAGGGGACAAGATCTATGATGACTACTACC
CTGACCTGAAGGGCAGGGTGCACTTCACCAGCAATGACCTGAAGTCAGG
GGATGCCAGCATCAATGTGACCAACCTGCAGCTGTCTGACATAGGCACC
TACCAGTGCAAGGTCAAGAAGGCCCCAGGGGTAGCCAACAAGAAGATCC
ACCTGGTGGTGCTGGTCAAGCCCTCAGGGGCCAGGTGCTATGTGGATGG
CTCTGAGGAGATAGGCTCTGACTTCAAGATCAAGTGTGAGCCCAAAGAG
GGCAGCCTGCCCCTGCAGTATGAGTGGCAGAAGCTGTCTGACAGCCAGA
AGATGCCCACTAGCTGGCTGGCTGGCAAGATGTGCCACCTGCAGAGGGC
TGTCAGGCCCCTGCCTGAGGCCACCTCAGCTGTCATCATTCACCCCTGG
GGCCCCTGCCTGCTGCCTACCTGGAAGGACATCCCCAGGCTGAGCATCA
CTAAGTACCAGGTCAAGACCCTCAATGCCCTGCTGAGGGTCAGGCTGAG
CCACCTGCTGAGG.

SEQ ID NO: 123 includes gdT ligand: 1-233; Free energy: −284.1; gdT CAI: 0.86860152001121; ORF count: 7.

SEQ ID NO: 124 is the sequence name for CDXAR ligand (6). It is a gdT Targeting ligand construct. It is a DNA sequence. The sequence is:

CTGAGCATCACCACGCCCGAGGAGATGATCGAGAAGGCCAAGGGCGAGA
CCGCCTACCTGCCCTGCAAGTTCACCCTGAGCCCCGAGGACCAGGGGCC
CCTGGACATCGAGTGGCTGATCTCGCCCGCGGACAACCAGAAGGTGGAC
CAGGTGATCATCCTGTACTCCGGGGACAAGATCTACGACGACTACTACC
CCGACCTGAAGGGGCGCGTGCACTTCACCTCCAACGACCTGAAGTCCGG
GGACGCCAGCATCAACGTGACCAACCTGCAGCTGTCCGACATCGGGACC
TACCAGTGCAAGGTGAAGAAGGCGCCCGGGGTCGCCAACAAGAAGATCC
ACCTGGTGGTGCTGGTGAAGCCCAGCGGGGCCAGGTGCTACGTGGACGG
GAGCGAGGAGATCGGGTCCGACTTCAAGATCAAGTGCGAGCCCAAAGAG
GGGTCCCTGCCCCTGCAGTACGAGTGGCAGAAGCTGTCCGACAGCCAGA
AGATGCCGACCTCCTGGCTGGCCGGGAAGATGTGCCACCTGCAGCGGGC
CGTGAGGCCCCTGCCTGAGGCCACCAGCGCCGTGATCATTCACCCCTGG
GGCCCCTGCCTGCTGCCAACATGGAAGGACATCCCGCGGCTGTCCATCA
CCAAGTACCAGGTGAAGACCCTGAACGCCCTGCTGCGGGTGCGGCTGTC
GCACCTGCTGCGG.

SEQ ID NO: 124 includes gdT ligand: 1-233; Free energy: −291.4; gdT CAI: 0.912300179663014; ORF count: 1.

SEQ ID NO: 125 is the sequence name for CDXAR ligand (57). It is a gdT Targeting ligand construct. It is a DNA sequence. The sequence is:

CTCTCGATCACCACACCCGAGGAGATGATCGAGAAGGCGAAGGGCGAGA
CCGCCTACTTGCCCTGCAAGTTCACCCTCTCGCCCGAGGACCAGGGGCC
CCTCGATATCGAGTGGCTCATCTCGCCCGCGGACAACCAAAAGGTGGAC
CAGGTGATCATCCTCTATAGTGGGGACAAGATCTACGACGACTACTACC
CCGATCTCAAGGGGCGCGTCCACTTCACCTCCAACGACCTCAAGAGCGG
GGACGCCTCGATCAACGTGACCAACCTCCAATTGAGCGACATCGGGACC
TACCAGTGCAAGGTGAAGAAGGCGCCCGGGGTCGCCAACAAGAAGATCC
ACCTCGTGGTGCTCGTGAAGCCCAGTGGGGCGCGGTGCTACGTGGATGG
GTCGGAGGAGATCGGGAGCGACTTCAAGATCAAGTGCGAGCCCAAAGAG
GGCTCGCTACCCCTACAATACGAGTGGCAGAAGTTGAGCGACTCCCAAA
AGATGCCCACTAGCTGGCTCGCGGGCAAGATGTGCCACCTCCAGCGCGC
CGTGCGACCCCTACCCGAGGCCACGAGCGCGGTCATCATACACCCCTGG
GGCCCCTGCCTACTCCCCACTTGGAAGGACATCCCGCGCCTCTCGATCA
CCAAGTACCAGGTGAAGACGCTCAACGCGCTCTTGCGCGTGCGCCTCTC
GCACCTCTTGAGG.

SEQ ID NO: 125 includes gdT ligand: 1-233; Free energy: −290.9; gdT CAI: 0.776501808272779; ORF count: 4.

SEQ ID NO: 126 is the sequence name for CDXAR ligand (56). It is a gdT Targeting ligand construct. It is a DNA sequence. The sequence is:

CTGAGCATCACCACGCCGGAGGAGATGATCGAGAAGGCGAAGGGCGAGA
CCGCGTACCTGCCCTGCAAGTTCACCCTGAGCCCGGAGGACCAGGGGCC
GCTGGACATCGAGTGGCTGATCAGCCCCGCGGACAACCAGAAGGTGGAC
CAGGTGATCATCCTGTACAGCGGGGACAAGATCTACGACGACTACTACC
CGGACCTGAAGGGGCGCGTGCACTTCACCTCCAACGACCTGAAGAGCGG
GGACGCCAGCATCAACGTGACCAACCTGCAGCTGAGCGACATCGGGACC
TACCAGTGCAAGGTGAAGAAGGCGCCCGGGGTCGCGAACAAGAAGATCC
ACCTGGTGGTGCTGGTGAAGCCCAGCGGGGCCCGGTGCTACGTGGACGG
GTCCGAGGAGATCGGGTCCGACTTCAAGATCAAGTGCGAGCCGAAAGAG
GGGTCCCTGCCGCTGCAGTACGAGTGGCAGAAGCTGAGCGACAGCCAGA
AGATGCCGACCAGCTGGCTGGCCGGGAAGATGTGCCACCTGCAGCGGGC
TGTCCGGCCGCTCCCTGAGGCTACCTCCGCGGTGATCATCCATCCCTGG
GGCCCCTGCCTGCTCCCGACCTGGAAGGACATCCCGCGGCTGAGCATCA
CCAAGTACCAGGTGAAGACCCTGAACGCGCTGCTCCGGGTCCGGCTGAG
CCACCTGCTCCGG.

SEQ ID NO: 126 includes gdT ligand: 1-233; Free energy: −287.5; gdT CAI: 0.835151871227941; ORF count: 2.

SEQ ID NO: 127 is the sequence name for CDXAR ligand (73). It is a gdT Targeting ligand construct. It is a DNA sequence. The sequence is:

CTCTCGATCACCACGCCCGAGGAGATGATCGAGAAGGCCAAGGGCGAGA
CCGCCTACTTGCCCTGCAAGTTCACCCTCTCGCCCGAGGACCAGGGGCC
CCTCGACATCGAGTGGCTCATCTCGCCCGCGGACAACCAGAAGGTGGAC
CAGGTGATCATCCTCTACTCGGGCGACAAGATCTACGACGACTACTACC
CCGACCTCAAGGGGCGCGTGCACTTCACGAGCAACGACCTCAAGAGCGG
GGACGCCTCGATCAACGTGACCAACCTCCAGCTCTCCGACATCGGGACC
TACCAGTGCAAGGTGAAGAAGGCGCCCGGGGTCGCCAACAAGAAGATCC
ACCTCGTGGTGTTGGTGAAGCCCAGCGGGGCGCGGTGCTACGTGGACGG
GAGCGAGGAGATCGGGAGCGACTTCAAGATCAAGTGCGAGCCCAAAGAG
GGGTCGCTCCCGCTCCAGTACGAGTGGCAGAAGCTGAGCGACTCCCAGA
AGATGCCCACGAGCTGGCTCGCGGGGAAGATGTGCCACCTCCAGCGCGC
GGTTCGCCCACTCCCCGAGGCCACGAGCGCGGTGATCATTCACCCATGG
GGCCCCTGCCTCTTGCCCACGTGGAAGGACATCCCGCGCCTCTCGATCA
CCAAGTACCAGGTGAAGACCCTCAACGCGCTCCTGCGCGTGCGCCTCTC
CCACTTGTTGAGG.

SEQ ID NO: 127 includes gdT ligand: 1-233; Free energy: −287.4; gdT CAI: 0.810082913563651; ORF count: 0.

SEQ ID NO: 128 is the sequence name for CDXAR ligand (63). It is a gdT Targeting ligand construct. It is a DNA sequence. The sequence is:

CTGAGCATCACGACGCCCGAGGAGATGATCGAGAAGGCCAAAGGGGAGA
CCGCCTACCTGCCCTGCAAGTTCACCCTGAGCCCCGAGGACCAGGGGCC
CCTGGACATCGAGTGGCTGATCTCCCCGGCGGACAACCAGAAGGTCGAC
CAGGTGATCATCCTGTACTCCGGGGACAAGATCTACGACGACTACTACC
CCGACCTGAAGGGCCGGGTGCACTTCACGTCGAACGACCTGAAGTCCGG
GGACGCCTCGATCAACGTGACCAACCTGCAGCTGTCCGACATCGGGACC
TACCAGTGCAAGGTGAAGAAGGCGCCCGGCGTGGCCAACAAGAAGATCC
ACCTGGTGGTCCTCGTGAAGCCCAGCGGGGCGCGGTGCTACGTGGACGG
CAGCGAGGAGATCGGGTCGGACTTCAAGATCAAGTGCGAGCCCAAAGAG
GGCAGCCTGCCCCTGCAGTACGAGTGGCAGAAGCTGTCCGACAGCCAGA
AGATGCCGACCTCCTGGCTGGCCGGCAAGATGTGCCACCTGCAGCGCGC
CGTGAGGCCACTCCCCGAGGCGACCAGCGCGGTGATCATTCACCCCTGG
GGCCCCTGCCTGCTGCCAACCTGGAAGGACATCCCGCGGCTGTCCATCA
CGAAGTACCAGGTGAAGACCCTGAACGCCCTGCTGCGCGTCCGGCTGAG
CCACCTGCTGCGC.

SEQ ID NO: 128 includes gdT ligand: 1-233; Free energy: −286; gdT CAI: 0.861258772692867; ORF count: 0.

SEQ ID NO: 129 is the sequence name for CD5 scFv. It is a gdT Targeting scFv construct. It is an AA sequence. The sequence is:

EIQLVQSGGGLVKPGGSVRISCAASGYTFTNYGMNWVRQAPGKGLEWMG
WINTHTGEPTYADSFKGRFTFSLDDSKNTAYLQINSLRAEDTAVYFCTR
RGYDWYFDVWGQGTTVTVSSGGGGSGGGGSGGGGSDIQMTQSPSSLSAS
VGDRVTITCRASQDINSYLSWFQQKPGKAPKTLIYRANRLESGVPSRFS
GSGSGTDYTLTISSLQYEDFGIYYCQQYDESPWTFGGGTKLEIK.

SEQ ID NO: 129 includes gdT scFv: 1-240; gdT VH: 119-133; gdT Linker: 119-133; gdT VL: 134-240.

SEQ ID NO: 130 is the sequence name for CD5 scFv. It is a gdT Targeting scFv construct. It is a DNA sequence. The sequence is:

GAGATCCAGCTGGTGCAGTCTGGGGGGGGGCTGGTGAAGCCTGGGGGGT
CAGTGAGGATCAGCTGTGCTGCCTCAGGGTACACCTTCACCAACTATGG
GATGAACTGGGTGAGGCAGGCCCCAGGGAAGGGGCTGGAGTGGATGGGG
TGGATCAACACCCACACTGGGGAGCCCACCTATGCTGACAGCTTCAAGG
GGAGGTTCACCTTCAGCCTGGATGACTCCAAGAACACAGCCTACCTGCA
GATCAACTCCCTGAGGGCTGAGGACACAGCTGTGTACTTCTGCACCAGG
AGGGGGTATGACTGGTACTTTGATGTGTGGGGGCAGGGGACCACAGTGA
CAGTGTCCTCTGGGGGGGGGGGGTCTGGGGGGGGGGGGTCTGGGGGGGG
GGGGTCTGACATCCAGATGACCCAGTCCCCCTCCAGCCTGTCTGCCTCA
GTGGGGGACAGGGTGACCATCACCTGCAGGGCCTCCCAGGACATCAACT
CCTACCTGTCCTGGTTCCAGCAGAAGCCTGGGAAGGCCCCCAAGACCCT
GATCTACAGGGCCAACAGGCTGGAGTCTGGGGTGCCCTCCAGGTTCTCA
GGGTCTGGGTCTGGGACAGACTACACCCTGACCATCTCCAGCCTGCAGT
ATGAGGACTTTGGGATCTACTACTGCCAGCAGTATGATGAGTCCCCCTG
GACCTTTGGGGGGGGGACCAAGCTGGAGATCAAG.

SEQ ID NO: 130 includes gdT scFv: 1-720; gdT VH: 355-399; gdT Linker: 355-399; gdT VL: 400-720; Free energy: −346.2; gdT CAI: 0.943955363575702; ORF count: 8.

SEQ ID NO: 131 is the sequence name for CD5 scFv (11). It is a gdT Targeting scFv construct. It is a DNA sequence. The sequence is:

GAGATCCAGCTGGTGCAGTCCGGCGGCGGCCTGGTGAAACCCGGCGGCT
CCGTGCGGATCTCCTGCGCCGCCTCCGGGTACACCTTCACCAACTACGG
GATGAACTGGGTGCGGCAGGCTCCCGGGAAGGGGCTGGAGTGGATGGGC
TGGATCAACACCCACACCGGGGAGCCCACATACGCCGACTCCTTCAAGG
GGCGCTTCACCTTCTCCCTGGACGACTCCAAGAACACCGCCTACCTGCA
GATCAACTCCCTGCGGGCCGAGGACACCGCCGTGTACTTCTGCACCCGG
CGCGGGTACGACTGGTACTTCGACGTGTGGGGCCAGGGGACCACCGTGA
CCGTGTCCTCTGGCGGCGGAGGTTCCGGCGGAGGAGGCTCCGGAGGAGG
CGGCAGCGACATCCAGATGACCCAGAGCCCCAGCAGCCTGTCCGCCTCC
GTCGGGGACCGGGTGACCATCACCTGCCGGGCCAGCCAGGACATCAACA
GCTACCTGTCCTGGTTCCAGCAGAAGCCCGGGAAGGCTCCCAAGACCCT
GATCTACCGGGCCAACCGGCTGGAGAGCGGGGTGCCCAGCCGGTTCAGC
GGGTCCGGGTCCGGGACCGACTACACCCTGACCATCAGCTCCCTGCAGT
ACGAGGACTTCGGGATCTACTACTGCCAGCAGTACGACGAGTCCCCATG
GACCTTCGGCGGCGGCACCAAGCTGGAGATCAAG.

SEQ ID NO: 131 includes gdT scFv: 1-720; gdT VH: 355-399; gdT Linker: 355-399; gdT VL: 400-720; Free energy: −345.9; gdT CAI: 0.885961949519618; ORF count: 1.

SEQ ID NO: 132 is the sequence name for CD5 scFv (9). It is a gdT Targeting scFv construct. It is a DNA sequence. The sequence is:

GAGATCCAGCTGGTGCAGTCCGGCGGCGGCCTGGTGAAACCCGGCGGCT
CCGTGCGGATCTCCTGCGCCGCCTCCGGGTACACCTTCACCAACTACGG
GATGAACTGGGTGCGGCAGGCTCCCGGGAAGGGGCTGGAGTGGATGGGC
TGGATCAACACCCACACCGGGGAGCCCACGTACGCCGACTCCTTCAAGG
GGCGCTTCACCTTCTCCCTCGACGACTCCAAGAACACCGCCTACCTGCA
GATCAACTCCCTGCGGGCCGAGGACACCGCCGTGTACTTCTGCACCCGG
CGCGGGTACGACTGGTACTTCGACGTGTGGGGCCAGGGGACCACCGTGA
CCGTGTCCTCCGGAGGCGGCGGCTCTGGAGGAGGCGGCTCTGGAGGCGG
AGGCTCCGACATCCAGATGACCCAGAGCCCCAGCTCCCTCTCCGCCTCC
GTCGGGGACCGGGTGACCATCACCTGCCGCGCCAGCCAGGACATCAACT
CCTACCTCTCCTGGTTCCAGCAGAAGCCCGGGAAGGCGCCCAAGACCCT
CATCTACCGCGCCAACCGGCTGGAGTCCGGGGTGCCCAGCCGCTTCTCC
GGGTCCGGGTCCGGGACCGACTACACCCTCACCATCTCCAGCCTGCAGT
ACGAGGACTTCGGGATCTACTACTGCCAGCAGTACGACGAGAGCCCCTG
GACCTTCGGCGGCGGCACCAAGCTGGAGATCAAG.

SEQ ID NO: 132 includes gdT scFv: 1-720; gdT VH: 355-399; gdT Linker: 355-399; gdT VL: 400-720; Free energy: −344.1; gdT CAI: 0.8842887077719; ORF count: 1.

SEQ ID NO: 133 is the sequence name for CD5 scFv (41). It is a gdT Targeting scFv construct. It is a DNA sequence. The sequence is:

GAAATCCAGCTGGTGCAGTCCGGCGGCGGCCTGGTGAAACCCGGCGGCT
CCGTGCGGATCAGCTGCGCGGCCAGCGGGTACACCTTTACGAACTACGG
GATGAACTGGGTGCGCCAGGCACCCGGCAAGGGGCTCGAATGGATGGGC
TGGATCAACACGCACACCGGGGAGCCAACCTACGCGGATAGCTTCAAGG
GGCGCTTCACGTTCAGCCTGGACGACTCGAAGAACACCGCCTACCTGCA
GATCAACTCGCTGCGCGCCGAGGACACCGCGGTGTACTTCTGCACGCGG
CGCGGGTACGACTGGTACTTCGACGTGTGGGGCCAGGGGACCACCGTGA
CGGTGTCCTCCGGAGGAGGCGGCTCCGGAGGAGGCGGCTCAGGAGGCGG
CGGATCGGACATCCAGATGACCCAGTCGCCCAGCAGCCTGTCGGCCAGC
GTGGGCGACCGGGTGACCATCACCTGCCGGGCGTCCCAGGATATCAACA
GCTACCTGTCGTGGTTCCAGCAGAAGCCCGGCAAGGCGCCCAAAACGCT
GATCTACCGGGCCAACCGGCTGGAAAGCGGGGTGCCCAGCCGGTTCAGC
GGGTCGGGCAGCGGGACGGACTACACGCTGACCATCAGCAGCCTGCAGT
ACGAGGACTTCGGGATATACTACTGCCAGCAGTACGACGAGTCGCCCTG
GACGTTCGGCGGAGGCACCAAGCTGGAAATCAAG.

SEQ ID NO: 133 includes gdT scFv: 1-720; gdT VH: 355-399; gdT Linker: 355-399; gdT VL: 400-720; Free energy: −342.6; gdT CAI: 0.78445426326926; ORF count: 1.

SEQ ID NO: 134 is the sequence name for CD5 scFv (21). It is a gdT Targeting scFv construct. It is a DNA sequence. The sequence is:

GAGATCCAGCTCGTGCAGTCCGGCGGCGGCCTCGTGAAACCCGGCGGCA
GCGTGCGCATCTCGTGCGCCGCGTCCGGGTACACGTTCACGAACTACGG
CATGAACTGGGTGCGGCAGGCACCCGGCAAGGGGCTGGAGTGGATGGGC
TGGATCAACACGCACACCGGCGAGCCCACGTACGCCGACTCGTTCAAGG
GGCGCTTCACGTTCAGCCTCGACGACTCCAAGAACACCGCGTACCTGCA
GATCAACAGCCTGCGCGCCGAGGACACCGCCGTGTACTTCTGCACGCGG
CGCGGGTACGACTGGTACTTCGACGTGTGGGGCCAGGGCACGACCGTGA
CCGTGTCGTCTGGCGGCGGTGGATCAGGCGGCGGAGGCTCAGGCGGTGG
AGGCTCCGACATCCAGATGACGCAGTCGCCGTCCAGCCTGTCCGCGTCC
GTCGGCGACCGCGTGACGATCACGTGCCGCGCGTCGCAGGACATCAACT
CGTACCTGTCGTGGTTCCAGCAGAAGCCCGGCAAGGCGCCCAAGACGCT
GATCTACCGCGCGAACCGGCTGGAGTCCGGCGTGCCGTCGCGGTTCAGC
GGGTCCGGGTCCGGCACCGACTACACGCTGACGATCAGCAGCCTGCAGT
ACGAGGACTTCGGCATCTACTACTGCCAGCAGTACGACGAGTCGCCGTG
GACGTTCGGCGGCGGCACGAAGCTGGAGATCAAG.

SEQ ID NO: 134 includes gdT scFv: 1-720; gdT VH: 355-399; gdT Linker: 355-399; gdT VL: 400-720; Free energy: −338.3; gdT CAI: 0.760359380692982; ORF count: 1.

SEQ ID NO: 135 is the sequence name for CD5 scFv (29). It is a gdT Targeting scFv construct. It is a DNA sequence. The sequence is:

GAGATCCAGCTCGTGCAGTCCGGCGGCGGCCTGGTCAAGCCTGGCGGCT
CCGTGCGCATCTCCTGCGCGGCCTCGGGGTACACCTTCACGAACTACGG
CATGAACTGGGTGCGCCAGGCCCCGGGGAAAGGCCTGGAGTGGATGGGC
TGGATCAACACGCACACCGGGGAGCCCACGTACGCCGACTCCTTCAAGG
GGCGCTTCACGTTCTCCCTGGACGACTCGAAGAACACGGCCTACCTGCA
GATCAACTCCCTGCGCGCCGAGGACACCGCGGTGTACTTCTGCACGCGC
CGGGGCTACGACTGGTACTTCGACGTCTGGGGCCAGGGGACGACGGTCA
CCGTGAGCTCAGGCGGCGGAGGAAGCGGAGGCGGAGGCTCAGGCGGAGG
CGGCTCGGACATCCAGATGACGCAGAGCCCGTCCTCGCTGAGCGCCTCC
GTGGGCGACCGCGTGACGATCACCTGCCGGGCGTCCCAGGACATCAACT
CCTACCTGTCGTGGTTCCAGCAGAAGCCCGGGAAGGCCCCGAAGACCCT
GATCTACCGGGCGAACCGCCTGGAGTCCGGCGTGCCCTCGCGGTTCTCC
GGGTCGGGCTCGGGGACGGACTACACGCTGACCATCTCCTCGCTGCAGT
ACGAGGACTTCGGCATCTACTACTGCCAGCAGTACGACGAGTCCCCGTG
GACCTTCGGCGGCGGCACGAAGCTGGAGATCAAG.

SEQ ID NO: 135 includes gdT scFv: 1-720; gdT VH: 355-399; gdT Linker: 355-399; gdT VL: 400-720; Free energy: −334.2; gdT CAI: 0.774216148765874; ORF count: 1.

SEQ ID NO: 136 is the sequence name for IL2r ligand. It is a gdT Targeting ligand construct. It is an AA sequence. The sequence is:

APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFCQSIISTLT.

SEQ ID NO: 136 includes gdT ligand: 1-333.

SEQ ID NO: 137 is the sequence name for IL2r ligand. It is a gdT Targeting ligand construct. It is a DNA sequence. The sequence is:

GCACCAACAAGCAGTAGCACCAAAAAGACGCAGCTTCAGTTAGAGCACC
TCCTACTCGACCTACAGATGATATTGAATGGTATTAATAACTACAAAAA
TCCTAAATTGACTCGAATGTTGACATTTAAATTTTATATGCCCAAAAAG
GCAACCGAACTCAAGCATCTGCAGTGCCTGGAGGAGGAACTCAAGCCAC
TTGAAGAGGTCCTGAACCTGGCTCAGTCAAAAAATTTTCATCTGCGCCC
CCGGGACTTAATCAGCAATATCAACGTGATTGTTCTGGAGCTCAAGGGG
TCTGAGACCACTTTTATGTGTGAATACGCTGATGAAACTGCGACAATCG
TCGAGTTCCTCAATAGATGGATCACTTTCTGTCAATCCATTATTAGCAC
CCTGACC.

SEQ ID NO: 137 includes gdT ligand: 1-459; Free energy: −92.6; gdT CAI: 0.749692927682812; ORF count: 2.

SEQ ID NO: 138 is the sequence name for IL2r ligand. It is a gdT Targeting ligand construct. It is a DNA sequence. The sequence is:

GCTCCTACCAGCTCCAGCACCAAGAAGACCCAGCTGCAGCTGGAGCACC
TGCTGCTGGACCTGCAGATGATCCTGAACGGGATCAACAACTACAAGAA
CCCCAAGCTGACCCGGATGCTGACCTTCAAGTTCTACATGCCCAAGAAG
GCCACCGAGCTGAAGCACCTGCAGTGCCTGGAGGAGGAGCTGAAGCCCC
TGGAGGAGGTGCTGAACCTGGCCCAGAGCAAGAACTTCCACCTGCGGCC
CCGGGACCTGATCAGCAACATCAACGTGATCGTGCTGGAGCTGAAGGGG
TCCGAGACCACCTTCATGTGCGAGTACGCCGACGAGACCGCCACCATCG
TGGAGTTCCTGAACCGCTGGATCACCTTCTGCCAGAGCATCATCTCCAC
GCTGACC.

SEQ ID NO: 138 includes gdT ligand: 1-459; Free energy: −140.2; gdT CAI: 0.948946971021626; ORF count: 0.

SEQ ID NO: 139 is the sequence name for IL2r ligand. It is a gdT Targeting ligand construct. It is a DNA sequence. The sequence is:

GCCCCGACCAGCAGCAGCACCAAGAAGACGCAGCTGCAGCTGGAGCACC
TGCTGCTGGACCTGCAGATGATCCTGAACGGGATCAACAACTACAAGAA
CCCCAAGCTGACCCGGATGCTGACCTTCAAGTTCTACATGCCCAAGAAG
GCGACCGAGCTGAAGCACCTGCAGTGCCTGGAGGAGGAGCTGAAGCCCC
TGGAGGAGGTGCTGAACCTGGCCCAGTCCAAGAACTTCCACCTGCGGCC
CCGGGACCTGATCAGCAACATCAACGTGATCGTGCTGGAGCTGAAGGGG
TCCGAGACCACCTTCATGTGCGAGTACGCGGACGAGACCGCCACAATCG
TGGAGTTCCTGAACCGCTGGATCACCTTCTGCCAGTCCATCATCAGCAC
CCTGACG.

SEQ ID NO: 139 includes gdT ligand: 1-459; Free energy: −142.5; gdT CAI: 0.925399648745741; ORF count: 0.

SEQ ID NO: 140 is the sequence name for IL2r ligand. It is a gdT Targeting ligand construct. It is a DNA sequence. The sequence is:

GCCCCAACCTCCTCCTCCACCAAGAAGACCCAGCTGCAGCTGGAGCACC
TGCTGCTGGACCTGCAGATGATCCTGAACGGGATCAACAACTACAAGAA
CCCCAAGCTGACCCGGATGCTCACCTTCAAGTTCTACATGCCCAAGAAG
GCCACCGAGCTGAAGCACCTGCAGTGCCTGGAGGAGGAGCTGAAGCCCC
TGGAGGAGGTGCTGAACCTGGCCCAGTCCAAGAACTTCCACCTGCGGCC
CCGGGACCTGATCTCCAACATCAACGTGATCGTGCTGGAGCTGAAGGGG
TCCGAGACCACCTTCATGTGCGAGTACGCAGATGAGACGGCTACAATCG
TGGAGTTCCTGAACAGGTGGATCACCTTCTGCCAGTCCATCATCTCCAC
CTTGACA.

SEQ ID NO: 140 includes gdT ligand: 1-459; Free energy: −139.3; gdT CAI: 0.966990185804835; ORF count: 1.

SEQ ID NO: 141 is the sequence name for IL2r ligand. It is a gdT Targeting ligand construct. It is a DNA sequence. The sequence is:

GCGCCCACGTCCTCCTCCACCAAGAAGACCCAGCTGCAGCTGGAGCACC
TGCTGCTGGACCTGCAGATGATCCTGAACGGGATCAACAACTACAAGAA
CCCCAAGCTGACCCGGATGCTGACCTTCAAGTTCTACATGCCCAAGAAG
GCCACCGAGCTGAAGCACCTGCAGTGCCTGGAGGAGGAGCTGAAGCCCC
TGGAGGAGGTGCTGAACCTGGCCCAGTCCAAGAACTTCCACCTGCGGCC
CCGGGACCTGATCTCCAACATCAACGTGATCGTGCTGGAGCTGAAGGGG
TCCGAGACCACCTTCATGTGCGAGTACGCCGACGAGACCGCCACCATCG
TGGAGTTCCTGAACCGCTGGATCACCTTCTGCCAGAGCATCATCAGCAC
CCTCACC.

SEQ ID NO: 141 includes gdT ligand: 1-459; Free energy: −136; gdT CAI: 0.957989396711122; ORF count: 0.

SEQ ID NO: 142 is the sequence name for IL2r ligand. It is a gdT Targeting ligand construct. It is a DNA sequence. The sequence is:

GCCCCTACCTCCTCCTCCACCAAGAAGACCCAGCTCCAGCTGGAGCACC
TCCTCCTGGACCTCCAGATGATCCTCAACGGGATCAACAACTACAAGAA
CCCCAAGCTCACCCGGATGCTGACCTTCAAGTTCTACATGCCCAAGAAG
GCCACCGAGCTGAAGCACCTCCAGTGCCTGGAGGAGGAGCTGAAGCCCC
TGGAGGAGGTGCTGAACCTGGCCCAGTCCAAGAACTTCCACCTCCGGCC
CAGGGACCTGATCTCCAACATCAACGTGATCGTCCTGGAGCTGAAGGGG
TCCGAGACCACCTTCATGTGCGAGTACGCCGATGAGACAGCCACCATCG
TGGAGTTCCTCAACAGGTGGATCACCTTCTGCCAGTCCATCATCAGCAC
CCTCACC.

SEQ ID NO: 142 includes gdT ligand: 1-459; Free energy: −135.6; gdT CAI: 0.950256495713545; ORF count: 1.

SEQ ID NO: 143 is the sequence name for IL15r ligand. It is a gdT Targeting ligand construct. It is an AA sequence. The sequence is:

NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS.

SEQ ID NO: 143 includes gdT ligand: 1-114.

SEQ ID NO: 144 is the sequence name for IL15r ligand. It is a gdT Targeting ligand construct. It is a DNA sequence. The sequence is:

AACTGGGTTAACGTGATCAGCGATCTGAAGAAGATTGAAGATCTCATAC
AATCCATGCACATCGACGCTACCCTGTATACAGAGTCCGACGTTCACCC
TAGCTGTAAGGTGACTGCCATGAAGTGCTTTTTACTGGAACTGCAGGTA
ATCAGTCTGGAGTCTGGTGATGCCTCAATTCACGACACGGTAGAGAATC
TAATAATCCTTGCCAACAACTCTTTGAGTTCCAATGGCAATGTGACAGA
ATCTGGCTGCAAGGAGTGTGAAGAGCTTGAAGAGAAAAACATTAAAGAG
TTCCTGCAATCCTTCGTGCATATAGTGCAGATGTTCATCAACACCTCG.

SEQ ID NO: 144 includes gdT ligand: 1-342; Free energy: −86.8; gdT CAI: 0.773146219024413; ORF count: 3.

SEQ ID NO: 145 is the sequence name for IL15r ligand (12). It is a gdT Targeting ligand construct. It is a DNA sequence. The sequence is:

AACTGGGTGAACGTGATCAGCGATCTGAAGAAGATCGAGGACCTGATCC
AGTCCATGCACATCGACGCTACCCTGTACACCGAGTCGGACGTTCACCC
CAGCTGCAAGGTGACCGCGATGAAGTGCTTCCTGCTCGAACTGCAGGTG
ATCAGCCTGGAGAGCGGGGACGCGAGCATCCACGATACGGTGGAGAACC
TGATCATCCTGGCCAACAACTCGCTCAGCTCGAACGGGAACGTGACCGA
GAGCGGGTGCAAGGAGTGCGAGGAGCTGGAGGAGAAGAACATCAAGGAG
TTCCTCCAGTCGTTCGTGCACATCGTGCAGATGTTCATCAACACCTCC.

SEQ ID NO: 145 includes gdT ligand: 1-342; Free energy: −125.8; gdT CAI: 0.877487412777548; ORF count: 0.

SEQ ID NO: 146 is the sequence name for IL15r ligand (31). It is a gdT Targeting ligand construct. It is a DNA sequence. The sequence is:

AACTGGGTGAACGTCATCTCCGACCTGAAGAAGATCGAGGATCTGATCC
AGTCGATGCACATCGACGCGACGCTCTACACCGAGTCGGACGTTCACCC
CTCGTGCAAGGTCACGGCGATGAAGTGCTTCCTCCTGGAGCTGCAGGTG
ATCTCCCTGGAGTCGGGCGACGCCTCGATCCACGACACGGTCGAGAACC
TGATCATCCTCGCGAACAACTCCCTCTCGTCCAACGGGAACGTGACCGA
GAGCGGGTGCAAGGAGTGCGAGGAGCTGGAGGAGAAGAACATCAAGGAG
TTCCTCCAGTCGTTCGTCCACATCGTCCAGATGTTCATCAACACCTCC.

SEQ ID NO: 146 includes gdT ligand: 1-342; Free energy: −124.3; gdT CAI: 0.830363621275029; ORF count: 0.

SEQ ID NO: 147 is the sequence name for IL15r ligand (29). It is a gdT Targeting ligand construct. It is a DNA sequence. The sequence is:

AACTGGGTGAACGTGATCAGCGACCTGAAGAAGATCGAGGACCTGATCC
AGTCCATGCACATCGACGCCACGCTGTACACCGAGTCCGACGTGCACCC
CAGCTGCAAGGTGACCGCCATGAAGTGCTTCCTGCTGGAGCTGCAGGTG
ATCTCCCTGGAGTCCGGGGACGCCTCCATCCACGACACCGTGGAGAACC
TGATCATCCTGGCCAACAACTCCCTGTCCTCCAACGGGAACGTGACCGA
GTCCGGGTGCAAGGAGTGCGAGGAGCTGGAGGAGAAGAACATCAAGGAG
TTCCTGCAGAGCTTCGTGCACATCGTGCAGATGTTCATCAACACGTCG.

SEQ ID NO: 147 includes gdT ligand: 1-342; Free energy: −123.2; gdT CAI: 0.953480352366457; ORF count: 0.

SEQ ID NO: 148 is the sequence name for IL15r ligand (29). It is a gdT Targeting ligand construct. It is a DNA sequence. The sequence is:

AATTGGGTGAACGTCATCTCCGACCTCAAGAAGATCGAGGACCTCATCC
AGTCCATGCACATCGACGCCACGCTCTACACGGAGTCCGACGTGCACCC
GTCCTGCAAGGTGACGGCCATGAAGTGCTTCCTGCTGGAGCTGCAGGTC
ATCTCCTTGGAGTCCGGGGACGCCTCCATCCACGACACCGTCGAGAACC
TCATCATCCTGGCCAACAACTCCTTGAGCTCCAACGGGAACGTGACGGA
GTCCGGCTGCAAGGAGTGCGAGGAGCTGGAGGAGAAGAACATCAAGGAG
TTCCTGCAGTCCTTCGTGCACATCGTGCAGATGTTCATCAACACGTCC.

SEQ ID NO: 148 includes gdT ligand: 1-342; Free energy: −122.3; gdT CAI: 0.905077015244385; ORF count: 0.

SEQ ID NO: 149 is the sequence name for IL15r ligand (32). It is a gdT Targeting ligand construct. It is a DNA sequence. The sequence is:

AACTGGGTGAACGTGATCTCCGACCTCAAGAAGATCGAGGACCTCATCC
AGTCGATGCACATCGACGCGACCCTCTACACGGAGAGCGACGTCCATCC
GAGCTGCAAGGTGACCGCGATGAAGTGCTTCCTCCTGGAGCTCCAGGTG
ATCTCCCTGGAGTCCGGGGACGCGAGCATCCACGACACCGTCGAGAACC
TGATCATCCTCGCGAACAACTCGCTCTCCTCGAACGGGAACGTCACCGA
GAGCGGCTGCAAGGAGTGCGAGGAGCTCGAAGAGAAGAACATCAAGGAG
TTCCTCCAGAGCTTCGTCCACATCGTCCAGATGTTCATCAACACGTCG.

SEQ ID NO: 149 includes gdT ligand: 1-342; Free energy: −122.1; gdT CAI: 0.814639976789479; ORF count: 0.

SEQ ID NO: 150 is the sequence name for B2m shRNA1. It is a HLA siRNA construct. It is a DNA sequence. The sequence is: GAATGGAGAGAGAATTGAA.

SEQ ID NO: 151 is the sequence name for CIITA shRNA7. It is a HLA siRNA construct. It is a DNA sequence. The sequence is: GCTCAGGCTAAGCTTGTACAA.

CCCCTCCCTTCAGGCCCCGCGCGATTCCGCCCCCAGTTCTGTGCCGGCC
AAGATCCCGGCTAGCGCCGCTATCATTGGTTAGTTCCAAGTTTGCCCGC
CCCTCTTCCTCCTCCTTTTTCCGCCCCCTCCCTCCCGCGGAAGCTGGGG
GCGCATGCGTAGAGGTGGACGCTCCCCTCCCCCGCCCGGGGTAACTGAG
GACTCCCGCGCGCGGACTCGCTGCGCCCCACCCTCCCTTTCCCCGGGGC
CGTCCGGAGAGCGGGGGCGAGCTTGAAAGTTCCAGAACGCTGCGGTGAG
TGCGTTATCGTGAGGCGGAGCGCGGTGGGGTGGGTGCGGAAGGGGGCGA
GGCCCGAGGAGTGGAGCCGGGCTTGTGATTGGGTCTTGTAAGGGCAGCC
GGGCGTCTATTGGCCGGGGAAGCCGTAATGGCAGGCAGCAGGGGCGGGC
CCCTTCTGGAAGGTTCTAAGATAGGGTATAAGAGGCAGGGTGGCGGGCG
GAAACCGGTCTCATTGAACTCGCCTGCAGCTCTTGGGTTTTTTGTGGCT
TCCTTCGTTATTGGAGCCAGGCCTACACCCCAGGTAAAACCTCTGCTCA
AGAGTTGGGTTG.

SEQ ID NO: 152 is MND promoter. It is a DNA sequence. The sequence is:

CCCCTCCCTTCAGGCCCCGCGCGATTCCGCCCCCAGTTCTGTGCCGGCC
AAGATCCCGGCTAGCGCCGCTATCATTGGTTAGTTCCAAGTTTGCCCGC
CCCTCTTCCTCCTCCTTTTTCCGCCCCCTCCCTCCCGCGGAAGCTGGGG
GCGCATGCGTAGAGGTGGACGCTCCCCTCCCCCGCCCGGGGTAACTGAG
GACTCCCGCGCGCGGACTCGCTGCGCCCCACCCTCCCTTTCCCCGGGGC
CGTCCGGAGAGCGGGGGCGAGCTTGAAAGTTCCAGAACGCTGCGGTGAG
TGCGTTATCGTGAGGCGGAGCGCGGTGGGGTGGGTGCGGAAGGGGGCGA
GGCCCGAGGAGTGGAGCCGGGCTTGTGATTGGGTCTTGTAAGGGCAGCC
GGGCGTCTATTGGCCGGGGAAGCCGTAATGGCAGGCAGCAGGGGCGGGC
CCCTTCTGGAAGGTTCTAAGATAGGGTATAAGAGGCAGGGTGGCGGGCG
GAAACCGGTCTCATTGAACTCGCCTGCAGCTCTTGGGTTTTTTGTGGCT
TCCTTCGTTATTGGAGCCAGGCCTACACCCCAGGTAAAACCTCTGCTCA
AGAGTTGGGTTG

SEQ ID NO: 154 is WPREmut. It is a DNA sequence. The sequence is:

ATCAACCTCTGGATTACAAAATTTGTGAAAGATTGACTGATATTCTTAA
CTATGTTGCTCCTTTTACGCTGTGTGGATATGCTGCTTTAATGCCTCTG
TATCATGCTATTGCTTCCCGTACGGCTTTCGTTTTCTCCTCCTTGTATA
AATCCTGGTTGCTGTCTCTTTATGAGGAGTTGTGGCCCGTTGTCCGTCA
ACGTGGCGTGGTGTGCTCTGTGTTTGCTGACGCAACCCCCACTGGCTGG
GGCATTGCCACCACCTGTCAACTCCTTTCTGGGACTTTCGCTTTCCCCC
TCCCGATCGCCACGGCAGAACTCATCGCCGCCTGCCTTGCCCGCTGCTG
GACAGGGGCTAGGTTGCTGGGCACTGATAATTCCGTGGTGTTGTCGGGG
AAATCATCGTCCTTTCCTTGGCTGCTCGCCTGTGTTGCCAACTGGATCC
TGCGCGGGACGTCCTTCTGCTACGTCCCTTCGGCTCTCAATCCAGCGGA
CCTCCCTTCCCGAGGCCTTCTGCCGGTTCTGCGGCCTCTCCCGCGTCTT
CGCTTTCGGCCTCCGACGAGTCGGATCTCCCTTTGGGCCGCCTCCCCGC
CTG

SEQ ID NO: 155 is 4Gs. It is an AA sequence. The sequence is: GGGGS.

In a variation, the present disclosure provides a composition comprising an isolated polynucleotide disclosed herein as any one of SEQ ID NOS: 1 through 155 or encoding an amino acid disclosed herein as any one of SEQ ID NOS: 1 through 155. We also disclose a polynucleotide comprising the nucleotide sequence of the full-length protein of the amino acid sequences disclosed as any one of SED ID NOS: 1 through 155. We also disclose a polynucleotide encoding a protein comprising a fragment of the amino acid sequence of any on of SEQ ID NOS:1 through 155 having biological activity, the fragment comprising eight contiguous amino acids of any one of SEQ ID NOS: 1 through 155; a polynucleotide which is an allelic variant of a polynucleotide of any of SEQ ID NOS: 1-155 or those disclosed above; a polynucleotide which encodes a species-homologue of the protein encoded by or disclosed as any one of SEQ ID NOS: 1 through 155 or any of those disclosed above; a polynucleotide that hybridizes under stringent conditions to any one of the polynucleotides specified in or encoded by SEQ ID NOS: 1 through 155 or any of those disclosed herein; and a polynucleotide that hybridizes under stringent conditions to any one of the polynucleotides specified or encoded by SEQ ID NOS: 1 through 155 and that has a length that is at least 25% of the length of the sequence encoded or encoded by SEQ ID NOS: 1 through 155.

Other systems, methods, features, and advantages of the disclosure will be, or will become, apparent to one with skill in the art upon examination of the following figures and detailed description. It is intended that all such additional systems, methods, features and advantages be included within this description, be within the scope of the invention, and be protected by the following claims.

The invention is not limited to the variations illustrated and described, as it also covers all equivalent implementations insofar as they do not depart form the spirit of the invention. Further, the invention is not yet limited to the combination of features as described herein but may be defined by any other combination of all of the individual features disclosed. Further, the invention is not yet limited to the sequence of method steps as described herein but may be defined by any other combination or order the steps disclosed. Any person skilled in the art of will recognize from the previous detailed description and from the figures and claims that modifications could be made to the disclosed embodiments of the invention without departing from the scope of the invention.

1. A therapeutic protein capable of biosynthesis and secretion by a gamma delta T cell, the therapeutic protein comprising, from N-terminus to C-terminus, a gamma delta T cell optimized signal peptide that is cleaved off prior to secretion, a tumor cell-binding protein domain, a linker, and a T cell binding protein.

2. The therapeutic protein of claim 1, where the signal peptide is selected from the group consisting of mSA, IL2, and hSCF.

3. The therapeutic protein of claim 1, where the tumor cell binding domain is a single chain antibody variable domain fragment or a tumor cell receptor ligand that binds one selected from the group consisting of SSTR2, PTK7, GD2, SSTR5, CD19, aVB3, CD110, and CD5.

4. The therapeutic protein of claim 1, where the tumor cell binding domain is selected from the group consisting of SSTR2 LH scFv, PTK7 HL scFv, SSTR2 HL scFv, CD19 LH scFv, GD2 HL scFv, GD2 LH scFv, Integrin aVB3 HL ScFv, 2×SST28 3×G4S, 2×SST28, 4×G2s, TPO (ligand), and SCF (ligand).

5. The therapeutic protein of claim 1, where the linker is selected from the group consisting of G4S, G4S-albumin-G4S and G4S-Fc-G4S.

6. The therapeutic protein of claim 1, where the gamma delta T cell binding domain is selected from the group consisting of (a) a single chain antibody variable domain fragment domain selected from a consisting of T cell receptor gamma chain binding, T cell receptor delta chain binding, CD3delta, CD3 epsilon, CDXAR, and JAML and (b) a binding domain is selected from the group consisting of gd-c (V1) HL scFv, gd-c (V6) HL scFv, Hum2 scFv, CD3 scFv, CDXAR ligand, and JAML scFv.

7. An engineered gamma delta T cell capable of secreting at least one therapeutic protein that has been expression cassette optimized, the therapeutic protein comprising, from N-terminus to C-terminus, a gamma delta T cell optimized signal peptide, tumor cell binding domain, linker, and T cell binding domain.

8. The engineered gamma delta T cell of claim 7, where the signal peptide is selected from the group consisting of mSA, IL2, and hSCF.

9. The engineered gamma delta T cell of claim 7, where the tumor cell binding domain is selected from a group consisting of (a) a single chain antibody variable domain fragment, (b) a tumor cell receptor ligand that binds one from the group consisting of SSTR2, PTK7, GD2, SSTR5, CD19, aVB3, CD110, and CD5, and (c) one selected from the group consisting of SSTR2 LH scFv, PTK7 HL scFv, SSTR2 HL scFv, CD19 LH scFv, GD2 HL scFv, GD2 LH scFv, Integrin aVB3 HL ScFv, 2×SST28 3×G4S, 2×SST28, 4×G2s, TPO, and SCF.

10. The engineered gamma delta T cell of claim 7, where the linker is selected from the group consisting of G4S, G4S-albumin-G4S and G4S-Fc-G4S.

11. The engineered gamma delta T cell of claim 7, where the gamma delta T cell binding domain is a single chain antibody variable domain fragment domain selected from a group consisting of (a) a binding domain that binds one from the group consisting of T cell receptor gamma chain binding, T cell receptor delta chain binding, CD3delta, CD3 epsilon, CDXAR, and JAML and (b) where the gamma delta T cell binding protein is selected from the group consisting of of gd-c (V1) HL scFv, gd-c (V6) HL scFv, Hum2 scFv, CD3 scFv, CDXAR ligand, and JAML scFv.

12. The engineered gamma delta T cell of claim 7, where the gamma delta T cell surface binding domain comprises an anti-CD3 scFv and the tumor cell surface binding domain is selected from the group consisting of a single chain antibody variable domain fragment binding one selected from the group consisting of SSTR2, PTK7, GD2, SSTR5, CD19, aVB3, CD110, and CD5.

13. The engineered gamma delta T cell of claim 7, where the gamma delta T cell surface binding domain comprises an anti-CD3 scFv and the tumor cell surface binding domain is selected from the group consisting of TPO, SCF and somatostatin ligands.

14. A recombinant viral vector encoding encoding a therapeutic protein capable of biosynthesis and secretion by a gamma delta T cell, the therapeutic protein comprising, from N-terminus to C-terminus, a gamma delta T cell optimized signal peptide that is cleaved off prior to secretion, a tumor cell-binding protein domain, a linker, and a T cell binding protein.

15. The recombinant viral vector of claim 14, where the signal peptide is selected from the group consisting of mSA, IL2, and hSCF.

16. The recombinant viral vector of claim 14, where the tumor cell binding domain is a single chain antibody variable domain fragment or a tumor cell receptor ligand that binds one from the group consisting of (a) one selected from the group consisting of SSTR2, PTK7, GD2, SSTR5, CD19, aVB3, CD110, and CD5 and (b) one selected from the group consisting of SSTR2 LH scFv, PTK7 HL scFv, SSTR2 HL scFv, CD19 LH scFv, GD2 HL scFv, GD2 LH scFv, Integrin aVB3 HL ScFv, 2×SST28 3×G4S, 2×SST28, 4×G2s, TPO (ligand), and SCF (ligand).

17. The recombinant viral vector of claim 14, where the linker is selected from the group consisting of G4S, G4S-albumin-G4S and G4S-Fc-G4S.

18. The recombinant viral vector of claim 14, where the gamma delta T cell binding domain is selected from the group consisting of (a) a single chain antibody variable domain fragment domain selected from a consisting of T cell receptor gamma chain binding, T cell receptor delta chain binding, CD3delta, CD3 epsilon, CDXAR, and JAML and (b) a binding domain is selected from the group consisting of gd-c (V1) HL scFv, gd-c (V6) HL scFv, Hum2 scFv, CD3 scFv, CDXAR ligand, and JAML scFv.

19. The recombinant viral vector of claim 14 that contains a transcriptional promoter active in gamma delta T cells selected from group consisting of HSP8, MND, SFFV, EF1alpha, and UBC promoters.

20. The recombinant viral vector of claim 14 that additionally encodes one or more shRNA sequences targeting at least one from the group consisting of beta-2-microglobulin and CIITA.

21. A recombinant synthetic mRNA encoding encoding a therapeutic protein capable of biosynthesis and secretion by a gamma delta T cell, the therapeutic protein comprising, from N-terminus to C-terminus, a gamma delta T cell optimized signal peptide that is cleaved off prior to secretion, a tumor cell-binding protein domain, a linker, and a T cell binding protein.

22. The recombinant synthetic mRNA of claim 21, where the signal peptide is selected from the group consisting of mSA, IL2, and hSCF.

23. The recombinant synthetic mRNA of claim 21, where the tumor cell binding domain is a single chain antibody variable domain fragment or a tumor cell receptor ligand that binds one from the group consisting of (a) one selected from the group consisting of SSTR2, PTK7, GD2, SSTR5, CD19, aVB3, CD110, and CD5 and (b) one selected from the group consisting of SSTR2 LH scFv, PTK7 HL scFv, SSTR2 HL scFv, CD19 LH scFv, GD2 HL scFv, GD2 LH scFv, Integrin aVB3 HL ScFv, 2×SST28 3×G4S, 2×SST28, 4×G2s, TPO (ligand), and SCF (ligand).

24. The recombinant synthetic mRNA of claim 21, where the linker is selected from the group consisting of G4S, G4S-albumin-G4S and G4S-Fc-G4S.

25. The recombinant viral vector or synthetic mRNA of claim 21, where the gamma delta T cell binding domain is selected from the group consisting of (a) a single chain antibody variable domain fragment domain selected from a consisting of T cell receptor gamma chain binding, T cell receptor delta chain binding, CD3delta, CD3 epsilon, CDXAR, and JAML and (b) a binding domain is selected from the group consisting of gd-c (V1) HL scFv, gd-c (V6) HL scFv, Hum2 scFv, CD3 scFv, CDXAR ligand, and JAML scFv.

26. The recombinant viral vector or synthetic mRNA of claim 21 that contains a transcriptional promoter active in gamma delta T cells selected from group consisting of HSP8, MND, SFFV, EF1alpha, and UBC promoters.

27. The recombinant viral vector or synthetic mRNA of claim 21 that additionally encodes one or more shRNA sequences targeting at least one from the group consisting of beta-2-microglobulin and CIITA.


SEQUENCE LISTING (155 sequences)

SEQ ID NO: 1 - protein, 518 aa, synthetic construct
MYRMQLLSCIALSLALVTNSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - DNA, 1554 bp, synthetic construct
  source 1..1554: mol_type = other DNA
atgtacaggatgcagctgctgtcctgcattgccctgtccctggccctggtgaccaactccgacattcaattgacacaaagcccggcaagcctggctgtgtccctgggccagcgggcgactatttcatgtaaagcaagtcagagcgtagactacgatggtgatagctatctgaactggtatcagcagattccaggacaacctcccaaattgctgatttacgacgcctcgaacctggtcagcgggattccaccacgattctctggaagcggcagtggaaccgattttacgctgaatatacacccagtggaaaaggtggacgctgcgacttatcattgtcagcagtctaccgaggacccatggaccttcggcgggggaacaaagctggagatcaagggtggtggtggaagcggtggtggtggatctggcggtggtggaagtcaggtgcagcttcagcagtccggagccgaactggtgcgccccggcagtagcgtgaagataagctgcaaggctagcggatacgctttctcttcctactggatgaactgggtgaagcagcgccctggccaagggcttgagtggatcggccagatatggcctggtgatggtgacaccaactataatggcaagtttaagggaaaggctacactcactgccgatgaaagttcatccactgcctacatgcagctctccagtctggcaagtgaagactctgctgtctatttctgcgctagacgagagactacaactgtcgggaggtactactatgcaatggattactggggtcagggaaccacagtgacagtgagctcaggaggtggtgggtctgacattaagctacaacagtctggcgccgagttggccaggcctggggccagcgtgaagatgtcttgcaagacctcaggttataccttcacgagatacaccatgcattgggtaaaacagagacctgggcaaggcttggaatggatcggctacatcaaccccagtcgcggatacacaaactacaatcagaaatttaaagacaaggcgactctcaccacagataagtcctcctctaccgcctacatgcagctgtcatctctcacaagcgaagactctgccgtgtattattgcgcaaggtattacgatgaccactattgtttagattattgggggcaaggaactacactcactgtcagctcagttgagggtggaagtgggggatctggtggttcaggcggatctggtggggtcgacgacatccagctgacccagagtcccgccatcatgtcagctagtcccggggagaaagtgactatgacatgcagggcatctagcagcgtttcgtacatgaattggtatcagcagaaatcaggtacctccccgaaacgttggatttatgacacgagcaaggttgcaagcggtgttccataccggttttcgggctccggttccggcacctcctactctctcacgatctccagtatggaagccgaggatgccgcaacctattactgtcagcaatggtcatccaatcccttaaccttcggagccggaacaaaactggagctcaaa

SEQ ID NO: 3 - protein, 19 aa, synthetic construct
MKWVTFISLLFLFSSSSRA

SEQ ID NO: 4 - protein, 25 aa, Homo sapiens
MKKTQTWILTCIYLQLLLFNPLVKT

SEQ ID NO: 5 - DNA, 57 bp, synthetic construct
  source 1..57: mol_type = other DNA
atgaaatgggttacttttattagtttattattcctgttcagctccagctccagggcc

SEQ ID NO: 6 - DNA, 75 bp, Homo sapiens
  source 1..75: mol_type = unassigned DNA
atgaagaaaactcaaacttggatactaacttgcatctacctgcagctgctgctcttcaaccccttggtgaagacg

SEQ ID NO: 7 - DNA, 750 bp, Synthetic construct
  source 1..750: mol_type = other DNA
gacatccagctcacccagagccccgcatccctggccgtgtccctggggcagcgcgcaaccatctcctgcaaggcttcccagtccgtggactacgacggggactcctacctgaactggtaccagcagatccccgggcagccaccgaagctgctgatctacgacgcctccaacctggtgtccgggattcctccgcggttctccgggagcgggtccgggaccgacttcaccctgaacatccatcccgtggagaaggtggacgccgccacctaccactgccagcagtccaccgaggacccctggaccttcggcggcggcaccaagctggagatcaaaggtggcggaggctccggtggcggaggttccggtggcggcggctcccaggtgcagctgcagcagtcaggggccgagctggtgaggcccgggagctccgtgaagatctcctgcaaggcctccgggtacgccttctcctcctactggatgaactgggtgaagcagaggcccgggcaggggctggagtggatcgggcagatctggcccggggacggggacaccaactacaacgggaagttcaaggggaaggctaccctcaccgccgacgagagctcctccaccgcctacatgcagctgagctccctcgcctccgaggactccgccgtgtacttctgcgcccggcgcgagaccaccaccgtggggcgctactactacgccatggactactggggccaggggaccaccgtgaccgtgagctcc

SEQ ID NO: 8 - DNA, 750 bp, synthetic construct
  source 1..750: mol_type = other DNA
gacatccagctgacccagtcgcccgctagcctggcggtctcgctgggccagcgagccaccatcagctgcaaggccagccagtcggtcgactacgacggcgacagctacctcaactggtaccagcagatccccggccagccaccgaagctgctgatctacgacgccagcaacctggtgagcgggataccaccgcggttctcggggtcggggtcggggaccgacttcaccctcaacatccatcccgtcgagaaggtcgacgccgccacctaccactgccagcagtcgaccgaggacccctggaccttcggcggcggcaccaagctggagatcaaaggtggcggtggctccggtggcggtggtagcggtggcggcggctcccaggtccagctccagcagtctggggccgagctggtgcgacccgggagctcggtcaagatcagctgcaaggccagcggctacgccttctccagctactggatgaactgggtcaagcagcgacccggccaggggctggagtggatcggccagatctggcccggcgacggcgacaccaactacaacggcaagttcaagggcaaggccacgctgaccgccgacgagtcgagctcgaccgcctacatgcagctgagctcgctcgccagcgaggactccgcggtctacttctgcgcccggcgcgagaccaccaccgtggggcgctactactacgcgatggactactggggccaggggaccacggtgaccgtgagctcg

SEQ ID NO: 9 - DNA, 750 bp, synthetic construct
  source 1..750: mol_type = other DNA
gacatccagctgacgcagtcgcccgcgtcgctcgccgtgtcgctcgggcaacgcgcgacgatctcgtgcaaggcgtcgcagtccgtcgactacgacggcgactcgtacctgaactggtaccagcagatccccgggcagccgccgaagctgctgatctacgacgcgagcaacctcgtgtccggcattccgccgcggttcagcggcagcggcagcggcaccgacttcacgctgaacattcaccccgtcgagaaggtcgacgccgcgacgtaccactgccagcagtccaccgaggacccgtggacgttcggcggcggcacgaagctggagatcaaaggtggcggcggttccggtggcggtggttccggtggcggcggctcgcaggtgcagctgcagcagtctggcgccgagctcgtgcgacccgggtcgtccgtgaagatctcgtgcaaggcgtccgggtacgcattctcgtcgtactggatgaactgggtgaagcagcgacccgggcaggggctggagtggatcgggcagatctggcccggcgacggcgacacgaactacaacggcaagttcaagggcaaggcgacgctgaccgccgacgagtcgtcgtccaccgcgtacatgcagctgtcgtcgctcgcgagcgaggacagcgccgtgtacttctgcgcgcggcgcgagacgacgaccgtcgggcgctactactacgcgatggactactggggccagggcacgaccgtgaccgtgtccagc

SEQ ID NO: 10 - DNA, 750 bp, synthetic construct
  source 1..750: mol_type = other DNA
gacatccagctgacccagagccccgcgagcctggccgtgagcctggggcagagggccaccatcagctgcaaggcgtcccagagcgtggactacgacggggacagctacctgaactggtaccagcagatccccgggcagccgccgaagctgctgatctacgacgcctccaacctggtgtccgggataccgccgcggttctccgggtccgggtccgggaccgacttcaccctgaacattcaccccgtggagaaggtggacgccgccacctaccactgccagcagagcaccgaggacccctggaccttcggcggcggcaccaagctggagatcaaaggtggcggaggctccggtggcggtggtagcggtggcggcggctcccaggtgcagctgcagcagtctggggccgagctggtgcgacccgggtccagcgtgaagatcagctgcaaggccagcgggtacgccttcagctcctactggatgaactgggtgaagcagaggcccgggcaggggctggagtggatcgggcagatctggcccggggacggggacaccaactacaacgggaagttcaaggggaaggccacgctgaccgccgacgagagcagctccaccgcctacatgcagctgtccagcctggcctccgaggacagcgccgtgtacttctgcgcccggcgcgagaccaccaccgtggggcgctactactacgccatggactactggggccaggggaccaccgtgaccgtgagctcc

SEQ ID NO: 11 - DNA, 750 bp, synthetic construct
  source 1..750: mol_type = other DNA
gacatccagctgacccagtcacccgctagcctggccgtgtccctgggccagcgagccacgatctcctgcaaggccagccagtccgtggactacgacggggactcctacctcaactggtaccagcagatccccggccagccaccgaagctgctgatctacgacgcctccaacctggtgagcgggattccgccgcggttcagcgggtccgggtccgggaccgacttcaccctcaacatccatcccgtggagaaggtggacgccgccacctaccactgccagcagtccaccgaggacccctggaccttcggcggcggcaccaagctggagatcaaaggcggtggtggctccggaggcggtggctctggtggcggcggctcccaggtgcagctccagcagagcggggctgagctggtgaggcccgggtcctccgtgaagatctcctgcaaggcctccgggtacgccttctccagctactggatgaactgggtgaagcagaggcccgggcaggggctggagtggatcgggcagatctggcccggggacggggacaccaactacaacgggaagttcaaggggaaggccacgctgaccgccgacgagagcagctccaccgcctacatgcagctgtccagcctggcctccgaggactccgccgtgtacttctgcgcccggcgcgagaccaccaccgtggggcgctactactacgccatggactactggggccaggggaccaccgtgaccgtgtccagc

SEQ ID NO: 12 - DNA, 729 bp, synthetic construct
  source 1..729: mol_type = other DNA
gatatcaagctccagcagtccggggctgagctggctaggcccggggcctccgtgaagatgtcctgcaagacctccgggtacaccttcaccaggtacaccatgcactgggtgaagcagaggcccgggcaggggctggagtggatcgggtacatcaaccccagccgcgggtacaccaactacaaccagaagttcaaggacaaggccaccttgaccaccgacaagtcctcctccaccgcctacatgcagctgagctccctgacctccgaggactccgccgtctactactgcgccaggtactacgacgaccactactgcctggactactggggccaggggactaccctgaccgtgagctccgtggaaggtggctccggcggctccggaggttccggaggctccggcggcgtggacgacatccagctgacccagtccccagctatcatgtccgccagccccggggagaaggtgaccatgacctgccgggcctcctcctccgtgagctacatgaactggtaccagcagaagtccgggacctctcccaagaggtggatctacgacacctccaaggtggcctccggggtcccctacaggttctccgggtccgggtccgggacctcctactccctgaccatctcctccatggaggccgaggacgccgccacctactactgccagcagtggagctccaatcccctgaccttcggggccgggaccaagctggagctgaag

SEQ ID NO: 13 - DNA, 729 bp, unidentified
  source 1..729: mol_type = other DNA
gacatcaagctccagcagtccggggctgagcttgctcgccccggggccagcgtgaagatgtcctgcaagacctcggggtacaccttcaccaggtacaccatgcactgggtgaagcagcgcccagggcagggcctggagtggatagggtacatcaaccccagccgcgggtacacaaactacaaccagaagttcaaggacaaggccacgctgaccacggacaagtcctcgtccacggcgtacatgcagctgtcctcgctgacctccgaggacagcgcggtgtactactgcgcgcggtactacgacgaccactactgcctggactactggggccaggggacaacgctgaccgtgagcagcgtggaaggcggcagcggcggcagcggaggtagcggaggctccggcggcgtggacgacatccagctcacccagagcccggcgatcatgtccgccagccccggggagaaggtgaccatgacctgccgggcgtcgtcctcggtgagctacatgaactggtaccagcagaagtccgggacctcgcccaagcgctggatctacgacaccagcaaggtggcctccggggtgccctaccgcttctccgggtcggggtcggggacatcgtactccctgaccatctccagcatggaggccgaggacgccgcgacgtactactgccagcagtggtcgtccaacccgctgaccttcggcgcggggacaaagctggagcttaag

SEQ ID NO: 14 - DNA, 729 bp, synthetic construct
  source 1..729: mol_type = other DNA
gacatcaagctgcagcagagcggcgctgagctcgcgcgaccaggcgcgagcgtgaagatgagctgcaagacgagcggctacacgttcacgcgctacacgatgcactgggtgaagcagcgtcccgggcaggggctggagtggatcggctacatcaacccgtcgcgcggctacacgaactacaaccagaagttcaaggacaaggcgacgctgacgaccgacaagagcagcagcaccgcgtacatgcagctgagctcgctgacgagcgaggacagcgccgtgtactactgcgcgcgctactacgacgaccactactgcctcgactactggggccagggcacgacgctgaccgtgagcagcgtcgaaggcggcagcggcggcagcggtggaagcggtggcagcggcggcgtcgacgacatccagctgacgcagtcgcccgcgatcatgagcgcgtcgcccggcgagaaggtgacgatgacgtgccgcgcgagcagcagcgtgtcgtacatgaactggtaccagcagaagagcggcacgtcgccgaagcgctggatctacgacacgtcgaaggtcgcgagcggcgtgccgtaccgcttcagcggcagcggcagcggcacgtcgtactcgctgacgatcagcagcatggaggccgaggacgccgcgacgtactactgccagcagtggagctcgaacccgctgacgttcggcgccggcacgaagctggagctgaag

SEQ ID NO: 15 - DNA, 729 bp, synthetic construct
  source 1..729: mol_type = other DNA
gacatcaagctgcagcagagcggcgcggaattagcgcgccccggggcgtccgtcaagatgtcctgcaagacctccgggtacaccttcacgcggtacaccatgcactgggtgaagcaacgccccgggcagggcctggagtggatcgggtacatcaacccgtcgcggggctacacgaactacaaccagaagttcaaggacaaggcgaccctcacgaccgacaagtcgagcagcacggcgtacatgcagctctcctcgctgaccagcgaggactccgcggtgtactactgcgcgcggtactacgacgaccactactgcctggactactggggccagggcaccacgctgaccgtcagctcggtggaaggcggctccggcggctccggaggttccggaggttccggcggagtggacgacatccagctcacccagagccccgcgattatgagcgcctcgcccggcgagaaggtcaccatgacctgccgggcgagcagctcggtgagctacatgaactggtaccagcagaagagcggcacgtcgcccaagcggtggatctacgataccagcaaggtggcgagcggcgtgccgtaccgcttctcggggagcgggtccggcacgtcgtacagcctgacgatcagcagcatggaggcggaggacgccgcgacgtactactgccagcagtggagcagcaacccgctgaccttcggggccggcacgaagctcgaactgaag

SEQ ID NO: 16 - DNA, 729 bp, synthetic construct
  source 1..729: mol_type = other DNA
gacatcaagctgcagcagtccggagccgagttggcacggcccggggcctccgtgaagatgtcctgcaagacgtccgggtacacgttcacccggtacaccatgcactgggtgaagcagcggcccgggcaggggctggaatggatcgggtacatcaaccccagccgcgggtacaccaactacaaccagaagttcaaggacaaggccacgctgaccaccgacaagtcgtcgtcgaccgcctacatgcagctgagctcgctgaccagcgaggacagcgccgtctactactgcgcccgctactacgacgaccactactgcctggactactggggccaggggaccacgctgacagtgagcagcgtggaaggcggctccggcggctccggaggttccggaggcagcggcggcgtcgacgacatccagctcacccagtccccggccatcatgtcggcgagccccggcgagaaggtgaccatgacgtgccgggcctccagctcggtgtcctacatgaactggtaccagcagaagtcggggaccagccccaagcggtggatctacgacaccagcaaggtggccagcggggtcccctaccgcttctcggggtccgggtccgggacctcgtactcgctgaccatctccagcatggaggccgaggacgccgccacctactactgccagcagtggtcctcgaacccgctcaccttcggggccggcaccaagctggagctgaag

SEQ ID NO: 17 - protein, 236 aa, synthetic construct
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 18 - DNA, 708 bp, synthetic construct
  source 1..708: mol_type = other DNA
gaggtgcagctggtgcagtccggaggaggtttagttcacccaggaggctcgctgcgactgtcctgtgcagggagcggctttaccttcagcacatatctgatgtactgggtgagacaggcccccggcaagacgctggagtgggtctcagctatcggatcaggtggcgacacctattacgctgatagcgtgaagggccggtttaccataagccgcgacaacgccaaaaatagtctttacctgcagatgaacagcctccgagcagaggatatggccgtctattactgcgctcggggactcgggtattgggggcagggcaccttggtgactgtctcttcagggggaggtggttcaggtggtggaggttctggcggcggcggttccgaaattgtactaacccaatctcctggcacacttagtctgtctcctggagaacgtgcaacgttgagctgccgcgctagccagtccgtgtcttcctcgtacctcgcctggtaccagcaaaagccagggcaagctcccaggttgctgatttacggagcctcaagtagggcgactggaatccctgacagattcagtgggagcgggtccgggactgattttactctcaccatttctagacttgagcccgaagacttcgccgtttattactgtcagcagtatggctcttccccgatgtacacattcggccagggcacaaaactggaaatcaag

SEQ ID NO: 19 - DNA, 708 bp, synthetic construct
  source 1..708: mol_type = other DNA
gaggtgcagctcgtgcagtccggcggaggcctcgtgcacccaggcggctccctgcgcctgtcctgcgccgggtcaggcttcaccttctccacgtacctcatgtactgggtgcggcaggccccagggaagaccctggagtgggtgtccgccatcgggtccggcggcgacacgtactacgccgactccgtgaaggggcgcttcaccatctcccgggacaacgccaagaactccctgtacctgcagatgaactccctgcgggccgaggacatggccgtgtactactgcgccaggggcctggggtactggggccaggggaccctcgtgaccgtgtcctccggcggcggaggctcaggaggaggaggctcaggaggcggcggcagcgagatcgtcctgacgcagagccccgggaccctgtccctgagccccggggagagggcgaccctgtcctgccgggccagccagtccgtgtcctcctcctacctggcctggtaccagcagaagcccgggcaagccccacgcctcctcatctacggggcttcctccagggccacgggcatccccgaccgcttctccgggtccgggtccgggacggacttcaccctgaccatctcccggctggagcccgaggacttcgccgtgtactactgccagcagtacgggtcctcgcccatgtacaccttcgggcaggggacgaagctggagatcaag

SEQ ID NO: 20 - DNA, 708 bp, synthetic construct
  source 1..708: mol_type = other DNA
gaggtccagctggtgcagtccggcggtggcctggtgcacccaggcggcagcctgcggctgtcgtgcgccggcagtgggttcaccttctccacctacctgatgtactgggtgcgccaggccccagggaagaccctggagtgggtgtccgccatcggctccggcggcgacacctactacgccgatagcgtcaaggggcgcttcaccatctcccgcgacaacgccaagaactccctctacctccagatgaactccctgcgggccgaggacatggccgtctactactgcgccaggggcctgggctactggggccaggggaccctggtgaccgtgtcctccggcggcggtggctctggtggtggtggctctggtggcggcggcagcgagatcgtgctgacccagtcgcccgggaccctgtcgctgtcgcccggcgagcgagcgaccctcagctgccgcgcgagccagagcgtgagctccagctacctcgcgtggtaccagcagaagcccgggcaagccccacggctgctgatctacggcgctagctccagggccaccggcatccccgatcgcttcagcggcagcggcagcgggaccgacttcaccctgacgatctcccggctggagcccgaggacttcgccgtctactactgccagcagtacggctccagccccatgtacaccttcggccaggggaccaagctggagatcaag

SEQ ID NO: 21 - DNA, 708 bp, synthetic construct
  source 1..708: mol_type = other DNA
gaggtgcagctggtgcagagcggcggaggcctggtgcacccaggcggcagcctgcggctgagctgcgctggctccggcttcaccttcagcacctacctgatgtactgggtgcggcaggccccaggcaagaccctggagtgggtgtccgccatcggcagcggcggcgacacctactacgccgacagcgtgaaggggcgcttcaccatcagccgggacaacgccaagaacagcctgtacctgcagatgaacagcctgcgggccgaggacatggccgtgtactactgcgccaggggcctgggctactggggccagggcaccctggtgaccgtgagcagcggcggcggtggctctggtggaggaggctctggtggcggcggctccgagatcgtgctgacccagagccccggcaccctgagcctgagccccggggagcgggctaccctgagctgccgggcttcccagagcgtgagcagctcctacctggcctggtaccagcagaagcccgggcaggcaccacggctgctgatctacggggctagcagccgggctaccggcatccccgaccgcttctccggcagcggcagcggcaccgacttcaccctgaccatctccaggctggagcccgaggacttcgccgtgtactactgccagcagtacggcagctctcccatgtacaccttcgggcagggcaccaagctggagatcaag

SEQ ID NO: 22 - DNA, 708 bp, synthetic construct
  source 1..708: mol_type = other DNA
gaggtccagctggtccagtccggcggcggcctggttcatcccggcggcagcctgcggctgagctgcgccgggtctggcttcaccttcagcacctacctgatgtactgggtccggcaggcgcccggcaagaccctggagtgggtgtcggccatcgggtccggcggcgacacctactacgccgactcggtcaagggccggttcaccatcagccgggacaacgccaagaacagcctgtacctgcagatgaacagcctgcgggccgaggacatggccgtctactactgcgcccgcgggctgggttactggggccaggggaccctggtgaccgtcagctccggcggcggcggatctggtggtggcggatctggtggcggcggctccgagatcgtgctgacccagtcgcccgggaccctgagcctgagccccggcgagcgggctaccctgagctgccgggccagccagtcggtcagctccagctacctggcctggtaccagcagaaacccggccaggctccccggctgctgatctacggggctagtagccgggccaccggcatccccgatcggttcagcgggtccgggtccgggaccgacttcaccctgaccatcagccggctggagcccgaggacttcgccgtctactactgccagcagtacgggtccagcccgatgtacaccttcggccaggggaccaagctggagatcaag

SEQ ID NO: 23 - DNA, 708 bp, synthetic construct
  source 1..708: mol_type = other DNA
gaggtgcagctggtgcagagcggcggaggcctggtgcacccaggcggctccctgcggctcagctgcgccggttccgggttcaccttttccacgtatctgatgtactgggtgcgccaggccccggggaagaccctggaatgggtctccgccatcggctccggcggcgacacctactacgccgactccgtgaaggggcgcttcaccatttcccgggacaacgcgaagaattccctgtacctgcagatgaacagtctccgcgccgaggacatggccgtgtactactgtgcccggggcctcggatactggggccaggggaccctggtcaccgtgtccagcggcggcggaggctcaggaggaggaggctcaggaggcggcggctccgagatcgtgttgacccagagccccgggaccctgtccctgagccccggggaacgggccacgctgagctgccgcgcctcgcagagcgtctcctcctcgtacctggcctggtaccagcagaagccggggcaggctcctcggctcctcatctacggggcctcttcccgggccacgggtatccccgacaggttcagcggctccgggtccgggaccgatttcaccttgaccattagccgcctggaacccgaggacttcgccgtgtactattgtcagcagtacgggtcctcgcccatgtacacctttggccaggggaccaagctggagatcaag

SEQ ID NO: 24 - protein, 142 aa, Homo sapiens
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSS

SEQ ID NO: 25 - DNA, 426 bp, synthetic construct
  source 1..426: mol_type = other DNA
gaggggatctgcaggaacagggtgaccaacaatgtgaaggatgtgaccaagctggtggccaacctgcccaaggactacatgatcaccctgaagtatgtgccagggatggatgtgctgcccagccactgctggatctctgagatggtggtgcagctgtctgactccctgacagacctgctggacaagttctccaacatctcagaggggctgtccaactactccatcattgacaagctggtgaacatagtggatgacctggtggagtgtgtgaaggagaactcctccaaggacctgaagaagtccttcaagtcccctgagcccaggctgttcacccctgaggagttcttcaggatcttcaacaggtccattgatgccttcaaggactttgtggtggcctctgagacctctgactgtgtggtgtcctca

SEQ ID NO: 26 - DNA, 426 bp, synthetic construct
  source 1..426: mol_type = other DNA
gaggggatctgccggaaccgggtgaccaacaacgtgaaggacgtgaccaagctggtggccaacctgcccaaggactacatgatcaccctgaagtacgtgcccgggatggacgtgctgcccagccactgctggatcagcgagatggtggtgcagctgtccgactccctgaccgacctgctggacaagttctccaacatcagcgaggggctgagcaactactccatcatcgacaagctggtgaacatcgtggacgacctggtggagtgcgtgaaggagaacagcagcaaggacctgaagaagtccttcaagagccccgagccccggctgttcacgcccgaggagttcttccggatcttcaaccggagcatcgacgccttcaaggacttcgtggtggccagcgagaccagcgactgcgtggtgtcctcc

SEQ ID NO: 27 - DNA, 426 bp, synthetic construct
  source 1..426: mol_type = other DNA
gaggggatctgccggaaccgggtgaccaacaatgtgaaggacgtcacaaagttggtcgccaatctgccgaaggactacatgattacgctgaagtacgtccccggaatggatgtgctgcccagccactgctggatttcggagatggtggtgcagctgtccgacagtctgaccgatctgctggacaagttcagcaacatctccgaagggctgtccaactacagcatcatcgataagctggtcaacatcgtcgacgatctggtggagtgcgtcaaagagaacagcagcaaagatctgaagaagtcgttcaaatcgccggagccgcggctgttcacaccggaggagttcttccggatcttcaatcggtcgatcgacgccttcaaagattttgtggtggccagcgaaaccagcgactgcgtcgtcagcagc

SEQ ID NO: 28 - DNA, 426 bp, synthetic construct
  source 1..426: mol_type = other DNA
gaggggatctgccggaaccgcgtgacgaacaacgtgaaggacgtcacgaagctcgtcgcgaacctgccgaaggactacatgatcaccctgaagtacgtccccgggatggacgtgctcccctcgcactgctggatctccgagatggtcgtccagctgtccgactccctgacggacctcctcgacaagttctccaacatctccgaggggctctccaactactcgatcatcgacaagctggtgaacatcgtggacgacctcgtggagtgcgtcaaggagaactcctcgaaggacctcaagaagagcttcaagtcgcccgagccgcggctcttcacgcccgaggagttcttccggatcttcaaccggagcatcgacgccttcaaggacttcgtggtggcctccgagacgtccgactgcgtcgtgtcgagc

SEQ ID NO: 29 - DNA, 426 bp, synthetic construct
  source 1..426: mol_type = other DNA
gaggggatctgccggaaccgggtgaccaacaacgtgaaggacgtcaccaagctggtcgccaacctgcccaaggactacatgatcaccttgaagtacgtgcccggcatggacgtcctgcccagccactgctggatctccgagatggtcgtccagctcagcgactccctgaccgacctcctcgacaagttctccaacatctccgaggggctcagcaactactccatcatcgacaagctcgtgaacatagtggatgacctcgtggagtgcgtgaaggagaacagctccaaggacttgaagaagtccttcaagtccccggagcccaggctgttcacgcccgaggagttcttcaggatcttcaaccgatccattgacgccttcaaggacttcgtggtggcctccgagaccagcgactgcgtggtgtcctcc

SEQ ID NO: 30 - DNA, 426 bp, synthetic construct
  source 1..426: mol_type = other DNA
gaggggatctgccgcaaccgcgtgacgaacaacgtgaaggacgtgacgaagctcgtggcgaacctgcccaaggactacatgatcaccctgaagtacgtccccgggatggacgtgctgcccagccactgctggatctccgagatggtggtgcagctgagcgacagcctgacggacctgctggacaagttcagcaacatctccgaggggctgagcaactacagcatcatcgacaagctggtgaacatcgtggacgacctggtggagtgcgtgaaggagaacagctccaaggacctgaagaagagcttcaagtcgcccgagccccggctgttcacgcccgaggagttcttccggatcttcaaccggagcatcgacgccttcaaggacttcgtcgtggcgagcgagacgtccgactgcgtcgtgtcctcc

SEQ ID NO: 31 - protein, 590 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGS

SEQ ID NO: 32 - DNA, 1770 bp, Homo sapiens
  source 1..1770: mol_type = unassigned DNA
gacgctcataagtccgaggtggcccatcgctttaaggatctgggtgaggaaaattttaaggcgctggtgttgattgcatttgcgcagtatttgcagcagtgtccattcgaggatcacgtgaagttagtgaacgaggtgactgagttcgcaaagacctgtgtcgcggatgagagcgctgagaactgtgataaatcccttcacaccctgttcggcgataaactttgcactgtggccacattgcgcgaaacttacggagagatggccgactgctgcgctaaacaggagcccgagaggaacgagtgcttcttacagcataaggatgacaaccccaatctgccaagactggtcaggcctgaagtggacgtcatgtgtacagccttccatgacaacgaggagacgttcctcaagaaatacttgtacgagatcgctcgacgtcacccttatttctacgcacctgaattgctgttttttgccaagcggtataaagcagctttcacagagtgctgccaggctgcagacaaggctgcctgcttgcttccaaaattggacgagctgagagatgagggcaaggcatcctcagctaaacagcggctgaaatgtgcctctctgcaaaagttcggggagcgggctttcaaggcctgggccgttgcgcggctgagccagagatttcccaaggccgagttcgctgaagtttcaaagctcgtcaccgaccttaccaaggttcatactgaatgttgccatggcgacctgctagaatgcgcagacgacagggcggatctggccaagtacatctgtgagaatcaagactctattagtagcaagctcaaagagtgttgcgagaagccattactcgaaaagtcccattgcatcgcagaggtagaaaacgatgaaatgcctgccgaccttcccagtctcgccgccgactttgtggagagcaaagatgtgtgcaaaaattacgcagaggccaaggatgtattcctggggatgtttctctacgagtatgccagaagacaccccgattatagtgtggtgctccttctgcgcttagccaaaacatacgagaccaccctggagaagtgttgtgcagccgccgatccacacgagtgctatgctaaggtctttgatgaatttaaaccactagtcgaagagccccagaatctcataaagcagaattgcgaactttttgaacagctgggcgaatataaattccaaaatgctctgctagtacgatataccaagaaagtgccccaagtaagtactcctacgttggttgaggtgagccgtaacctggggaaggtgggatcaaagtgctgtaaacaccctgaagcgaaaaggatgccgtgcgcagaagattacctttctgtggtactcaaccagctctgcgtgttacacgagaagactccggtgtctgaccgagttacaaaatgttgcaccgagtcactcgtcaatcgccggccctgcttcagcgctctagaagtcgacgaaacctacgtgcctaaggaattcaacgccgagacgttcacctttcacgccgacatctgtactctctccgagaaggagaggcagattaagaaacaaacagcactggtggaactggttaaacacaaaccaaaggctacaaaggaacagctgaaagcggttatggatgacttcgccgcctttgtcgaaaagtgttgtaaagcagacgataaagaaacatgttttgctgaagaaggtaagaaactggtcgccgcctcgcaggcagctctgggactgggaggtggtgggtct

SEQ ID NO: 33 - DNA, 1770 bp, synthetic construct
  source 1..1770: mol_type = other DNA
gacgcccacaagtccgaggtggcccacaggttcaaggacctgggcgaggagaacttcaaggccctggtgctgatcgccttcgcccagtacctgcagcagtgccccttcgaggaccacgtgaagttggtgaacgaggtgaccgagttcgccaagacctgcgtggccgacgagagcgccgagaactgcgacaagtccctgcacaccctgttcggggacaagctgtgcaccgtggccacgctgcgggagacctacggggagatggccgactgctgcgccaagcaggagcccgagcggaacgagtgcttcctgcagcacaaggacgacaaccccaacctgccccgactggtccggcccgaggtggacgtgatgtgcaccgccttccacgacaacgaggagaccttcctgaagaagtacctgtacgagatcgcccggcggcacccctacttctacgcgcccgagctgctgttcttcgccaagcggtacaaggccgccttcaccgagtgctgccaggccgccgacaaggccgcctgcctgctgcccaagctggacgagctgcgggacgaggggaaggcctccagcgccaagcagcggctgaagtgcgcgtccctgcagaagttcggggagcgggccttcaaggcttgggccgtggcccggctgtcccaacggttccccaaggccgagttcgccgaggtgagcaagttggtgaccgacctgaccaaggtgcacaccgagtgctgccacggggacctgctggagtgcgccgacgaccgggccgatctggccaagtacatctgcgagaaccaggacagcatcagctccaagctgaaggagtgctgcgagaagcccctgctggagaagtcccactgcatcgccgaggtggagaacgacgagatgccggccgatctgccctcgctggccgccgacttcgtggagagcaaggacgtgtgcaagaactacgccgaggccaaggacgtgttcctggggatgttcctgtacgagtacgcccggcggcaccccgactactccgtggtgctgctgctgcggctggccaagacctacgagaccaccttggagaagtgctgcgccgcggccgatccgcacgagtgctacgccaaggtgttcgacgagttcaagcccctggtggaggagccccagaacctgatcaagcagaactgcgagctgttcgagcagctcggggagtacaagttccagaacgccctgctggtgcggtacaccaagaaggtgccccaggtgagcaccccgaccctggtggaggtgagccggaacctggggaaggtcgggtccaagtgctgcaagcaccccgaggccaagcggatgccctgcgccgaggactacctgtccgtggtgctgaaccagctgtgcgtgctgcacgagaagacgcccgtgtccgaccgggtgaccaagtgctgcaccgagagcttggtgaaccggcggccctgcttcagcgccctggaggtggacgagacctacgtgcccaaggagttcaacgccgagaccttcaccttccacgccgacatctgcaccctgagcgagaaggagcggcagatcaagaagcagacggccctggtggagctggtgaagcacaagcccaaggccaccaaggagcagctgaaggccgtgatggacgacttcgccgccttcgtggagaagtgctgcaaggccgacgacaaggagacctgcttcgccgaagagggcaagaagttggtggccgcgtcccaggccgcactgggtctgggaggtggtgggtct

SEQ ID NO: 34 - DNA, 1770 bp, synthetic construct
  source 1..1770: mol_type = other DNA
gacgcccacaagagcgaggtggcgcaccgcttcaaggacctgggcgaggagaacttcaaggcgctggtgctgatcgcgttcgcccagtacctccagcagtgccccttcgaggaccacgtgaagctggtgaacgaggtgaccgagttcgccaagacctgcgtggccgacgagagcgccgagaactgcgacaagagcctccacaccctgttcggggacaagctgtgcaccgtggccacgctgcgcgagacctacggggagatggccgactgctgcgccaagcaggagcccgagcgcaacgagtgcttcctccagcacaaggacgacaaccccaacctaccccgtctggtgcgccccgaggtggacgtgatgtgcaccgcgttccacgacaacgaggagaccttcctcaagaagtacctctacgagatcgcgcggcggcacccctacttctacgcgcccgagctgctgttcttcgccaagcgctacaaggccgcgttcaccgagtgctgccaggccgccgacaaggccgcgtgcctgctccccaagctcgacgagctgcgcgacgaggggaaggcgtcgagcgccaagcagaggctcaagtgcgcctcgctccagaagttcggggagcgcgcgttcaaggcgtgggccgtggcgaggctctcccagcggttccccaaggccgagttcgccgaggtgagcaagctggtgaccgacctcaccaaggtgcacaccgagtgctgccacggggacctgctggagtgcgccgacgaccgcgccgatctcgccaagtacatctgcgagaaccaggacagcatctcctccaagctcaaggagtgctgcgagaagcccctgctggagaagagccactgcatcgccgaggtggagaacgacgagatgccagccgatctcccctcgctcgccgccgacttcgtggagagcaaggacgtgtgcaagaactacgccgaggccaaggacgtgttcctggggatgttcctctacgagtacgcgcggcggcaccccgactacagcgtggtgctgctgctgcggctcgccaagacctacgagaccacgctggagaagtgctgcgccgccgccgatccccacgagtgctacgccaaggtgttcgacgagttcaagcccctggtggaggagccccagaacctgatcaagcagaactgcgagctgttcgagcagctcggggagtacaagttccagaacgcgctgctggtgcgctacaccaagaaggtgccccaggtgagcaccccgaccctggtggaggtgagccgcaacctggggaaggtcgggagcaagtgctgcaagcaccccgaggccaagcggatgccctgcgccgaggactacctctcggtggtgctcaaccagctgtgcgtgctccacgagaagaccccggtgagcgacagggtgaccaagtgctgcaccgagtcgctggtgaaccggaggccctgcttctccgcgctggaggtggacgagacctacgtgcccaaggagttcaacgccgagaccttcaccttccacgccgacatctgcaccctctccgagaaggagcgccagatcaagaagcagaccgcgctggtggagctggtgaagcacaagcccaaggccaccaaggagcagctcaaggcggtgatggacgacttcgccgcgttcgtggagaagtgctgcaaggccgacgacaaggagacctgcttcgccgaagaggggaagaagctggtggccgcgagccaagccgctctggggctgggaggtggtgggtct

SEQ ID NO: 35 - DNA, 1770 bp, synthetic construct
  source 1..1770: mol_type = other DNA
gacgcccacaagtctgaggtcgcccatcggttcaaggatctcggcgaggagaacttcaaggccctcgtgctgatcgccttcgcgcagtacctccagcagtgccccttcgaggaccacgtgaagctggtcaacgaggtgaccgagttcgccaagacctgcgtggccgacgagagcgccgagaactgcgataagagcctgcacaccctgttcggcgacaagctgtgcaccgtggcgaccctcagggagacctacggggagatggccgactgctgcgccaagcaggagcccgagcggaacgagtgcttcctgcagcacaaggatgacaaccccaatctgccccgcttggtgcgccccgaggtggacgtgatgtgcaccgcgttccacgacaacgaggagaccttcctgaagaagtacctctacgagatcgccaggcggcacccctacttctacgctcccgagctgctgttcttcgccaagcggtacaaggcggccttcacggagtgctgccaggcggccgacaaggcggcctgcctgctgcccaagctggacgagctgcgcgacgaggggaaggccagcagcgccaagcagagactgaagtgcgcctctctgcagaagttcggcgagcgggccttcaaggcgtgggccgtcgctcggctctcccagcggttccccaaggcggagttcgccgaggtgtccaagctggtgaccgacctgaccaaggtgcacaccgagtgctgccacggggacctcctggagtgcgccgacgatcgggccgacctggcgaagtacatctgcgagaaccaggatagcatcagctccaagctgaaggagtgctgcgagaagcccctgctggagaagagccactgcatcgccgaggtggagaacgacgagatgcccgcagatctgcccagcctggcggccgacttcgtggagagcaaggatgtctgcaagaattacgccgaggcgaaggacgtgttcctcgggatgttcctctacgagtacgcccggcggcaccccgattacagcgtggtgctcctcctgcggctcgccaagacctacgagacaaccctggagaagtgctgcgctgccgccgatccccacgagtgctacgccaaggtgttcgacgagttcaagcccctcgtggaggagccccagaatctcatcaagcagaactgcgagctgttcgagcagctcggggagtacaagttccagaacgccctgctggtgaggtacaccaagaaggtgccccaggtgagcaccccgaccctcgtggaggtgagccgcaacctggggaaggtcggctctaagtgctgcaagcaccccgaggccaagcggatgccctgcgcggaggactacctgtccgtggtgctcaaccagctgtgcgtgctgcacgagaagacacccgtgtccgaccgggtgaccaagtgctgcacggagtccctggtgaaccggcggccctgcttctccgctctggaggtcgacgagacctacgtgcccaaggagttcaacgccgagacgttcaccttccacgcggatatctgcacgctgagcgagaaggagaggcagatcaagaagcagacggccctcgtggagctcgtgaagcacaagcccaaggccaccaaggagcagctgaaggccgtgatggacgacttcgcggccttcgtggagaagtgctgcaaggcggacgacaaggagacctgcttcgccgaggaaggcaagaagctggtggccgccagtcaggctgccctggggctcggaggtggtgggtct

SEQ ID NO: 36 - DNA, 1770 bp, synthetic construct
  source 1..1770: mol_type = other DNA
gacgcccacaagtccgaggtggcgcaccgcttcaaggacctcggggaagagaacttcaaggccctggtgctgatcgccttcgcgcagtacctgcagcagtgccccttcgaggaccacgtgaagctcgtgaacgaggtcacggagttcgccaagacctgcgtcgccgacgagagcgccgagaactgcgacaagagcctccacaccctgttcggggacaagctctgcaccgtggcgacgctgcgggagacctacggcgagatggcggactgctgcgccaagcaggagcccgagcgcaacgagtgcttcctgcagcacaaggacgacaaccccaacctgccgcggctcgtgaggcccgaggtcgacgtgatgtgcaccgcgttccacgacaacgaggagacgttcctgaagaagtacctctacgagatcgcgaggcggcacccctacttctacgccccggagctgctgttcttcgcgaagcgctacaaggccgccttcacggagtgctgccaggccgccgacaaggccgcgtgcctgctgccgaagctcgacgagctccgggacgagggcaaggcctccagcgccaagcagcgcctcaagtgcgcctcgctgcagaagttcggggagcgggccttcaaggcctgggccgtggcgcggctgtcacagcggttccccaaggccgagttcgccgaggtctcgaagctcgtgacggacctcaccaaggtccacaccgagtgctgccacggggacctcctggagtgcgccgacgacagggccgacctcgcgaagtacatctgcgagaaccaggactccatcagctccaagctcaaggagtgctgcgagaagcccctgctggagaagtcccactgcatcgccgaggtcgagaacgacgagatgcccgcggatctcccgagcctcgccgcggacttcgtggagtccaaggacgtctgcaagaactacgccgaggccaaggacgtgttcctggggatgttcctgtacgagtacgcgaggcggcaccccgactactccgtggtgctgctgctgcggctcgccaagacctacgagacgaccctggagaagtgctgcgccgccgcagatccccacgagtgctacgccaaggtcttcgacgagttcaagcccctggtggaggagccccagaacctcatcaagcagaactgcgagctcttcgagcagctcggggagtacaagttccagaacgcgctgctggtgcggtacaccaagaaggtgccccaggtctcgaccccgaccctggtggaggtgtcccggaacctcgggaaggtcgggtccaagtgctgcaagcaccccgaggcgaagcggatgccctgcgccgaggactacctctccgtggtgctgaaccagctctgcgtgctgcacgagaagaccccggtgtcggaccgcgtcacgaagtgctgcaccgagtccctggtgaaccggaggccctgcttcagcgccctggaggtcgacgagacctacgtccccaaggagttcaacgccgagaccttcacgttccacgccgacatctgcaccctgtccgagaaggagcgccagatcaagaagcagacggccctggtggagctcgtgaagcacaagcccaaggccaccaaggagcagctcaaggccgtgatggacgacttcgcggccttcgtggagaagtgctgcaaggccgacgacaaggagacctgcttcgccgaggaaggcaagaagctcgtggcggcgtcccaggcggctctgggactcggaggtggtgggtct

SEQ ID NO: 37 - DNA, 1770 bp, synthetic construct
  source 1..1770: mol_type = other DNA
gacgcccacaagtccgaggtggcgcaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagtccgccgagaactgcgacaagtccctgcacaccctgttcggggacaagctgtgcaccgtggccacgctgcgggagacctacggggagatggccgactgctgcgccaagcaggagcccgagcggaacgagtgcttcctgcagcacaaggacgacaaccccaacctgcccaggctggtgcggcccgaggtggacgtgatgtgcaccgccttccacgacaacgaggagaccttcctgaagaagtacctgtacgagatcgcccggcggcacccctacttctacgcgcccgagctgctgttcttcgccaagcggtacaaggccgccttcaccgagtgctgccaggccgccgacaaggccgcctgcctgctgcccaagctggacgagctgcgggacgaggggaaggcctcctccgccaagcagcggctgaagtgcgccagcctgcagaagttcggggagcgggccttcaaggcttgggccgtggcccggctgtcccagaggttccccaaggccgagttcgccgaggtgtccaagctggtgaccgacctgaccaaggtgcacaccgagtgctgccacggggacctgctggagtgcgccgacgaccgggctgacctggccaagtacatctgcgagaaccaggactccatctcctccaagctgaaggagtgctgcgagaagcccctgctggagaagtcccactgcatcgccgaggtggagaacgacgagatgccggctgacctgcccagcctggccgccgacttcgtggagtccaaggacgtgtgcaagaactacgccgaggccaaggacgtgttcctggggatgttcctgtacgagtacgcccggcggcaccccgactactccgtggtgctgctgctgcggctggccaagacctacgagacgaccctggagaagtgctgcgccgccgccgatccccacgagtgctacgccaaggtgttcgacgagttcaagcccctggtggaggagccccagaacctgatcaagcagaactgcgagctgttcgagcagctgggcgagtacaagttccagaacgccctgctggtgcggtacaccaagaaggtgccccaggtgagcaccccgaccctggtggaggtgtcccggaacctggggaaggtcgggtccaagtgctgcaagcaccccgaggccaagcggatgccctgcgccgaggactacctgtccgtggtgctgaaccagctgtgcgtgctgcacgagaagacgcccgtgtccgaccgggtgaccaagtgctgcaccgagtccctggtgaaccggcggccctgcttcagcgccctggaggtggacgagacctacgtgcccaaggagttcaacgccgagaccttcaccttccacgccgacatctgcaccctgtccgagaaggagcggcagatcaagaagcagacggccctggtggagctggtgaagcacaagcccaaggccaccaaggagcagctgaaggccgtgatggacgacttcgccgccttcgtggagaagtgctgcaaggccgacgacaaggagacctgcttcgccgaagaggggaagaagctggtggccgccagccaggctgccctgggactgggaggtggtgggtct

SEQ ID NO: 38 - protein, 244 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMKYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRAD

SEQ ID NO: 39 - DNA, 732 bp, synthetic construct
  source 1..732: mol_type = other DNA
gaagtgcagttgctgcagtctggacccgagctggagaagcctggggcttcagtgatgataagttgtaaagcgagcggctcttcgttcacgggatacaacatgaattgggtaagacagaacatcggaaagtccctcgaatggattggtgcaatcgatccatactatgggggcacaagctataatcagaagtttaaaggcagggccactctgaccgtcgacaaatcctcatccaccgcttatatgcacttaaagagtcttacttctgaagacagcgccgtttattactgcgtgtcaggcatgaagtactggggtcaagggacaagcgtgaccgtcagttccggtggtggtggaagcggtggtggtggatctggcggtggtggaagtgatgtagtgatgacccagactcctctgagtctgcccgttagtttaggtgaccaggccagcatcagctgcaggtccagccagtcattggttcatagaaacggcaatacctacctgcactggtacctgcaaaaacccgggcaatctcctaaacttctcatacacaaggtctctaaccgtttctcgggggtcccggatcggtttagcggatcaggctccggaacagattttacgttgaagatttctcgcgtggaggccgaggaccttggtgtgtatttctgttcccagtccactcatgtgccaccactgacattcggcgctgggaccaaactcgaactaaagcgagcagac

SEQ ID NO: 40 - DNA, 732 bp, synthetic construct
  source 1..732: mol_type = other DNA
gaggtccagctcctccagtccgggcccgagctggagaagcccggggcctccgtgatgatctcctgcaaggcctccgggtcctccttcaccgggtacaacatgaactgggtccgccagaacatcgggaagagcctggagtggatcggggccatcgacccctactacggaggcacctcctacaaccagaagttcaagggccgggccactctgaccgtggacaagtcctcctccaccgcctacatgcacctgaagagcctgacctccgaggactccgccgtctactactgcgtctccgggatgaagtactggggccaggggacctccgtgaccgtctcctccggaggaggaggctccggaggaggaggctccggaggaggaggctccgacgtggtgatgacccagacccctctgagcctgcccgtgagcctcggggaccaggcctccatcagctgcaggagctcccagagcctggtccacaggaacgggaacacctacctccactggtacctccagaagcccgggcagagccccaagctcctgatccacaaggtctccaacaggttctccggggtccccgaccgcttcagcgggtccgggtccgggaccgacttcaccctgaagatctccagagtggaggccgaggacctcggggtctacttctgctcccagagcacccacgtgcctcctctgaccttcggggccgggaccaagctggagctcaagagggccgac

SEQ ID NO: 41 - DNA, 732 bp, synthetic construct
  source 1..732: mol_type = other DNA
gaggtccagctgctgcagtcggggcccgagctggagaagcccggggcctccgtcatgatctcgtgcaaggcctccgggtcctcgttcaccgggtacaacatgaactgggtgcgccagaacatcgggaagtcgctggagtggatcggggccatcgacccctactacggcggcaccagctacaaccagaagttcaaggggcgcgccacgctgacggtggacaagtcgtcgtcgaccgcctacatgcacctgaagtcgctgacgtcggaggactccgccgtctactactgcgtcagcgggatgaagtactggggccaggggacctcggtcaccgtgtcctccggcggcggaggaagcggaggaggaggctccggcggaggaggctccgacgtcgtgatgacgcagaccccgctgtcgctcccggtgtccctcggggaccaggcctccatcagctgccggagctcgcagtccctggtgcaccggaacgggaacacctacctccactggtacctgcagaagccggggcagtcgcccaagctgctgatccacaaggtgtccaacaggttctccggggtccccgaccgcttcagcgggagcgggagcgggaccgacttcaccctgaagatctcccgcgtggaggccgaggacctcggggtctacttctgcagccagagcacccacgtgccaccgctgaccttcggcgccggcaccaagctggagctcaagcgggccgac

SEQ ID NO: 42 - DNA, 732 bp, synthetic construct
  source 1..732: mol_type = other DNA
gaggtccagctgctccagtccgggcccgagctggagaagcccggggcctcggtgatgatcagctgcaaggcctccgggtcgagcttcaccgggtacaacatgaactgggtccggcagaacatcgggaagtcgctggagtggatcggggcgatcgacccctactacggcggcaccagctacaaccagaagttcaaggggcgcgcgaccctgaccgtcgacaagtcgagctcgaccgcctacatgcacctgaagtcgctgacctccgaggactccgcggtctactactgcgtgagcgggatgaagtactggggccaggggacctcggtgaccgtgagctccggcggcggcggatctggtggtggcggttccggcggtggcggttccgacgtggtgatgacccagaccccgctctcgctcccggtctcgctcggggaccaggcctcgatcagctgccggagctcccagtcgctggtccaccggaacgggaacacctacctccactggtacctccagaagcccgggcagtcgcccaagctgctgatccacaaggtgagcaaccgcttctccggggtccccgaccgcttctccgggtccgggtccgggaccgacttcaccctgaagatctcccgggtcgaggccgaggacctcggggtctacttctgctcccagtcgacccacgtgccgccactgaccttcggggccgggaccaagctggagctgaagcgggccgac

SEQ ID NO: 43 - DNA, 732 bp, synthetic construct
  source 1..732: mol_type = other DNA
gaggtgcagctgctgcagtccggccccgagctggagaagcccggggccagcgtgatgatcagctgcaaggccagcgggtccagcttcacggggtacaacatgaactgggtgcggcagaacatcgggaagagcctggagtggatcggggccatcgacccgtactacggcggcacgtcgtacaaccagaagttcaaggggcgcgccacgctgaccgtggacaagtccagcagcacggcctacatgcacctcaagtcgctgaccagcgaggacagcgcggtgtactactgcgtgtccggcatgaagtactggggccaggggaccagcgtgacggtgagcagcggcggtggtggcagcggtggtggcggtagcggcggtggtggcagcgacgtggtgatgacccagacgcccctcagcctgccggtcagcctgggcgaccaggccagcatttcgtgccgcagctcgcagagcctggtgcaccgcaacgggaacacctacctgcactggtacctgcagaagcccgggcagagccccaagctgctgatccacaaggtgtccaaccgcttcagcggggtgcccgaccgcttcagcgggtcgggcagcgggacggacttcaccctgaagatcagccgcgtggaggccgaggacctcggggtgtacttctgctcgcagagcacccacgtgccgccgcttaccttcggggccggcaccaagctggagctcaagcgggccgac

SEQ ID NO: 44 - DNA, 732 bp, synthetic construct
  source 1..732: mol_type = other DNA
gaggtccagctcctccagtccgggcccgagctggagaagcccggggcctccgtgatgatctcctgcaaggcctccgggtcctcgttcaccgggtacaacatgaactgggtgaggcagaacatcgggaagagcctggagtggatcggggccatcgacccctactacggcggaacctcgtacaaccagaagttcaaggggagagccacgctgaccgtggacaagtcctcgtccaccgcgtacatgcacctgaagagcctgacctccgaggactccgccgtctactactgcgtctccgggatgaagtactggggacaggggacctccgtgaccgtctcctccggcggaggaggctcaggcggaggaggctccggcggaggaggctccgacgtggtgatgacccagactcccctctccctgcccgtctccctcggcgaccaggcgagcatctcctgccggtcctctcagtccctcgtccaccggaacgggaacacctacctccactggtacctgcagaagcccgggcagtcgcccaagctgctcatccacaaggtgagcaaccgcttctccggggtccccgaccgcttctccgggtccgggtccgggaccgacttcaccctgaagatctcccgggtcgaggccgaggacctcggcgtctacttctgctcccagtctacccacgttcctccgctgaccttcggcgcggggacgaagctggagctgaagagggccgat

SEQ ID NO: 45 - protein, 241 aa, synthetic construct
EVQLEESGGGLVKPGGSLKLSCAASGFAFSSYDMSWVRQIPEKRLEWVAKVSSGGGSTYYLDTVQGRFTISRDNAKNTLYLQMSSLNSEDTAMYYCARHNYGSFAYWGQGTLVTVSAAKGGGGSGGGGSGGGGSELVMTQTPATLSVTPGDSVSLSCRASQSISNHLHWYQQKSHESPRLLIKYASQSISGIPSRFSGSGSGTDFTLSINSVETEDFGMYFCQQSNSWPHTFGGGTKLEIK

SEQ ID NO: 46 - DNA, 723 bp, synthetic construct
  source 1..723: mol_type = other DNA
gaggtgcagctggaggagtctggaggtggcctggtgaagccaggaggcagcctgaagctgagctgtgctgcctctgggtttgccttcagctcctatgacatgagctgggtgaggcagatccctgagaagaggctggagtgggtagctaaggtgagctctggaggtggcagcacctactacctggacacagtgcagggcaggttcaccatcagcagggacaatgctaagaacaccctgtacctgcagatgagcagcctgaactctgaggacacagctatgtactactgtgccaggcacaactatgggtcctttgcctactggggccaggggaccctggtgacagtgtctgcagctaaaggtggaggtggctctggaggtggaggctctggaggtggaggctctgagctggtgatgacccagaccccagctaccctgagtgtgaccccaggggactctgtgtccctgagctgcagggccagccagtccatctccaaccacctgcactggtaccagcagaagtcccatgagagccccaggctgctgatcaagtatgcctctcagtccatctctggcatccccagcaggttctcagggtcagggtcagggacagacttcaccctgagcatcaactctgtggagacagaggactttgggatgtacttctgccagcagtccaactcctggccccacacctttggaggtggcaccaagctggagatcaag

SEQ ID NO: 47 - DNA, 723 bp, synthetic construct
  source 1..723: mol_type = other DNA
gaggtgcagctggaggagtccggcggcggcttggtgaagcccggcggctccctgaagctgtcctgcgccgcctccgggttcgccttctccagctacgacatgtcctgggtgcggcagatccccgagaagcggctggagtgggtcgccaaggtgtcctccggcggcggctccacctactacctggacaccgtgcaggggcgcttcaccatctcccgggacaacgccaagaacaccctgtacctgcagatgagctccctgaacagcgaggacaccgccatgtactactgcgcccggcacaactacgggtccttcgcctactggggccaggggaccctggtgaccgtgtccgccgccaaaggaggcggcggctccggaggaggtggctcaggcggcggaggcagcgagctggtgatgacccagaccccggccacgctgtccgtgacgcccggggactccgtgtccctgagctgccgggccagccagtccatcagcaaccacctgcactggtaccagcagaagtcccacgagagcccgcggctgctgatcaagtacgccagccagtccatctccggcatccccagccggttctccgggtccgggtccgggaccgacttcaccctgtccatcaactccgtggagaccgaggacttcgggatgtacttctgccagcagagcaacagctggccccacaccttcggcggcggcaccaagctggagatcaag

SEQ ID NO: 48 - DNA, 723 bp, synthetic construct
  source 1..723: mol_type = other DNA
gaagtccagctggaggagagcggcggtggcctcgtgaagcccggcggttcgctcaagctgagctgcgcggccagcgggttcgccttcagctcgtacgacatgagctgggtgcggcagatccccgagaagcgcctggagtgggtcgccaaggtgtcctccggcggcggctcgacgtactacctggacaccgtgcaggggcgcttcacgatcagccgggacaacgcgaagaacaccctgtacctccagatgtcctcgctgaactccgaggacaccgcgatgtactactgcgcgcggcacaactacgggagcttcgcctactggggccagggcaccctggtcaccgtgagcgcggcgaaaggcggtggcggcagtggtggcggaggctctggcggcggtggctccgagctggtgatgacccagaccccggcgacgctctccgtgacgcccggggactccgtgagcctgtcctgccgggcctcgcagagcatctccaaccacctgcactggtaccagcagaagagccacgagtcgccccggctgctcatcaagtacgcctcgcagagcatctccgggattccctcgcggttctccgggagcggctcggggacggacttcacgctgtccatcaactccgtggagaccgaggacttcgggatgtacttctgccagcagagcaactcctggccgcacaccttcggcggcggcaccaagctggagatcaag

SEQ ID NO: 49 - DNA, 723 bp, synthetic construct
  source 1..723: mol_type = other DNA
gaggtccagctggaggaatccggcggcggcctcgtgaagccaggcggcagcctgaagctgtcgtgcgcggcctccgggttcgccttcagctcgtacgacatgagctgggtgcgccagatccccgagaagcgcctggagtgggtcgccaaggtgtcctccggcggcggcagcacgtactacttggacaccgtgcaggggcgcttcacgatctcgcgggacaacgcgaagaacacgctgtacctccagatgtcctcgctgaactcggaggacaccgcgatgtactactgcgcgcggcacaactatgggtccttcgcctactggggccaggggaccctggtgaccgtgagcgcggccaaaggtggcggcggctcaggtggcggaggcagcggtggcggaggcagcgagctcgtgatgacgcagacgccggcgaccctcagtgtgaccccaggggactccgtcagcctgagctgccgggcgtcccagtcgatctcgaaccacctgcactggtaccagcagaagtcccacgagagccccaggttgctcatcaagtacgccagccagtcgatctccgggattccctctcggttctcggggtccggcagcgggaccgacttcacgctgtcgatcaacagcgtggagaccgaggacttcgggatgtacttctgccagcagtccaacagttggccccacacgttcggcggcggcaccaagctggagatcaag

SEQ ID NO: 50 - DNA, 723 bp, synthetic construct
  source 1..723: mol_type = other DNA
gaggtccagctggaggagtccggcggaggcctggtcaagcccggcggcagcctgaagctgtcctgcgccgcctccggcttcgccttcagcagctacgacatgtcctgggtccggcagatcccggagaagcggctggagtgggtcgccaaggtcagctccggcggcggcagcacctactacctggacaccgtccagggccggttcaccatcagccgggacaacgccaagaacaccctgtacctgcagatgtccagcctgaactccgaggacacggccatgtactactgcgcccggcacaactacggcagcttcgcctactggggccaggggaccctggtgaccgtgtcggccgccaaaggcggaggcggctcaggaggcggaggttccggcggcggaggctccgagctggtcatgacccagacgccggccacgctgtccgtgacgcccggcgactccgtcagcctgtcctgccgggccagccagtccatcagcaaccacctgcactggtaccagcagaagtcccacgagtcgccccggctgctgatcaagtacgccagccagtccatctccggcatccccagccggttctccgggtccgggtccgggaccgacttcaccctgtccatcaactccgtcgagaccgaggacttcggcatgtacttctgccagcagtccaacagctggccgcacaccttcggcggcggcaccaagctggagatcaag

SEQ ID NO: 51 - DNA, 723 bp, synthetic construct
  source 1..723: mol_type = other DNA
gaggtccagctggaggagtccggaggaggactcgtgaagcccggaggctccctgaagctctcctgcgccgcctccgggttcgccttctcctcctacgacatgtcctgggtccggcagatccccgagaagaggctggagtgggtcgcgaaggtctcctccggaggaggctcgacctactatctcgacacggtccagggccggttcacgatctcccgggacaacgcgaagaacacgctctacctccagatgtcgagcctgaactccgaggacaccgcgatgtactactgcgcccggcacaactacgggagcttcgcctactggggccaggggactctcgtgaccgtctccgccgcgaaaggcggaggaggctccggaggaggaggctccggaggaggaggctccgagctcgtgatgacccagaccccggcgaccctctccgtgacgcccggggactccgtctccctctcctgtcgcgcgagccagtcgatctcgaaccatctccactggtaccagcagaagagtcacgagtctccccggctcctgatcaagtacgcgagccagtcgatctccgggattccctcgcggttctccgggtccgggtccgggaccgacttcacgctctcgatcaactccgtcgagaccgaggacttcgggatgtacttctgccagcagtcgaactcctggccccacacgttcggcggcggcacgaagctggagatcaag

SEQ ID NO: 52 - protein, 248 aa, synthetic construct
DIVMTQSPDSLAVSLGERATINCKSSQSLLNSRNRKNYLAWYQQKPDQSPKLLIYWASTRESGVPDRFSGSGSGTDFTLTISSLQAEDVAVYYCKQSYYLWTFGGGTKVEIKGGGGSGGGGSGGGGSEVQLVESGGGLVQPGGSLRLSCAASGFTFSDYGMAWFRQAPGKGLEWVSFISNLGYSIYYADSVKGRFTISRDNAKNSLYLQMNSLRAEDTAVYYCARAPYDYDSFDPMDYWGQGTLVTVS

SEQ ID NO: 53 - DNA, 744 bp, synthetic construct
  source 1..744: mol_type = other DNA
gacattgtgatgacccagagcccagactccctggctgtgagcctaggggagagggccaccatcaactgcaagtcctctcagagcctcctcaactccaggaacaggaagaactacctggcctggtaccagcagaagccagaccagagccccaagctgctcatctactgggcctccaccagggagtctggggtgcctgacaggttctctgggtctgggtctgggactgacttcaccctgaccatcagctccctgcaggctgaggatgtggctgtgtactactgcaagcagagctactacctgtggacctttggtggaggcaccaaggtggagatcaaaggaggtggaggctctggtggtggaggctctggtggtggaggctctgaggtgcagctggtggagtctggtggtggcctggtccagccaggaggctccctgaggctgagctgtgctgcctctgggttcaccttctcagactatgggatggcctggttcaggcaggccccagggaagggcctggagtgggtgagcttcatctccaacctgggctactccatctactatgctgactctgtgaagggcaggttcaccatcagcagggacaatgccaagaacagcctgtacctgcagatgaactccctgagggctgaggacacagctgtgtactactgtgccagggccccatatgactatgacagctttgaccccatggactactggggccaggggaccctggtgactgtgagc

SEQ ID NO: 54 - DNA, 744 bp, synthetic construct
  source 1..744: mol_type = other DNA
gacatcgtgatgacccagagccccgactccctggccgtgagcctcggggagagggccacgatcaactgcaagagctcccagagcctgctcaactcccggaaccggaagaactacctggcctggtaccagcagaagcccgaccagtcgcccaagctgctcatctactgggcctccaccagggagagcggggtgcccgaccgcttctccgggagcgggtccgggaccgacttcaccctgaccatctcctctctgcaggccgaggacgtggccgtgtactactgcaagcagagctactacctgtggacattcggcggcggcaccaaggtggagatcaaaggcggcggcggcagtggtggaggaggcagcggcggcggtggctcagaggtgcagctggtggagagcggaggcggcctggtgcagccaggcggctctctgcgcctgtcctgcgccgcctctgggttcaccttctccgactacgggatggcctggttccgccaggccccagggaaggggctggagtgggtgtccttcatctccaacctggggtacagcatctactacgccgactccgtgaagggccggttcaccatcagccgggacaacgccaagaacagcctgtacctgcagatgaactccctgcgcgccgaggacaccgccgtgtactactgcgcccgggctccctacgactacgacagcttcgaccccatggactactggggccaggggacactggtcaccgtgtcc

SEQ ID NO: 55 - DNA, 744 bp, synthetic construct
  source 1..744: mol_type = other DNA
gacatcgtgatgacgcagagccccgactctttggcggtgagcctcggggagagggccaccatcaactgcaagtcgtcgcagagcctcctcaacagccgcaaccggaagaactacctcgcctggtatcagcagaagcccgaccaaagccccaagctgctgatctactgggcctccacacgggagtctggggtgcccgaccgcttcagcggcagcggcagcggcaccgacttcaccctcaccatcagcagcctgcaggcggaggatgtggcggtgtactactgcaaacagtcctactatctgtggaccttcggcggcggcacaaaggtggagatcaaaggcggcggcggcagtggaggaggaggcagcggcggcggaggctctgaggtgcagctggtggagagcggcggcggtttggtgcagcccggcggcagcctccgactcagctgcgccgcctccggcttcaccttctccgactacgggatggcgtggtttcggcaggccccggggaaggggttggagtgggtgagcttcatctccaacttggggtacagcatctactacgccgactctgtgaaggggcgcttcaccatcagccgcgacaacgccaaaaacagcctctatctgcagatgaacagcctccgcgctgaggacacagcggtgtactactgcgcccgcgccccatacgactacgactccttcgaccccatggactactggggtcaggggacgttggtgacggtgtcg

SEQ ID NO: 56 - DNA, 744 bp, synthetic construct
  source 1..744: mol_type = other DNA
gacatcgtcatgacccagtcacctgacagcctggccgtcagcctgggcgaacgggccaccatcaactgtaagtcatctcagagcctgctgaacagccggaaccggaagaactacctggcctggtatcagcagaagcctgatcagtcacctaagctgctgatctactgggcctcaaccagagagtccggcgtgcctgacaggttcagcgggtccgggtccgggaccgacttcaccctgaccatcagcagcctgcaggccgaggacgtggccgtctattactgtaagcagtcttattacctgtggaccttcggcggcggcaccaaggtcgagatcaaaggcggcggcggctcaggtggtggtggctccggcggcggtggctctgaggtccagctggtcgagtccggcggtggcctggtccagccaggcggcagcctgaggctgtcctgcgccgcctccggcttcaccttctctgactacggcatggcctggttcagacaggccccaggcaagggcctggagtgggtcagcttcatcagcaacctgggctacagcatctattacgccgacagcgtcaagggccggttcaccatcagccgggacaacgccaagaacagcctgtacctgcagatgaacagcctgagggccgaggacacggccgtctattactgcgcccgggccccttatgactatgacagctttgaccccatggactactggggccaggggaccctggtgaccgtgtca

SEQ ID NO: 57 - DNA, 744 bp, synthetic construct
  source 1..744: mol_type = other DNA
gacatcgtgatgacccagagccccgactccctggccgtgtccctcggggagagggccaccatcaactgcaagagctcccagtccctgctgaactcccggaaccggaagaactacctggcctggtaccagcagaagcccgaccagagccccaagctgctgatctactgggccagcaccagggagagcggggtgcccgaccgcttctccgggtccgggtccgggaccgacttcaccctgaccatcagctccctgcaggccgaggacgtggccgtgtactactgcaagcagagctactacctgtggaccttcggcggcggcaccaaggtggagatcaaaggcggcggcggctcaggaggaggtggctccggcggcggaggctctgaggtgcagctggtggagagcggcggcggactggtgcagccaggcggctccctgcggctgagctgcgccgcctctgggttcaccttctccgactacgggatggcctggttccggcaggccccagggaaggggctggagtgggtgagcttcatctccaacctggggtactccatctactacgccgactccgtgaaggggcgcttcaccatctcccgggacaacgccaagaactccctgtacctgcagatgaactccctgcgggccgaggacaccgcggtgtactactgcgcccgggccccgtacgactacgacagcttcgaccccatggactactggggccaggggaccctggtgaccgtgagc

SEQ ID NO: 58 - DNA, 744 bp, synthetic construct
  source 1..744: mol_type = other DNA
gatatcgtgatgacccagtccccggactccctggcagtgtccctcggggagcgggccaccatcaactgcaagagctcccagtccctgctgaactcccggaaccggaagaactacctggcctggtaccagcagaagcccgaccagtccccgaagctgctgatctactgggccagcacccgggaatccggggtgcccgaccgcttctccgggtccgggtccgggaccgacttcaccctgaccatcagctccctgcaggccgaggacgtggcagtgtactactgcaagcagtcctactacctgtggaccttcggcggcggcaccaaggtggagatcaaaggcggcggcggctctggaggaggaggctccggcggcggaggttccgaggtgcagctggtggagtccggcggaggactggtgcagcccggcggctccctgcgactgtcctgcgccgcctccgggttcaccttctccgactacgggatggcctggttccggcaggctcccgggaaggggctggagtgggtgtccttcatctccaacctggggtactccatctactacgccgactccgtgaagggccggttcaccatctcccgggacaacgccaagaactccctgtacctgcagatgaactccctgcgggccgaggacaccgcagtgtactactgcgcccgggccccgtacgactacgactccttcgaccccatggactactggggccaggggaccctggtgaccgtgtcc

SEQ ID NO: 59 - protein, 71 aa, synthetic construct
SANSNPAMAPRERKAGCKNFFWKTFTSCGGGGSGGGGSGGGGSSANSNPAMAPRERKAGCKNFFWKTFTSC

SEQ ID NO: 60 - DNA, 258 bp, synthetic construct
  source 1..258: mol_type = other DNA
agcgcgaacagcaacccggcgatggcgccgcgcgaacgcaaagcgggctgcaaaaactttttttggaaaacctttaccagctgcggcggcggcggcagcggcggcggcggcagcggcggcggcggcagcagcgcgaacagcaacccggcgatggcgccgcgcgaacgcaaagcgggctgcaaaaactttttttggaaaacctttaccagctgcggcggcggcggcagcggcggcggcggcagcggcggcggcggcagc

SEQ ID NO: 61 - DNA, 258 bp, synthetic construct
  source 1..258: mol_type = other DNA
tctgccaactccaaccccgctatggctcccagggagcggaaggccgggtgcaagaacttcttctggaagaccttcacctcctgcggaggcggaggctcaggaggcggaggctccggaggaggcggctcctccgccaactccaaccccgctatggctcccagggagcggaaggccgggtgcaagaacttcttctggaagaccttcacctcctgcggaggcggaggctccggaggaggcggctcaggaggcggcggcagc

SEQ ID NO: 62 - DNA, 258 bp, synthetic construct
  source 1..258: mol_type = other DNA
tccgccaactccaaccccgctatggctccccgggagcggaaggctgggtgcaagaacttcttctggaagaccttcacctcctgcggaggcggaggttccggaggcggaggttccggcggaggcggctcctccgccaactccaaccccgcgatggctcccagggagcggaaggccgggtgcaagaacttcttctggaagaccttcacctcctgcggaggcggaggttccggcggaggaggttccggaggcggcggctcc

SEQ ID NO: 63 - DNA, 258 bp, synthetic construct
  source 1..258: mol_type = other DNA
agcgccaactccaaccccgctatggctccccgggagcgcaaggctgggtgcaagaacttcttctggaagaccttcacctcctgcggaggcggaggctctggaggcggaggctctggcggaggcggctcctccgccaactccaaccccgcgatggctcccagggagcgcaaggccgggtgcaagaacttcttctggaagaccttcacctcctgcggaggcggaggctctggcggaggaggctctggaggcggcggctcc

SEQ ID NO: 64 - DNA, 258 bp, synthetic construct
  source 1..258: mol_type = other DNA
tccgccaactccaacccggccatggctccccgggagaggaaggccgggtgcaagaacttcttctggaagaccttcacctcctgcggaggaggaggaagcggaggaggaggaagcggaggaggaggctcctcggccaactccaacccggccatggctccccgggagaggaaggccgggtgcaagaacttcttctggaagaccttcacctcctgcggaggaggaggaagcggaggaggaggaagcggaggaggaggatct

SEQ ID NO: 65 - DNA, 258 bp, synthetic construct
  source 1..258: mol_type = other DNA
tccgccaactccaaccccgctatggctccccgggagcggaaggctgggtgcaagaacttcttctggaagaccttcacctcctgtggcggcggcggatctggcggaggaggctctggcggaggcggctcctccgccaactccaaccccgctatggctcccagggagcggaaggccgggtgcaagaacttcttctggaagaccttcacctcctgtggcggcggcggatctggcggaggaggctctggcggaggcggaagc

SEQ ID NO: 66 - protein, 84 aa, synthetic construct
SANSNPAMAPRERKAGCKNFFWKTFTSCGGSGGSGGSGGSGGSANSNPAMAPRERKAGCKNFFWKTFTSCGGSGGSGGSGGSGG

SEQ ID NO: 67 - DNA, 252 bp, synthetic construct
  source 1..252: mol_type = other DNA
agcgcgaacagcaacccggcgatggcgccgcgcgaacgcaaagcgggctgcaaaaactttttttggaaaacctttaccagctgcggcggcagcggcggcagcggcggcagcggcggcagcggcggcagcgcgaacagcaacccggcgatggcgccgcgcgaacgcaaagcgggctgcaaaaactttttttggaaaacctttaccagctgcggcggcagcggcggcagcggcggcagcggcggcagcggcggc

SEQ ID NO: 68 - DNA, 252 bp, synthetic construct
  source 1..252: mol_type = other DNA
tccgccaactccaaccccgcaatggctccccgggagaggaaggccgggtgcaagaacttcttctggaagaccttcaccagctgcggaggctccggaggctccggaggctccggaggctccggaggctccgccaactccaaccccgcaatggctccccgggagaggaaggccgggtgcaagaacttcttctggaagaccttcaccagctgcggaggctccggaggctccggaggctccggaggctccggaggc

SEQ ID NO: 69 - DNA, 252 bp, synthetic construct
  source 1..252: mol_type = other DNA
tctgccaattccaaccccgcgatggcccctcgggagaggaaggccgggtgcaagaacttcttctggaagaccttcacctcctgcggaggctccggaggctccggaggctccggaggctccggaggctctgccaattccaaccccgcgatggcccctcgggagaggaaggccgggtgcaagaacttcttctggaagaccttcacctcctgcggaggctccggaggctccggaggctccggaggctccggaggc

SEQ ID NO: 70 - DNA, 252 bp, synthetic construct
  source 1..252: mol_type = other DNA
tccgcgaactccaaccccgcgatggctccccgggagaggaaggccgggtgcaagaacttcttctggaagacgttcacgtcctgcggaggctccggaggctccggaggctccggaggctccggaggctccgcgaactccaaccccgcgatggctccccgggagaggaaggccgggtgcaagaacttcttctggaagacgttcacgtcctgcggaggctccggaggctccggaggctccggaggctccggagga

SEQ ID NO: 71 - DNA, 252 bp, synthetic construct
  source 1..252: mol_type = other DNA
agcgccaactccaaccccgcgatggctcccagggagcggaaggccgggtgcaagaacttcttctggaagaccttcacctcctgcggaggctccggaggctccggaggctccggaggctccggaggctccgccaactccaaccccgcgatggctcccagggagcggaaggccgggtgcaagaacttcttctggaagaccttcacctcctgcggaggctccggaggctccggaggctccggaggctccggtggc

SEQ ID NO: 72 - DNA, 252 bp, synthetic construct
  source 1..252: mol_type = other DNA
tccgccaactccaaccccgctatggcccctagggagaggaaggccgggtgcaagaacttcttctggaagaccttcacctcctgcggaggctccggaggctccggaggctccggaggctccggaggctccgccaactccaaccccgctatggcccctagggagaggaaggccgggtgcaagaacttcttctggaagaccttcacctcctgcggaggctccggaggctccggaggctccggaggctccggagga

SEQ ID NO: 73 - protein, 28 aa, synthetic construct
SANSNPAMAPRERKAGCKNFFWKTFTSC

SEQ ID NO: 74 - DNA, 84 bp, synthetic construct
  source 1..84: mol_type = other DNA
tccgccaactccaacccggccatggctccccgggagaggaaggccgggtgcaagaacttcttctggaagaccttcaccagctgc

SEQ ID NO: 75 - DNA, 84 bp, synthetic construct
  source 1..84: mol_type = other DNA
agcgccaacagcaaccccgctatggctcccagggagcgcaaggccgggtgcaagaacttcttctggaagaccttcacctcttgc

SEQ ID NO: 76 - DNA, 84 bp, synthetic construct
  source 1..84: mol_type = other DNA
agcgcgaacagcaacccggccatggcccctcgcgagcgaaaggccgggtgcaagaacttcttctggaagaccttcacctcgtgc

SEQ ID NO: 77 - DNA, 84 bp, synthetic construct
  source 1..84: mol_type = other DNA
agcgccaactccaacccggctatggcgcccagggagaggaaggccggctgcaagaacttcttctggaagaccttcacctcctgc

SEQ ID NO: 78 - DNA, 84 bp, synthetic construct
  source 1..84: mol_type = other DNA
agtgcgaactcgaacccggccatggcgcccagagagcgcaaggccgggtgcaagaactttttctggaaaacgttcacatcgtgc

SEQ ID NO: 79 - DNA, 84 bp, synthetic construct
  source 1..84: mol_type = other DNA
agcgcgaactcgaacccggccatggctccgcgcgagcgtaaggccgggtgcaagaactttttctggaaaaccttcacgagttgt

SEQ ID NO: 80 - protein, 171 aa, synthetic construct
SPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQGRTTAHKDPNAIFLSFQHLLRGKVRFLMLVGGSTLCVRRAPPTTAVPSRTSLVLTLNELG

SEQ ID NO: 81 - DNA, 513 bp, synthetic construct
  source 1..513: mol_type = other DNA
tccccagctccaccagcctgtgacctgagggtgctgagcaagctcctgagggactcccatgtgctgcacagcaggctgagccagtgccctgaggtgcatcccctgccaacccctgtgctgctgccagctgtggacttctcccttggggagtggaagacccagatggaggagaccaaggcccaggacatccttggggctgtgaccctgctgctggaaggggtgatggctgccaggggccagctggggccaacctgcctcagctccctgctggggcagctgtcagggcaggtgaggctgctgctgggagccctgcagtccctgctggggacccagggcaggaccacagcccacaaggaccccaatgccatcttcctgagcttccagcacctgctgaggggcaaggtgaggttcctgatgctggttggaggcagcaccctgtgtgtcaggagagctccaccaaccacagctgtgcccagcaggaccagcctggtgctgaccctgaatgagcttgga

SEQ ID NO: 82 - DNA, 513 bp, synthetic construct
  source 1..513: mol_type = other DNA
agccccgcaccacctgcctgcgacctgcgggtgctgtccaagctgctgcgggacagccacgtgctgcacagcaggctgtcccagtgccccgaggtgcacccactgcccacgcccgtgctgctgcccgctgtggacttctccctgggcgagtggaagacacagatggaggagaccaaggcccaggacatcctgggcgccgtgaccctgctgctggaaggggtgatggccgccagagggcagctggggccaacgtgcctgtcctcactgctggggcagctgtccgggcaggtgcggctgctgctgggcgccctgcagtccctgctgggcacccaggggcgcaccacagctcacaaggaccccaacgccatcttcctgtccttccagcacctgctgcggggcaaggtgcggttcctgatgctggtcggcggcagcaccctgtgcgtgcgcagggcaccaccgaccacagctgtgcccagcaggacctcactggtgctgaccctgaacgagctgggc

SEQ ID NO: 83 - DNA, 513 bp, synthetic construct
  source 1..513: mol_type = other DNA
agcccggctcctccggcctgcgacctgcgcgtgctgagcaagctcctgcgggactcgcacgtgctgcactcgcgcctgagccagtgccccgaggtgcatcccctgcctaccccggtgctcctgcccgcggtggacttctcgctcggggagtggaagacccagatggaggagaccaaggcccaggacatactcggggccgtgaccctgctcctggaaggggtcatggcagctcggggccagctcgggcctacgtgcctgagctccctgctcgggcagctgtccgggcaggtccggctcctgctcggggccctgcagagcctgctcgggacccagggccggaccacggctcacaaggacccgaacgcgatcttcctgagcttccagcacctgctccggggcaaggtcaggttcctgatgctggtcggaggctcgaccctgtgcgtgcgcagggctcctccgaccacggccgtgccctcgcgcacgagcctggtcctgaccctgaacgagctcggg

SEQ ID NO: 84 - DNA, 513 bp, synthetic construct
  source 1..513: mol_type = other DNA
tctccggctccgcctgcctgcgacctgcgggtgctgtcgaagctgctgcgggacagccacgtcctccacagccgcctgagccagtgcccggaggtgcacccgctgcctacgccggtgctgctgccggccgtggacttcagcctcggggagtggaagacgcagatggaggagaccaaggcccaggacatcctcggggccgtgaccctgctcctggaaggggtgatggcagcgcgagggcagctggggcctacctgcctcagctccctgctggggcagctgtcggggcaggtgcggctgctgctcggggccctgcagtccctgctcgggacccagggccggaccacagcccacaaggaccccaacgccatcttcctctccttccagcacctgctccggggcaaggtccggttcctgatgctggtcggcggcagcaccctgtgcgtgagacgggctccgcctaccacggccgtgccctcgcgcacgagcctggtcctgaccctgaacgagctcggg

SEQ ID NO: 85 - DNA, 513 bp, synthetic construct
  source 1..513: mol_type = other DNA
agccccgcgccgccagcgtgcgatctgcgcgtgctgagcaagctgctgcgcgactcgcacgtgctgcactcgcggctctcgcagtgccccgaggtgcacccgctgcccacacccgtgctgctgcccgcggtggacttctcgctcggcgagtggaagacgcagatggaggagacgaaagcgcaggacatcctcggcgcggtgacgctgctgctcgaaggcgtgatggctgctcgcgggcagctcgggcctacgtgcctgagctcgctgctcgggcagctgagcgggcaggtgcggctgctgctcggcgcgctgcagtcgctgctcggcacgcaggggcgcaccacagcgcacaaggacccgaacgcgatcttcctgagcttccagcacctgctgcgcgggaaggtgcgcttcctgatgctcgtcggcggcagcacgctgtgcgtgcgcagagcgcctccgaccaccgcggtgccctcgcgcacctcgctcgtgctcacgctgaacgagctcggc

SEQ ID NO: 86 - DNA, 513 bp, synthetic construct
  source 1..513: mol_type = other DNA
agccccgctccgccagcctgcgacctgcgggtgctgagcaagctgctgcgggacagccacgtgctgcacagccggctgagccagtgccccgaggtgcacccgctgcccacgcccgtgctgctgcccgctgtggacttcagcctgggcgagtggaagacccagatggaggagaccaaggcccaggacatcctgggcgccgtgaccctgctgctggaaggggtgatggccgctaggggccagctggggcctacctgcctgagcagcctgctggggcagctgagcgggcaggtgcggctgctgctgggcgccctgcagagcctgctgggcacccaggggcgcaccacagcccacaaggaccccaacgccatcttcctgagcttccagcacctgctgcggggcaaggtgcgcttcctgatgctggtcggcggcagcaccctgtgcgtgcgcagggctccgcctaccaccgccgtgcccagccgcaccagcctggtgctgaccctgaacgagctcggc

SEQ ID NO: 87 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 681 bp, synthetic construct
  source 1..681: mol_type = other DNA
gacaagacacacacctgccccccatgtcccgcgcctgaactcctgggggggccctctgtcttcctgttcccaccgaagccgaaagacacccttatgatatctcggacccctgaggtaacttgtgtagtagttgatgtgtctcacgaagatccagaagtgaagtttaattggtatgtggatggagtggaagttcacaacgctaagaccaagcccagggaggaacaatacaattccacataccgagttgtttcagtgctcactgtgttgcatcaggactggctcaacggcaaggagtataaatgcaaagtcagtaacaaagccctgcccgcccccatcgagaaaaccatctcgaaggcaaaaggccagcctcgcgagccacaggtctacaccctgcctccctcacgtgacgagctgacaaagaatcaggtcagcttgacgtgtttagtgaaaggattttatccaagcgacattgccgtggaatgggagagcaatggccagcctgagaacaattacaaaacaactccacctgtgctcgacagtgatgggtctttcttcttatactccaaactaacggtcgataagagcagatggcagcaaggtaacgtgttttcctgctcagtgatgcatgaggctctgcataaccactatactcagaagtcccttagtctgagcccgggtaag

SEQ ID NO: 89 - DNA, 681 bp, synthetic construct
  source 1..681: mol_type = other DNA
gataagacccacacctgtccgccgtgccccgcacccgagcttctcggcggccccagcgtgttcctgtttccgccgaagcccaaggacaccctgatgatctcccggacgcccgaggtgacctgcgtggtggtggacgtgtcccacgaggaccccgaggtgaagttcaactggtacgtggacggggtcgaggtgcacaacgccaagaccaagccccgcgaggagcagtacaactccacctaccgggtcgtgtccgtgctgaccgtgctgcaccaggactggctgaacgggaaggagtacaagtgcaaggtgtccaacaaggccctgcccgcgcccatcgagaagaccatctccaaggccaaggggcagcccagggagccccaggtgtacaccctgcctccgtcccgggacgagctgaccaagaaccaggtgtccctgacctgcctggtgaaggggttctaccccagcgacatcgccgtggagtgggagtccaacgggcagcccgagaacaactacaagaccacgccgccggtgctggactccgacgggagcttcttcctgtactccaagctgaccgtggacaagtcccggtggcagcaggggaacgtgttcagctgcagcgtgatgcacgaggccctgcacaaccactacacccagaagtccctgagcctgagccccgggaag

SEQ ID NO: 90 - DNA, 681 bp, synthetic construct
  source 1..681: mol_type = other DNA
gacaagacgcacacgtgtccgccgtgccccgcacccgaactgctcggcggccccagcgtgttcctgtttccgccgaagcccaaggacacgctgatgatctcgcgcacgcccgaggtgacgtgcgtcgtcgtcgacgtgtcgcacgaggaccccgaggtgaagttcaactggtacgtcgacggcgtcgaggtgcacaacgccaagacgaagccgcgcgaggagcagtacaacagcacgtaccgcgtcgtgagcgtgctgaccgtgctgcaccaggactggctgaacggcaaggagtacaagtgcaaggtgagcaacaaggcgctgcccgcgccgatcgagaagacgatcagcaaggccaaggggcagccacgcgagccgcaggtgtacacgctgccgccgtcgcgcgacgagctgacgaagaaccaggtgtcgctgacgtgcctcgtgaagggcttctaccccagcgacatcgccgtcgagtgggagagcaacgggcagcccgagaacaactacaagacgacgccgccggtgctcgacagcgacggcagcttcttcctgtacagcaagctgaccgtcgacaagtcgcgctggcagcagggcaacgtgttcagctgcagcgtgatgcacgaggcgctgcacaaccactacacgcagaagtcgctgtcgctgtcgcccggcaag

SEQ ID NO: 91 - DNA, 681 bp, synthetic construct
  source 1..681: mol_type = other DNA
gacaagacgcacacctgtccgccgtgcccggctcccgaactgctcggcggcccgtccgtcttcctgtttccgccgaagccgaaggacaccctgatgatcagccggacgccggaggtgacctgcgtcgtcgtcgacgtcagccacgaggaccccgaggtcaagttcaactggtacgtcgacggcgtcgaggtccacaacgccaagacgaagccccgcgaggagcagtacaactcgacgtaccgggtcgtctccgtcctgaccgtcctccaccaggactggctgaacggcaaggagtacaagtgcaaggtcagcaacaaggccctgccggccccgatcgagaagacgatctcgaaggccaagggccagcctcgggagccccaggtctacacgctgccgccgtcccgggacgagctgaccaagaaccaggtcagcctgacctgcctggtcaagggcttctacccgtccgacatcgccgtcgagtgggagtcgaacggccagcccgagaacaactacaagacgacgccgccggtcctggactccgacggcagcttcttcctgtacagcaagctgaccgtcgacaagtcccggtggcagcagggcaacgtcttcagctgctccgtcatgcacgaggccctgcacaaccactacacccagaagtccctgtccctgtcgcccggcaag

SEQ ID NO: 92 - DNA, 681 bp, synthetic construct
  source 1..681: mol_type = other DNA
gacaagacccatacctgtccgccgtgcccggctccggaactgctcggcggcccgtccgtcttcctgtttccgccgaagccgaaggacaccctgatgatcagccggacgccggaggtgacctgcgtcgtcgtcgacgtcagccacgaggacccggaggtcaagttcaactggtacgtcgacggcgtcgaggtccacaacgccaagaccaagccccgggaagagcagtacaacagcacctaccgggtcgtgtccgtcctgaccgtcctgcaccaggactggctgaacggcaaggagtacaagtgcaaggtcagcaacaaggccctgccggccccgatcgagaagaccatcagcaaggccaagggccagcccagggagccgcaggtctacaccctgccgccgtcccgggacgagctgaccaagaaccaggtcagcctgacctgcctggtcaagggcttctacccgtccgacatcgccgtcgaatgggagtccaacggccagccggagaacaactacaagacgacgccgccggtcctggactccgacggcagcttcttcctgtacagcaagctgaccgtcgacaagtcccggtggcagcagggcaacgtcttcagctgctccgtcatgcacgaggccctgcacaaccactacacccagaagtccctgtccctgtcgcccggcaag

SEQ ID NO: 93 - DNA, 681 bp, synthetic construct
  source 1..681: mol_type = other DNA
gacaagacgcacacgtgtccgccgtgcccagccccggagcttctcggcggcccctcggtgttcctgtttccgccgaagcccaaggacacgctgatgatctcccggaccccggaggtgacctgcgtcgtcgtcgacgtgtcccacgaggaccccgaggtgaagttcaactggtacgtcgacggcgtcgaggtgcacaacgcgaagacgaagccccgcgaggagcagtacaactccacgtaccgcgtcgtctccgtgctcaccgtgctgcaccaggactggctcaacggcaaggagtacaagtgcaaggtgtccaacaaggcgctgcccgcgcccatcgagaagaccatctccaaggccaaggggcagccccgggaaccccaggtgtacacgctgccgccgagccgcgacgagctgaccaagaaccaggtgtccctgacctgcctcgtcaaggggttctacccctcggacatcgccgtggagtgggagtccaacgggcagcccgagaacaactacaagacgacgccgccggtgctcgactccgacgggtccttcttcctctactcgaagctcaccgtggacaagtcccggtggcagcagggcaacgtgttcagctgctccgtgatgcacgaggcgctgcacaaccactacacgcagaagtccctgtccctgagccccgggaag

SEQ ID NO: 94 - protein, 238 aa, synthetic construct
IQLVQSGAEVKKPGASVKVSCKASGYTFTRYTMHWVRQAPGQGLEWIGYINPSRGYTNYNQKFKDRATLTTDKSTSTAYMELSSLRSEDTAVYYCARYYDDHYCLDYWGQGTLVTVSSGGSGGSGGSGGSGGDIQLTQSPSSLSASVGDRVTITCRASSSVSYMNWYQQKPGKAPKRWIYDTSKVASGAPSRFTGSGSGTDYTLTISSLQPEDFATYYCQQWSSNPLTFGGGTKLEIK

SEQ ID NO: 95 - DNA, 714 bp, synthetic construct
  source 1..714: mol_type = other DNA
atccagctggtgcagtccggggccgaggtgaagaagcccggggcctccgtgaaggtgtcctgcaaggcctccgggtacaccttcacccggtacaccatgcactgggtgaggcaggctcccgggcaggggctggagtggatcgggtacatcaaccccagccgcgggtacaccaactacaaccagaagttcaaggaccgggccacgctgaccaccgacaagtccacctccaccgcctacatggagctgagctccctgcggtccgaggacaccgccgtgtactactgcgcccggtactacgacgaccactactgcctggactactggggccaggggaccctggtgaccgtgtcctccggcggctcaggcggctcaggaggctcaggcggctcaggcggcgacatccagctgacccagagccccagctccctgtccgcctccgtcggggaccgggtgaccatcacctgccgggcctcctcctccgtgtcctacatgaactggtaccagcagaagcccgggaaggcgcccaagcggtggatctacgacacctccaaggtggcctccggggctccctcacggttcaccgggtccgggtccgggaccgactacaccctgaccatcagctccctgcagcccgaggacttcgccacctactactgccagcagtggtcctccaacccgctgaccttcggcggcggcaccaagctggagatcaag

SEQ ID NO: 96 - DNA, 714 bp, synthetic construct
  source 1..714: mol_type = other DNA
atccagctcgtgcagagcggggccgaggtgaagaagcccggggcgagcgtgaaggtgtcgtgcaaggcgagcgggtacaccttcacgcgctacaccatgcactgggtgcgccaagctcccgggcaggggctggagtggatcgggtacatcaacccctcgcgcgggtacaccaactacaaccagaagttcaaggaccgcgccacgctcaccaccgacaagtccacctccaccgcctacatggagctctcctcgctgcgctccgaggacaccgcggtgtactactgcgcgcgctactacgacgaccactactgcctcgactactggggccaggggaccctcgtgaccgtgtcgagtggtggtagtggtggtagtggtggtagtggtggtagtggtggcgacatccagctcacccagagccccagctcgttgagcgcgagcgtcggggaccgcgtgaccatcacgtgccgcgcctcgtcgagcgtgagctacatgaactggtaccagcagaagcccgggaaggccccgaagaggtggatctacgacacctccaaggtggcgagcggggctccatcgcgcttcacggggagcgggagcgggaccgactacaccctcaccatctcctcgctccagcccgaggacttcgccacctactactgccagcagtggagctccaacccgctcaccttcggcggcggcaccaagttggagatcaag

SEQ ID NO: 97 - DNA, 714 bp, synthetic construct
  source 1..714: mol_type = other DNA
atccagctggtgcagagcggcgccgaggtgaagaagcccggggcctcggtgaaggtctcctgcaaggcctcggggtacaccttcacccggtacaccatgcactgggtgcgccaagccccagggcagggcctggagtggatcgggtacatcaaccccagccgcgggtacaccaactacaaccagaagttcaaggaccgggccacgctgaccacggacaagtccacgtccacggcgtacatggagctgagctccctgcgctccgaggacaccgcggtgtactactgcgcccggtactacgacgaccactactgcctggactactggggccaggggaccctggtcaccgtgtcctccggaggctccggaggctccggaggctccggaggctccggaggcgacatccagctgacccagagccccagctccctcagcgcctccgtcggggaccgggtgaccatcacctgccgcgcctcgtcctcggtgtcctacatgaactggtaccagcagaagcccgggaaggccccgaagcggtggatctacgacacgtccaaggtcgcctccggggctccgtcgaggttcaccggctccggctccgggacggactacaccctgaccatctcctcgctgcagcccgaggacttcgccacgtactactgccagcagtggtcctcgaatcccctgaccttcggcggcggcacgaagctggagattaag

SEQ ID NO: 98 - DNA, 714 bp, synthetic construct
  source 1..714: mol_type = other DNA
atccagctggtccagtccggggccgaggtgaagaagcccggggcctccgtgaaggtctcctgcaaggcctccgggtacaccttcacccggtacaccatgcactgggtccggcaggctccagggcaggggctggagtggatcgggtacatcaaccccagccgcgggtacaccaactacaaccagaagttcaaggaccgggccacgctgaccaccgacaagtccacctccaccgcctacatggagctgagctccctgcggtccgaggacaccgccgtctactactgcgcccggtactacgacgaccactactgcctggactactggggccaggggaccctggtgaccgtctcctccggaggctccggaggctccggaggctccggaggctccggaggcgacatccagctgacccagagccccagctccctctccgcctccgtcggggaccgggtgaccatcacctgccgggcctcctcctccgtctcctacatgaactggtaccagcagaagcccgggaaggccccgaagcggtggatctacgacacctccaaggtggcctccggggcccctagcaggttcaccgggtccgggtccgggaccgactacaccctgaccatctccagcctccagcccgaggacttcgccacctactactgccagcagtggtcctccaacccgctgaccttcggcggcggcaccaagctggagatcaag

SEQ ID NO: 99 - DNA, 714 bp, synthetic construct
  source 1..714: mol_type = other DNA
atccagctggtgcagtccggggccgaggtgaagaagcccggggcctccgtgaaggtgtcctgcaaggcctccgggtacaccttcacccggtacaccatgcactgggtgaggcaggctcccgggcaggggctggagtggatcgggtacatcaacccctcgcgcgggtacaccaactacaaccagaagttcaaggaccgggccacgctgaccaccgacaagagcacctccaccgcctacatggagctgtccagcctgcggtccgaggacaccgccgtgtactactgcgcccggtactacgacgaccactactgcctggactactggggccaggggaccctggtgaccgtgtccagcggcggctctggcggctctggaggctccggcggctctggaggcgacatccagctgacccagtccccgtccagcctgtccgccagcgtcggggaccgggtgaccatcacctgccgggcctcctccagcgtgtcctacatgaactggtaccagcagaagcccgggaaggcgcccaagcggtggatctacgacacctccaaggtggcctccggggctcccagccgtttcaccgggtccgggtccgggaccgactacaccctgaccatctccagcctgcagcccgaggacttcgccacctactactgccagcagtggtccagcaacccgctgaccttcggcggcggcaccaagctggagatcaag

SEQ ID NO: 100 - DNA, 714 bp, synthetic construct
  source 1..714: mol_type = other DNA
atccagctcgtccagtccggggccgaggtgaagaagcccggggcgtccgtgaaggtgtcctgcaaggcgtccgggtacacgttcacccggtacacgatgcactgggtccgtcaggctcccgggcaggggctggagtggatcgggtacatcaacccgtcccgcgggtacacgaactacaaccagaagttcaaggaccgggcgaccctgacgaccgacaagtccacgtccaccgcgtacatggagctgtcgtccctgcggtccgaggacaccgccgtgtactactgcgcccggtactacgacgaccactactgcctcgactactggggccaggggaccctcgtgaccgtgtcctccggaggttccggcggctccggaggatctggcggctccggcggcgacatccagctgacccagtcgccctcgtccctgtccgcgtccgtcggggaccgggtgacgatcacgtgccgggcgtcctcctccgtgtcctacatgaactggtaccagcagaagcccgggaaggccccgaagaggtggatctacgacacgtccaaggtcgcgtccggggctccatcccggttcaccgggtccgggtccgggaccgactacaccctgacgatctcctcgctccagcccgaggacttcgcgacgtactactgccagcagtggtcctcgaatcccctgacgttcggcggcggcacgaagctggagatcaag

SEQ ID NO: 101 - protein, 244 aa, synthetic construct
QVQLQQSGPGLVKPSQTLSLTCAISGDSVSSNRAAWNWIRQSPSKGLEWLGRTYYRSKWYNEYAASVKSRMSINPDTSKNQFSLQLNSVTPEDTALYYCARDLWELREACDIWGQGTMVTVSSGGSGGSGGSGGSGGDIVMTQSPSFLSTFVGDRVTITCRASQGISSYLAWYQQKPGKVPKLLIYVASTLQSGVPSRFSGSGSGTEFTLTISSLQPEDFATYYCQQLNSYPFTFGPGTKVDIK

SEQ ID NO: 102 - DNA, 732 bp, synthetic construct
  source 1..732: mol_type = other DNA
caggtgcagctgcagcagtccgggcccgggctggtgaagcccagccagaccctgagcctgacctgcgccatcagcggggactccgtcagctccaaccgggccgcctggaactggatcaggcagtccccgtccaaggggctggagtggctcggccggacctactaccgctccaagtggtacaacgagtacgccgcctccgtgaagtcccggatgagcatcaaccccgatacctccaagaaccagttcagcctgcagctgaactccgtgacgcccgaggatacggccctgtactactgcgccagggacctgtgggagctgcgcgaggcctgcgacatctggggccaggggaccatggtgaccgtgtcctccggcggctcaggcggctcaggtggctccggcggatcaggcggcgacatcgtgatgacccagtccccatccttcctgtccaccttcgtcggggaccgggtgaccatcacctgccgggccagccaggggatcagctcctacctggcctggtaccagcagaagcccgggaaggtgcccaagctgctgatctacgtggcctcaaccctgcagtccggggtgcccagccggttcagcgggtccgggtccgggaccgagttcaccctgaccatcagctccctgcagcccgaggacttcgccacctactactgccagcagctgaactcctaccccttcaccttcgggcccgggaccaaggtggacatcaag

SEQ ID NO: 103 - DNA, 732 bp, synthetic construct
  source 1..732: mol_type = other DNA
caggtgcagctgcagcagtccgggcccgggctggtgaagccttcccagaccctctccctcacctgcgccatctccggggattccgtgtcctccaaccgcgccgcctggaactggatcaggcagtccccttccaaggggctggagtggctggggcgcacgtactaccgctccaagtggtacaacgagtacgccgcttccgtgaagtcccgcatgagcatcaaccccgatacctccaagaaccagttctccctgcagctgaactccgtgaccccggaggataccgcgctgtactactgcgcccgggacctgtgggagctgcgggaagcctgcgacatctggggccaggggaccatggtgaccgtgtcctccggaggctccggaggctccggaggttccggaggctccggcggcgacatcgtgatgacgcagtccccttccttcctgagcaccttcgtaggggaccgcgttaccatcacctgcagggcttcccaggggatctcctcctacctcgcatggtaccagcagaagcccgggaaggttcccaagctgctgatctacgtggcttccacgctgcagtccggggtgccttcccggttctccgggagcgggagcgggacggagttcaccctcaccatcagctccctgcagcccgaggacttcgcaacgtactactgccagcagctgaactcctaccccttcaccttcgggcccgggacgaaggtggacatcaag

SEQ ID NO: 104 - DNA, 732 bp, synthetic construct
  source 1..732: mol_type = other DNA
caagtgcagctgcagcagagcgggcccgggctggtgaagccgtcgcagacgctgtcgctgacgtgcgccatcagcggcgacagcgtgagcagcaaccgcgccgcgtggaactggatcaggcagtcgcccagcaaggggctggagtggctggggcgcacgtactaccgcagcaagtggtacaacgagtacgccgccagcgtgaagagccgcatgagcatcaaccccgacaccagcaagaaccagttctcgctgcagctgaacagcgtgacgcccgaggacaccgcgctgtactactgcgcgcgcgacttgtgggagctgcgcgaggcgtgcgacatctggggccagggcaccatggtgaccgtgagcagcggcggcagtggtggcagcggtggtagcggcggtagcggcggcgacatcgtgatgacgcagtcgccgtcgttcttgagcacgttcgtgggcgaccgcgtgaccatcacgtgccgcgcgtcgcagggcatcagcagctacttggcgtggtaccagcagaagcccggcaaggtgcccaagctgctgatctacgtggccagcacgctgcagagcggcgtgccgtcgcgcttcagcggcagcggcagcggcaccgagttcacgctgaccatcagcagcctgcagcccgaggacttcgccacgtactactgccagcagctgaacagctacccgttcacgttcgggcccggcaccaaggtggacatcaag

SEQ ID NO: 105 - DNA, 732 bp, synthetic construct
  source 1..732: mol_type = other DNA
caggtgcagctgcagcagagcggccccgggctcgtgaagccgtcgcagaccctgagcctcacctgcgccatctccggggactccgtgtcctcgaaccgcgccgcgtggaactggattcggcagagccccagcaagggcctggagtggctggggcgcacctactaccgctccaagtggtacaacgagtacgccgcctcggtgaagtcccggatgagcatcaaccccgacacctcgaagaaccagttctcgctgcagctgaactccgtgaccccggaggacacggcgctgtactactgcgcccgggacctctgggagctccgcgaggcctgcgacatctggggccaggggaccatggtcaccgtgtcctccggaggctccggaggctccggaggctccggaggctccggaggcgacatcgtgatgacccagagccccagcttcctgagcaccttcgtcggggaccgggtcaccatcacgtgccgcgcgtcccaggggatctcctcgtacctggcctggtaccagcagaagcccgggaaggtgccgaagctgctgatctacgtggcctcgacgctgcagtccggggtcccgagccgcttcagcggctccgggtccgggaccgagttcaccctgaccatctcgtcgctgcagcccgaggacttcgccacgtactactgccagcagctgaacagctaccccttcaccttcggccccgggaccaaggtggacatcaag

SEQ ID NO: 106 - DNA, 732 bp, synthetic construct
  source 1..732: mol_type = other DNA
caggtgcagctgcagcagagcgggcccgggctggtgaagcccagccagaccctgagcctgacctgcgccatctccggggacagcgtgagcagcaaccgggccgcctggaactggatcaggcagagccccagcaaggggctggagtggctggggcgcacatactaccgctccaagtggtacaatgagtatgccgccagcgtgaagagccgcatgagcatcaaccccgacacctccaagaaccagttcagcctgcagctgaacagcgtgacgcccgaggacactgccctgtactactgcgccagggacctgtgggagctgcgcgaggcctgcgacatctggggccagggcaccatggtgacagtgtcctccggcggctcaggaggctccggaggctctggcggctcaggcggcgacatcgtgatgacccagagccccagcttcctgagcaccttcgtgggcgacagggtgaccatcacctgcagggccagccagggcatcagcagctacctggcctggtaccagcagaagcccggcaaggtgcccaagctgctgatctatgtggccagcaccctgcagagcggggtgcccagccgcttcagcggcagcggcagcggcacagagttcaccctgaccatcagcagcctgcagcccgaggacttcgccacatactactgccagcagctgaacagctaccccttcaccttcgggcccggcaccaaggtggacatcaag

SEQ ID NO: 107 - DNA, 732 bp, synthetic construct
  source 1..732: mol_type = other DNA
caggtgcagctgcagcagagcgggcccgggctcgtgaagccctcgcagaccctctccctcacgtgcgcgatctccggggactccgtgtcctccaaccgcgccgcgtggaactggatacggcagagcccctcgaaggggctggagtggctggggcgcacgtactaccgctccaagtggtacaacgagtacgccgcctccgtgaagtcccgcatgagcatcaaccccgacacctccaagaaccagttctccctgcagctgaactccgtgactcccgaggacaccgcgctgtactactgcgcgcgggacctgtgggagctgcgcgaggcgtgcgacatctggggacaggggaccatggtgaccgtgtcctccggaggaagcggaggaagcggaggaagcggaggaagcggaggagacatcgtgatgacgcagtccccttccttcctctccaccttcgtgggagaccgcgtgaccatcacgtgccgcgcttcccaggggatctcctcctacctcgcgtggtaccagcagaagcccgggaaggtgcccaagctcctcatctacgtggcctccacgctgcagagcggggtgccctcgcgcttctccgggagcgggagcgggacggagttcaccctcaccatctcttccctgcagcccgaggacttcgccacgtactactgccagcagctgaactcctaccccttcaccttcgggcccgggacgaaggtggacatcaag

SEQ ID NO: 108 - protein, 248 aa, synthetic construct
EVQLLESGGGLVKPGGSLRLSCAASRFTLSSYDMNWVRQAPGKGLEWVSSISSSSSYIYYADSVKGRFTISRDNAKNSLYLQMNSLRAEDTAVYYCARDRGVGGTDYYYYGLDVWGQGTTVTVSSGGSGGSGGSGGSGGEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPPYTFGQGTKVEIK

SEQ ID NO: 109 - DNA, 744 bp, synthetic construct
  source 1..744: mol_type = other DNA
gaggtgcagctgctggagagcggcggcggcctggtgaaacccggcggctccctgcggctgtcctgcgccgcctccaggttcaccctgtccagctacgacatgaactgggtgaggcaggctcccgggaaggggctggagtgggtgtcctccatctcctccagctccagctacatctactacgccgattccgtgaaggggagattcaccatctccagggacaacgccaagaactccctgtacctgcagatgaactccctgcgggccgaggacaccgccgtgtactactgcgccagggacaggggcgttggcggcaccgactactactactacgggctggacgtgtggggccaggggaccaccgtgaccgtgtccagcggcggctctggcggctctggaggctctggcggctctggcggcgagatcgtgatgacccagtctcccgggaccctgtccctgtctcccggggagagggctaccctgtcctgcagggccagccagtccgtgtcctccagctacctggcctggtaccagcagaaacccgggcaggctccccggctgctgatctacggggcctcttccagggccaccggcatccccgacaggttctccgggtccgggtccgggaccgacttcaccctgaccatctccaggctggagcccgaggacttcgccgtgtactactgccagcagtacgggtcctctccaccttacaccttcgggcaggggaccaaggtggagatcaag

SEQ ID NO: 110 - DNA, 744 bp, synthetic construct
  source 1..744: mol_type = other DNA
gaggtccagctcctggagagcggaggaggcctggtgaagcccggaggctccctgaggctctcctgcgccgcctccaggttcaccctctcctcctacgacatgaactgggtgaggcaggccccggggaaggggctggagtgggtctcctccatctcctcctcctcctcctacatctactacgccgactccgtgaaggggcgcttcaccatctcccgggacaacgccaagaactccctctacctccagatgaactccctgagggccgaggacacggccgtctactactgcgcccgggaccgaggggtaggaggcaccgactactactactacgggctggacgtctggggccaggggaccaccgtgaccgtctcctccggaggctccggaggctccggaggctccggaggctccggaggagagatcgtgatgacccagagccccgggaccctctccctgagccccggggagagggctaccctctcctgccgggccagccagagcgtctcctcctcctacctggcctggtaccagcagaagccggggcaggcccctaggctcctgatctacggggcctcctctagggccaccggcatcccggaccgcttctccgggtccgggtccgggaccgacttcaccctcaccatctcccggctggagccggaggacttcgccgtctactactgccagcagtacgggtcctctcctccttacaccttcgggcaggggaccaaggtggagatcaag

SEQ ID NO: 111 - DNA, 744 bp, synthetic construct
  source 1..744: mol_type = other DNA
gaggtgcagctgctggagtccggcggcggcctggtgaaacccggcggcagcctgcggctgtcctgcgcggcaagccgcttcacgctgtccagctacgacatgaactgggtgcgccaggcacccggcaaggggctggagtgggtgtccagcatatccagctcgtcaagctacatatactacgcggacagcgtgaagggccggtttaccatctcgcgggataacgccaagaacagcctgtacctgcagatgaacagcctgcgggccgaggacaccgcggtgtactactgcgcaagggaccgcggggtaggcggcacggattactactactacgggctggacgtgtggggccaggggaccaccgtgacggtgtcctccggcggctcaggcggttccggtggctctggcggctcaggcggcgagattgtcatgacgcagtcacccggcacgcttagcctgtcgcccggggaacgcgccacgctgtcctgccgggccagccagtcggtgtccagcagctacctggcgtggtaccagcagaaacccggccaggcgccccggctgcttatctacggggcgtctagccgggcaaccggcatcccggaccgcttcagcgggtcgggcagcgggacggacttcacgctgacaatcagccggctggagcccgaggacttcgcggtgtactactgccagcagtacggcagctcgccgccttacacgtttggccagggcaccaaggtggaaatcaag

SEQ ID NO: 112 - DNA, 744 bp, synthetic construct
  source 1..744: mol_type = other DNA
gaggtccagctgctggagagcggcggcggcctggtcaaacccggcggctcgctgcggctgagctgcgccgccagcaggttcaccctgagctcctacgacatgaactgggtgcgccaggccccaggcaaggggctggagtgggtgagctcgatcagctcgtcgtcgagctacatctactacgccgacagcgtcaaggggcgcttcaccatctcgcgcgacaacgccaagaactcgctctacctccagatgaactcgctgcgggccgaggacaccgccgtctactactgcgcccgagatcgcggggttggcggcaccgactactactactacgggctcgacgtgtggggccaggggaccaccgtgaccgtgagctccggcggctctggcggctcaggtggtagcggcggctctggcggcgagatcgtgatgacccagagccccgggaccttgtcgctgagccccggcgagagggccacgctgagctgccgggccagccagagcgtgagctccagctacctggcctggtaccagcagaaacccggccaggcgccccggctgttgatctacggggcctcatctcgggccaccggcatccccgacaggttctcggggtcggggtcggggaccgacttcaccctgaccatctcgcggctggagcccgaggacttcgccgtctactactgccagcagtacgggagctcgccgccgtacaccttcggccaggggaccaaggtcgagatcaag

SEQ ID NO: 113 - DNA, 744 bp, synthetic construct
  source 1..744: mol_type = other DNA
gaggtgcagctgctggagtccggcggcggcctggtgaaacccggcggctccctgcggctgtcctgcgccgccagcaggttcaccctctcctcctacgacatgaactgggtgcggcaggccccagggaagggcctggagtgggtgagcagcatcagcagcagcagcagctacatctactacgccgacagcgtcaaggggcgcttcaccatcagcagggacaacgccaagaacagcctgtacctgcagatgaacagcctccgggccgaggacacggccgtgtactactgcgcccgtgatcgtggcgtcggcggcaccgactactactactacgggctggacgtgtggggccaggggacgaccgtgaccgtctcctccggcggctccggaggctctggtggttctggcggctccggcggcgagatcgtcatgacccagagccccgggaccctgtccctgagccccggggagagggccacgctctcctgccgggccagccagtccgtgtcctcctcctacctggcctggtaccagcagaaacccggccaggcgcccaggctgctgatctacggggcctcttctcgcgccacgggcatccccgaccgcttctccgggagcggctccgggacggacttcaccctgaccatcagccgcctggagcccgaggacttcgccgtgtactactgccagcagtacgggtcctcgccgccgtacaccttcgggcaggggacgaaggtggagatcaag

SEQ ID NO: 114 - DNA, 744 bp, synthetic construct
  source 1..744: mol_type = other DNA
gaggtgcagctgctggagagcggcggcggcctcgtgaaacccggcggctcgctgcggctctcgtgcgccgcttcgcgcttcacgctctcgtcgtacgacatgaactgggtgcggcaggctcccgggaaggggctggagtgggtctcgtcgatctcgtcgtcgtcgagctacatctactacgccgactccgtgaaggggcgcttcacgatctcgcgcgacaacgcgaagaactcgctctacctgcagatgaactcgctgcgcgccgaggacaccgccgtctactactgcgctcgcgatcgcggagtcggcggcaccgactactactactacgggctcgacgtctggggccaggggacgaccgtgaccgtgtcgagcggcggaagcggcggaagcggaggaagcggcggaagcggcggcgagatcgtgatgacgcagtcgcccgggacgctctcgctctcgcccggcgagcgcgctacgctctcgtgccgcgcgtcgcagagcgtctcgtcgagctacctcgcgtggtaccagcagaaacccgggcaggcgccgcggctgcttatctacggcgctagctctcgcgcgaccggcatccccgaccgcttctccggctccggctccgggaccgacttcacgctgacgatctcgcggctggagcccgaggacttcgccgtctactactgccagcagtacggctcgtcgccgccgtacacgttcgggcaggggacgaaggtcgagatcaag

SEQ ID NO: 115 - protein, 234 aa, synthetic construct
DVQLVESGAELVRPGASKLSCKALAYTFTDYEMHWVKQTPVHGLEWIGIIHPGSGGTVYNQKFKGKATLTADKSSSTAYMELSSLTSEDSTVYYCTRRRYYGSSYNWYFDVWGAGNGGSGGSGGSGGSGGVLTQSPASLAASVGETVTITCRASENIYYSLAWYQQKQGKSPQLLIYNANSLEDGVPSRFSGSGSGTQYSLKINSMQPEDTATYFCEQTYDVPLTFGAGTKLEL

SEQ ID NO: 116 - DNA, 702 bp, synthetic construct
  source 1..702: mol_type = other DNA
gatgtccagctggtggagtctggggctgagctggtgaggcctggggccagcaagctgtcctgcaaggccctggcctacaccttcacagactatgagatgcactgggtgaagcagaccccagtgcatgggctggagtggattgggatcatccatccaggctctggtggcacagtctacaaccagaagttcaaggggaaggccacactcacagctgacaagtccagctccacagcctacatggagctgtccagcctgacctctgaggactccacagtctactactgcaccaggaggaggtactatggctccagctacaactggtactttgatgtgtggggagctgggaatggtggctctggtggctctggtggctctggtggctctggtggagtgctgacccagtccccagccagcctggctgcctctgttggggagacagtcaccatcacctgcagggcctctgagaacatctactacagcctggcctggtaccagcagaagcaggggaagtctccccagctgctgatctacaatgccaacagcctggaggatggggtgcccagcaggttctctgggtctgggtctggcacccagtactccctcaagatcaacagcatgcagccagaggacacagccacctacttctgtgagcagacctatgatgtgcccctcacctttggggctggcaccaagctggagctc

SEQ ID NO: 117 - DNA, 702 bp, synthetic construct
  source 1..702: mol_type = other DNA
gacgtgcagctggtggagtccggggctgagctggtgaggcccggggcctccaagctgagctgcaaggccctggcctacaccttcaccgactacgagatgcactgggtgaagcagaccccggtgcacgggctggagtggatcgggatcatccatcccgggtccggcggcaccgtgtacaaccagaagttcaaggggaaggccacgctgaccgccgacaagtccagctccaccgcctacatggagctgtccagcctgacctccgaggactccaccgtgtactactgcacccggcggcggtactacgggtcctcctacaactggtacttcgacgtgtggggcgccgggaacggcggatcaggcggctccggaggctcaggaggctccggcggcgtgctgacccagagccccgctagcctggccgcctccgtcggggagaccgtgaccatcacctgccgggcctccgagaacatctactactccctggcctggtaccagcagaagcaggggaagtctccccagctgctgatctacaacgccaactccctggaggacggggtgcccagccggttctccgggtccgggtccgggacccagtacagcctgaagatcaacagcatgcagcccgaggacaccgccacctacttctgcgagcagacctacgacgtgcccctgaccttcggggccgggaccaagctggagctg

SEQ ID NO: 118 - DNA, 702 bp, synthetic construct
  source 1..702: mol_type = other DNA
gatgtccagctcgtggagtccggggctgagctcgtgaggcccggggcctcgaagctgagctgcaaggccctggcctacaccttcacggactacgagatgcactgggtgaagcagaccccggtgcacggcctggagtggatcgggatcattcaccccgggtccggcggcaccgtgtacaaccagaagttcaagggcaaggccacgctgaccgcggacaagtcctcgtccacggcgtacatggagctgagctccctgacctccgaggactccacggtgtactactgcacacggcggcggtactacgggagctcctacaactggtacttcgacgtctggggcgccgggaacggaggctctggaggctccggaggctctggaggctccggcggcgtcctgacccagagccccgcatccctggcggcctccgtcggggagaccgtgaccatcacctgcagggcctcggagaacatctactactccctggcgtggtaccagcagaagcaggggaagtccccgcagctcctgatctacaacgccaactccctggaggacggggtgccctcgcggttctcggggtccgggtccgggacccagtactccctgaagatcaactccatgcagcccgaggacaccgcgacgtacttctgcgagcagacctacgacgtgcccctgaccttcggcgccgggacaaagctggagctc

SEQ ID NO: 119 - DNA, 702 bp, synthetic construct
  source 1..702: mol_type = other DNA
gatgtgcagctggtggagtccggggctgagctggtgaggcccggggcctccaagctgtcctgcaaggccctggcctacaccttcaccgactacgagatgcactgggtgaagcagacgcccgtgcacgggctggagtggatcgggatcattcaccccgggtccggcggcaccgtgtacaaccagaagttcaaggggaaggccacgctgaccgccgacaagtcctcctccaccgcctacatggagctgagctccctgacctccgaggactccaccgtgtactactgcacccggcggcggtactacgggtcctcctacaactggtacttcgacgtgtggggcgccgggaacggcggatctggcggctccggcggatctggcggctctggcggcgtgctgacccagagccccgcttccctggccgcctccgtcggggagaccgtgaccatcacctgccgggcctccgagaacatctactactccctggcctggtaccagcagaagcaggggaagtctccccagctgctgatctacaacgccaactccctggaggacggggtgcccagccggttctccgggtccgggtccgggacccagtactccctgaagatcaactccatgcagcccgaggacaccgccacctacttctgcgagcagacctacgacgtgcccctgaccttcggggccgggaccaagctggagctg

SEQ ID NO: 120 - DNA, 702 bp, synthetic construct
  source 1..702: mol_type = other DNA
gacgtgcagctggtggagtccggggccgagctggtacggccaggcgccagcaagctgtcctgcaaggcgctcgcctacaccttcaccgactacgagatgcactgggtgaagcagacgcccgtgcacgggctggagtggatcgggatcatacaccccgggtccggcggcaccgtctacaaccagaagttcaaggggaaggctacccttacggccgacaagtcctcctctaccgcctacatggagctgtccagcctgacgagcgaggactctaccgtctactactgcacccgcaggcggtactacgggtccagctacaactggtacttcgacgtgtggggcgccgggaacggcggtagtggcggtagcggcggtagtggcggtagcggcggcgtgctgacccagtcgcccgctagcctggccgctagcgtcggggagaccgtcaccatcacctgccgggcgagcgagaacatctactactcgctcgcctggtaccagcagaagcaggggaagagcccgcagctgctgatctataacgccaacagcctggaggacggggtgcccagccggttcagcgggagcgggagcgggacccagtactccctcaagatcaacagcatgcagccggaggataccgccacctacttctgcgagcagacgtacgacgtgcccctcacgttcggggccggcaccaagctggagctg

SEQ ID NO: 121 - DNA, 702 bp, synthetic construct
  source 1..702: mol_type = other DNA
gacgtgcagctcgtggagagcggcgcagagcttgtgcgccccggggcctccaagctgtcctgcaaggcgctcgcctacaccttcacggactacgagatgcactgggtgaagcagacccctgtgcacggcctggagtggatcgggatcattcaccccgggtcaggaggcaccgtgtacaaccagaagttcaaggggaaggccacgctgaccgccgacaagtcctcctccacggcctacatggagctctcctcgctgacctccgaggactccacggtgtactactgcacgcggaggaggtactacgggtcctcctacaactggtacttcgacgtgtggggcgcggggaatggcggctcaggaggctcaggaggctcaggaggctcaggcggcgtgctcacgcagagccccgcaagcctcgcggcgtccgtcggggagaccgtgacgatcacctgcagggcctccgagaacatctactactccctcgcgtggtaccagcagaagcaggggaagtcccctcagctcctcatctacaacgcgaactccctggaggacggggtgccctcaaggttctccgggtcggggagcggcacgcagtactccctgaagatcaactccatgcagcccgaggacaccgcgacgtacttctgcgagcagacgtacgacgtgcccctcaccttcggggccggcacgaagctcgaactg

SEQ ID NO: 122 - protein, 233 aa, synthetic construct
LSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAGKMCHLQRAVRPLPEATSAVIIHPWGPCLLPTWKDIPRLSITKYQVKTLNALLRVRLSHLLR

SEQ ID NO: 123 - DNA, 699 bp, synthetic construct
  source 1..699: mol_type = other DNA
ctgagcatcactacccctgaggagatgattgagaaggctaagggtgagacagcctacctgccctgcaagttcaccctgagccctgaggaccaggggcccctggacattgagtggctgatcagcccagctgacaaccagaaggtggaccaggtcatcatcctgtactcaggggacaagatctatgatgactactaccctgacctgaagggcagggtgcacttcaccagcaatgacctgaagtcaggggatgccagcatcaatgtgaccaacctgcagctgtctgacataggcacctaccagtgcaaggtcaagaaggccccaggggtagccaacaagaagatccacctggtggtgctggtcaagccctcaggggccaggtgctatgtggatggctctgaggagataggctctgacttcaagatcaagtgtgagcccaaagagggcagcctgcccctgcagtatgagtggcagaagctgtctgacagccagaagatgcccactagctggctggctggcaagatgtgccacctgcagagggctgtcaggcccctgcctgaggccacctcagctgtcatcattcacccctggggcccctgcctgctgcctacctggaaggacatccccaggctgagcatcactaagtaccaggtcaagaccctcaatgccctgctgagggtcaggctgagccacctgctgagg

SEQ ID NO: 124 - DNA, 699 bp, synthetic construct
  source 1..699: mol_type = other DNA
ctgagcatcaccacgcccgaggagatgatcgagaaggccaagggcgagaccgcctacctgccctgcaagttcaccctgagccccgaggaccaggggcccctggacatcgagtggctgatctcgcccgcggacaaccagaaggtggaccaggtgatcatcctgtactccggggacaagatctacgacgactactaccccgacctgaaggggcgcgtgcacttcacctccaacgacctgaagtccggggacgccagcatcaacgtgaccaacctgcagctgtccgacatcgggacctaccagtgcaaggtgaagaaggcgcccggggtcgccaacaagaagatccacctggtggtgctggtgaagcccagcggggccaggtgctacgtggacgggagcgaggagatcgggtccgacttcaagatcaagtgcgagcccaaagaggggtccctgcccctgcagtacgagtggcagaagctgtccgacagccagaagatgccgacctcctggctggccgggaagatgtgccacctgcagcgggccgtgaggcccctgcctgaggccaccagcgccgtgatcattcacccctggggcccctgcctgctgccaacatggaaggacatcccgcggctgtccatcaccaagtaccaggtgaagaccctgaacgccctgctgcgggtgcggctgtcgcacctgctgcgg

SEQ ID NO: 125 - DNA, 699 bp, synthetic construct
  source 1..699: mol_type = other DNA
ctctcgatcaccacacccgaggagatgatcgagaaggcgaagggcgagaccgcctacttgccctgcaagttcaccctctcgcccgaggaccaggggcccctcgatatcgagtggctcatctcgcccgcggacaaccaaaaggtggaccaggtgatcatcctctatagtggggacaagatctacgacgactactaccccgatctcaaggggcgcgtccacttcacctccaacgacctcaagagcggggacgcctcgatcaacgtgaccaacctccaattgagcgacatcgggacctaccagtgcaaggtgaagaaggcgcccggggtcgccaacaagaagatccacctcgtggtgctcgtgaagcccagtggggcgcggtgctacgtggatgggtcggaggagatcgggagcgacttcaagatcaagtgcgagcccaaagagggctcgctacccctacaatacgagtggcagaagttgagcgactcccaaaagatgcccactagctggctcgcgggcaagatgtgccacctccagcgcgccgtgcgacccctacccgaggccacgagcgcggtcatcatacacccctggggcccctgcctactccccacttggaaggacatcccgcgcctctcgatcaccaagtaccaggtgaagacgctcaacgcgctcttgcgcgtgcgcctctcgcacctcttgagg

SEQ ID NO: 126 - DNA, 699 bp, synthetic construct
  source 1..699: mol_type = other DNA
ctgagcatcaccacgccggaggagatgatcgagaaggcgaagggcgagaccgcgtacctgccctgcaagttcaccctgagcccggaggaccaggggccgctggacatcgagtggctgatcagccccgcggacaaccagaaggtggaccaggtgatcatcctgtacagcggggacaagatctacgacgactactacccggacctgaaggggcgcgtgcacttcacctccaacgacctgaagagcggggacgccagcatcaacgtgaccaacctgcagctgagcgacatcgggacctaccagtgcaaggtgaagaaggcgcccggggtcgcgaacaagaagatccacctggtggtgctggtgaagcccagcggggcccggtgctacgtggacgggtccgaggagatcgggtccgacttcaagatcaagtgcgagccgaaagaggggtccctgccgctgcagtacgagtggcagaagctgagcgacagccagaagatgccgaccagctggctggccgggaagatgtgccacctgcagcgggctgtccggccgctccctgaggctacctccgcggtgatcatccatccctggggcccctgcctgctcccgacctggaaggacatcccgcggctgagcatcaccaagtaccaggtgaagaccctgaacgcgctgctccgggtccggctgagccacctgctccgg

SEQ ID NO: 127 - DNA, 699 bp, synthetic construct
  source 1..699: mol_type = other DNA
ctctcgatcaccacgcccgaggagatgatcgagaaggccaagggcgagaccgcctacttgccctgcaagttcaccctctcgcccgaggaccaggggcccctcgacatcgagtggctcatctcgcccgcggacaaccagaaggtggaccaggtgatcatcctctactcgggcgacaagatctacgacgactactaccccgacctcaaggggcgcgtgcacttcacgagcaacgacctcaagagcggggacgcctcgatcaacgtgaccaacctccagctctccgacatcgggacctaccagtgcaaggtgaagaaggcgcccggggtcgccaacaagaagatccacctcgtggtgttggtgaagcccagcggggcgcggtgctacgtggacgggagcgaggagatcgggagcgacttcaagatcaagtgcgagcccaaagaggggtcgctcccgctccagtacgagtggcagaagctgagcgactcccagaagatgcccacgagctggctcgcggggaagatgtgccacctccagcgcgcggttcgcccactccccgaggccacgagcgcggtgatcattcacccatggggcccctgcctcttgcccacgtggaaggacatcccgcgcctctcgatcaccaagtaccaggtgaagaccctcaacgcgctcctgcgcgtgcgcctctcccacttgttgagg

SEQ ID NO: 128 - DNA, 699 bp, synthetic construct
  source 1..699: mol_type = other DNA
ctgagcatcacgacgcccgaggagatgatcgagaaggccaaaggggagaccgcctacctgccctgcaagttcaccctgagccccgaggaccaggggcccctggacatcgagtggctgatctccccggcggacaaccagaaggtcgaccaggtgatcatcctgtactccggggacaagatctacgacgactactaccccgacctgaagggccgggtgcacttcacgtcgaacgacctgaagtccggggacgcctcgatcaacgtgaccaacctgcagctgtccgacatcgggacctaccagtgcaaggtgaagaaggcgcccggcgtggccaacaagaagatccacctggtggtcctcgtgaagcccagcggggcgcggtgctacgtggacggcagcgaggagatcgggtcggacttcaagatcaagtgcgagcccaaagagggcagcctgcccctgcagtacgagtggcagaagctgtccgacagccagaagatgccgacctcctggctggccggcaagatgtgccacctgcagcgcgccgtgaggccactccccgaggcgaccagcgcggtgatcattcacccctggggcccctgcctgctgccaacctggaaggacatcccgcggctgtccatcacgaagtaccaggtgaagaccctgaacgccctgctgcgcgtccggctgagccacctgctgcgc

SEQ ID NO: 129 - protein, 240 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 130 - DNA, 720 bp, synthetic construct
  source 1..720: mol_type = other DNA
gagatccagctggtgcagtctgggggggggctggtgaagcctggggggtcagtgaggatcagctgtgctgcctcagggtacaccttcaccaactatgggatgaactgggtgaggcaggccccagggaaggggctggagtggatggggtggatcaacacccacactggggagcccacctatgctgacagcttcaaggggaggttcaccttcagcctggatgactccaagaacacagcctacctgcagatcaactccctgagggctgaggacacagctgtgtacttctgcaccaggagggggtatgactggtactttgatgtgtgggggcaggggaccacagtgacagtgtcctctggggggggggggtctggggggggggggtctggggggggggggtctgacatccagatgacccagtccccctccagcctgtctgcctcagtgggggacagggtgaccatcacctgcagggcctcccaggacatcaactcctacctgtcctggttccagcagaagcctgggaaggcccccaagaccctgatctacagggccaacaggctggagtctggggtgccctccaggttctcagggtctgggtctgggacagactacaccctgaccatctccagcctgcagtatgaggactttgggatctactactgccagcagtatgatgagtccccctggacctttgggggggggaccaagctggagatcaag

SEQ ID NO: 131 - DNA, 720 bp, synthetic construct
  source 1..720: mol_type = other DNA
gagatccagctggtgcagtccggcggcggcctggtgaaacccggcggctccgtgcggatctcctgcgccgcctccgggtacaccttcaccaactacgggatgaactgggtgcggcaggctcccgggaaggggctggagtggatgggctggatcaacacccacaccggggagcccacatacgccgactccttcaaggggcgcttcaccttctccctggacgactccaagaacaccgcctacctgcagatcaactccctgcgggccgaggacaccgccgtgtacttctgcacccggcgcgggtacgactggtacttcgacgtgtggggccaggggaccaccgtgaccgtgtcctctggcggcggaggttccggcggaggaggctccggaggaggcggcagcgacatccagatgacccagagccccagcagcctgtccgcctccgtcggggaccgggtgaccatcacctgccgggccagccaggacatcaacagctacctgtcctggttccagcagaagcccgggaaggctcccaagaccctgatctaccgggccaaccggctggagagcggggtgcccagccggttcagcgggtccgggtccgggaccgactacaccctgaccatcagctccctgcagtacgaggacttcgggatctactactgccagcagtacgacgagtccccatggaccttcggcggcggcaccaagctggagatcaag

SEQ ID NO: 132 - DNA, 720 bp, synthetic construct
  source 1..720: mol_type = other DNA
gagatccagctggtgcagtccggcggcggcctggtgaaacccggcggctccgtgcggatctcctgcgccgcctccgggtacaccttcaccaactacgggatgaactgggtgcggcaggctcccgggaaggggctggagtggatgggctggatcaacacccacaccggggagcccacgtacgccgactccttcaaggggcgcttcaccttctccctcgacgactccaagaacaccgcctacctgcagatcaactccctgcgggccgaggacaccgccgtgtacttctgcacccggcgcgggtacgactggtacttcgacgtgtggggccaggggaccaccgtgaccgtgtcctccggaggcggcggctctggaggaggcggctctggaggcggaggctccgacatccagatgacccagagccccagctccctctccgcctccgtcggggaccgggtgaccatcacctgccgcgccagccaggacatcaactcctacctctcctggttccagcagaagcccgggaaggcgcccaagaccctcatctaccgcgccaaccggctggagtccggggtgcccagccgcttctccgggtccgggtccgggaccgactacaccctcaccatctccagcctgcagtacgaggacttcgggatctactactgccagcagtacgacgagagcccctggaccttcggcggcggcaccaagctggagatcaag

SEQ ID NO: 133 - DNA, 720 bp, synthetic construct
  source 1..720: mol_type = other DNA
gaaatccagctggtgcagtccggcggcggcctggtgaaacccggcggctccgtgcggatcagctgcgcggccagcgggtacacctttacgaactacgggatgaactgggtgcgccaggcacccggcaaggggctcgaatggatgggctggatcaacacgcacaccggggagccaacctacgcggatagcttcaaggggcgcttcacgttcagcctggacgactcgaagaacaccgcctacctgcagatcaactcgctgcgcgccgaggacaccgcggtgtacttctgcacgcggcgcgggtacgactggtacttcgacgtgtggggccaggggaccaccgtgacggtgtcctccggaggaggcggctccggaggaggcggctcaggaggcggcggatcggacatccagatgacccagtcgcccagcagcctgtcggccagcgtgggcgaccgggtgaccatcacctgccgggcgtcccaggatatcaacagctacctgtcgtggttccagcagaagcccggcaaggcgcccaaaacgctgatctaccgggccaaccggctggaaagcggggtgcccagccggttcagcgggtcgggcagcgggacggactacacgctgaccatcagcagcctgcagtacgaggacttcgggatatactactgccagcagtacgacgagtcgccctggacgttcggcggaggcaccaagctggaaatcaag

SEQ ID NO: 134 - DNA, 720 bp, synthetic construct
  source 1..720: mol_type = other DNA
gagatccagctcgtgcagtccggcggcggcctcgtgaaacccggcggcagcgtgcgcatctcgtgcgccgcgtccgggtacacgttcacgaactacggcatgaactgggtgcggcaggcacccggcaaggggctggagtggatgggctggatcaacacgcacaccggcgagcccacgtacgccgactcgttcaaggggcgcttcacgttcagcctcgacgactccaagaacaccgcgtacctgcagatcaacagcctgcgcgccgaggacaccgccgtgtacttctgcacgcggcgcgggtacgactggtacttcgacgtgtggggccagggcacgaccgtgaccgtgtcgtctggcggcggtggatcaggcggcggaggctcaggcggtggaggctccgacatccagatgacgcagtcgccgtccagcctgtccgcgtccgtcggcgaccgcgtgacgatcacgtgccgcgcgtcgcaggacatcaactcgtacctgtcgtggttccagcagaagcccggcaaggcgcccaagacgctgatctaccgcgcgaaccggctggagtccggcgtgccgtcgcggttcagcgggtccgggtccggcaccgactacacgctgacgatcagcagcctgcagtacgaggacttcggcatctactactgccagcagtacgacgagtcgccgtggacgttcggcggcggcacgaagctggagatcaag

SEQ ID NO: 135 - DNA, 720 bp, synthetic construct
  source 1..720: mol_type = other DNA
gagatccagctcgtgcagtccggcggcggcctggtcaagcctggcggctccgtgcgcatctcctgcgcggcctcggggtacaccttcacgaactacggcatgaactgggtgcgccaggccccggggaaaggcctggagtggatgggctggatcaacacgcacaccggggagcccacgtacgccgactccttcaaggggcgcttcacgttctccctggacgactcgaagaacacggcctacctgcagatcaactccctgcgcgccgaggacaccgcggtgtacttctgcacgcgccggggctacgactggtacttcgacgtctggggccaggggacgacggtcaccgtgagctcaggcggcggaggaagcggaggcggaggctcaggcggaggcggctcggacatccagatgacgcagagcccgtcctcgctgagcgcctccgtgggcgaccgcgtgacgatcacctgccgggcgtcccaggacatcaactcctacctgtcgtggttccagcagaagcccgggaaggccccgaagaccctgatctaccgggcgaaccgcctggagtccggcgtgccctcgcggttctccgggtcgggctcggggacggactacacgctgaccatctcctcgctgcagtacgaggacttcggcatctactactgccagcagtacgacgagtccccgtggaccttcggcggcggcacgaagctggagatcaag

SEQ ID NO: 136 - protein, 133 aa, synthetic construct [sequence omitted]

SEQ ID NO: 137 - DNA, 399 bp, synthetic construct
  source 1..399: mol_type = other DNA
gcaccaacaagcagtagcaccaaaaagacgcagcttcagttagagcacctcctactcgacctacagatgatattgaatggtattaataactacaaaaatcctaaattgactcgaatgttgacatttaaattttatatgcccaaaaaggcaaccgaactcaagcatctgcagtgcctggaggaggaactcaagccacttgaagaggtcctgaacctggctcagtcaaaaaattttcatctgcgcccccgggacttaatcagcaatatcaacgtgattgttctggagctcaaggggtctgagaccacttttatgtgtgaatacgctgatgaaactgcgacaatcgtcgagttcctcaatagatggatcactttctgtcaatccattattagcaccctgacc

SEQ ID NO: 138 - DNA, 399 bp, synthetic construct
  source 1..399: mol_type = other DNA
gctcctaccagctccagcaccaagaagacccagctgcagctggagcacctgctgctggacctgcagatgatcctgaacgggatcaacaactacaagaaccccaagctgacccggatgctgaccttcaagttctacatgcccaagaaggccaccgagctgaagcacctgcagtgcctggaggaggagctgaagcccctggaggaggtgctgaacctggcccagagcaagaacttccacctgcggccccgggacctgatcagcaacatcaacgtgatcgtgctggagctgaaggggtccgagaccaccttcatgtgcgagtacgccgacgagaccgccaccatcgtggagttcctgaaccgctggatcaccttctgccagagcatcatctccacgctgacc

SEQ ID NO: 139 - DNA, 399 bp, synthetic construct
  source 1..399: mol_type = other DNA
gccccgaccagcagcagcaccaagaagacgcagctgcagctggagcacctgctgctggacctgcagatgatcctgaacgggatcaacaactacaagaaccccaagctgacccggatgctgaccttcaagttctacatgcccaagaaggcgaccgagctgaagcacctgcagtgcctggaggaggagctgaagcccctggaggaggtgctgaacctggcccagtccaagaacttccacctgcggccccgggacctgatcagcaacatcaacgtgatcgtgctggagctgaaggggtccgagaccaccttcatgtgcgagtacgcggacgagaccgccacaatcgtggagttcctgaaccgctggatcaccttctgccagtccatcatcagcaccctgacg

SEQ ID NO: 140 - DNA, 399 bp, synthetic construct
  source 1..399: mol_type = other DNA
gccccaacctcctcctccaccaagaagacccagctgcagctggagcacctgctgctggacctgcagatgatcctgaacgggatcaacaactacaagaaccccaagctgacccggatgctcaccttcaagttctacatgcccaagaaggccaccgagctgaagcacctgcagtgcctggaggaggagctgaagcccctggaggaggtgctgaacctggcccagtccaagaacttccacctgcggccccgggacctgatctccaacatcaacgtgatcgtgctggagctgaaggggtccgagaccaccttcatgtgcgagtacgcagatgagacggctacaatcgtggagttcctgaacaggtggatcaccttctgccagtccatcatctccaccttgaca

SEQ ID NO: 141 - DNA, 399 bp, synthetic construct
  source 1..399: mol_type = other DNA
gcgcccacgtcctcctccaccaagaagacccagctgcagctggagcacctgctgctggacctgcagatgatcctgaacgggatcaacaactacaagaaccccaagctgacccggatgctgaccttcaagttctacatgcccaagaaggccaccgagctgaagcacctgcagtgcctggaggaggagctgaagcccctggaggaggtgctgaacctggcccagtccaagaacttccacctgcggccccgggacctgatctccaacatcaacgtgatcgtgctggagctgaaggggtccgagaccaccttcatgtgcgagtacgccgacgagaccgccaccatcgtggagttcctgaaccgctggatcaccttctgccagagcatcatcagcaccctcacc

SEQ ID NO: 142 - DNA, 399 bp, synthetic construct
  source 1..399: mol_type = other DNA
gcccctacctcctcctccaccaagaagacccagctccagctggagcacctcctcctggacctccagatgatcctcaacgggatcaacaactacaagaaccccaagctcacccggatgctgaccttcaagttctacatgcccaagaaggccaccgagctgaagcacctccagtgcctggaggaggagctgaagcccctggaggaggtgctgaacctggcccagtccaagaacttccacctccggcccagggacctgatctccaacatcaacgtgatcgtcctggagctgaaggggtccgagaccaccttcatgtgcgagtacgccgatgagacagccaccatcgtggagttcctcaacaggtggatcaccttctgccagtccatcatcagcaccctcacc

SEQ ID NO: 143 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 144 - DNA, 342 bp, synthetic construct
  source 1..342: mol_type = other DNA
aactgggttaacgtgatcagcgatctgaagaagattgaagatctcatacaatccatgcacatcgacgctaccctgtatacagagtccgacgttcaccctagctgtaaggtgactgccatgaagtgctttttactggaactgcaggtaatcagtctggagtctggtgatgcctcaattcacgacacggtagagaatctaataatccttgccaacaactctttgagttccaatggcaatgtgacagaatctggctgcaaggagtgtgaagagcttgaagagaaaaacattaaagagttcctgcaatccttcgtgcatatagtgcagatgttcatcaacacctcg

SEQ ID NO: 145 - DNA, 342 bp, synthetic construct
  source 1..342: mol_type = other DNA
aactgggtgaacgtgatcagcgatctgaagaagatcgaggacctgatccagtccatgcacatcgacgctaccctgtacaccgagtcggacgttcaccccagctgcaaggtgaccgcgatgaagtgcttcctgctcgaactgcaggtgatcagcctggagagcggggacgcgagcatccacgatacggtggagaacctgatcatcctggccaacaactcgctcagctcgaacgggaacgtgaccgagagcgggtgcaaggagtgcgaggagctggaggagaagaacatcaaggagttcctccagtcgttcgtgcacatcgtgcagatgttcatcaacacctcc

SEQ ID NO: 146 - DNA, 342 bp, synthetic construct
  source 1..342: mol_type = other DNA
aactgggtgaacgtcatctccgacctgaagaagatcgaggatctgatccagtcgatgcacatcgacgcgacgctctacaccgagtcggacgttcacccctcgtgcaaggtcacggcgatgaagtgcttcctcctggagctgcaggtgatctccctggagtcgggcgacgcctcgatccacgacacggtcgagaacctgatcatcctcgcgaacaactccctctcgtccaacgggaacgtgaccgagagcgggtgcaaggagtgcgaggagctggaggagaagaacatcaaggagttcctccagtcgttcgtccacatcgtccagatgttcatcaacacctcc

SEQ ID NO: 147 - DNA, 342 bp, synthetic construct
  source 1..342: mol_type = other DNA
aactgggtgaacgtgatcagcgacctgaagaagatcgaggacctgatccagtccatgcacatcgacgccacgctgtacaccgagtccgacgtgcaccccagctgcaaggtgaccgccatgaagtgcttcctgctggagctgcaggtgatctccctggagtccggggacgcctccatccacgacaccgtggagaacctgatcatcctggccaacaactccctgtcctccaacgggaacgtgaccgagtccgggtgcaaggagtgcgaggagctggaggagaagaacatcaaggagttcctgcagagcttcgtgcacatcgtgcagatgttcatcaacacgtcg

SEQ ID NO: 148 - DNA, 342 bp, synthetic construct
  source 1..342: mol_type = other DNA
aattgggtgaacgtcatctccgacctcaagaagatcgaggacctcatccagtccatgcacatcgacgccacgctctacacggagtccgacgtgcacccgtcctgcaaggtgacggccatgaagtgcttcctgctggagctgcaggtcatctccttggagtccggggacgcctccatccacgacaccgtcgagaacctcatcatcctggccaacaactccttgagctccaacgggaacgtgacggagtccggctgcaaggagtgcgaggagctggaggagaagaacatcaaggagttcctgcagtccttcgtgcacatcgtgcagatgttcatcaacacgtcc

SEQ ID NO: 149 - DNA, 342 bp, synthetic construct
  source 1..342: mol_type = other DNA
aactgggtgaacgtgatctccgacctcaagaagatcgaggacctcatccagtcgatgcacatcgacgcgaccctctacacggagagcgacgtccatccgagctgcaaggtgaccgcgatgaagtgcttcctcctggagctccaggtgatctccctggagtccggggacgcgagcatccacgacaccgtcgagaacctgatcatcctcgcgaacaactcgctctcctcgaacgggaacgtcaccgagagcggctgcaaggagtgcgaggagctcgaagagaagaacatcaaggagttcctccagagcttcgtccacatcgtccagatgttcatcaacacgtcg

SEQ ID NO: 150 - DNA, 19 bp, synthetic construct
  source 1..19: mol_type = other DNA
gaatggagagagaattgaa

SEQ ID NO: 151 - DNA, 21 bp, synthetic construct
  source 1..21: mol_type = other DNA
gctcaggctaagcttgtacaa

SEQ ID NO: 152 - DNA, 600 bp, synthetic construct
  source 1..600: mol_type = other DNA
cccctcccttcaggccccgcgcgattccgcccccagttctgtgccggccaagatcccggctagcgccgctatcattggttagttccaagtttgcccgcccctcttcctcctcctttttccgccccctccctcccgcggaagctgggggcgcatgcgtagaggtggacgctcccctcccccgcccggggtaactgaggactcccgcgcgcggactcgctgcgccccaccctccctttccccggggccgtccggagagcgggggcgagcttgaaagttccagaacgctgcggtgagtgcgttatcgtgaggcggagcgcggtggggtgggtgcggaagggggcgaggcccgaggagtggagccgggcttgtgattgggtcttgtaagggcagccgggcgtctattggccggggaagccgtaatggcaggcagcaggggcgggccccttctggaaggttctaagatagggtataagaggcagggtggcgggcggaaaccggtctcattgaactcgcctgcagctcttgggttttttgtggcttccttcgttattggagccaggcctacaccccaggtaaaacctctgctcaagagttgggttg

SEQ ID NO: 153 - DNA, 600 bp, synthetic construct; identical to SEQ ID NO: 152
  source 1..600: mol_type = other DNA

SEQ ID NO: 154 - DNA, 591 bp, synthetic construct
  source 1..591: mol_type = other DNA
atcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 155 - protein, 5 aa, synthetic construct
GGGGS